(19) United States
(12) Patent Application Publication
(10) Pub. No.: US 2024/0424216 A1
(43) Pub. Date: Dec. 26, 2024

(54) AUTO-INJECTOR AND RELATED METHODS OF USE

(71) Applicant: Regeneron Pharmaceuticals, Inc., Tarrytown, NY (US)

(72) Inventors: Daniel HALBIG, Ballston Lake, NY (US); Alexander ROUMANIDAKIS, Tarrytown, NY (US); Bryan GRYGUS, Clifton Park, NY (US); Danielle Laiacona, Tarrytown, NY (US); Sindhuja Kuchibhatla, Tarrytown, NY (US); John Burke, Cambridge (GB); Daniel O’Connell-Govan, Cambridge (GB)

(73) Assignee: Regeneron Pharmaceuticals, Inc., Tarrytown, NY (US)

(21) Appl. No.: 18/748,815

(22) Filed: Jun. 20, 2024

Related U.S. Application Data
(60) Provisional application No. 63/509,214, filed on Jun. 20, 2023.
(60) Provisional application No. 63/587,389, filed on Oct. 2, 2023.

(51) Int. Cl. A61M 5/20

(52) U.S. Cl. CPC A61M 5/2046; A61M 2005/2006

(57) ABSTRACT

In one instance, disclosed herein is an auto-injector, the auto-injector including: a housing; a container disposed within the housing, the container configured to store a medicament; a canister disposed within the housing, the canister configured to release a pressurized medium to expel the medicament from the container; a shroud coupled to the housing, the shroud configured to move between a first position and a second position relative to the housing to cause the pressurized medium to be released by the canister; and a mandrel assembly disposed within the housing, the mandrel assembly configured to urge the shroud outward relative to the housing toward the first position after the pressurized medium is released by the canister.

### CROSS-REFERENCE TO RELATED APPLICATIONS

This application claims priority to U.S. Provisional Application No. 63/509,214, filed on Jun. 20, 2023 and U.S. Provisional Application No. 63/587,389, filed on Oct. 2, 2023, the entirety of which is incorporated by reference herein.

### FIELD OF DISCLOSURE

Aspects of the present disclosure relate to devices and methods for delivering a fluid from a needle into a user using mechanisms, e.g., a pressurized medium, which automatically control injection of the needle and fluid. More specifically, embodiments of the present disclosure relate to auto-injectors and methods for delivering a dose of a medicament to a user by exposing an injection needle outside of an auto-injector while medicament is released via the injection needle and automatically concealing the injection needle within the auto-injector after medicament has been released via the injection needle.

### INTRODUCTION

In various available auto-injectors, upon activation by a user, a needle is deployed, and fluid is delivered from the needle into the user. After completion of fluid delivery, the needle may be retracted for user comfort, needle safety, and positive perception of the product. However, many auto-injectors require separate user actions for inserting and removing the needle. Additionally, many auto-injectors must be secured to the user for extended periods of time, which may be an inconvenience for the user.

### BRIEF DESCRIPTION OF THE DRAWINGS

The accompanying drawings, which are incorporated into and constitute a part of this specification, illustrate various exemplary embodiments and, together with the description, serve to explain principles of the disclosed embodiments. The drawings show different aspects of the present disclosure and, where appropriate, reference numerals illustrating like structures, components, materials, and/or elements in different figures are labeled similarly. It is understood that various combinations of the structures, components, and/or elements in various embodiments, other than those specifically shown, are contemplated and are within the scope of the present disclosure.

There are many embodiments described and illustrated herein. The described devices and methods are neither limited to any single aspect nor embodiment thereof, nor to any combinations and/or permutations of such aspects and/or embodiments. Moreover, each of the aspects of the described inventions, and/or embodiments thereof, may be employed alone or in combination with one or more of the other aspects of the described inventions and/or embodiments thereof. For the sake of brevity, certain permutations and combinations are not discussed and/or illustrated separately herein.

FIG. 1 is a perspective view of an auto-injector, according to an example of the disclosure.

FIGS. 2-3 are perspective views of a chassis and a container of the auto-injector of FIG. 1, according to an example of the disclosure.

FIGS. 4-7 are partial perspective views of a cap and a shroud of the auto-injector of FIG. 1, according to an example of the disclosure.

FIGS. 8-9 are perspective views of a shroud of the auto-injector of FIG. 1, according to an example of the disclosure.

FIG. 10 is a perspective view of an actuator and a canister of the auto-injector of FIG. 1, according to an example of the disclosure.

FIG. 11 is a cross-sectional view of an actuator and a canister of the auto-injector of FIG. 1, according to an example of the disclosure.

FIGS. 12A-12B are cross-sectional views of an actuator and a canister of the auto-injector of FIG. 1 moving from a pre-activated state to an activated state, according to an example of the disclosure.

FIGS. 13A-13B are cross-sectional views of a canister of the auto-injector of FIG. 1 moving from a pre-activated state to an activated state, according to an example of the disclosure.

FIG. 14 is a cross-sectional view of a shroud and a chassis of the auto-injector of FIG. 1, according to an example of the disclosure.

FIG. 15 is a cross-sectional view of a canister and a carrier of the auto-injector of FIG. 1, according to an example of the disclosure.

FIGS. 16A-16B are perspective views of an indicator of the auto-injector of FIG. 1 moving from a pre-activated state to an activated state, according to an example of the disclosure.

FIGS. 17A-17B are perspective views of an indicator of the auto-injector of FIG. 1 moving from a pre-activated state to an activated state, according to an example of the disclosure.

FIGS. 18A-18C are cross-sectional views of a slidable piston of the auto-injector of FIG. 1 moving between multiple states, according to an example of the disclosure.

FIGS. 19A-19C are cross-sectional views of a mandrel assembly and a collar of the auto-injector of FIG. 1 moving between multiple states, according to an example of the disclosure.

FIGS. 20A-20B are cross-sectional views of a carrier of the auto-injector of FIG. 1 moving from a pre-activated state to an activated state, according to an example of the disclosure.

FIGS. 21A-21C are perspective views of a chassis and a mandrel assembly of the auto-injector of FIG. 1 moving between multiple states, according to an example of the disclosure.

FIGS. 22A-22C are cross-sectional views of a shroud and a slidable piston of the auto-injector of FIG. 1 moving between multiple states, according to an example of the disclosure.

FIGS. 23A-23B are perspective views of a chassis and an actuator of the auto-injector of FIG. 1 moving a pre-activated state to an early-lockout state, according to an example of the disclosure.

FIGS. 24A-24B are cross-sectional views of a mandrel assembly of the auto-injector of FIG. 1 moving a pre-activated state to an early-lockout state, according to an example of the disclosure.

FIGS. 25A-25B are cross-sectional views of a shroud and a valve assembly of the auto-injector of FIG. 1, according to an example of the disclosure.

FIG. 26 is a perspective view of an actuator and a carrier of the auto-injector of FIG. 1, according to an example of the disclosure.

FIG. 27 is a perspective view of an actuator and a shroud of the auto-injector of FIG. 1, according to an example of the disclosure.

FIG. 28 is a cross-sectional view of an indicator of the auto-injector of FIG. 1, according to an example of the disclosure.

FIGS. 29-30 are perspective views of a slidable piston of the auto-injector of FIG. 1 moving from a pre-activated state to an activated state, according to an example of the disclosure.

FIG. 31 is a cross-sectional side view of an actuator and a carrier of the auto-injector of FIG. 1, according to an example of the disclosure.

FIG. 32 is a cross-sectional view of a canister of the auto-injector of FIG. 1, according to an example of the disclosure.

FIG. 33 is a cross-sectional view of a shroud and a carrier of the auto-injector of FIG. 1, according to an example of the disclosure.

FIG. 34 is a perspective view of a valve assembly of the auto-injector of FIG. 1 having a horizontal configuration, according to an example of the disclosure.

FIG. 35 is a perspective view of a valve assembly of the auto-injector of FIG. 1 having a vertical configuration, according to an example of the disclosure.

FIGS. 36-42 are partial views of components of a valve assembly of the auto-injector of FIG. 1, according to an example of the disclosure.

FIG. 43 is a schematic view of a pressure sensing system of the auto-injector of FIG. 1, according to an example of the disclosure.

FIGS. 44-46 are schematic views of a drive system of the auto-injector of FIG. 1, according to an example of the disclosure.

FIGS. 47-50 are perspective views of the auto-injector of FIG. 1 including a secondary indicator, according to an example of this disclosure.

FIGS. 51-52 are schematic views of the auto-injector of FIGS. 47-50, according to an example of this disclosure.

FIGS. 53A-53C are side views of the auto-injector of FIGS. 47-50 with the secondary indicator indicating a dose state, according to an example of this disclosure.

FIGS. 54-55 are partial views of the auto-injector of FIGS. 47-50 positioned against an injection site, according to an example of this disclosure.

FIGS. 56-57 are perspective views of another exemplary auto-injector, according to an example of this disclosure.

FIG. 58 is a schematic view of the auto-injector of FIGS. 56-57 including a secondary indicator, according to an example of this disclosure.

FIGS. 59-60 are perspective views of another exemplary auto-injector, according to an example of this disclosure.

FIGS. 61-62 are schematic views of the auto-injector of FIGS. 59-60, according to an example of this disclosure.

FIGS. 63-64 are perspective views of another exemplary auto-injector, according to an example of this disclosure.

FIGS. 65-66 are schematic views of the auto-injector of FIGS. 63-64, according to an example of this disclosure.

FIGS. 67-68 are side views of another exemplary auto-injector, according to an example of this disclosure.

FIGS. 69-70 are perspective views of another exemplary auto-injector, according to an example of this disclosure.

FIGS. 71-72 are schematic views of the auto-injector of FIGS. 69-70, according to an example of this disclosure.

FIG. 73 is a side view of the auto-injector of FIGS. 69-70, according to an example of this disclosure.

FIGS. 74-75 are perspective views of another exemplary auto-injector, according to an example of this disclosure.

FIGS. 76-77 are schematic views of the auto-injector of FIGS. 74-75, according to an example of this disclosure.

FIG. 78 is a side view of the auto-injector of FIGS. 74-75, according to an example of this disclosure.

FIGS. 79-80 are perspective views of another exemplary auto-injector, according to an example of this disclosure.

FIGS. 81-82 are schematic views of the auto-injector of FIGS. 79-80, according to an example of this disclosure.

FIG. 83 is a side view of the auto-injector of FIGS. 79-80, according to an example of this disclosure.

FIGS. 84-85 are perspective views of another exemplary auto-injector, according to an example of this disclosure.

FIGS. 86-87 are schematic views of the auto-injector of FIGS. 84-85, according to an example of this disclosure.

FIGS. 88-89 are perspective views of another exemplary auto-injector, according to an example of this disclosure.

FIG. 90 is a side view of the auto-injector of FIGS. 88-89 including a secondary indicator, according to an example of this disclosure.

FIG. 91 is a side view of another exemplary auto-injector, according to an example of this disclosure.

FIGS. 92-93 are schematic views of the auto-injector of FIG. 91, according to an example of this disclosure.

FIG. 94 is a perspective view of another exemplary auto-injector, according to an example of this disclosure.

FIG. 95 is a perspective view of another exemplary auto-injector, according to an example of this disclosure.

FIG. 96 is a perspective view of another exemplary auto-injector, according to an example of this disclosure.

FIG. 97 is a partial view of the auto-injector of FIGS. 63-64, according to an example of this disclosure.

FIG. 98 is a partial view of the auto-injector of FIGS. 79-80, according to an example of this disclosure.

FIG. 99 is a partial view of the auto-injector of FIGS. 84-85, according to an example of this disclosure.

FIG. 100 is a partial view of the auto-injector of FIG. 91, according to an example of this disclosure.

FIG. 101 is a perspective view of an exemplary auto-injector, according to an example of the disclosure.

FIGS. 102A-102C are schematic views of a drive system of the auto-injector of FIG. 101, according to an example of the disclosure.

FIG. 103 is an exploded perspective view of the auto-injector of FIG. 101, according to an example of the disclosure.

FIG. 104 is a cross-sectional perspective view of a portion of the auto-injector of FIG. 101, according to an example of the disclosure.

FIGS. 105A-105B are partial perspective views of the auto-injector of FIG. 101, according to an example of the disclosure.

FIG. 106 is a partial perspective view of an activator of the auto-injector of FIG. 101, according to an example of the disclosure.

FIG. 107 is a partial side view of the activator of FIG. 107, according to an example of the disclosure.

FIGS. 108A-108D are partial views of a keyed arrangement of the activator of FIG. 107, according to an example of the disclosure.

FIGS. 109-110 are cross-sectional views of the activator of FIG. 107 moving between a plurality of positions, according to an example of the disclosure.

FIGS. 111A-111C are schematic illustrations of portions of a needle mechanism of the auto-injector of FIG. 101, according to an example of the disclosure.

FIGS. 112-113 are perspective views of a peel tab of the auto-injector of FIG. 101, according to an example of the disclosure.

FIGS. 114-115 are cross-sectional views of the peel tab of FIGS. 112-113 coupled to the auto-injector of FIG. 101, according to an example of the disclosure.

FIGS. 116-117 are partial perspective views of the peel tab of FIGS. 112-113 coupled to the auto-injector of FIG. 101, according to an example of the disclosure.

FIG. 118 is a cross-sectional view of the peel tab of FIGS. 112-113 coupled to the auto-injector of FIG. 101, according to an example of the disclosure.

FIGS. 119A-119B are perspective views of a carrier of the auto-injector of FIG. 101, according to an example of the disclosure.

FIGS. 120A-120B are perspective views of another exemplary carrier of the auto-injector of FIG. 101, according to an example of the disclosure.

FIG. 121 is a perspective view of the carrier of FIGS. 119A-119B, according to an example of the disclosure.

FIG. 122 is a perspective view of an activator of the auto-injector of FIG. 101, according to an example of the disclosure.

FIG. 123 is a perspective view of a gear of the auto-injector of FIG. 101, according to an example of the disclosure.

FIG. 124 is a perspective view of a driver of the auto-injector of FIG. 101, according to an example of the disclosure.

FIGS. 125-126 are side views of the driver of FIG. 126, according to an example of the disclosure.

FIGS. 127-129 are perspective views of a button of the auto-injector of FIG. 101, according to an example of the disclosure.

FIGS. 130-132 are perspective views of a shuttle actuator and an indicator slide of the auto-injector of FIG. 101, according to an example of the disclosure.

FIG. 133 is a perspective view of a fluid conduit of the auto-injector of FIG. 101, according to an example of the disclosure.

FIG. 134 is a perspective view of the fluid conduit of FIG. 133 and the driver of FIGS. 124-126 assembled to a sterile connector of the auto-injector of FIG. 101, according to an example of the disclosure.

FIG. 135 is a perspective view of a carrier of the auto-injector of FIG. 101 coupled to the gear of FIG. 123, the driver of FIGS. 124-126, the fluid conduit of FIG. 133, and the shuttle actuator of FIGS. 130-132, according to an example of the disclosure.

FIG. 136 is an exploded perspective view of a valve assembly of the auto-injector of FIG. 101, according to an example of the disclosure.

FIGS. 137-141 are perspective views of the valve assembly of FIG. 136, according to an example of the disclosure.

FIG. 142 is a perspective view of a diaphragm of the valve assembly FIG. 136, according to an example of the disclosure.

FIG. 143 is a cross-sectional side view of the diaphragm of FIG. 142, according to an example of the disclosure.

FIG. 144 is a cross-sectional side view of the diaphragm of FIG. 142 disposed within the valve assembly of FIG. 136, according to an example of the disclosure.

FIGS. 145A-145B are side schematic views of a vent system of the valve assembly of FIG. 136, according to an example of the disclosure.

FIGS. 146A-146D are cross-sectional views of a mandrel assembly, according to an example of the disclosure.

FIGS. 147A and 147B are perspective and cross-sectional views, respectively, of lockout windows, according to an example of the disclosure.

FIGS. 148A-148C are cross-sectional views of the mandrel assembly, according to an example of the disclosure.

FIGS. 149A-149C are schematic views of a drive system, according to an example of the disclosure.

FIG. 150 is a perspective view of a valve assembly, according to an example of the disclosure.

FIGS. 151A and 151B are cross-sectional views of an actuator and an activator, according to an example of the disclosure.

FIG. 152 is a perspective view of an auto-injector, according to an example of the disclosure.

FIGS. 153A and 153B are side views of an actuator and a pivot joint, respectively, according to an example of the disclosure.

FIG. 154 is a perspective view of an activator and a valve assembly, according to an example of the disclosure.

FIG. 155 is a perspective view of a shroud and a chassis, according to an example of the disclosure.

FIGS. 156A and 156B are perspective and cross-sectional views, respectively, of a shroud and a chassis, according to an example of the disclosure.

FIGS. 157A and 157B are perspective and cross-sectional views, respectively, of a shroud and a chassis, according to an example of the disclosure.

FIG. 158 is a cross-sectional view of an auto-injector, according to an example of the disclosure.

FIGS. 159A-159C are side views of an auto-injector, according to an example of the disclosure.

FIGS. 160A and 160B are cross-sectional views of a shroud and a cap, according to an example of the disclosure.

There are many embodiments described and illustrated herein. The present disclosure is neither limited to any single aspect nor embodiment thereof, nor to any combinations and/or permutations of such aspects and/or embodiments. Each of the aspects of the present disclosure, and/or embodiments thereof, may be employed alone or in combination with one or more of the other aspects of the present disclosure and/or embodiments thereof. For the sake of brevity, many of those combinations and permutations are not discussed separately herein.

Notably, for simplicity and clarity of illustration, certain aspects of the figures depict the general structure and/or manner of construction of the various embodiments. Descriptions and details of well-known features and techniques may be omitted to avoid unnecessarily obscuring other features. Elements in the figures are not necessarily drawn to scale; the dimensions of some features may be exaggerated relative to other elements to improve understanding of the example embodiments. For example, one of ordinary skill in the art appreciates that the cross-sectional views are not drawn to scale and should not be viewed as representing proportional relationships between different components. The cross-sectional views are provided to help illustrate the various components of the depicted assembly, and to show their relative positioning to one another.

### DETAILED DESCRIPTION

Reference will now be made in detail to examples of the present disclosure, which are illustrated in the accompanying drawings. Wherever possible, the same reference numbers will be used throughout the drawings to refer to the same or like parts. Embodiments of the present disclosure may be used with any type of fluid-containing products, such as liquid drug substances, liquid placebos, or other liquids that may be dispensed in a dose form. In the discussion that follows, terms “about,” “approximately,” “substantially,” and the like, when used in describing a numerical value, denote a variation of +/−10% of that value, unless specified otherwise.

As used herein, the terms “comprises,” “comprising,” “includes,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” Notably, an embodiment or implementation described herein as an “example” or “exemplary” is not to be construed as preferred or advantageous, for example, over other embodiments or implementations; rather, it is intended reflect or indicate the embodiment(s) is/are one “example,” rather than “ideal.” As used herein, the terms “top,” “bottom,” “upper,” “lower,” “lateral,” and “radial” refer to a location (or portion of a device) relative to an arrangement of the device depicted in the drawings. The terms “vertical,” “vertically,” “horizontal,” “horizontally,” “upwards,” “downwards,” “laterally,” and “radially” refer to a direction or an orientation relative to an arrangement of the device depicted in the drawings. In addition, the terms “first,” “second,” and the like, herein do not denote any order, quantity, or importance, but rather are used to distinguish an element, a structure, a step or a process from another. Moreover, the terms “a” and “an” herein do not denote a limitation of quantity, but rather denote the presence of one or more of the referenced items.

Some conventional auto-injectors may require multiple user interactions to self-administer a drug, including, e.g., separate user interactions for deploying a needle and subsequently retracting the needle after drug delivery. These additional steps can increase complexity of self-administration of drugs, introduce user errors, and cause user discomfort. Accordingly, the present disclosure is directed to various embodiments of an injection device (e.g., auto-injector) that simplifies self-administration of drugs, or other therapeutic agents, by a user. Specifically, according to certain embodiments, the auto-injector may not require any additional user interaction to withdraw a needle once the needle is subcutaneously inserted into the user. Thus, auto-injectors of the present disclosure are simplified to help prevent misuse or user error. Additionally, some conventional auto-injectors require multiple components and user operations to administer a drug, including, various spring or motor mechanisms. These additional components can increase complexity of manufacture and introduce mechanical faults or user error. Accordingly, the present disclosure is directed to various embodiments of an injection device (e.g., auto-injector) that simplifies and refines administration of drugs, or other therapeutic agents. The auto-injectors of the present disclosure may include one or more components that may be substantially similar to those described in International PCT Application No. PCT/US2020/040729, published as WO 2021/003409, and International PCT Application No. PCT/US2021/065567, published as WO 2022/147166, each of which are incorporated by reference.

In at least some embodiments, a handheld auto-injector may require a user to hold the auto-injector against the user's skin for the entirety of an injection procedure. In some embodiments, a handheld auto-injector according to this disclosure may be configured to deliver a medicament volume of less than 3.5 mL (or a medicament volume from about 0.5 mL to about 4.0 mL, about 1.0 mL to about 3.5 mL, about 3.0 mL, about 3.1 mL, about 3.2 mL, about 3.3 mL, about 3.4 mL, about 3.5 mL). The medicament volume may be referred to herein as a “dose.” Furthermore, handheld auto-injectors according to the present disclosure may be configured to complete an injection procedure, as measured from 1) a point at which that the user places the auto-injector onto the skin to 2) a point at which the user removes the auto-injector from the skin after completion of an injection, in less than about 30 seconds, less than about 25 seconds, less than about 20 seconds, less than about 15 seconds, or less than about 10 seconds. As described herein, an early-lift event may occur when the user removes the auto-injector from the skin prior to completion of the injection. The auto-injectors of the present disclosure may be configured and operable to initiate a lockout in response to the occurrence of an early-lift event or a completion of dose delivery, thereby inhibiting further use and/or reactivation of the auto-injector for preserving user safety.

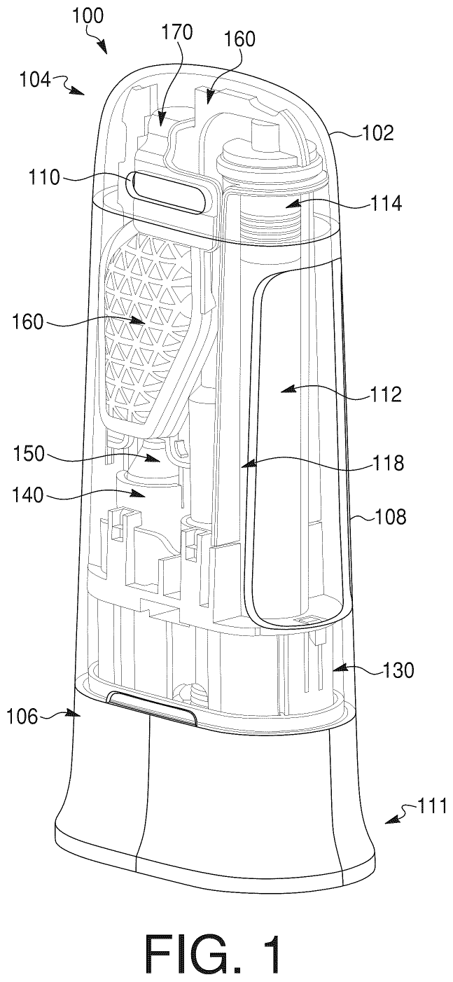

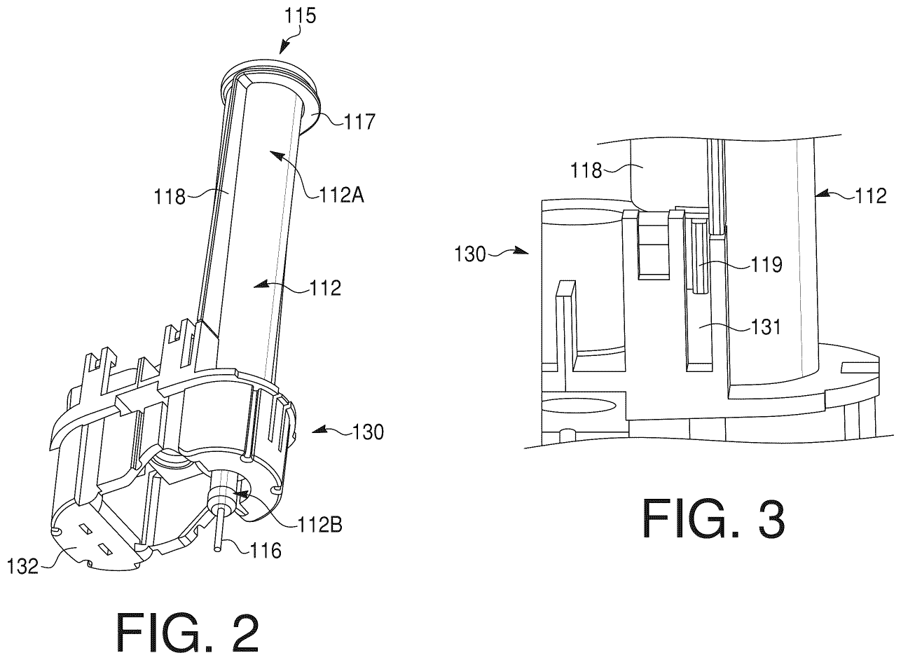

Referring now to FIG. 1, an exemplary auto-injector 100 is depicted in accordance with an example of the present disclosure. Auto-injector 100 may include a housing 102 having a longitudinal length defined between a top end 104 and a bottom end 106, and a cap 111 coupled to bottom end 106. Cap 111 may be removably coupled to bottom end 106, and bottom end 106 may define a user-engaging surface along an exterior interface of bottom end 106 through which a needle (see FIGS. 2-3) may be deployed from and retracted into housing 102. Top end 104 may define a user interface surface from which a user may control auto-injector 100, such as manually grasping housing 102 during use of auto-injector 100. It should be appreciated that housing 102 is depicted in FIG. 1 as partially transparent for illustrative purposes to show the internal components of auto-injector 100.

Auto-injector 100 may include a container 112, a chassis 130, a carrier 140, a canister 150 (e.g., a fluid source), a valve assembly 160, and an indicator assembly 170 housed between top end 104 and bottom end 106 of housing 102. Housing 102 may include one or more windows for facilitating visual inspection of the internal components of auto-injector 100 during use, such as to visually observe a current operating state of auto-injector 100. In the example, housing 102 may include a first window 108 formed along an exterior surface of housing 102 and extending vertically between top end 104 and bottom end 106 along a portion of housing 102 that is aligned with container 112. Accordingly, container 112 and a piston 114 disposed therein may be visually observed from an exterior of housing 102 via first window 108, and specifically the contents of container 112 and a relative position of piston 114 may be visually inspected through first window 108. Auto-injector 100 may include a shield 118 disposed within housing 102 and positioned about container 112 to obstruct visualization of the remaining internal components of auto-injector from first window 108.

Still referring to FIG. 1, container 112 may be sized and shaped to store a nominal value of a medicament. The “nominal volume” (also called the “specified volume,” or “specified capacity”) of a container refers to the container's maximum capacity, as identified by the container's manufacturer or a safety standards organization. A manufacturer or a safety standards organization may specify a container's nominal volume to indicate that the container can be filled with that volume of fluid (either aseptically or not) and be closed, stoppered, sterilized, packaged, transported, and/or used while maintaining container closure integrity, and while maintaining the safety, sterility, and/or aseptic nature of the fluid contained inside. In determining the nominal volume of a container, a manufacturer or a safety standards organization may also take into account variability that occurs during normal filling, closing, stoppering, packaging, transportation, and administration procedures. As an example, a prefillable syringe may be either hand- or machine-filled with up to its nominal volume of fluid, and may then be either vent tube- or vacuum-stoppered, without the filling and stoppering machinery and tools touching and potentially contaminating the contents of the syringe. Alternatively, the stopping machinery and tools may be sterile or aseptic, and are able to contact the contents of the syringe and/or the syringe itself without resulting in any contamination.

Container 112 may have about a 5.0 mL nominal volume in some examples, although any other suitable nominal volume (e.g., from about 0.5 mL to about 50.0 mL, or from about 2.0 mL to about 10.0 mL, or from about 3.0 mL to about 6.0 mL, or from about 1.0 mL to about 3.0 mL, or from about 2.0 mL to about 5.0 mL, or another suitable range) also may be utilized depending on the drug to be delivered. In other examples, container 112 may have a nominal volume greater than or equal to about 0.5 mL, or greater than or equal to about 2.0 mL, or greater than or equal to about 3.0 mL, or greater than or equal to about 4.0 mL, or greater than or equal to about 5.0 mL. Container 112 may contain and preserve a drug for injection into a user, and may help maintain sterility of the drug. In one embodiment, container 112 may be configured to deliver a delivered quantity of medicament (e.g., from about 0.5 mL to about 4.0 mL, about 1.0 mL to about 3.5 mL, about 3.0 mL, about 3.1 mL, about 3.2 mL, about 3.3 mL, about 3.4 mL, about 3.5 mL, greater than about 1.0 mL, greater than about 2.0 mL, greater than about 3.0 mL, greater than about 4.0 mL, greater than about 5.0 mL, greater than about 10.0 mL, greater than about 20.0 mL or another delivered quantity).

The delivered quantity may be less than the nominal volume of container 112. Furthermore, in order to deliver the delivered quantity of medicament to a user, container 112 itself may be filled with a different quantity of medicament than the delivered quantity (i.e., a filled quantity). The filled quantity may be an amount of medicament greater than the delivered quantity to account for medicament that cannot be transferred from container 112 to the user due to, e.g., dead space in container 112. Thus, while container 112 may have a nominal volume of 5 mL, the filled quantity and delivered quantity of medicament may be less than 5 mL.

In one embodiment, when container 112 is used in a handheld auto-injector, the delivered quantity of medicament from container 112 may be from about 0.5 mL to about 4.0 mL, about 1.0 mL to about 3.5 mL, about 3.0 mL, about 3.1 mL, about 3.2 mL, about 3.3 mL, about 3.4 mL, about 3.5 mL. The delivered quantity of medicament may be related to the viscosity of the medicament and the hand-held nature of auto-injector 100. That is, in at least some embodiments, at certain viscosities, higher volumes of medicament may prohibit the ability of auto-injector 100 to complete an injection procedure in less than an acceptable amount of time, e.g., less than about 30 seconds. Thus, the delivered quantity of medicament from auto-injector 100 may be set such that an injection procedure, measured from 1) the point in time at which auto-injector 100 is placed onto a user's skin, to 2) the point in time at which auto-injector 100 is removed from the skin, is less than about 30 seconds or less than about another time period (e.g., less than about 25 seconds, less than about 20 seconds, less than about 15 seconds, or less than about 10 seconds).

When the delivered quantity and/or viscosity of the medicament is too high, auto-injector 100 may not be able to function as a handheld auto-injector, since the time required to complete the injection procedure may be higher than commercially or clinically acceptable for handheld devices. Again, as stated above, in embodiments where container 112 is used in a hand-held auto-injector, regardless of the nominal volume of container 112, the delivered quantity of medicament from container 112 may be set such that the injection procedure as defined above is completed in a relatively short period of time (so as to avoid the need for additional features to attach auto-injector 100 to the user so that auto-injector 100 is a wearable auto-injector).

However, it is contemplated that various embodiments of the present disclosure may relate to wearable auto-injectors that deliver relatively large quantities of medicament (e.g., greater than about 3.5 mL) and/or have relatively longer injection procedure times as opposed to handheld auto-injectors (e.g., longer than about 30 seconds, longer than about 1 minute, longer than about 2 minutes, longer than about 5 minutes, or longer than about 1 hour) to complete an injection procedure as measured from 1) the point in time at which the auto-injector is placed onto a user's skin, to 2) the point in time at which the auto-injector is removed from the skin.

Container 112 may have about a 13 mm diameter neck, about a 45 mm length, and an internal diameter of about 19.05 mm. In another embodiment, container 112 may be a standard 3 mL container having an 8 mm crimp top, a 9.7 mm inner diameter, and a 64 mm length. In further embodiments, container 112 may have a length of about 64 mm to 74 mm, such as, for example, about 69.3 mm+0.15 mm (excluding a length of the neck of container 112 at second end 374). In embodiments including the neck, container 112 may have a length ranging from about 65 mm to 75 mm, such as, for example, about 70.8 mm+0.4 mm. These values are merely exemplary, and other suitable dimensions may be utilized as appropriate. In some examples, container 112 may be formed using conventional materials, and may be shorter than existing devices, which can help auto-injector 100 remain cost-effective and small. In some embodiments, container 112 may be a shortened ISO 10 mL cartridge. Auto-injectors of the present disclosure may be configured to deliver highly viscous liquid to a patient. For example, auto-injector 100 of the present disclosure may be configured to deliver liquid having a viscosity from about 0 cP to about 100 cP, from about 5 cP to about 45 cP, from about 10 cP to about 40 cP, from about 15 cP to about 35 cP, from about 20 cP to about 30 cP, or about 25 cP.

Still referring to FIG. 1, container 112 may include a piston 114 movably disposed within a cavity of container 112. Piston 114 may be movable by a pressurized fluid expelled from a fluid source, such as, e.g., canister 150. As described further herein, the pressurized fluid (e.g., gas) expelled from canister 150 may translate piston 114 relative to container 112 vertically along a longitudinal axis of container 112 towards bottom end 112B. The movement of piston 114 towards a bottom end 112B of container 112 may cause the piston to act against the contents within container 112 (e.g., drugs, medications, medicaments, etc.). In some embodiments, auto-injectors of the present disclosure may be oriented such that canister 150 and the piston within container 112 are offset, or are otherwise not longitudinally aligned with one another.

As best seen in FIG. 2, shield 118 may be coaxially coupled onto container 112. Shield 118 may include a top flange 117 that is disposed about and interfaces with a top flange 115 of container 112. Shield 118 may generally have a semi-circular configuration that extends about an exterior surface of container 112 and along a portion of container 112 that is positioned internal to housing 102 and opposite of an opposing portion of container 112 that is positioned adjacent to first window 108 (see FIG. 1). Container 112 may be coupled to chassis 130, and may include a top end 112A adjacent to top flange 115 and a bottom end 112B that is opposite of top end 112A and that extends through a bottom end 132 of chassis 130. Container 112 may include a needle 116 coupled to bottom end 112B, and needle 116 may be in fluid communication with a fluid (e.g., medicament) stored in container 112. As seen in FIG. 3, shield 118 may include a protrusion 119 (e.g. a tab, a finger, etc.) that is received within a slot 131 on chassis 130, thereby coupling shield 118 to chassis 130. In this instance, shield 118 may be fixed to each of container 112 and chassis 130. With protrusion 119 received within slot 131 and top flange 117 coupled to top flange 115, auto-injector 100 may be configured to prevent inadvertent movement (e.g., translation, rotation, etc.) of shield 118 relative to container 112 and chassis 130. Shield 118 may be sized, shaped, and/or otherwise configured to partially cover container 112, thereby inhibiting visual access into an interior of housing 102 via first window 108. In other words, shield 118 may define an opening that is sized, shaped, and/or otherwise configured to reveal container 112 via first window 108 while inhibiting visual exposure of the other components within housing 102. In some embodiments, shield 118 may be formed of an opaque material with a predefined color (e.g., teal) to prevent visual accessibility into housing 102 via first window 108. In other embodiments, shield 118 may be omitted entirely.

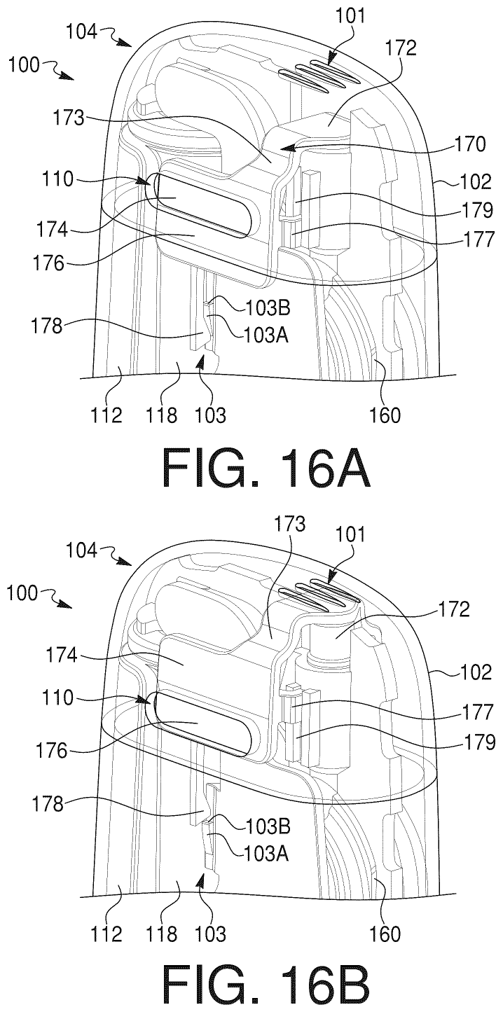

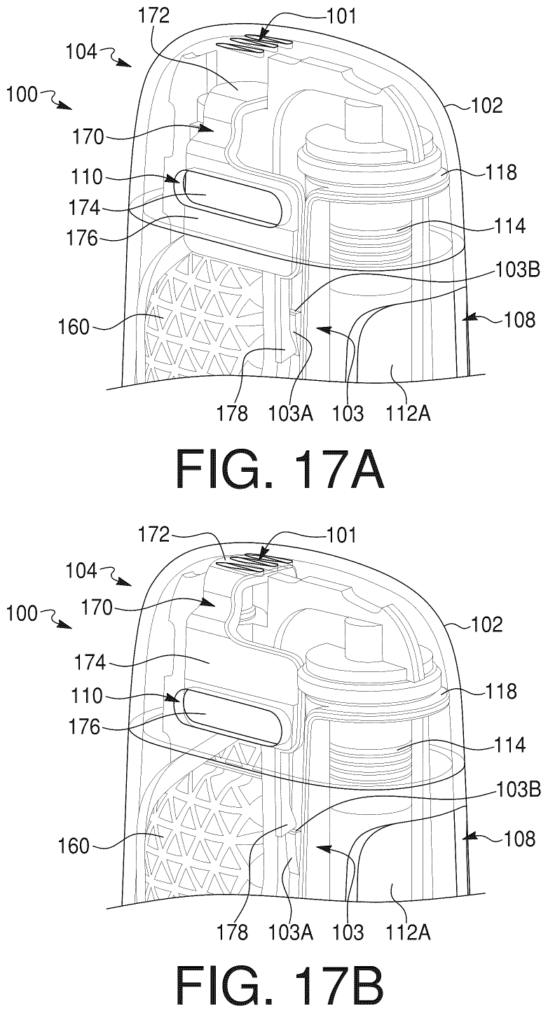

Referring back to FIG. 1, housing 102 may further include a second window 110 formed along an exterior surface of housing 102 and extending along a portion of housing 102 that is aligned with a portion of indicator assembly 170. Accordingly, indicator assembly 170 may be visually observed from an exterior of housing 102 via second window 110. As described herein, indicator assembly 170 may be configured to move from a first position towards a second position relative to second window 110 in response to an activation of auto-injector 100, thereby visually indicating a change in operating state of auto-injector 100 through second window 110, and particularly a dose state of auto-injector 100 (see FIGS. 16A-17B).

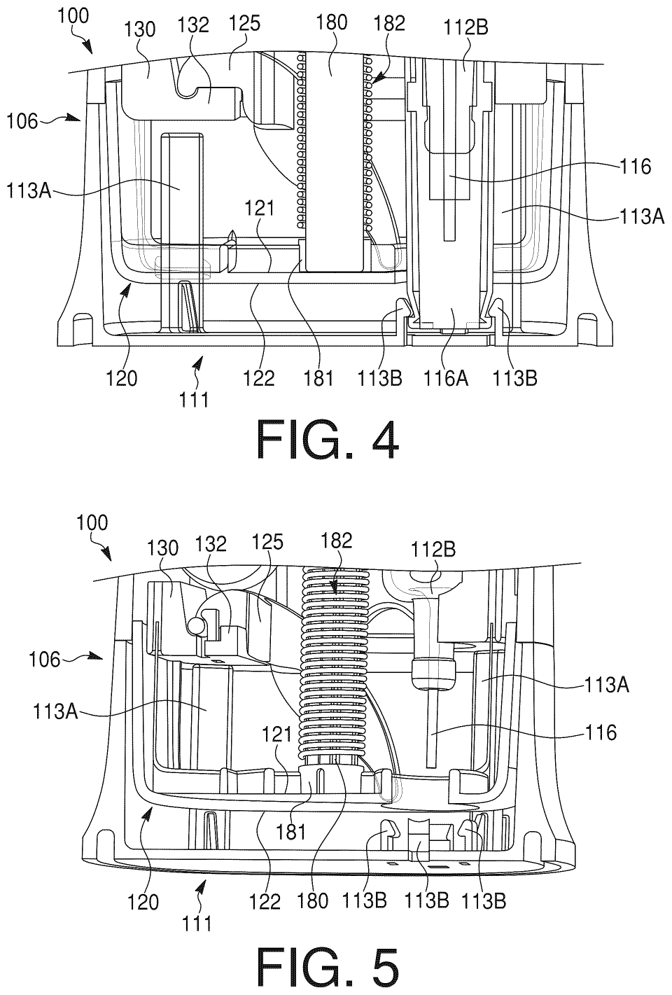

Referring now to FIG. 4, auto-injector 100 may include a shroud 120 at bottom end 106 of housing 102. Shroud 120 may be disposed inside of cap 111 when cap 111 is coupled to bottom end 106 of housing 102. Auto-injector 100 may further include an actuator 125 (e.g., a movable lever) and a mandrel assembly 180 each disposed within shroud 120, and particularly against an interior surface 121 of shroud 120. Shroud 120 may include a receptacle 181 along interior surface 121 that is sized, shaped, and/or otherwise configured to receive and/or securely hold an end of mandrel assembly 180. Accordingly, receptacle 181 may be operable to securely couple mandrel assembly 180 to shroud 120 such that mandrel assembly 180 may be movable within housing 102 in response to a simultaneous movement of shroud 120 relative to housing 102. Actuator 125 and mandrel assembly 180 may be coupled to respective portions of chassis 130 as described herein. Each of actuator 125 and mandrel assembly 180 may be configured to move in response to shroud 120 moving relative to housing 102 to transition auto-injector 100 from a pre-activated state to an activated state by initiating release of a pressurized medium throughout auto-injector 100.

Mandrel assembly 180 may include a biasing mechanism 182 disposed about an exterior of mandrel assembly 180. Biasing mechanism 182 may be configured to urge mandrel assembly 180 towards a predefined direction, bias mandrel assembly 180 towards a predefined configuration, generate a tactile feedback during movement of shroud 120, generate resistance against an upwards retraction of shroud 120 into housing 102, and more. In the example, biasing mechanism 182 may be configured to bias shroud 120 and mandrel assembly 180 towards an extended (downwards) position relative to housing 102 when biasing mechanism 182 is in an expanded configuration. As described herein, biasing mechanism 182 may be configured to generate a controlled feedback against shroud 120 as shroud 120 is actuated and depressed in a relatively upwards direction into housing 102, thereby causing biasing mechanism 182 to transition from the expanded configuration towards a compressed configuration. Biasing mechanism 182 may include various suitable devices, including but not limited to, a spring. In some embodiments, biasing mechanism 182 may be omitted entirely.

Cap 111 may include one or more ribs 113A extending vertically upward from an inner lower surface of cap 111 towards chassis 130. The one or more ribs 113A may be configured to abut against bottom end 132 of chassis 130 when cap 111 is coupled to bottom end 106 of housing 102. With ribs 113A abutting against bottom end 132, cap 111 may be configured to inhibit inadvertent movement of chassis 130 relative to housing 102 to prevent accidental activation of auto-injector 100. Stated differently, ribs 113A may be configured to push chassis 130 in an upward direction when cap 111 is coupled to housing 102, thereby locking chassis 130 into a fixed position relative to the internal components of housing 102, such as canister 150 to prevent inadvertent release of the pressurized medium stored therein.

Still referring to FIG. 4, auto-injector 100 may include a needle cover 116A removably coupled to bottom end 112B of container 112 with needle 116 disposed inside needle cover 116A. Accordingly, needle cover 116A may be configured to enclose needle 116 when coupled thereto. Needle cover 116A may be sized, shaped, and/or otherwise configured to extend outwardly from shroud 120, and particularly through a bottom surface 122 of shroud 120, such that needle cover 116A may be at least partially disposed inside of cap 111. Cap 111 may include one or more clips 113B (e.g., tabs, fingers, detents, etc.) extending vertically upward from an inner lower surface of cap 111 towards bottom surface 122 of shroud 120. The one or more clips 113B may be configured to engage the portion of needle cover 116A extending outwardly (e.g., downward) from shroud 120 and into cap 111.

Additionally, the one or more clips 113B may be configured to fixedly attach needle cover 116A to cap 111 such that needle cover 116A may be removable from bottom end 112B of container 112 upon decoupling cap 111 from bottom end 106 of housing 102, thereby exposing needle 116 within shroud 120. As best seen in FIG. 5, cap 111 may include a plurality of clips 113B configured to engage needle cover 116B. It should be understood that needle cover 116B is omitted in FIG. 5 for illustrative purposes only. In the example, needle cover 116B may include a narrowed portion and/or a distal neck defining one or more exterior surfaces that are sized, shaped, and/or otherwise configured to interface with clips 113B for securely coupling needle cover 116B to clips 113B. Stated differently, needle cover 116B may include an interface along an exterior end of needle cover 116B that may be engaged by clips 113B.

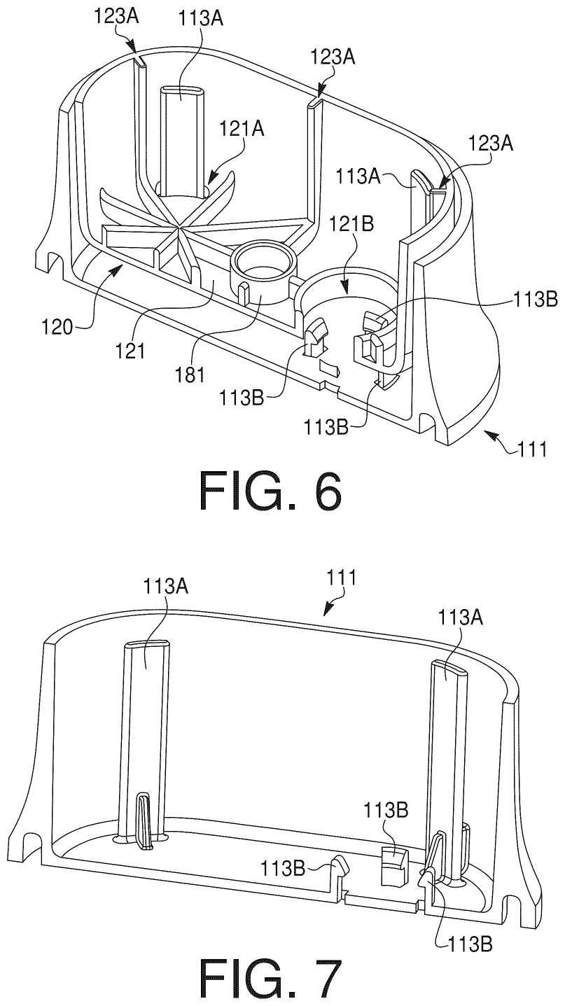

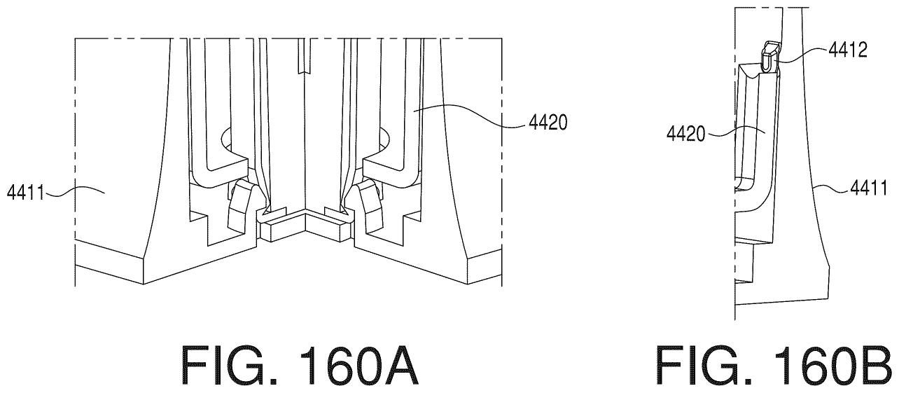

FIG. 6 depicts cap 111 with the one or more ribs 113A extending through corresponding openings 121A formed on interior surface 121 of shroud 120 and the plurality of clips 113B aligned with an opening 121B on interior surface 121. It should be appreciated that upon decoupling cap 111 from bottom end 106 of housing 102 and needle cover 116A from needle 116, needle 116 may extend through opening 121B upon moving shroud 120 vertically upward towards housing 102 along a vertical path. FIG. 7 depicts cap 111 in isolation from shroud 120. It should be appreciated that cap 111 is partially depicted in FIGS. 4-6 such that cap 111 may include additional ribs 113A and/or clips 113B than those shown and described herein. In some instances, as depicted in FIGS. 160A and 160B, cap 4411 includes one or more retention elements 4412 configured to engage with shroud 4420 to assist in securing or partially securing cap 4411 onto shroud 4420. Retention elements 4412 may include protrusions, bumps, ridges, or any other suitable feature.

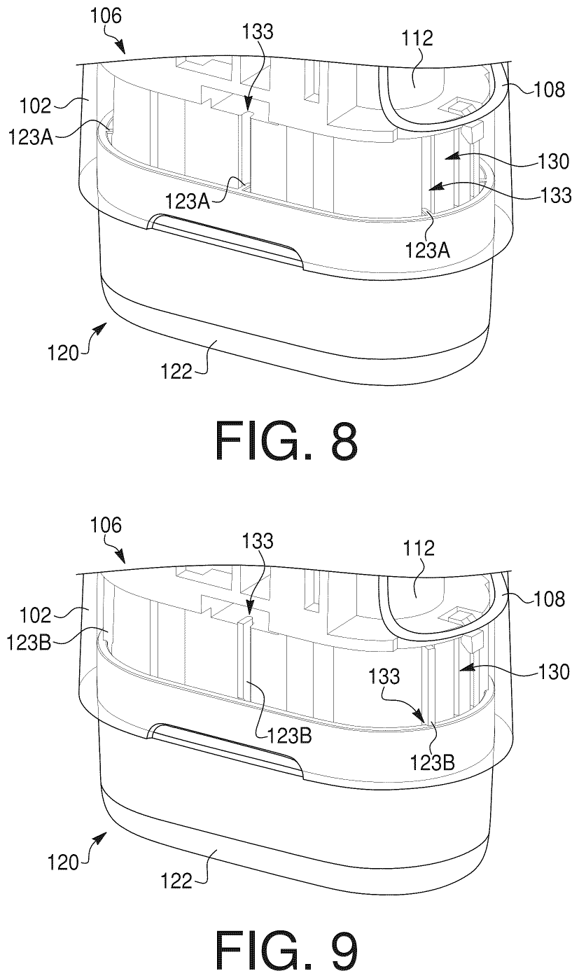

Referring now to FIGS. 8-9, housing 102 is depicted with cap 111 omitted such that shroud 120 is exposed at bottom end 106. Shroud 120 may be movably coupled to chassis 130 between one or more positions for activating auto-injector 100. Referring specifically to FIG. 8, shroud 120 may include one or more guide ribs 123A extending radially inward from interior surface 121 of shroud 120 (see FIG. 6), and chassis 130 may include one or more guide channels 133 formed along an exterior surface of chassis 130. It should be appreciated that chassis 130 may include a number of guide channels 133 that correspond to a number of guide ribs 123A on shroud 120, and vice versa. Guide channels 133 may be sized, shaped, and/or otherwise configured to receive the one or more guide ribs 123A. Guide channels 133 may be further configured to limit a direction of motion of shroud 120 relative to chassis 130 during use of auto-injector 100, and particularly during a stroke of shroud 120 for expelling a fluid from container 112. For example, guide channels 133 may be configured to limit movement of shroud 120 relative to housing 102 and/or chassis 130 to a vertical direction, thereby inhibiting lateral movement of shroud 120 relative to housing 102 and/or chassis 130. In the example shown in FIG. 8, guide ribs 123A may be flush with a top surface of shroud 120. In the example shown in FIG. 9, guide ribs 123B may extend vertically upward from the top surface of shroud 120 to provide enhanced stabilization of shroud 120 at a beginning of a stroke relative to guide ribs 123A of FIG. 8.

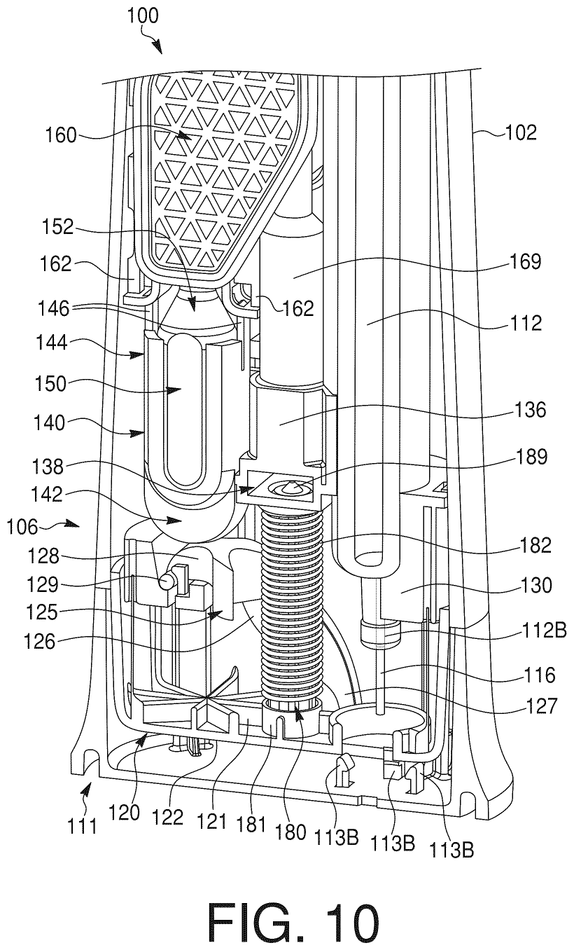

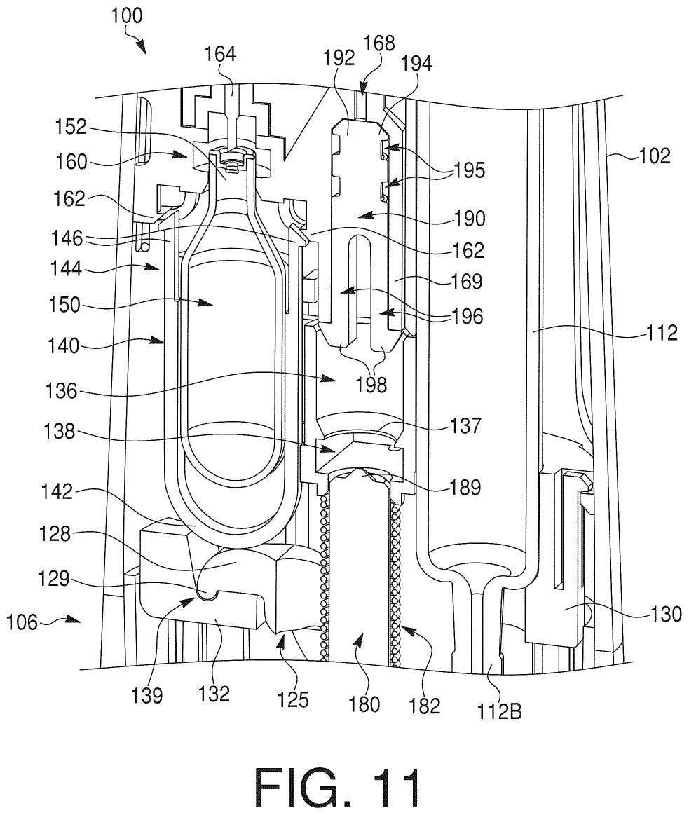

Referring now to FIGS. 10-11, actuator 125 may include a body 126 defined by a first end 127 that is movably coupled to shroud 120 and a second end 128 that is movably coupled to chassis 130 and carrier 140. Actuator 125 may include a pin 129 at second end 128 that is received within a recess 139 of chassis 130. Pin 129 may be configured to move (e.g., pivot, rotate, etc.) within recess 139, thereby allowing a corresponding movement of actuator 125 relative to chassis 130. In other words, a connection between pin 129 and recess 139 may define a pivot joint for actuator 125, such that actuator 125 is configured to move (e.g., pivot) within housing 102 about the pivot joint defined by pin 129 and recess 139. As described herein, actuator 125 may be configured to move in response to shroud 120 being depressed upward into housing 102 along a vertical path. Second end 128 may be sized, shaped, and/or otherwise configured to abut against a bottom end 142 of carrier 140. As described herein, actuator 125 may be configured to push carrier 140 vertically upward relative to housing 102 in response to second end 128 pushing against bottom end 142 as actuator 125 pivots about pin 129. In some embodiments, carrier 140 may be omitted entirely such that actuator 125 may be configured to push directly against canister 150.

It should be understood that FIGS. 10-11 depict shroud 120 in a first position and auto-injector 100 in a pre-activated state, with only first end 127 of actuator 125 in contact with interior surface 121 of shroud 120. As described herein, actuator 125 may be sized, shaped, and/or otherwise configured to contact interior surface 121 at additional portions of body 126 as shroud 120 moves towards a second position and auto-injector 100 transitions to an activated state. In the example, second end 128 may have a curved configuration that corresponds to a curved configuration of carrier 140 at bottom end 142, such that movement of actuator 125 relative to chassis 130 may cause the curved surface of second end 128 to push against the corresponding curved surface of bottom end 142. In other examples, second end 128 and/or bottom end 142 may have various other suitable shapes, sizes, and/or configurations than those shown and described herein without departing from a scope of this disclosure.

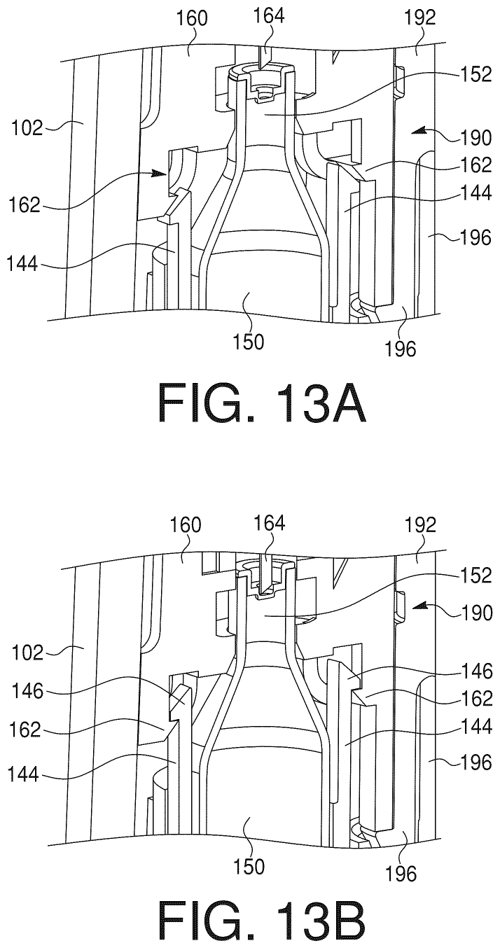

Carrier 140 may be sized, shaped, and/or otherwise configured to receive canister 150. In the example, canister 150 is fixed relative to carrier 140 such that canister 150 is configured to move within housing 102 in response to a corresponding movement (e.g. translation) of carrier 140 relative to housing 102, such as in response to actuator 125 pushing against bottom end 142. Carrier 140 may include a pair of retention mechanisms 146 extending outwardly (e.g., vertically upward) from a top end 144 of carrier 140 that is opposite of bottom end 142. Each of the pair of retention mechanisms 146 may include, but are not limited to, a clip, a hand, a finger, a tab, a protrusion, a detent, and more. Carrier 140 may be disposed within housing 102 relatively below valve assembly 160, and valve assembly 160 may include a pair of retention mechanisms 162 positioned adjacent to top end 144. Retention mechanisms 162 may be configured to engage retention mechanisms 146 in response to carrier 140 moving upward relative to housing 102 and towards valve assembly 160 as auto-injector 100 moves from the pre-activated state to the activated state (see FIGS. 13A-13B). Each of the pair of retention mechanisms 162 may include, but are not limited to, a corresponding clip, hand, finger, tab, protrusion, or detent that is configured to mate with retention mechanisms 146. In the example, retention mechanisms 146, 162 may each include a ramped and/or angled surface that is configured to interact with one another as carrier 140 is moved in a longitudinally upwards direction towards valve assembly 160, as described in further detail herein.

Still referring to FIGS. 10-11, canister 150 may be disposed inside carrier 140 with a neck 152 extending outwardly (e.g., upward) from top end 144. In the pre-activated state of auto-injector 100, neck 152 may be positioned relatively below and in alignment with a piercing mechanism 164 of valve assembly 160. Piercing mechanism 164 may include a needle, an activator pin, and various other suitable devices configured to puncture a seal and/or a valve of canister 150 at neck 152 for fluidly coupling valve assembly 160 to canister 150. As described herein, valve assembly 160 may be configured to receive a pressurized medium (e.g., gas) from canister 150 in response to piercing mechanism 164 puncturing the seal at neck 152. It should be appreciated that canister 150 is not in fluid communication with valve assembly 160 when auto-injector 100 is in the pre-activated state and prior to carrier 140 moving canister 150 upwards towards piercing mechanism 164. Valve assembly 160 may include a release outlet 169 that is positioned downstream and in fluid communication with piercing mechanism 164, such that at least a portion of the pressurized medium received in valve assembly 160 from canister 150 may be released from valve assembly 160 via release outlet 169.

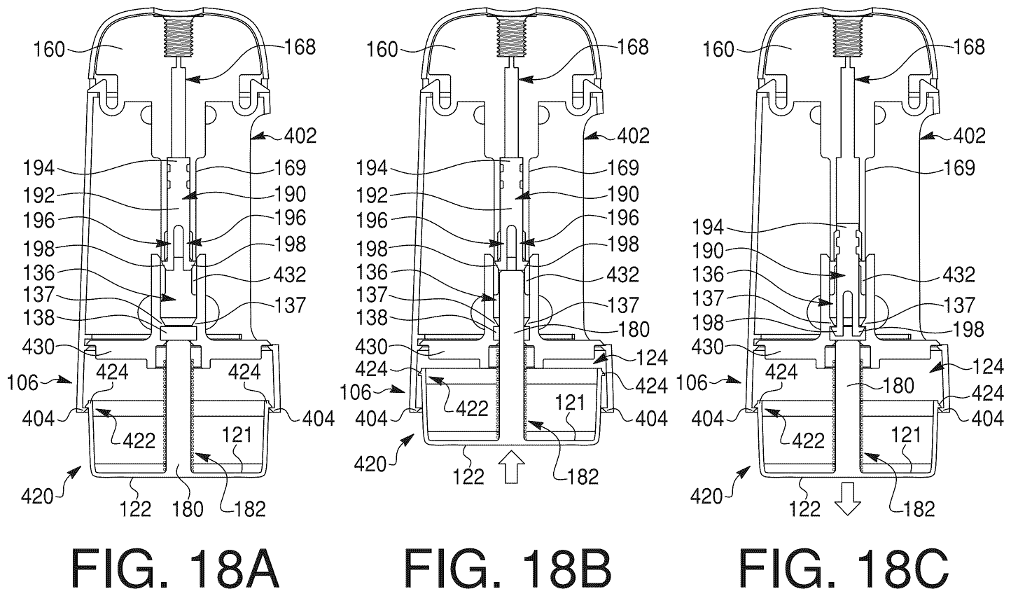

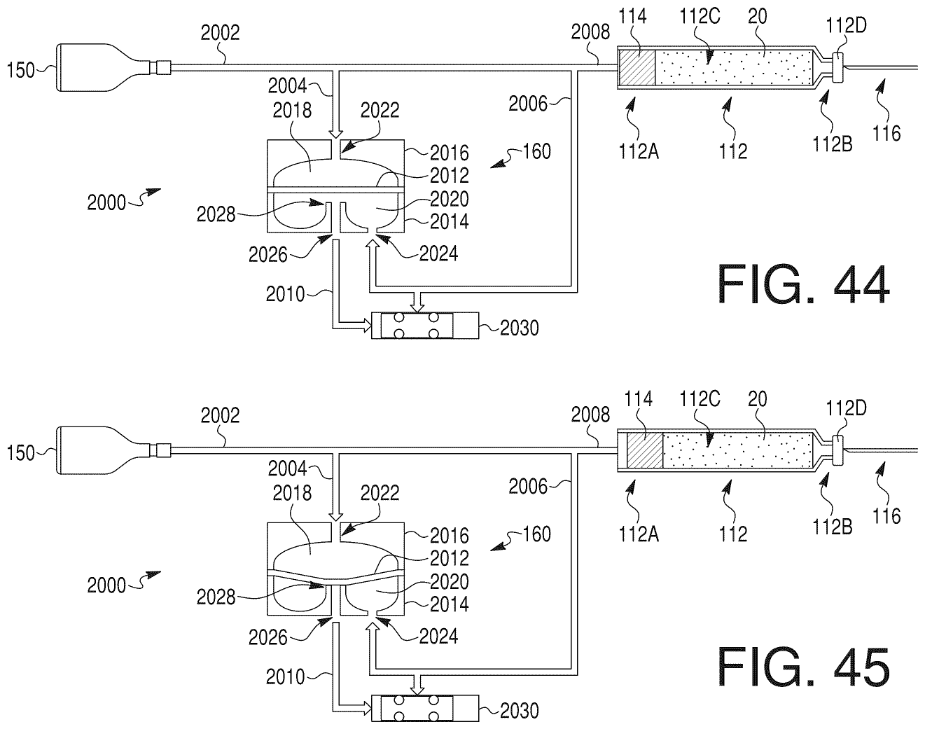

As best seen in FIG. 11, auto-injector 100 may include an outlet channel 168 in fluid communication with release outlet 169, and a slidable piston 190 movably disposed within release outlet 169. Slidable piston 190 may include a dump valve that is configured and operable as a venting system of auto-injector 100, as described and shown in further detail in FIGS. 44-46 (see venting system 2030). As described herein, at least a portion of the pressurized medium received from canister 150 may enter release outlet 169 via outlet channel 168 and cause slidable piston 190 to move (e.g., in a downward direction) relative to release outlet 169. Slidable piston 190 may have a body 192 with a longitudinal length extending between an upper end 194 and a lower end defined by a pair of snap arms 196. Body 192 may include one or more recesses 195 formed along upper end 194 that are sized, shaped, and/or otherwise configured to receive a seal (e.g., O-ring) therein to inhibit the pressurized medium received from outlet channel 168 from passing along an exterior surface of slidable piston 190. Accordingly, the pressurized medium received in release outlet 169 may be operable to push against body 192 at upper end 194, thereby causing slidable piston 190 to translate within release outlet 169, such as in a downward direction, from a first (upper) position towards a second (lower) position (see FIGS. 18A-18C). Each of the pair of snap arms 196 may include a retention mechanism 198 at a terminal (lower) end of snap arms 196. Retention mechanisms 198 may include, but are not limited to, a clip, a hand, a finger, a tab, a protrusion, and more. Retention mechanisms 198 may be disposed within a first (upper) chamber 136 of chassis 130, with first chamber 136 being disposed relatively below and in fluid communication with release outlet 169.

Chassis 130 may further include a second (lower) chamber 138 that is disposed relatively below first (upper) chamber 136 and above mandrel assembly 180. In the example, each of first chamber 136 and second chamber 138 may have a substantially similar cross-sectional dimension and/or internal diameter. Chassis 130 may further include an inner lip 137 disposed between first chamber 136 and second chamber 138. Inner lip 137 may extend radially inwards relative to an interior surface of first chamber 136 and second chamber 138, such that inner lip 137 may define a portion between first chamber 136 and second chamber 138 that has a relatively smaller cross-sectional dimension and/or internal diameter. As described herein, the pair of retention mechanisms 198 may be configured to engage inner lip 137 in response to the pressurized gas received in release outlet 169 pushing slidable piston 190 relatively downward into first chamber 136 with at least a portion of snap arms 196 extending into second chamber 138. With retention mechanisms 198 engaged with inner lip 137, chassis 130 may be configured to inhibit slidable piston 190 from returning to its initial position within release outlet 169.

In this instance, the pressurized medium received in release outlet 169 may be directed around body 192 of slidable piston 190 and released into first chamber 136 and second chamber 138. In some embodiments, an interior surface of release outlet 169, such as along a bottom portion adjacent to first chamber 136, may include a cutout, a channel, a recess, and/or a cavity that is depressed in a radially-outwards direction relative to a central longitudinal axis of release outlet 169, such that release outlet 169 may include an internal diameter that is greater along the bottom portion relative to a top portion of release outlet 169. Accordingly, the pressurized medium received in release outlet 169 may be directed around body 192 of slidable piston 190 via the interior surface along the bottom portion that includes the cutout prior to being released into first chamber 136 and second chamber 138. Second chamber 138 may be in fluid communication with an interior cavity of housing 102 that is maintained at atmospheric pressure, such that the pressurized medium received at second chamber 138 is effectively vented to eliminate pressure buildup within housing 102. In some embodiments, housing 102 may include one or more vent holes 101 (see FIG. 1) for maintaining the interior cavity of housing 102 at atmospheric pressure and facilitating depressurization of auto-injector 100.

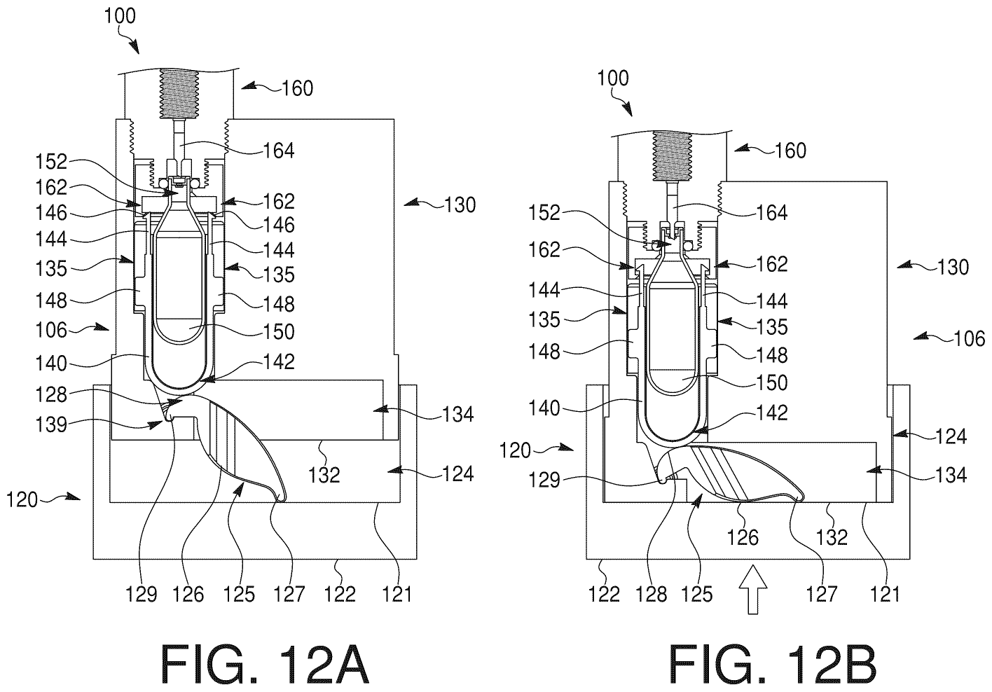

Referring now to FIGS. 12A-12B, one or more components of auto-injector 100 are depicted as auto-injector 100 transitions between a pre-activated state (FIG. 12A) to an activated state (FIG. 12B). It should be appreciated that one or more other components of auto-injector 100 are omitted from FIGS. 12A-12B for illustrative purposes only. Referring initially to FIG. 12A, shroud 120 is in a first position in which shroud 120 is maintained in an extended position relative to chassis 130 with interior surface 121 of shroud 120 axially offset from bottom end 132 of chassis 130. In this instance, actuator 125 may be positioned at least partially within an interior cavity 124 of shroud 120 and an interior cavity 134 of chassis 130, with only first end 127 of body 126 in contact with interior surface 121. Carrier 140 and canister 150 may be in a respective first position with retention mechanisms 146 disengaged from retention mechanisms 162, and piercing mechanism 164 separated from neck 152 (see FIG. 13A).

In the example, chassis 130 may include a channel 135 disposed about carrier 140, and carrier 140 may include a pair of arms 148 extending laterally (radially) outward from carrier 140. Channel 135 may be sized, shaped, and/or otherwise configured to receive arms 148. In the example, channel 135 may define a cylindrical cavity for receiving carrier 140 therein. Channel 135 may be configured to guide carrier 140 in an upwards direction towards valve assembly 160 in response to movement of actuator 125 against bottom end 142, thereby inhibiting inadvertent lateral movement of carrier 140 within chassis 130. In other embodiments, channel 135 and/or arms 148 may be omitted entirely from auto-injector 100. In further embodiments, carrier 140 may be omitted such that canister 150 is coupled directly to each of chassis 130 and valve assembly 160.

Referring to FIG. 12B, shroud 120 may be moved along a vertical path from the first position towards a second position in which shroud 120 is depressed upwards to a retracted position relative to chassis 130, with interior surface 121 in contact with bottom end 132, such as in response to bottom surface 122 of shroud 120 interfacing with a subject (e.g., a user of auto-injector 100). In this instance, actuator 125 may be moved (e.g., pivoted) about the pivot joint defined by pin 129 and recess 139 to transition auto-injector 100 to the activated state. As actuator 125 is moved towards the second position, shroud 120 pushes against first end 127 of body 126, causing second end 128 to abut against bottom end 142 of carrier 140. Second end 128 may define a lever-bearing surface of actuator 125 which is configured to redirect the force generated by movement of shroud 120 to carrier 140 and canister 150. Carrier 140 may be moved upwards relative to chassis 130 in response to second end 128 pushing against bottom end 142, thereby translating canister 150 towards valve assembly 160 until piercing mechanism 164 punctures the seal of neck 152. Accordingly, the pressurized medium stored in canister 150 may be released into valve assembly 160 via piercing mechanism 164.

It should be appreciated that shroud 120, actuator 125, chassis 130, carrier 140, and/or mandrel assembly 180 may be configured and operable to only require a low holding force for actuating auto-injector 100 such that auto-injector 100 minimizes the necessary force for initiating delivery of the dose. Stated differently, one or more components of auto-injector 100 may be sized, shaped, and/or otherwise configured to minimize a force necessary for depressing shroud 120 against a subject's skin during use of auto-injector 100 in delivering a dose to the subject.

Additionally, the pair of retention mechanisms 146 may be moved upward towards valve assembly 160 as carrier 140 and canister 150 are simultaneously moved within housing 102, thereby causing retention mechanisms 146 to engage the corresponding retention mechanisms 162 (see FIG. 13B). In this instance, valve assembly 160 may be configured to fixedly secure canister 150 thereto in response to valve assembly 160 coupling with carrier 140. In some embodiments, at least a portion of the pressurized medium expelled from canister 150 may generate a blowback force onto canister 150, causing canister 150 to move downwards and away from valve assembly 160. The engagement between retention mechanisms 146 and retention mechanisms 162 may inhibit and/or at least partially minimize a downward retraction of canister 150 relative to valve assembly 160, thereby maintaining fluid communication between canister 150 and valve assembly 160.

In some embodiments, auto-injector 100 may be configured to lock one or more components by inhibiting movement of said one or more components, such as shroud 120, upon an occurrence of an early-lift of shroud 120 during use of auto-injector 100. In the instance of an early-lift event of shroud 120, auto-injector 100 may be configured to inhibit reactivation and/or further use of auto-injector 100 to preserve user safety. An early-lift event may be generally defined as the occurrence of an early, untimely, and/or premature release of shroud 120 by a user as auto-injector 100 is transitioned from the pre-activated state towards the activated state, or during injection of the medicament from auto-injector 100 while operating in the activated state. In this instance, auto-injector 100 may be configured and operable to lock one or more components (e.g., shroud 120) from further movement relative to housing 102, thereby inhibiting further use and/or reactivation of auto-injector 100. For instance, auto-injector 100 may lock a relative position of shroud 120 to either an extended (downwards) configuration or a retracted (upwards) configuration relative to housing 102, thereby inhibiting further actuation and/or movement of shroud 120. It should be appreciated that locking shroud 120 in the retracted configuration relative to housing 102 may be to an extent such that needle 116 remains disposed inside shroud 120, thereby inhibiting exposure of needle 116 from an exterior of auto-injector 100.

In one example, auto-injector 100 may include a timer mechanism that may be operable to determine the occurrence of an early-lift event. The timer mechanism may provide a buffer period to allow for a user of auto-injector 100 to quickly perform an early-lift before auto-injector 100 is locked out. For instance, the timer mechanism may be programmed to allow for a quick adjustment of auto-injector 100 without locking auto-injector 100 when such adjustment does not exceed a predefined threshold (e.g., one second). Such an adjustment may include a physical removal of auto-injector 100 against a user's skin after initial activation, such as for reasons due to discomfort or accidental removal. Upon the occurrence of an early-lift event, i.e., removal of shroud 120 from a skin of the user for a duration that exceeds the predetermined threshold as determined by the timer mechanism, auto-injector 100 may be configured to lock one or more components thereby inhibiting further movement of shroud 120 relative to housing 102.

Upon completing delivery of a dose from auto-injector 100, one or more components of auto-injector 100, such as slidable piston 190, mandrel assembly 180, and/or biasing mechanisms 182, may be configured to interact with shroud 120 to lockout auto-injector 100 from reactivation. For example, as described herein in further detail, slidable piston 190 may be configured to push mandrel assembly 180 in a downwards direction upon exiting release outlet 169 and entering first chamber 136 and second chamber 138 within which mandrel assembly 180 was received. In this instance, mandrel assembly 180 may be configured to push shroud 120 outwardly in the downwards direction relative to housing 102. Additionally and/or alternatively, biasing mechanism 182 may be configured to urge mandrel assembly 180 in the downwards direction, thereby further pushing shroud 120 out of housing 102, in response to biasing mechanism 182 automatically moving from the compressed configuration towards the expanded configuration, thereby automatically moving shroud 120 from the second position towards the first position. It should be appreciated that the biasing mechanism 182 is configured to automatically move after a threshold amount of energy is released, such as from canister 150. In this instance, with bottom surface 122 of shroud 120 positioned on an exterior surface of a subject's (e.g. a patient) skin, shroud 120 may be configured to push against the exterior surface thereby causing housing 102 to lift off of the subject's skin in an upwards direction. Accordingly, auto-injector 100 may be operable to push itself off of the subject's skin to signal completion of the dose delivery via a visual feedback of the housing 102 automatically lifting itself from the skin due to the extension of shroud 120.

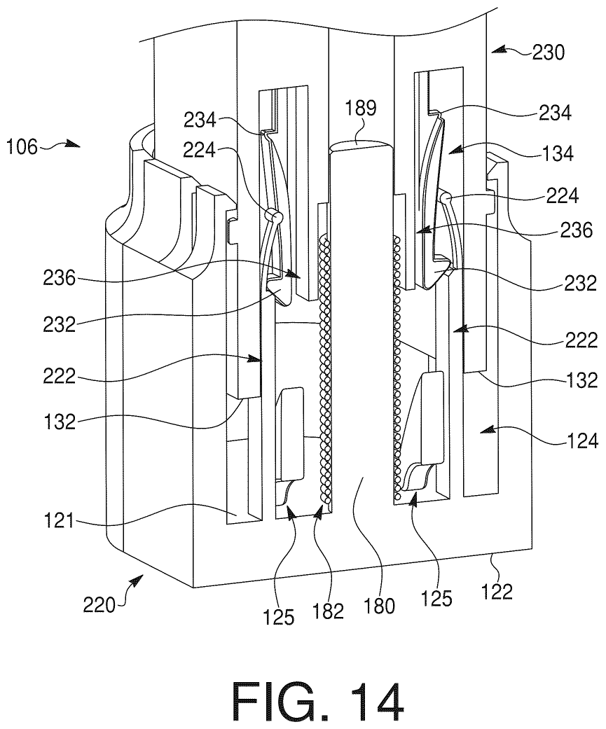

Referring now to FIG. 14, another exemplary shroud 220 and chassis 230 are depicted. It should be appreciated that shroud 220 and chassis 230 may be incorporated into auto-injector 100 in a substantially similar manner as shroud 120 and chassis 130 shown and described above, respectively, except for the differences explicitly noted herein. For example, shroud 220 may include a pair of flexible ribs 222 extending vertically upward through interior cavity 124 with a pin 224 positioned at a terminal (upper) end of each of the pair of flexible ribs 222. Chassis 230 may include a pair of arms 232 and a pair of pin guides 234 disposed within interior cavity 134 and relatively above bottom end 132. Pin guides 234 may be formed along a rear surface of arms 232, and may be sized, shaped, and/or otherwise configured to receive pins 224 thereon for securely coupling shroud 220 to chassis 230. Pin guides 234 may define an angled and/or ramped surface of arms 232 that guides pins 224 into a channel 236 that is configured to secure pin 224 therein.

In the pre-activated state of auto-injector 100, as seen in FIG. 14, the pair of flexible ribs 222 are in contact with the pair of arms 232 with each pin 224 positioned relatively below the corresponding pin guide 234. In response to shroud 220 moving upwards relative to chassis 230, flexible ribs 222 may translate along arms 232 until each pin 224 is aligned with pin guide 234. It should be appreciated that flexible ribs 222 may be configured to move and/or flex in a lateral (radial) direction as shroud 220 pushes flexible ribs 222 against arms 232. For example, flexible ribs 222 may be urged in a radially-outwards direction as shroud 220 is moved upwards relative to chassis 230, and subsequently allowed to flex radially-inwards when pins 224 are aligned with pin guides 234. In this instance, shroud 220 may be in a second position and pin guides 234 may be configured to receive pins 224 along the rear surface of arms 232. As shroud 220 moves partially downward relative to chassis 230, pin guides 234 may wedge pins 224 within the respective channel 236 that is at least partially defined by a lateral (interior) surface of arms 232. In other words, pin guides 234 may guide pins 224 into channels 236, which are positioned radially inward of arms 232. Channels 236 may have a cross-sectional dimension that is less than a cross-sectional dimension of pins 224. Accordingly, pins 224 may be securely fixed inside channels 236, thereby locking shroud 220 to chassis 230 and preventing further downward movement of shroud 220 relative to chassis 230.

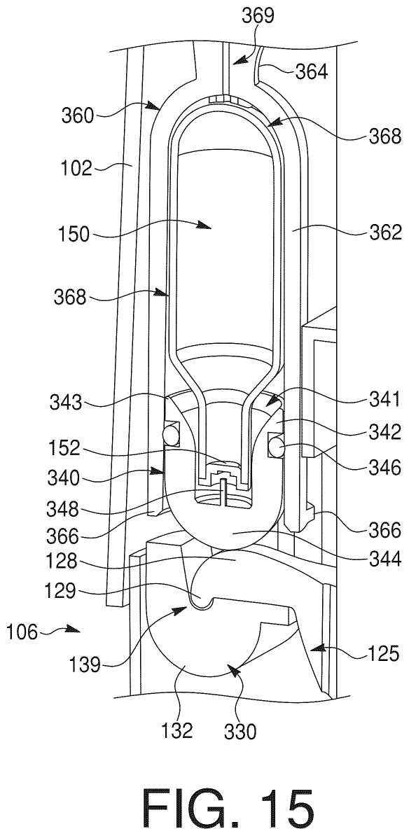

Referring now to FIG. 15, another exemplary chassis 330, carrier 340, and valve assembly 360 are depicted. It should be appreciated that chassis 330, carrier 340, and valve assembly 360 may be incorporated into auto-injector 100 in a substantially similar manner as chassis 130, carrier 140, and valve assembly 160 shown and described above, respectively, except for the differences explicitly noted herein. For example, carrier 340 may be at least partially disposed within valve assembly 360, and neck 152 of canister 150 may be received within carrier 340. As described herein, carrier 340 may be configured to move relative to canister 150 and/or valve assembly 360 in response to actuator 125 pushing carrier 340 in an upward direction into valve assembly 360 and towards canister 150.

Valve assembly 360 may include a chamber 362 defining an interior lumen that is sized, shaped, and/or otherwise configured to receive canister 150 and carrier 340. Chamber 362 may include a top end 364 and a bottom end 366 that is open, with canister 150 positioned adjacent to top end 364 relative to carrier 340, and carrier 340 positioned adjacent to bottom end 366 relative to canister 150. At least a portion of carrier 340 may extend outwardly from chamber 362 via the opening at bottom end 366 and may be in contact with actuator 125. In the example, a cross-sectional dimension of the interior lumen of chamber 362 may be greater than the cross-sectional dimension of canister 150 received therein, thereby forming a gap 368 between an interior surface of chamber 362 and an exterior surface of canister 150. As described herein, a pressurized medium released from canister 150 may be received within chamber 362 and guided towards an outlet 369 of chamber 362 at top end 364 via gap 368.

Still referring to FIG. 15, carrier 340 may have a longitudinal length defined between a top end 342 and a bottom end 344, in which top end 342 may include an opening 341 to receive neck 152 and bottom end 344 may be closed and in contact with second end 128 of actuator 125. Opening 341 at top end 342 may be sized and/or shaped such that top end 342 defines an opening that has a greater cross-sectional dimension than neck 152, such that a gap is formed between an interior surface of top end 342 and an exterior surface of neck 152 received therein. In some embodiments, an edge 343 of top end 342 that defines opening 341 may be angled and/or tapered in a radially-outward direction relative to a central axis of carrier 340, thereby defining a greater gap between top end 342 and neck 152 along edge 343. As described herein, carrier 340 may be configured to receive a pressurized medium stored in canister 150, and release said pressurized medium into valve assembly 360 via opening 341.

Carrier 340 may include a recess and/or cavity along the longitudinal length of carrier 340 between top end 342 and bottom end 344. The recess and/or cavity may be sized, shaped, and/or otherwise configured to receive a fastener 346 (e.g., an O-ring) therein. Fastener 346 may be configured to fluidly seal carrier 340 to valve assembly 360 and inhibit the pressurized medium released from canister 150 into carrier 340 from exiting valve assembly 360. Carrier 340 may further include a piercing mechanism 348 (e.g., a needle, an activator pin, etc.) extending upwards from bottom end 344. Piercing mechanism 348 may be disposed relatively below neck 152 when canister 150 is coupled to carrier 340, and configured to move upwards to puncture the seal of neck 152 in response to second end 128 pushing against bottom end 344 when actuator 125 pivots about the pivot joint defined between pin 129 and recess 139.

In this instance, carrier 340 may be biased in an upwards direction relative to canister 150 and valve assembly 360, such that piercing mechanism 348 punctures the seal at neck 152 thereby releasing the pressurized medium stored in canister 150 into carrier 340. Carrier 340 may be configured to release the pressurized medium into the interior lumen of chamber 362 via opening 341 at top end 342. Fastener 346 may be configured to inhibit release of the pressurized medium out of chamber 362 through the opening at bottom end 366, such that the pressurized medium is directed towards top end 364 via gap 368 until received at outlet 369. In some embodiments, the interior lumen of chamber 362 may include one or more grooves along gap 368, thereby increasing a spatial void for the pressurized medium to travel along from carrier 340 to outlet 369.

Referring to FIGS. 16A-16B, indicator assembly 170 may be disposed within housing 102 adjacent to top end 104. In the example, indicator assembly 170 may include a piston 172, a first indicator surface 174, a second indicator surface 176, a first arm 177, and a second arm 178. Piston 172 may be at least partially received within a portion of valve assembly 160 such that piston 172 is in fluid communication with valve assembly 160. In the example, piston 172 may be configured to move (e.g., translate) in response to valve assembly 160 receiving the pressurized medium released from canister 150. More specifically, upon the pressurized medium equalizing between two internal chambers of valve assembly 160, at least a portion of the pressurized medium received within valve assembly 160 may be configured to propel piston 172 relative to valve assembly 160 and/or housing 102. For example, piston 172 may be configured to move from a first position (FIG. 16A) prior to valve assembly 160 receiving the pressurized medium, towards a second position (FIG. 16B) after valve assembly 160 receives the pressurized medium. It should be understood that movement of piston 172 may cause a corresponding movement of the remaining components of indicator assembly 170, including first indicator surface 174, second indicator surface 176, first arm 177, and second arm 178. Piston 172 may be integrally formed with first indicator surface 174, second indicator surface 176, first arm 177, and second arm 178. For example, piston 172 may form a unitary body with first indicator surface 174 via an intermediate arm 173 connecting piston 172 to first indicator surface 174, such that movement of piston 172 provides a simultaneous movement of first indicator surface 174 second indicator surface 176, first arm 177, and second arm 178.

Indicator assembly 170 may be disposed within housing 102 to at least partially overlap with second window 110. In the first position, piston 172 may be substantially disposed within valve assembly 160 and first indicator surface 174 may be aligned with second window 110 when auto-injector 100 is in the pre-activated state (FIG. 16A). In the second position, piston 172 may be moved relative to housing 102 towards top end 104 and second indicator surface 176 may be aligned with second window 110 when auto-injector 100 is in the activated state (FIG. 16B). First indicator surface 174 and second indicator surface 176 may move relative to second window 110 as a result of piston 172 moving relative to housing 102 in response to the pressurized medium within valve assembly 160 pushing against piston 172. Accordingly, indicator assembly 170 may be configured to generate a visual feedback of a current operating state of auto-injector 100 based on a relative position of first indicator surface 174 and second indicator surface 176 relative to second window 110. Each of first indicator surface 174 and second indicator surface 176 may define a graphical interface that include one or more stickers, markings, coloring, and/or indicia that is distinct from the other. For example, first indicator surface 174 may include a first color (e.g., white) and second indicator surface 176 may include a second color (e.g., green) that is different than the first color. The distinct coloring of first indicator surface 174 and second indicator surface 176, visible through second window 110 when aligned thereto, may indicate the current operating status of auto-injector 100 to a user.

Still referring to FIGS. 16A-16B, indicator assembly 170 may further include a first arm 177 extending radially outwards from first indicator surface 174 and a second arm 178 extending downwards from second indicator surface 176. It should be understood that a relative position, size, and/or shape of first arm 177 and/or second arm 178 may vary from that shown and described herein without departing from a scope of this disclosure. Accordingly, first arm 177 and/or second arm 178 may be located at various other suitable locations along indicator assembly 170 and/or within housing 102. In the example, first arm 177 may be sized, shaped, and/or otherwise configured to move (e.g., translate) within a track 179 formed within housing 102, and second arm 178 may be sized, shaped, and/or otherwise configured to move (e.g., translate) along a ramp 103 formed within housing 102.

Track 179 may define a channel having a vertical configuration, and may be configured to limit a direction of motion of indicator assembly 170 relative to housing 102 during use of auto-injector 100, such as between the pre-activated state of auto-injector 100 (FIG. 16A) and the activated state of auto-injector 100 (see FIG. 16B). For example, track 179 may be configured to limit movement of indicator assembly 170 relative to housing 102 to a vertical direction, thereby inhibiting lateral movement of indicator assembly 170 relative to housing 102, by constraining movement of first arm 177 received therein. Ramp 103 may include an inclined and/or angled surface 103A that may be configured to at least partially deflect second arm 178 in a radially-outwards direction during use of auto-injector 100, such as between the pre-activated state of auto-injector 100 (FIG. 16A) and the activated state of auto-injector 100 (see FIG. 16B). For example, second arm 178 may be at least partially flexible and ramp 103 may be configured to flex and/or bend second arm 178 laterally outwards away from the other internal components of auto-injector 100 as second arm 178 translates along angled surface 103A. Ramp 103 may deflect second arm 178 as second arm 178 translates vertically along angled surface 103A and until second arm 178 is moved beyond a terminal (upper) end 103B of angled surface 103A.

Still referring to FIGS. 16A-16B, upon moving beyond terminal end 103B, second arm 178 may be configured to flex and/or bend laterally inwards relative to housing 102, thereby returning towards its original, undeflected state. Indicator assembly 170 may be configured to generate an audible feedback (e.g., a click) upon second arm 178 contacting a portion of ramp 103 positioned beyond (e.g., relatively above) angled surface 103A after second arm 178 translates over terminal end 103B. In other words, second arm 178 may snap back towards its original position upon moving beyond angled surface 103A, thereby contacting a portion of ramp 103 with sufficient force to generate an audible sound in response to the interaction. It should be appreciated that second arm 178 may be positioned relative to ramp 103 for generating the audible feedback simultaneously as second indicator surface 176 is aligned with second window 110, such that indicator assembly 170 may be configured to generate an audible and visual feedback once auto-injector 100 completes delivery of a dose. Terminal end 103B may be configured to inhibit further movement of indicator assembly 170 due to an abutment against second arm 178. For example, as best seen in FIG. 16B, terminal end 103B may form an impediment that is in contact against second arm 178 when second arm 178 is positioned above terminal end 103B, thereby inhibiting second arm 178 from translating in a downwards direction towards angled surface 103A.

In other embodiments, as seen in FIGS. 17A-17B, first indicator surface 174 and second indicator surface 176 may include a similar marking, color, and/or indicia. Accordingly, alignment of either first indicator surface 174 or second indicator surface 176 with second window 110 does not generate a visual feedback to a user of auto-injector 100 of the current operating state. In this instance, indicator assembly 170 may be configured to only generate an audible feedback that is indicative of the current operating state of auto-injector 100, resulting from the interaction between second arm 178 and ramp 103. In this embodiment, second window 110 may be omitted entirely from housing 102.

Referring now to FIGS. 18A-18C, another exemplary housing 402, shroud 420, and chassis 430 are depicted. It should be appreciated that housing 402, shroud 420, and chassis 430 may be incorporated into auto-injector 100 in a substantially similar manner as housing 102, shroud 120, and chassis 130 shown and described above, respectively, except for the differences explicitly noted herein. For example, housing 402 may include one or more clips 404 (e.g., tabs, fingers, etc.) formed along bottom end 106, and shroud 420 may include one or more corresponding clips 424 (e.g., tabs, fingers, etc.) formed along a top edge 422 of shroud 420 for mating with clips 404. The one or more clips 404 may be configured to engage the one or more clips 424 when shroud 420 is extended downward relative to housing 402 to inhibit shroud 420 from decoupling from housing 402. As described herein, clips 404 and clips 424 may be collectively configured to lockout shroud 420 relative to housing 402 on completing delivery of a dose from auto-injector 100 to inhibit reactivation.

In the example, mandrel assembly 180 may be integrally attached to shroud 420 with biasing mechanism 182 disposed about mandrel assembly 180. Biasing mechanism 182 may extend between opposing ends that are in contact with each of chassis 430 and shroud 420, thereby biasing shroud 420 to an extended position away from chassis 430. Biasing mechanism 182 may be configured to apply a resistance against shroud 420, and in particular an upwards movement of shroud 420 towards chassis 430. As described herein, biasing mechanism 182 may be further configured to extend shroud 420 outwardly from within housing 402 when shroud 420 is depressed into housing 402 to the activated state.

Chassis 430 may include one or more flexible spacers 432 disposed within first chamber 136. The one or more flexible spacers 432 may be disposed about an interior surface of first chamber 136, and may define a cross-sectional dimension that is relatively smaller than the interior lumen of first chamber 136. Flexible spacers 432 may be configured to abut against retention mechanisms 198 of slidable piston 190, thereby maintaining slidable piston 190 within release outlet 169 and inhibiting movement of slidable piston 190 outwards from valve assembly 160. In the example, flexible spacers 432 may have an angled, tapered, and/or ramped interface and retention mechanisms 198 may have a corresponding interface that is configured to engage with the interface of flexible spacer 432. In other words, flexible spacers 432 and retention mechanisms 198 may have a generally ramped engagement with one another. As described herein, flexible spacers 432 may be configured to deform and/or compress radially-outwards in response to slidable piston 190 translating into first chamber 136 in response to the pressurized medium from valve assembly 160 entering release outlet 169 via outlet channel 168 (see FIGS. 18B-18C). As described herein, chassis 430 may be configured to lock slidable piston 190 inside first chamber 136 and second chamber 138 in response to inner lip 137 engaging retention mechanisms 198, thereby inhibiting movement of mandrel assembly 180 into first chamber 136 and second chamber 138. In this instance, shroud 420 may be locked out and prevented from retracting into housing 402 and towards chassis 430.

In the pre-activated state shown in FIG. 18A, shroud 420 may be in a first position with mandrel assembly 180 positioned below chassis 430, and particularly below each of first chamber 136 and second chamber 138. Biasing mechanism 182 may be in an expanded configuration, thereby pushing shroud 420 in a downwards direction away from chassis 430. Upon depressing shroud 420 in an upwards direction towards chassis 430 to transition auto-injector 100 to the activated state, as seen in FIG. 18B, mandrel assembly 180 may move relatively upwards and extend through first chamber 136 and second chamber 138. In this instance, biasing mechanism 182 may be transitioned to a compressed configuration between shroud 420 and chassis 430. In some embodiments, mandrel assembly 180 may be positioned adjacent to and/or in contact with slidable piston 190 when shroud 420 is fully depressed in the second position. Upon receiving the pressurized medium within valve assembly 160, at least a portion of the pressurized medium may be guided to outlet channel 168. The pressurized medium received through outlet channel 168 may encounter upper end 194 of slidable piston 190, thereby urging slidable piston 190 downwards relative to release outlet 169.

Referring to FIG. 18C, after completing delivery of a dose from auto-injector 100, the force applied to shroud 420 may be released thereby allowing biasing mechanism 182 to expand, causing shroud 420 to return towards the first position. Additionally and/or alternatively, shroud 420 may be returned towards the first position in response to slidable piston 190 pushing mandrel assembly 180 in a downwards direction as the pressurized medium received from outlet channel 168 urges slidable piston 190 out of release outlet 169. Clips 404 and clips 424 may be collectively configured to lockout shroud 420 relative to housing 402 upon completing delivery of the dose from auto-injector 100 to inhibit reactivation. In response to biasing mechanism 182 returning towards the expanded configuration and shroud 420 moving downwards relative to chassis 430, mandrel assembly 180 may be urged out of first chamber 136 and second chamber 138. With first chamber 136 and second chamber 138 vacated by mandrel assembly 180, the pressurized medium encountering upper end 194 may propel slidable piston 190 in a downwards direction into first chamber 136 and second chamber 138. It should be appreciated that a force applied to slidable piston 190 by the pressurized medium from outlet channel 168 may be relatively greater than a resistive strength and/or force of flexible spacers 432 against retention mechanisms 198 for maintaining slidable piston 190 within release outlet 169. Accordingly, slidable piston 190 may be configured to move (e.g., translate) relative to release outlet 169 and extend through first chamber 136 and second chamber 138. In this instance, flexible spacers 432 may be deformed and/or move compressed radially-outwards to allow receipt of slidable piston 190 through first chamber 136.

Slidable piston 190 may be urged through first chamber 136 and at least a portion of snap arms 196 may extend into second chamber 138. In some embodiments, snap arms 196 may be biased radially-inwards towards one another as slidable piston 190 extends into first chamber 136 and through flexible spacers 432. Snap arms 196 may be further biased in a radially-inwards direction as snap arms 196 extend through inner lip 137 and into second chamber 138. It should be appreciated that snap arms 196 may move radially-outwards relative to one another, to return to a neutral position, upon entering second chamber 138. With retention mechanisms 198 disposed within second chamber 138 and engaged against inner lip 137, slidable piston 190 may be inhibited from moving relative to chassis 430. Stated differently, inner lip 137 may be configured to couple slidable piston 190 to chassis 430 in response to engaging retention mechanisms 198 within second chamber 138, thereby locking slidable piston 190 to chassis 430 at the end of dose delivery from auto-injector 100. The pressurized medium received in release outlet 169 may pass along an exterior of slidable piston 190 and exit release outlet 169 into first chamber 136. With first chamber 136 open to an interior cavity of housing 402 that is maintained at an atmospheric pressure, auto-injector 100 may be configured to vent the pressurized medium.

Still referring to FIG. 18C, with slidable piston 190 fixed to chassis 430, shroud 420 may be inhibited from further (upwards) movement relative to chassis 430, thereby preventing additional activation of auto-injector 100. Specifically, as snap arms 196 are positioned within first chamber 136 and second chamber 138 when retention mechanisms 198 engage inner lip 137, mandrel assembly 180 is inhibited from moving upwards relative to chassis 430 and entering first chamber 136 or second chamber 138, such as in response to a corresponding movement of shroud 420. Accordingly, slidable piston 190 may be configured to inhibit subsequent activations of auto-injector 100 by obstructing movement of mandrel assembly 180 and shroud 420. It should be appreciated that slidable piston 190 may be configured to extend mandrel assembly 180, and shroud 420 coupled to mandrel assembly 180, to a sufficient extent such that needle 116 (see FIGS. 5 and 10) is disposed within shroud 420 and effectively covered.

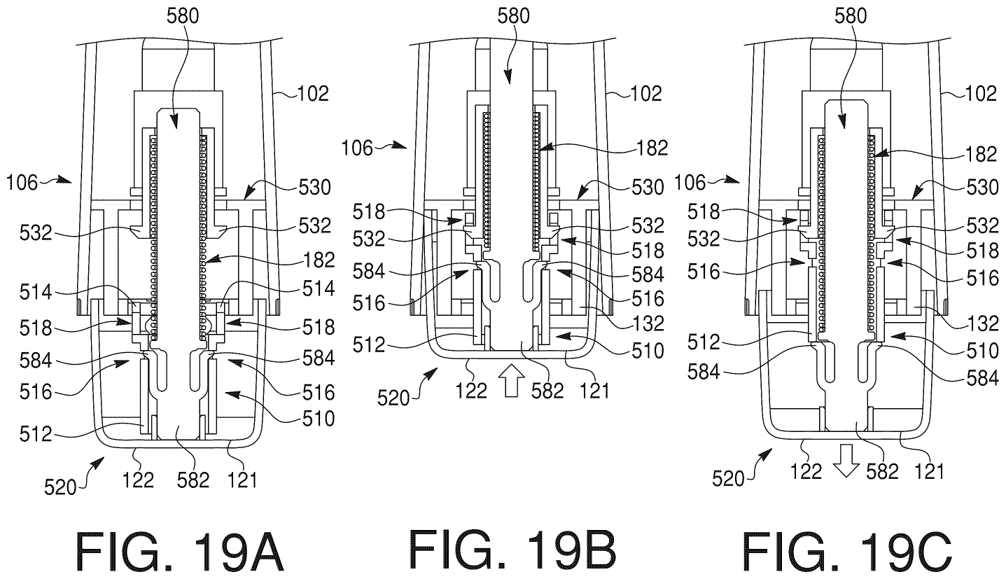

Referring now to FIGS. 19A-19C, another exemplary shroud 520, chassis 530, and mandrel assembly 580 are depicted. It should be appreciated that shroud 520, chassis 530, and mandrel assembly 580 may be incorporated into auto-injector 100 in a substantially similar manner as shroud 120, chassis 130, and mandrel assembly 180 shown and described above, respectively, except for the differences explicitly noted herein. For example, chassis 530 may include a pair of retention mechanisms 532 that are disposed about mandrel assembly 580. Retention mechanisms 532 may include, but are not limited to, a clip, a hand, a finger, a tab, a protrusion, a detent, and more. Chassis 530 may further include a collar 510 coupled at bottom end 132 of chassis 530. Collar 510 may extend relatively downwards from bottom end 132 with mandrel assembly 580 at least partially disposed through each of collar 510 and chassis 530.

In particular, collar 510 may have a longitudinal length defined between a lower end 512 and an upper end 514. Lower end 512 may be coupled to and/or positioned against interior surface 121 of shroud 520, and upper end 514 may be coupled to bottom end 132. Collar 510 may include a pair of first openings 516 and a pair of second openings 518 positioned between lower end 512 and upper end 514, with second openings 518 positioned adjacent to upper end 514 relative to first openings 516. Second openings 518 may be sized, shaped, and/or otherwise configured to receive retention mechanisms 532 for securely coupling collar 510 to chassis 530. Retention mechanisms 532 may be partially flexible and configured to move and/or flex radially-inwards relative to a central axis of chassis 530, such as in response to mandrel assembly 580 moving (e.g. translating) collar 510 within housing 102 and relative to chassis 530, for selectively coupling and/or decoupling chassis 530 to collar 510. As described herein, collar 510 may be configured to move relative to chassis 530 in response to a corresponding movement of shroud 520 and mandrel assembly 580.

Still referring to FIGS. 19A-19C, mandrel assembly 580 may include a pair of retention mechanisms 584 that are positioned relatively adjacent to a lower end 582 of mandrel assembly 580. Retention mechanisms 584 may include, but are not limited to, a clip, a hand, a finger, a tab, a protrusion, a detent, and more. First openings 516 may be sized, shaped, and/or otherwise configured to receive retention mechanisms 584 for securely coupling collar 510 to mandrel assembly 580. Retention mechanisms 584 may be partially flexible and configured to move and/or flex radially-inwards relative to a central axis of mandrel assembly 580, such as in response to mandrel assembly 580 moving (e.g. translating) within housing 102 and relative to collar 510, for selectively coupling and/or decoupling mandrel assembly 580 to collar 510. As described herein, mandrel assembly 580 may be configured to move relative to collar 510 in response to a corresponding movement of shroud 520.

Referring specifically to FIG. 19A, with auto-injector 100 in the pre-activated state, shroud 520 may be in a first position relative to housing 102 in which retention mechanisms 584 are within first openings 516, thereby removably coupling mandrel assembly 580 to collar 510. In this instance, retention mechanisms 532 may be positioned relatively over (and decoupled from) second openings 518. With mandrel assembly 580 coupled to shroud 520, shroud 520 may be configured to move mandrel assembly 580 upwards relative to housing 102 as shroud 520 is pushed in an upwards direction into housing 102. Accordingly, collar 510 may simultaneously move in the upwards direction with mandrel assembly 580 and shroud 520 due to the engagement between retention mechanisms 584 and first openings 516. In other words, mandrel assembly 580 may be configured to pull collar 510 vertically upward with retention mechanisms 584 received within first openings 516. It should be appreciated that biasing mechanism 182 may be transitioned from an expanded configuration (FIG. 19A) to a compressed configuration (FIG. 19B) as shroud 520 pushes mandrel assembly 580 upwards relative to housing 102.

Referring now to FIG. 19B, with auto-injector 100 in the activated state, shroud 520 may be in a second position relative to housing 102 in which mandrel assembly 580 repositions collar 510 relative to chassis 530 such that second openings 518 are aligned with retention mechanisms 532. In this instance, retention mechanisms 532 may be received within second openings 518, thereby coupling chassis 530 to collar 510. It should be appreciated that chassis 530 may be configured such that retention mechanisms 532 are deflected and/or flexibly bent in a radially-inwards direction upon upper end 514 encountering retention mechanisms 532 as collar 510 moves upwards into chassis 530. Upper end 514 may cause retention mechanisms 532 to deflect radially-inwards until retention mechanisms 532 are positioned in alignment with second openings 518. In this instance, retention mechanisms 532 may bend radially-outwards into second openings 518, thereby returning retention mechanisms 532 to a neutral state.

Referring now to FIG. 19C, auto-injector 100 may transition to a lockout state in which shroud 520 may be returned towards the first position relative to housing 102. For example, after completing delivery of a dose from auto-injector 100, the force applied to shroud 520 may be released thereby allowing biasing mechanism 182 to expand, causing shroud 520 to return towards the first position. Specifically, biasing mechanism 182 may be configured to push mandrel assembly 580 downwards relative to housing 102, and mandrel assembly 580 may be configured to push shroud 520 in response to mandrel assembly 580 contacting the interior (bottom) surface of shroud 520. With chassis 530 coupled to collar 510 via the engagement of retention mechanisms 532 and second openings 518, chassis 530 may be configured to inhibit movement of collar 510 as mandrel assembly 580 and shroud 520 are pushed in a downwards direction relative to housing 102. Stated differently, collar 510 may be securely fixed to chassis 530 due to an engagement between second openings 518 and retention mechanisms 532.

Accordingly, retention mechanisms 584 may be configured to move and/or flex radially-inwards towards one another as mandrel assembly 580 moves (e.g. translates) in the downwards direction relative to housing 102 and collar 510. In this instance, retention mechanisms 584 may be configured to exit first openings 516, thereby decoupling mandrel assembly 580 from collar 510. It should be appreciated that biasing mechanism 182 may expand to an extent such that mandrel assembly 580 is pushed through collar 510 until retention mechanisms 584 are positioned relatively below lower end 512. Retention mechanisms 584 may bend radially-outwards upon exiting collar 510 at lower end 512, thereby returning retention mechanisms 584 to a neutral state. In this instance, with retention mechanisms 584 deflected outwards and abutting against lower end 512, collar 510 may be configured to inhibit retention mechanisms 584 from reentering collar 510. Accordingly, an axial position of mandrel assembly 580 relative to collar 510 may be fixed. With mandrel assembly 580 positioned against the inner surface of shroud 520, shroud 520 may be inhibited from moving in the upwards direction, thereby locking shroud 520 and prevent subsequent activation of auto-injector 100.

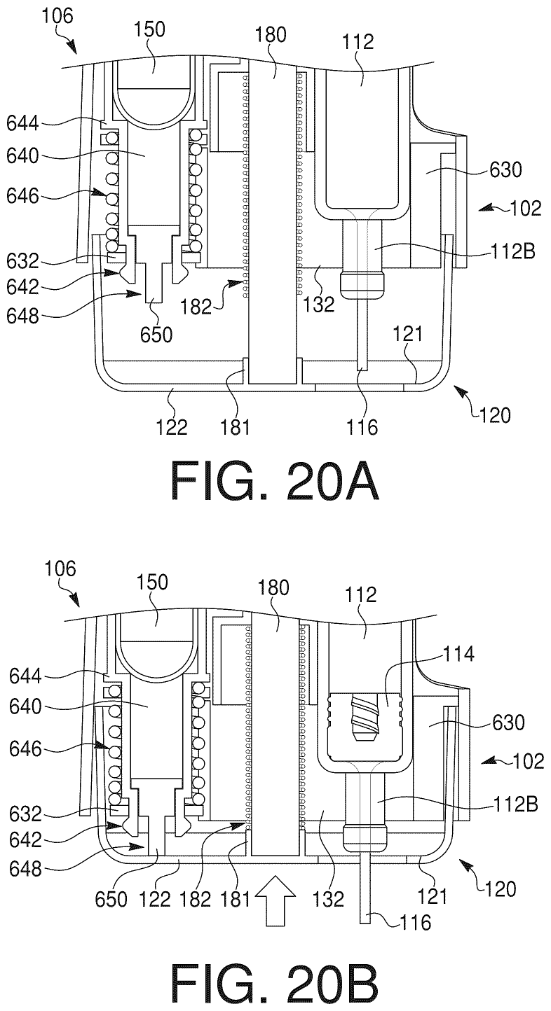

Referring now to FIGS. 20A-20B, another exemplary chassis 630 and carrier 640 are depicted. It should be appreciated that chassis 630 and carrier 640 may be incorporated into auto-injector 100 in a substantially similar manner as chassis 130 and carrier 140 shown and described above, respectively, except for the differences explicitly noted herein. For example, chassis 630 may include a lower flange 632 positioned adjacent to bottom end 132 of chassis 630, and lower flange 632 may be at least partially open for receiving a portion of carrier 640 therethrough. Carrier 640 may include a pair of retention mechanisms 642 that are sized, shaped, and/or otherwise configured to extend into lower flange 632. Specifically, retention mechanisms 642 may be engaged with lower flange 632, thereby removably coupling carrier 640 to chassis 630. In the example, retention mechanisms 642 may include, but are not limited to, a clip, a hand, a finger, a tab, a protrusion, a detent, and more.

Carrier 640 may further include an upper flange 644 and a biasing mechanism 646 (e.g., a spring) that is disposed between lower flange 632 and upper flange 644. Biasing mechanism 646 may be maintained in a compressed configuration when retention mechanisms 642 are engaged with lower flange 632. As described herein, biasing mechanism 646 may be configured to urge carrier 640 in an upwards direction relative to lower flange 632, upon transitioning from a compressed configuration (FIGS. 20A-20B) to an expanded configuration (not shown), in response to retention mechanisms 642 disengaging lower flange 632.

Canister 150 may be disposed within carrier 640 at a position relatively above upper flange 644, such that canister 150 may be biased upwards within housing 102 by biasing mechanism 646. Carrier 640 may include a plug 648 disposed between the pair of retention mechanisms 642, and plug 648 may include a stem 650 extending outwardly from between retention mechanisms 642 in a downwards direction towards interior surface 121 of shroud 120. It should be appreciated that stem 650 may have a longitudinal length that is relatively greater than a corresponding longitudinal length of retention mechanisms 642, such that stem 650 may be positioned closer to interior surface 121 relative to retention mechanisms 642. Plug 648 may be fixed relative to retention mechanisms 642, such that plug 648 is immovable relative to retention mechanisms 642. As described herein, carrier 640 may be configured to move within housing 102 and relative to chassis 630 in response to a corresponding movement of plug 648.

Referring specifically to FIG. 20A, with auto-injector 100 in a pre-activated state, shroud 120 may be in a first position in which interior surface 121 of shroud 120 is offset from stem 650. Biasing mechanism 182 may be in an expanded configuration while biasing mechanism 646 may be in a compressed configuration. In this instance, carrier 640 may be coupled to chassis 630 by the engagement between retention mechanisms 642 and lower flange 632, such that a relative position of canister 150 (disposed within carrier 640) may be fixed relative to chassis 630.

Referring now to FIG. 20B, with auto-injector 100 transitioned to the activated state in response to shroud 120 moving towards a second position in which interior surface 121 may abut against stem 650. In this instance, shroud 120 may be configured to push against plug 648, thereby causing carrier 640 to move in an upwards direction relative to chassis 630. In particular, a force applied to plug 648 by an upwards movement of shroud 120 may cause retention mechanisms 642 to deflect and/or bend radially-inward towards one another, thereby decoupling retention mechanisms 642 from lower flange 632. In this instance, carrier 640 may be movable relative to chassis 630 in an upwards direction in response to biasing mechanism 646 transitioning towards the expanded configuration and urging upper flange 644 upwards. By pushing carrier 640 in an upwards direction relative to housing 102, canister 150 may be simultaneously moved into fluid communication with the valve assembly (not shown) disposed relatively above canister 150 as described in detail above, thereby activating release of the pressurized medium from canister 150.

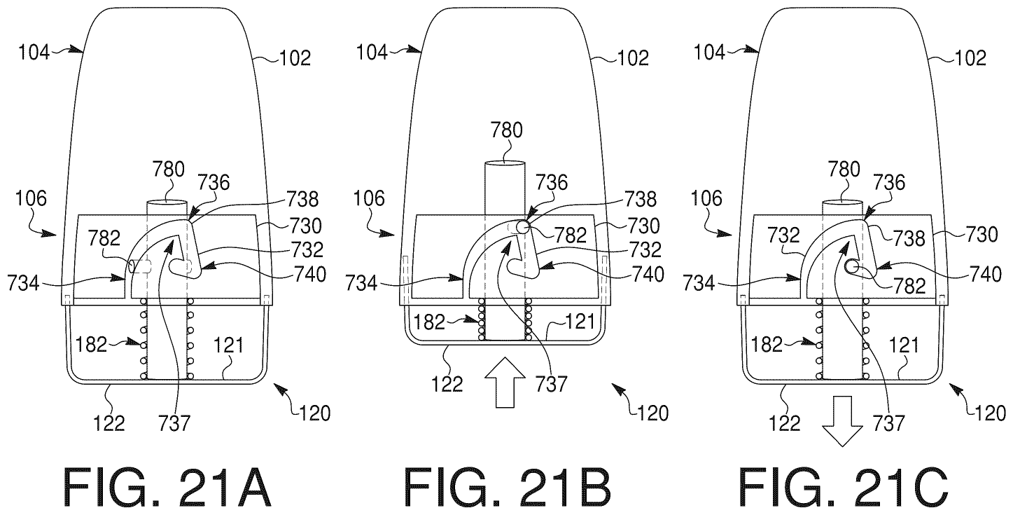

Referring now to FIGS. 21A-21C, another exemplary chassis 730 and mandrel assembly 780 are depicted. It should be appreciated that chassis 730 and mandrel assembly 780 may be incorporated into auto-injector 100 in a substantially similar manner as chassis 130 and mandrel assembly 180 shown and described above, respectively, except for the differences explicitly noted herein. For example, chassis 730 may include a cam path 732 that is sized, shaped, and/or otherwise configured to receive a peg 782 of mandrel assembly 780. Cam path 732 may define a travel pathway for peg 782 such that chassis 730 may be configured to control movement of mandrel assembly 780 during use of auto-injector 100. As described herein, mandrel assembly 780 may be configured to translate and rotate relative to chassis 730 as peg 782 moves through the travel pathway defined by cam path 732. In some embodiments, cam path 732 may include an open channel that is formed within a wall and/or body of chassis 730. Cam path 732 may include a longitudinal length extending between opposing ends that defines the travel pathway for peg 782. With mandrel assembly 780 coupled to and/or in contact with interior surface 121 of shroud 120, chassis 730 may be configured to control a movement and/or a position of shroud 120 via mandrel assembly 780.

As described herein, chassis 730 may be configured to lock shroud 120 by inhibiting movement of mandrel assembly 780 upon an occurrence of an early-lift of shroud 120 during use of auto-injector 100. An early-lift event may be generally defined as the occurrence of an early, untimely, and/or premature release of shroud 120 by a user as auto-injector 100 is transitioned from the pre-activated state to the activated state. Alternatively, an early-lift event may be defined as an early, untimely, and/or premature release of shroud 120 by a user as auto-injector 100 after commencement of the activated state but prior to completing a full injection delivery of the medicament from auto-injector 100. In this instance, chassis 730 may be configured to lock shroud 120 from further movement relative to housing 102, thereby inhibiting further use of auto-injector 100.

Still referring to FIGS. 21A-21C, cam path 732 may include one or more pathways and/or portions for guiding movement of peg 782 relative to chassis 730 during various operating stages of auto-injector 100, thereby controlling a position of shroud 120 relative to housing 102. As described above, cam path 732 may define an open channel formed along a wall and/or body of chassis 730. In the example, cam path 732 may include a first pathway 734, a second pathway 736, and a third pathway 740 each having a shape and/or configuration that is operable to control a relative position of peg 782. First pathway 734 may have a longitudinal length that defines a linear portion of the open channel of cam path 732 that extends upwards along chassis 730, and second pathway 736 may have a longitudinal length that defines a curved portion of the open channel of cam path 732 that extends laterally from first pathway 734. Second pathway 736 may have a terminal end 738 that is opposite of first pathway 734, and terminal end 738 may define an angled (e.g., about ninety degree) portion of cam path 732. Third pathway 740 may have a longitudinal length that defines a linear portion of the open channel of cam path 732 that extends downwards from terminal end 738.

It should be appreciated that cam path 732 may include additional and/or fewer pathways, or pathways of various other suitable shapes and/or configurations, without departing from a scope of this disclosure. In the example, first pathway 734 may be configured for receiving peg 782 within cam path 732 and maintaining mandrel assembly 780 in a first position relative to chassis 730 at a pre-activation state of auto-injector 100. Second pathway 736 may be configured for receiving peg 782 from first pathway 734 and maintaining mandrel assembly 780 in a second position relative to chassis 730 at an activation state of auto-injector 100, such as in response to a rotation of peg 782 and/or mandrel assembly 780 in a first direction relative to chassis 730 and cam path 732. Second pathway 736 may be configured for receiving peg 782 from first pathway 734, such as in response to a rotation of peg 782 and/or mandrel assembly 780 in the first direction relative to chassis 730 and cam path 732. Third pathway 740 may be configured for receiving peg 782 from second pathway 736, such as in response to a rotation of peg 782 and/or mandrel assembly 780 in a second direction relative to chassis 730 and cam path 732 that is different than the first direction. In this instance, third pathway 740 may be configured for locking mandrel assembly 780 in a third position relative to chassis 730 at a delivery completion state of auto-injector 100.

Referring specifically to FIG. 21A, with auto-injector 100 in a pre-activation state, shroud 120 may be in a first position in which shroud 120 is fully extended from housing 102 and peg 782 is disposed within cam path 732 along a portion of first pathway 734. In this instance, biasing mechanism 182 of mandrel assembly 780 may be in an expanded configuration such that biasing mechanism 182 may be configured to urge mandrel assembly 780 in a downwards direction relative to chassis 730, thereby pushing shroud 120 towards the first position. It should be understood that first pathway 734 may be sized, shaped, and/or otherwise configured to define a channel that has a cross-sectional dimension that is substantially similar to a cross-sectional dimension of peg 782. In other words, first pathway 734 does not include any excess clearance and/or void to accommodate early exit of peg 782 from first pathway 734. Accordingly, a movement of peg 782 along first pathway 734 may be constrained to traveling along a full extent of first pathway 734 prior to entering second pathway 736. Due to a size, shape, and/or configuration of first pathway 734, chassis 730 may be configured to rotate mandrel assembly 780 in the first direction within housing 102 in response to shroud 120 moving in an upwards direction towards the second position and peg 782 moving through first pathway 734 to second pathway 736.

Referring now to FIG. 21B, with auto-injector 100 in an activation state, shroud 120 may be in a second position in which shroud 120 is retracted into housing 102 and peg 782 is disposed within cam path 732 along a portion of second pathway 736. In this instance, biasing mechanism 182 may be moved to a compressed configuration in response to shroud 120 moving in the upwards direction and causing mandrel assembly 780 to translate vertically into housing 102. In the example, it should be appreciated that mandrel assembly 780 may be configured to translate vertically and rotate simultaneously as shroud 120 is moved relative to chassis 730. Peg 782 may be positioned at a terminal end 738 of second pathway 736 when the dose from auto-injector 100 has been fully expelled. Accordingly, second pathway 736 may inhibit further movement of peg 782 when peg 782 is positioned at terminal end 738, thereby indicating completion of the dose delivery. As described herein, second pathway 736 may be sized, shaped, and/or otherwise configured to urge peg 782 towards third pathway 740 upon peg 782 being positioned at terminal end 738.

It should be understood that second pathway 736 may be sized, shaped, and/or otherwise configured to define a channel that has a cross-sectional dimension that is substantially greater than a cross-sectional dimension of peg 782. In other words, second pathway 736 includes an excess clearance and/or void 737 to accommodate an early exit (release) of peg 782 from second pathway 736, such as in response to an occurrence of an early-lift of shroud 120 during use of auto-injector 100. Accordingly, a movement of peg 782 (e.g., rotation) along second pathway 736 may not be constrained to traveling along a full extent of second pathway 736, such as to terminal end 738, prior to entering third pathway 740. Instead, cam path 732 may be sized and/or shaped along second pathway 736 to guide peg 782 towards third pathway 740 upon an early-lift of shroud 120 if auto-injector 100 is not fully transitioned to the activated state via the void 737. Void 737 may define an alternative path for peg 782 between second pathway 736 and third pathway 740. As described below, peg 782 may be fixedly secured within cam path 732 upon arriving in third pathway 740, irrespective of whether peg 782 translates to terminal end 738.

Referring now to FIG. 21C, with auto-injector 100 in a delivery completion state, shroud 120 may be automatically returned towards the first position in which shroud 120 is extended from housing 102 and peg 782 is disposed within cam path 732 along a portion of third pathway 740. In this instance, biasing mechanism 182 may automatically return (e.g., move) towards the expanded configuration such that biasing mechanism 182 may be configured to urge mandrel assembly 780 in the downwards direction relative to chassis 730, thereby pushing shroud 120 towards the first position. In this instance, mandrel assembly 780 and/or peg 782 may be configured to rotate in the second direction that is different than the first direction. In the example, third pathway 740 may define a closed end of cam path 732, such that subsequent activation of auto-injector 100 is inhibited in response to third pathway 740 locking peg 782 at the closed end of cam path 732. In other words, with peg 782 inhibited from further movement when received within third pathway 740, mandrel assembly 780 may be locked to chassis 730, thereby inhibiting movement of shroud 120 relative to housing 102. In the example, the first direction may include a clockwise direction and the second direction may include a counter clockwise direction.

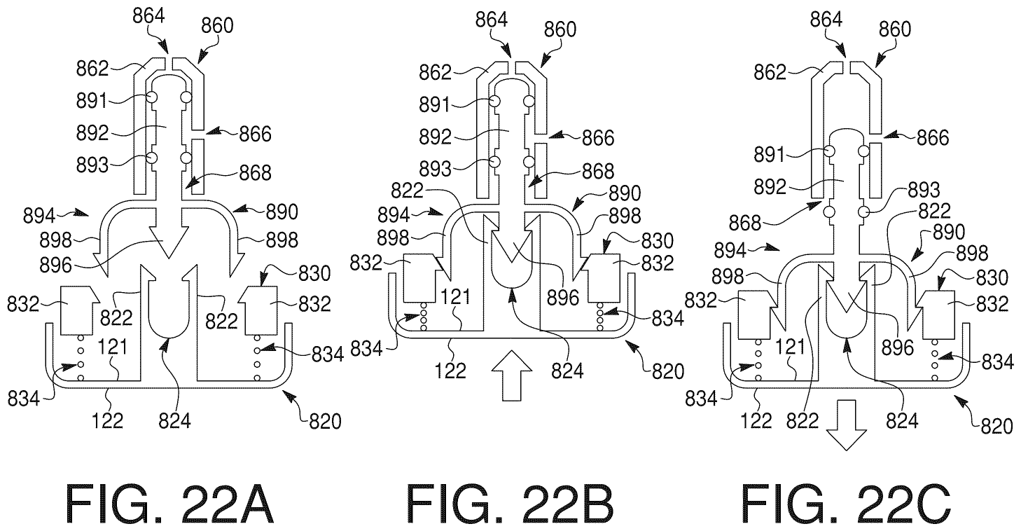

Referring now to FIGS. 22A-22C, another exemplary shroud 820, chassis 830, valve assembly 860, and slidable piston 890 are depicted. It should be appreciated that shroud 820, chassis 830, valve assembly 860, and slidable piston 890 may be incorporated into auto-injector 100 in a substantially similar manner as shroud 120, chassis 130, valve assembly 160, and slidable piston 190 shown and described above, respectively, except for the differences explicitly noted herein. For example, shroud 820 may include a pair of retention mechanisms 822 extending outwardly from interior surface 121 of shroud 820 in an upwards direction towards chassis 830. The pair of retention mechanisms 822 may be spaced apart from one another by a cavity 824 formed therebetween. In some embodiments, retention mechanisms 822 may include, but are not limited to, a clip, a hand, a finger, a tab, a protrusion, a detent, and more. As described herein, cavity 824 may be sized, shaped, and/or otherwise configured to receive at least a portion of slidable piston 890 therein in response to shroud 820 engaging slidable piston 890.

Chassis 830 may include a pair of retention mechanisms 832 that are movably coupled to shroud 820 via a biasing mechanism 834. Biasing mechanism 834 may be coupled to shroud 820 along interior surface 121. As described herein, biasing mechanism 834 may be configured to urge shroud 820 away from, and particularly in a downwards direction relative to, chassis 830 when in an expanded configuration. Biasing mechanism 834 may be configured to transition from the expanded configuration to a compressed configuration (FIG. 22B) in response to shroud 820 moving in an upwards direction towards chassis 830. Valve assembly 860 may include a body 862 having a first port 864, a second port 866, and a lower opening 868. First port 864 may be in fluid communication with a low pressure line of auto-injector 100, second port 866 may be in fluid communication with a venting line and/or opening of auto-injector 100, and lower opening 868 may be sized, shaped, and/or otherwise configured to receive at least a portion of slidable piston 890 within body 862 via lower opening 868.

As described herein, slidable piston 890 may be slidably received within valve assembly 860, and specifically an upper end 892 of slidable piston 890 may be movably disposed within body 862. Slidable piston 890 may include a first seal 891 and a second seal 893 that are positioned along upper end 892. When slidable piston 890 is in a first position relative to valve assembly 860 (see FIG. 22A), first seal 891 may be disposed between first port 864 and second port 866, and second seal 893 may be disposed between second port 866 and lower opening 868. In this instance, first port 864 may be fluidly decoupled from second port 866 due to a position of first seal 891 within body 862 therebetween, and second port may be fluidly decoupled from lower opening 868 due to a position of second seal 893 within body 862 therebetween. As described herein, slidable piston 890 may be configured to selectively establish fluid communication between first port 864 and second port 866 in response to moving relative to body 862 and repositioning first seal 891 and/or second seal 893.

Still referring to FIGS. 22A-22C, slidable piston 890 may include a lower end 894 that is positioned opposite of upper end 892. Lower end 894 may include a plurality of retention mechanisms that are each size, shaped, and/or otherwise configured to attach slidable piston 890 to one or more other components of auto-injector 100, such as shroud 820 and chassis 830. In the example, slidable piston 890 may include a central retention mechanism 896 extending outwardly from lower end 894 and a pair of lateral retention mechanisms 898 disposed radially-outwards from central retention mechanism 896. In some embodiments, central retention mechanism 896 may include, but is not limited to, a clip, a hand, a finger, a tab, a protrusion, a hook, and more. The pair of lateral retention mechanisms 896 may include, but are not limited to, a clip, a hand, a finger, a tab, a protrusion, a detent, and more. When in the first position of slidable piston 890, with upper end 892 disposed within valve assembly 860, lower end 894 may extend downward from body 862 via lower opening 868 of valve assembly 860. Accordingly, retention mechanisms 896, 898 are disposed outside of body 862 when slidable piston 890 is in the first position relative to valve assembly 860.

Referring specifically to FIG. 22A, with auto-injector 100 in a pre-activated state, shroud 820 may be in a first position in which biasing mechanism 834 is in the expanded state such that shroud 820 is decoupled from slidable piston 890. Further, with slidable piston 890 in a respective first position relative to valve assembly 860, chassis 830 is decoupled from slidable piston 890. In response to moving shroud 820 in an upwards direction towards chassis 830, such as in response to applying a force against bottom surface 122 to push shroud 820 in the upwards direction, shroud 820 may be configured to compress biasing mechanism 834. In this instance, retention mechanisms 822 may be moved towards slidable piston 890 and into engagement with central retention mechanism 896, thereby coupling shroud 820 to slidable piston 890.

Referring now to FIG. 22B, auto-injector 100 may be in an activated state with shroud 820 translated towards a second position in which slidable piston 890 is securely coupled to shroud 820. Accordingly, slidable piston 890 may be configured to move with shroud 820. It should be appreciated that chassis 830 may remain fixed relative to shroud 820 as shroud 820 moves towards chassis 830 and the second position. Upon releasing the force applied to shroud 820 against bottom surface 122, biasing mechanism 834 may be configured to return towards the expanded configuration, thereby pushing shroud 820 in a downwards direction away from chassis 830. In other words, shroud 820 may be automatically returned towards the first position. In this instance, with slidable piston 890 coupled to shroud 820, biasing mechanism 834 may be further configured to pull slidable piston 890 at least partially out of body 862 through lower opening 868.

In some embodiments, shroud 820 may be configured such that retention mechanism 822 may be configured to engage central retention mechanism 896 prior and/or during the transition of auto-injector 100 from the pre-activated state (FIG. 22A) to the activated state (FIG. 22B). In this instance, shroud 820 may be coupled to slidable piston 890 to accommodate an early exit (release) of shroud 820, such as in response to an occurrence of an early-lift of shroud 820 during use of auto-injector 100. As described herein, with shroud 820 coupled to slidable piston 890, biasing mechanism 834 may be configured to lock shroud 820 to chassis 830 upon completing the activation state or upon an early-lift of shroud 820 when auto-injector 100 is not fully transitioned to the activated state.

Referring to FIG. 22C, auto-injector 100 may be transitioned towards a delivery completed state with shroud 820 automatically moving towards or returned towards the first position and slidable piston 890 moved from a respective first position towards a second position. Shroud 820 may be configured to pull slidable piston 890 in a downwards direction to an extent such that lateral retention mechanisms 898 may interface with retention mechanisms 832, thereby securely coupling slidable piston 890 to chassis 830. In this instance, slidable piston 890 may be maintained in the second position due to its attachment to each of shroud 820 and chassis 830, thereby locking shroud 820 and slidable piston 890 to the respective positions. It should be understood that shroud 820 and slidable piston 890 may be positioned at the respective locations shown in FIG. 22C upon the occurrence of an early-lift of shroud 820, such that further activation of auto-injector 100 is prevented.

Additionally, as slidable piston 890 extends out of body 862 via lower opening 868, upper end 892 may move relative to first port 864 and second port 866 with first seal 891 positioned relatively below second port 866 and second seal 893 positioned relatively below lower opening 868. In other words, second seal 893 may be positioned outside of body 862. In this instance, slidable piston 890 may be configured to fluidly couple first port 864 to second port 866 due to a position of upper end 892 within body 862, and particularly a location of first seal 891 relative to body 862. Accordingly, the low pressure line in fluid communication with second port 866 may direct the pressurized gas received therein to the vent opening and/or line at first port 864. Additionally, slidable piston 890 may be configured to vent any pressurized medium received from low pressure line between first seal 891 and second seal 893 (via second port 866 from when slidable piston 890 was in the first position) out of valve assembly 860 via lower opening 868 due to a position of second seal 893 relatively below lower opening 868.

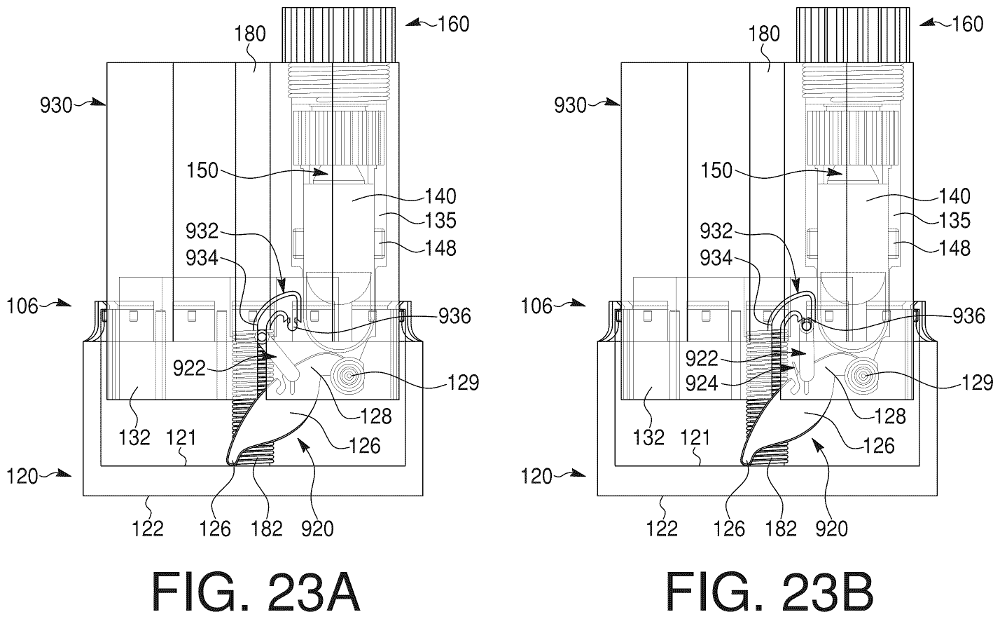

Referring now to FIGS. 23A-23B, another exemplary actuator 925 and chassis 930 are depicted. It should be appreciated that actuator 925 and chassis 930 may be incorporated into auto-injector 100 in a substantially similar manner as actuator 125 and chassis 130 shown and described above, respectively, except for the differences explicitly noted herein. For example, actuator 925 may include a lever 922 that is movably coupled to body 126. Chassis 930 may include a cam path 932 defined by a first pathway 934 and a second pathway 936. In the example, first pathway 934 may define an angled and/or ramped surface along cam path 932 and second pathway 936 may define a closed end of cam path 932. In other words, first pathway 934 may have a longitudinal length that defines a curved portion of an open channel of cam path 932 that extends laterally along chassis 930. In some examples, first pathway 934 may be generally U-shaped. Second pathway 936 may be positioned at a terminal end of first pathway 734. Cam path 932 may be sized, shaped, and/or otherwise configured to slidably receive at least a portion of lever 922 therein. In other words, lever 922 may be coupled to and movably relative to each of body 126 and cam path 932. Actuator 920 may further include a biasing mechanism 924 (e.g., a torsion spring) coupled to body 126 and positioned adjacent to lever 922. Biasing mechanism 924 may be configured to urge lever 922 to move along cam path 932 from first pathway 934 towards second pathway 936.

Referring to FIG. 23A, with auto-injector 100 in a pre-activated state, shroud 120 may be in a first position with lever 922 positioned along first pathway 934 and biasing mechanism 182 in an expanded configuration. In response to moving shroud 120 in an upwards direction towards chassis 930, actuator 920 may be configured to move thereby pivoting lever 922 relative to body 126 and translating lever 922 relative to cam path 932. In the example, lever 922 may be configured to move (e.g., pivot, rotate, translate, etc.) through the curved portion of first pathway 934. Similar to cam path 732 shown and described above (FIGS. 21A-21C), first pathway 934 may include a terminal end defining a full stroke of shroud 120 for delivering a complete dose, and second pathway 936 may include an excess clearance and/or void defining an alternate path for lever 922 upon the occurrence of an early-lift event. In this instance, an early-lift of shroud 120 may cause lever 922 to travel along the alternate path of second pathway 936. In either instance, chassis 930 may be configured to lock shroud 120 in response to lever 922 being received at a closed end of cam path 932 along second pathway 936, as seen in FIG. 23B. It should be appreciated that biasing mechanism 182 may be configured to automatically return shroud 120 towards the first position by urging shroud 120 downwards relative to chassis 930.

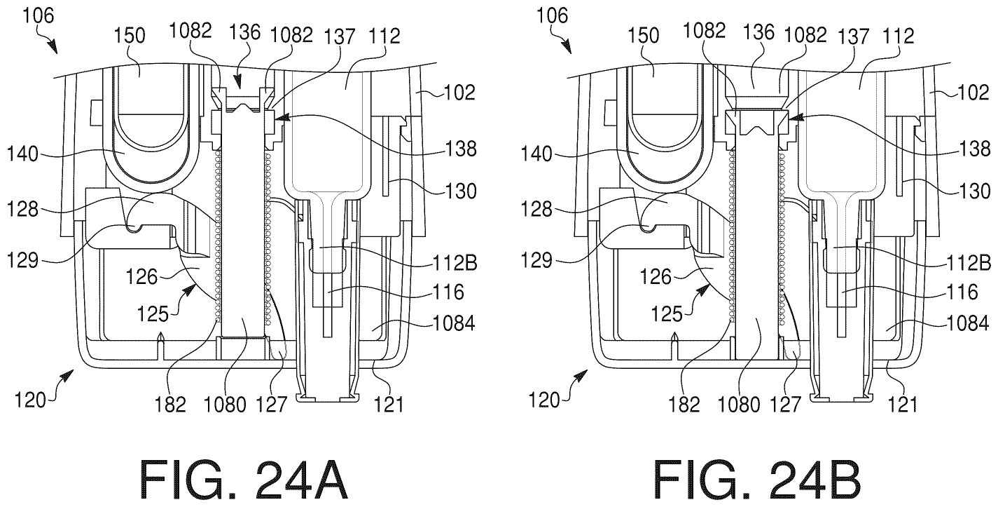

Referring now to FIGS. 24A-24B, another exemplary mandrel assembly 1080 is depicted. It should be appreciated that mandrel assembly 1080 may be incorporated into auto-injector 100 in a substantially similar manner as mandrel assembly 180 shown and described above, respectively, except for the differences explicitly noted herein. For example, as seen in FIG. 24A, mandrel assembly 1080 may include a pair of retention mechanisms 1082 that may be disposed within first chamber 136 when shroud 120 is in a first position and auto-injector 100 is in the pre-activated state. The pair of retention mechanisms 1082 may include, but are not limited to, a clip, a hand, a finger, a tab, a protrusion, a detent, and more. In this instance, mandrel assembly 1080 may be offset from interior surface 121 of shroud 120 and received on a ledge 1084 that is positioned relatively above interior surface 121. Mandrel assembly 1080 may be configured to rotate in response to shroud 120 moving towards a second position and auto-injector 100 transitioning to an activated state (not shown).

As seen in FIG. 24B, in response to mandrel assembly 1080 rotating, mandrel assembly 1080 may translate in a downwards direction such that mandrel assembly 1080 may be received along interior surface 121. Retention mechanisms 1082 may be configured to move from first chamber 136 to second chamber 138 in response to retention mechanisms 1082 deflecting radially-inwards towards one another in response to abutting against inner lip 137. Upon entering second chamber 138, retention mechanisms 1082 may be configured to deflect and/or flexibly bend in a radially-outwards direction, thereby locking mandrel assembly 1080 relative to chassis 130. With mandrel assembly 1080 received on interior surface 121, mandrel assembly 1080 may be configured to inhibit further movement of shroud 120 relative to chassis 130. It should be appreciated that mandrel assembly 1080 may be configured to exit ledge 1084 and abut against interior surface 121 upon occurrence of a complete dose delivery or an early-lift event. Thus, an early-lift of shroud 120 may cause mandrel assembly 1080 to downwardly against interior surface 121. In either instance, chassis 130 may be configured to lock shroud 120 in response to mandrel assembly 1080 being received against interior surface 121 and retention mechanisms 1082 being locked within second chamber 138.

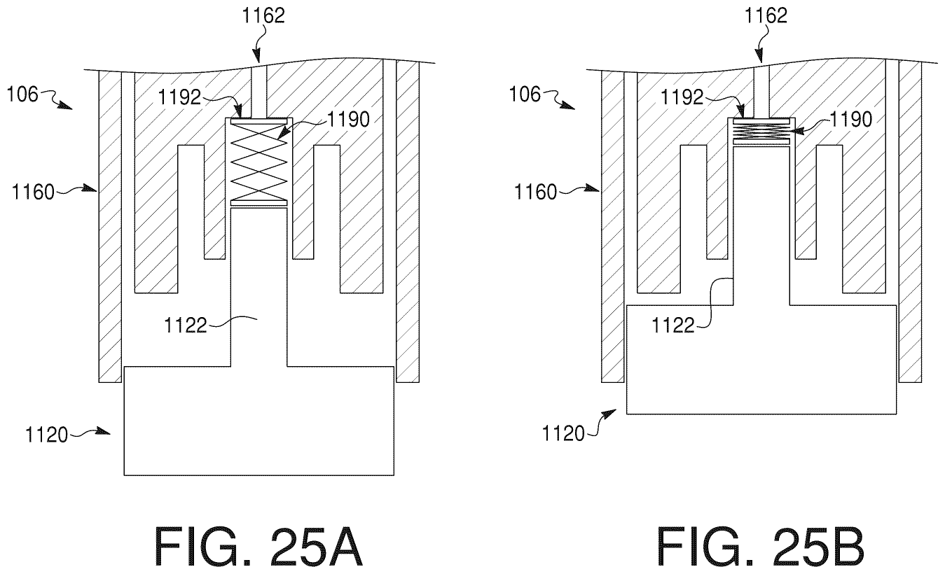

Referring now to FIGS. 25A-25B, another exemplary shroud 1120, valve assembly 1160, and slidable piston 1190 are depicted. It should be appreciated that shroud 1120, valve assembly 1160, and slidable piston 1190 may be incorporated into auto-injector 100 in a substantially similar manner as shroud 120, valve assembly 160, and slidable piston 190 shown and described above, respectively, except for the differences explicitly noted herein. For example, shroud 1120 may include a stem 1122 that is sized, shaped, and/or otherwise configured to abut against slidable piston 1190. In particular, shroud 1120 may be configured to push stem 1122 against slidable piston 1190, thereby sealing a port 1162 of valve assembly 1160 with slidable piston 1190. In some embodiments, slidable piston 1190 may include a compliant seal 1192 at an upper end of slidable piston 1190, and compliant seal 1192 may be configured to seal port 1162 upon shroud 1120 pushing stem 1122 against slidable piston 1190 with a sufficient sealing force, as seen in FIG. 25B. In some embodiments, slidable piston 1190 may include a spring and/or other biasing mechanism, such that slidable piston 1190 may be transitioned from an expanded configuration (FIG. 25A) towards a compressed configuration (FIG. 25B).

In the example, port 1162 may be in fluid communication with a low pressure line of auto-injector 100. By creating a face seal on port 1162, a necessary sealing force is minimized as port 1162 may be generally small in size, such as, for example, having a diameter of about 1 millimeter. Upon the occurrence of an early-lift of shroud 1120, compliant seal 1192 may lose contact with port 1162, thereby allowing pressurized medium from the low pressure line fluidly coupled thereto to push against shroud 1120 in a downwards direction, thereby locking shroud 1120. It should be appreciated that compliant seal 1192 does not seal a port that is in fluid communication with a vent line and/or opening of valve assembly 1160. In some embodiments, auto-injector 100 may include a secondary indicator that is configured to generate a notification for lifting shroud 1120 properly to transition to the activated state while avoiding an early-lift occurrence.

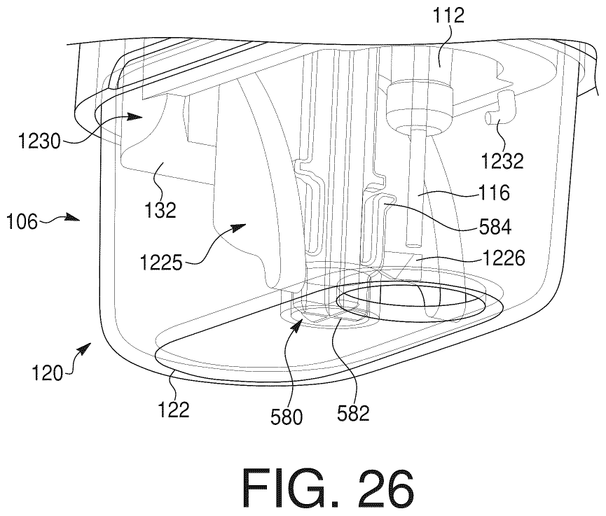

Referring now to FIG. 26, another exemplary actuator 1125 and chassis 1230 are depicted. It should be appreciated that actuator 1125 and chassis 1230 may be incorporated into auto-injector 100 in a substantially similar manner as actuator 125 and chassis 130 shown and described above, respectively, except for the differences explicitly noted herein. For example, actuator 1125 may include one or more retention mechanisms 1226 and chassis 1230 may include one or more corresponding retention mechanisms 1232. Upon moving shroud 120 and transitioning auto-injector 100 to the activated state, retention mechanism 1226 may be configured to engage retention mechanism 1232. In this instance, it should be understood that retention mechanisms 584 of mandrel assembly 580 may be disposed relatively above retention mechanism 1226. Retention mechanisms 584 may be laterally moveable and/or deflectable in response to engaging one or more other components of auto-injector 100. In some embodiments, retention mechanisms 584 may be biased radially-outwards relative to lower end 582 of mandrel assembly 580. Each of retention mechanisms 584, 1226, 1232 may include, but are not limited to, an impediment, a tab, a protrusion, a finger, a clip, a detent, and/or various other mechanisms.

In the event of an early-lift occurrence of shroud 120, mandrel assembly 580 may be configured to move relative to actuator 1225 such that retention mechanism 584 may translate in a downwards direction until retention mechanisms 584 are disposed relatively below retention mechanism 1226. It should be appreciated that retention mechanism 584 may be configured to deflect and/or bend radially-inwards towards mandrel assembly 580 when moving downwards by retention mechanism 1226 before flexing radially-outwards to a neutral state upon extending below retention mechanism 1226. With retention mechanism 584 positioned below and abutting against retention mechanism 1226, mandrel assembly 580 may be configured to lock shroud 120 from further movement relative to chassis 1230.

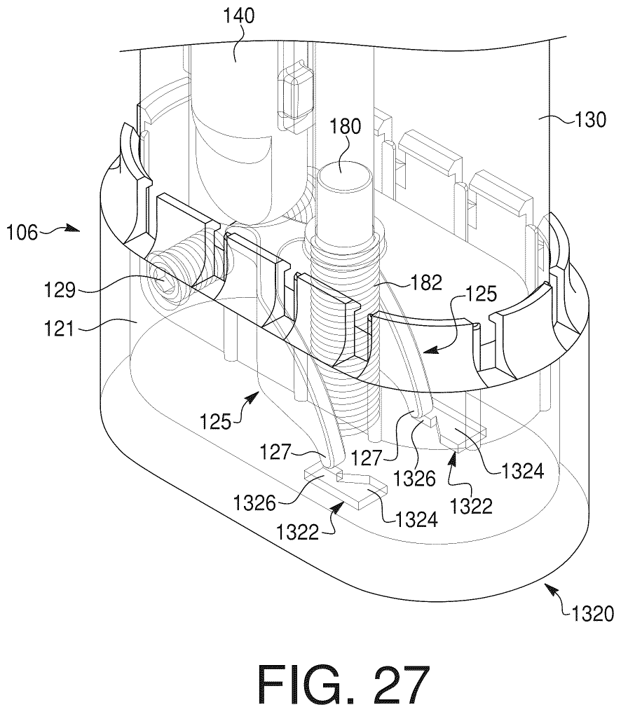

Referring now to FIG. 27, another exemplary shroud 1320 is depicted. It should be appreciated that shroud 1320 may be incorporated into auto-injector 100 in a substantially similar manner as shroud 120 shown and described above, respectively, except for the differences explicitly noted herein. For example, shroud 1320 may include a pocket 1322 formed along interior surface 121. Pocket 1322 may include an enlarged end 1324 and a narrowed end 1326 that are positioned adjacent to one another along interior surface 121. Pocket 1322 may be positioned along interior surface 121 of shroud 1320 in alignment with actuator 125. Pocket 1322 may be sized, shaped, and/or otherwise configured to receive first end 127 of actuator 125.

In response to moving shroud 1320 in an upwards direction towards chassis 130, actuator 125 may be configured to translate relative to interior surface 121 and over pocket 1322 until first end 127 is received within enlarged end 1324 upon auto-injector 100 transitioning from a pre-activated state towards an activated state. It should be appreciated that pocket 1322 may be sized, shaped, and/or otherwise configured to inhibit receipt of first end 127 directly within narrowed end 1326, such as from a location directly above narrowed end 1326. Stated differently, first end 127 may translate directly over narrowed end 1326 as actuator 125 slides across interior surface 121 without entering into narrowed end 1326 due to a top opening of narrowed end 1326 along interior surface 121 being relatively smaller than a cross-sectional dimension of first end 127. As such, first end 127 may translate directly over narrowed end 1326 without being received in narrowed end 1326 despite narrowed end 1326 being opened directly beneath first end 127.

As described herein, a lateral opening of narrowed end 1326 along a side of narrowed end 1326 that is adjacent to enlarged end 1324 may be sized and/or shaped to have a cross-sectional dimension that is sufficient to receive first end 127 therethrough. Upon occurrence of an early-lift of shroud 1320, interior surface 121 may move relative to actuator 125 such that first end 127 is snapped into narrowed end 1326 from enlarged end 1324, thereby locking actuator 125 relative to shroud 1320. It should be appreciated that pocket 1322 may be configured to inhibit release of first end 127 from narrowed end 1326 upon receipt therein from enlarged end 1324. In this instance, shroud 1320 may be inhibited from movement relative to chassis 130 due to a fixed engagement between actuator 125 and pocket 1322, and particularly first end 127 being securely engaged inside narrowed end 1326.

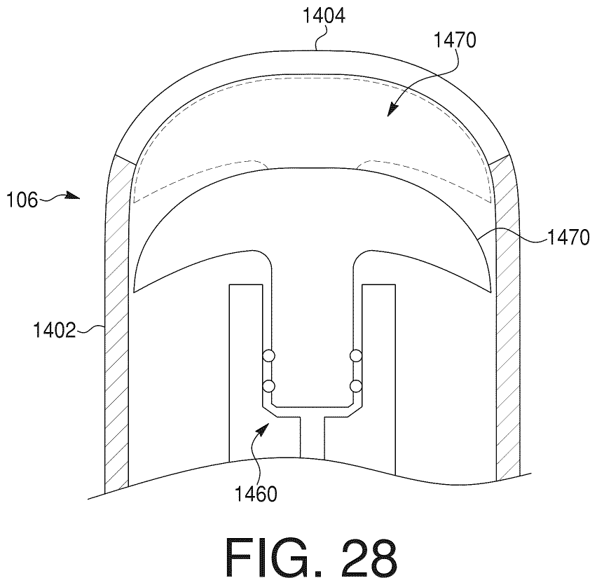

Referring now to FIG. 28 another exemplary housing 1402, valve assembly 1460, and indicator assembly 1470 are depicted. It should be appreciated that housing 1402, valve assembly 1460, and indicator assembly 1470 may be incorporated into auto-injector 100 in a substantially similar manner as housing 102, valve assembly 160, and indicator assembly 170 shown and described above, respectively, except for the differences explicitly noted herein. For example, housing 1402 may include a window 1404 along top end 104, and window 1404 may define an opaque wall of housing 1402 at top end 104. Indicator assembly 1470 may be disposed within housing 1402 and offset from window 1404 such that indicator assembly 1470 is not visible through window 1404 when auto-injector 100 is in a pre-activated state.

Indicator assembly 1470 may be fluidly coupled to valve assembly 1460 such that indicator assembly 1470 may be configured to translate within housing 1402, and particularly in an upwards direction towards top end 104, in response to valve assembly 1460 receiving pressurized medium. Accordingly, upon a complete dose delivery from auto-injector 100, the pressurized medium may urge indicator assembly 1470 towards window 1404 such that indicator assembly 1470 may be visible from an exterior of housing 1402, thereby generating a visual feedback of the dose delivery. In some embodiments, indicator assembly 1470 may be configured to abut against an interior surface of window 1404, thereby generating a tactile feedback of the dose delivery.

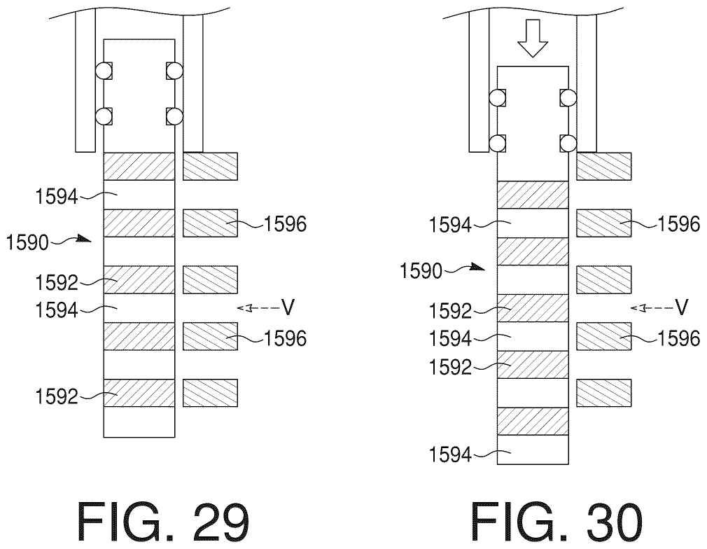

Referring now to FIGS. 29-30, another exemplary slidable piston 1590 is depicted. It should be appreciated that slidable piston 1590 may be incorporated into auto-injector 100 in a substantially similar manner as slidable piston 190 shown and described above, respectively, except for the differences explicitly noted herein. For example, slidable piston 1590 may include one or more first indicator surfaces 1592 and one or more second indicator surfaces 1594 disposed along an exterior of slidable piston 1590. Each of first indicator surfaces 1592 and second indicator surfaces 1594 may include one or more markings, coloring, and/or indicia that is distinct from the other. For example, first indicator surfaces 1592 may include a first color (e.g., green) and second indicator surfaces 1594 may include a second color (e.g., white) that is different than the first color. Slidable piston 1590 and/or housing 102 may include one or more obstructions features 1596.

Referring specifically to FIG. 29, in a pre-activated state of auto-injector 100, slidable piston 1590 may be positioned such that first indicator surfaces 1592 are aligned with obstruction features 1596 and second indicator surfaces 1594 are offset from obstruction features 1596, such that first indicator surfaces 1592 are not visible from a viewpoint V that is offset from and coincides with a space and/or gap between adjacent obstruction features 1596, and second indicator surfaces 1594 are visible from viewpoint V. In this instance, second indicator surfaces 1594 may be operable to indicate a pre-activation state of auto-injector 100 when offset from obstruction features 1596 and visible via viewpoint V. In some embodiments, obstruction features 1596 may be a surface and/or a wall of auto-injector 100.

Referring now to FIG. 30, upon auto-injector 100 completing a dose delivery and venting, slidable piston 1590 may be configured to move relative to housing 102 such that first indicator surfaces 1592 are visible from viewpoint V and second indicator surfaces 1594 are no longer visible from viewpoint V. In this instance, first indicator surfaces 1592 may be operable to indicate a complete delivery state of auto-injector 100 when offset from obstruction features 1596 and visible. It should be appreciated that viewpoint V may be from a standpoint and/or perspective of a user of auto-injector 100 during use of auto-injector 100, such as from an exterior of auto-injector 100.

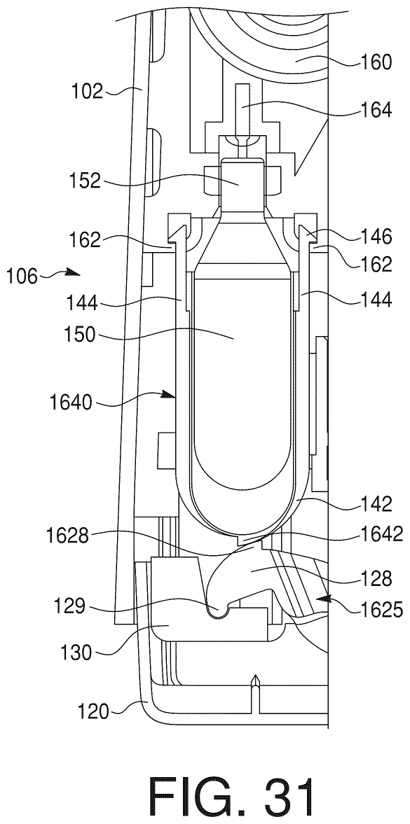

Referring now to FIG. 31, another exemplary actuator 1625 and carrier 1640 are depicted. It should be appreciated that actuator 1625 and carrier 1640 may be incorporated into auto-injector 100 in a substantially similar manner as actuator 125 and carrier 140 shown and described above, respectively, except for the differences explicitly noted herein. For example, actuator 1625 may include a protrusion 1628 along second end 128 and carrier 1640 may include a corresponding protrusion 1642 along bottom end 142. Each of protrusion 1628 and protrusion 1642 may include a step, a tab, an abutment, and/or various other suitable extensions along the respective end of actuator 1625 and carrier 1640. In a first position, protrusion 1642 may be positioned to a left of protrusion 1628 prior to movement of actuator 1625 relative to carrier 1640. In a second position, protrusion 1642 may be positioned to a right of protrusion 1628 after movement of actuator 1625 relative to carrier 1640.

Protrusion 1628 may be configured to engage protrusion 1642 in response to actuator 1625 moving relative to chassis 130 and abutting against carrier 1640. In this instance, actuator 1625 may be inhibited from returning to an initial position relative to chassis 130 upon protrusion 1642 abutting against protrusion 1628, thereby securely fixing actuator 1625 and causing shroud 120 to be locked from movement relative to housing 102. In this instance, shroud 120 may be locked in the retracted position relative to housing 102. In other embodiments, protrusion 1628 and protrusion 1642 may be collectively configured to reduce a blowback force onto shroud 120 as generated at canister 150 in response the pressurized medium being released into valve assembly 160.

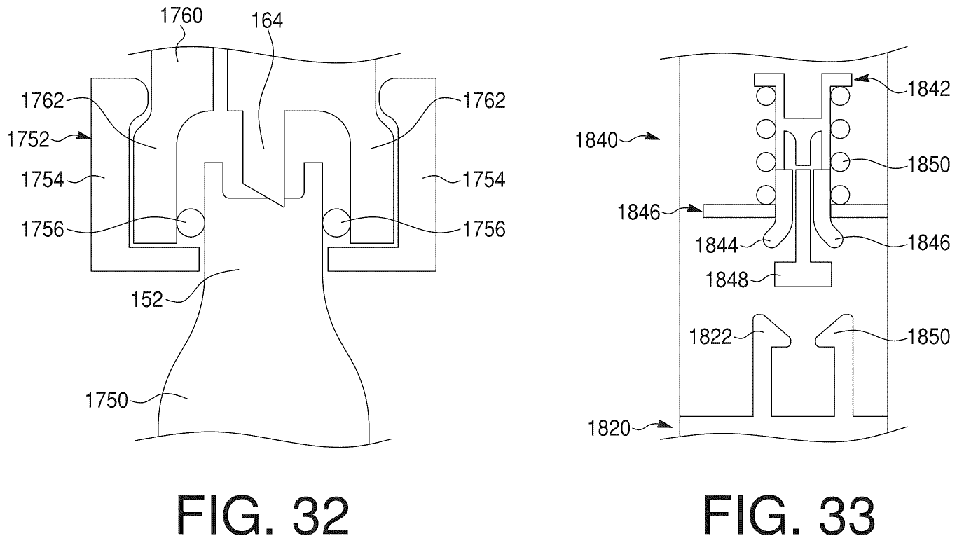

Referring now to FIG. 32, another exemplary canister 1750 and valve assembly 1760 are depicted. It should be appreciated that canister 1750 and valve assembly 1760 may be incorporated into auto-injector 100 in a substantially similar manner as canister 150 and valve assembly 1760 shown and described above, respectively, except for the differences explicitly noted herein. For example, canister 1750 may include a retainer cap 1752 having a scaling surface 1754 that is coupled to neck 152, and a seal 1756 (e.g., an O-ring) disposed about neck 152. Valve assembly 1760 may include an interface surface 1762 disposed about piercing mechanism 164. Each of retainer cap 1752 and seal 1756 may be configured to fluidly coupling canister 1750 to valve assembly 160. For example, sealing surface 1754 may be configured to contact an exterior of interface surface 1762 and seal 1756 may be configured to contact an interior of interface surface 1762. Accordingly, retainer cap 1752 and seal 1756 may define a two-part sealing system for canister 1750. In some embodiments, sealing surface 1754 may be configured to clip onto interface surface 1762, and retainer cap 1752 may provide sufficient resistance for holding against pressure applied to seal 1756 from the pressurized medium released from canister 1750.

Referring now to FIG. 33, another exemplary shroud 1820 and carrier 1840 are depicted. It should be appreciated that shroud 1820 and carrier 1840 may be incorporated into auto-injector 100 in a substantially similar manner as shroud 120 and carrier 140 shown and described above, respectively, except for the differences explicitly noted herein. For example, shroud 1820 may include a pair of retention mechanisms 1822 (e.g., clips, hooks, arms, tabs, etc.), and carrier 1840 may include a movable body 1842 having a pair of arms 1844, a fixed body 1846, a locking pin 1848, and a biasing mechanism 1850 (e.g., a spring) disposed between movable body 1842 and fixed body 1846. Locking pin 1848 may be disposed between the pair of arms 1844, and the pair of arms 1844 may be disposed within an opening through fixed body 1846. The pair of arms 1844 may be at least partially flexible and locking pin 1848 may be configured to fix the pair of arms 1844 to fixed body 1846 when disposed between arms 1844. In other words, with locking pin 1848 disposed between arms 1844, locking pin 1848 may be configured to bend arms 1844 radially-outwards relative to one another. With arms 1844 received through an opening of fixed body 1846, locking pin 1848 may be configured to push the pair of arms 1844 against fixed body 1846, thereby securely coupling movable body 1842 to fixed body 1846.

Shroud 1820 may be disposed relatively below carrier 1840, and retention mechanisms 1822 may be configured to engage locking pin 1848 upon movement of shroud 1820 in an upwards direction towards carrier 1840. Upon retention mechanisms 1822 engaging locking pin 1848, an occurrence of an early-lift event may cause shroud 820 to move in a downwards direction relative to carrier 1840. With retention mechanisms 1822 engaged with locking pin 1848, shroud 1820 may be configured to pull locking pin 1848 downwards relative to fixed body 1846, thereby decoupling locking pin 1848 from between the pair of arms 1844. In this instance, with locking pin 1848 no longer position in between the pair of arms 1844, a clearance gap may be formed between the pair of arms 1844 to allow for lateral movement of arms 1844 towards one another and into the clearance gap. The pair of arms 1844 may be configured to bend radially-inwards towards one another due to the clearance gap formed therebetween due to the removal of locking pin 1848. As such, a force applied to movable body 1842 by biasing mechanism 1850 may cause arms 1844 to bend towards one another, thereby minimizing a cross-sectional profile of arms 1844 to allow arms 1844 to extend through the opening of fixed body 1846. As biasing mechanism 1850 expands, the pair of arms 1844 may be configured to move (e.g., translate) vertically through the opening of fixed body 1846 in an upwards direction with movable body 1842 as biasing mechanism 1850 expands. Biasing mechanism 1850 may be fixed in the expanded configuration, thereby locking out auto-injector 100 from subsequent use.

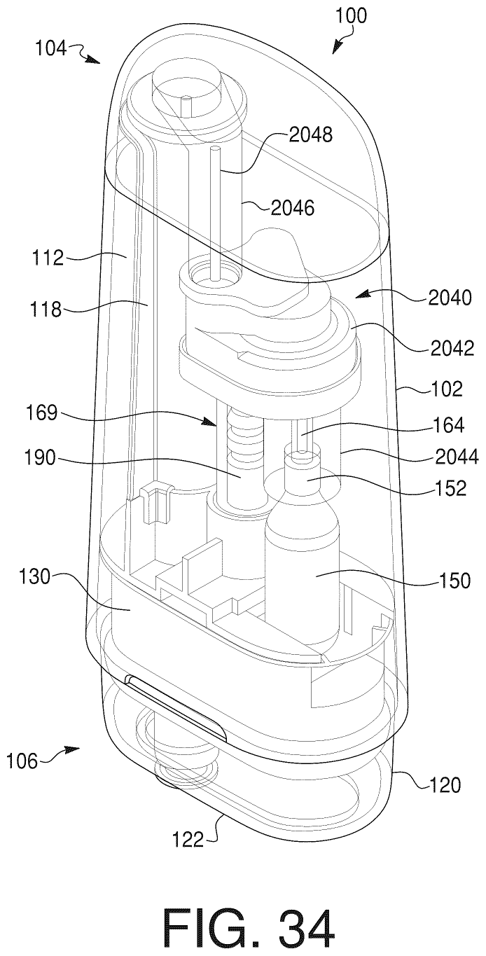

Referring to FIG. 34, an exemplary valve assembly 2040 of auto-injector 100 is depicted. It should be appreciated that valve assembly 2040 may be incorporated into auto-injector 100 in a substantially similar manner as valve assembly 160 shown and described above except for the differences explicitly noted herein. For example, valve assembly 2040 may include a valve body 2042 having a first (top) portion 2044 and a second (bottom) portion 2046 that are arranged within housing 102 in a compact configuration, thereby providing a reduced cross-sectional profile of housing 102 relative to valve assembly 160.

In the example, first (top) portion 2044 may be configured to receive at least a portion of canister 150, such as neck 152, to facilitate fluid communication with piercing mechanism 164. Valve assembly 2040 may include release outlet 169 with slidable piston 190 movably disposed therein. Second (bottom) portion 2046 may be disposed vertically above first (top) portion 2044, such that an arrangement of valve body 2042 may be configured to form a reduced cross-sectional profile within housing 102 relative to valve assembly 160 shown and described above. In this instance, diaphragm 2012 of valve assembly 2040 (not shown) may be oriented within valve body 2042 in a horizontal configuration such that diaphragm 2012 is arranged to move vertically along its axis of action. Stated differently, a body of diaphragm 2012 may be arranged along a horizontal plane that is transverse relative to a longitudinal axis of housing 102.

Still referring to FIG. 34, auto-injector 100 may omit carrier 140 entirely such that canister 150 may be moved into fluid communication with valve assembly 2040 directly by actuator 125 (not shown). By positioning canister 150 and slidable piston 190 within first (top) portion 2044 of valve body 2042, valve assembly 2040 may be operable to provide a smaller sealed area and higher strength relative to valve assembly 160. Second (bottom) portion 2046 may be configured to interface with container 112, thereby reducing a height of housing 102. Second (bottom) portion 2046 may be in fluid communication with container 112 via a conduit 2048.

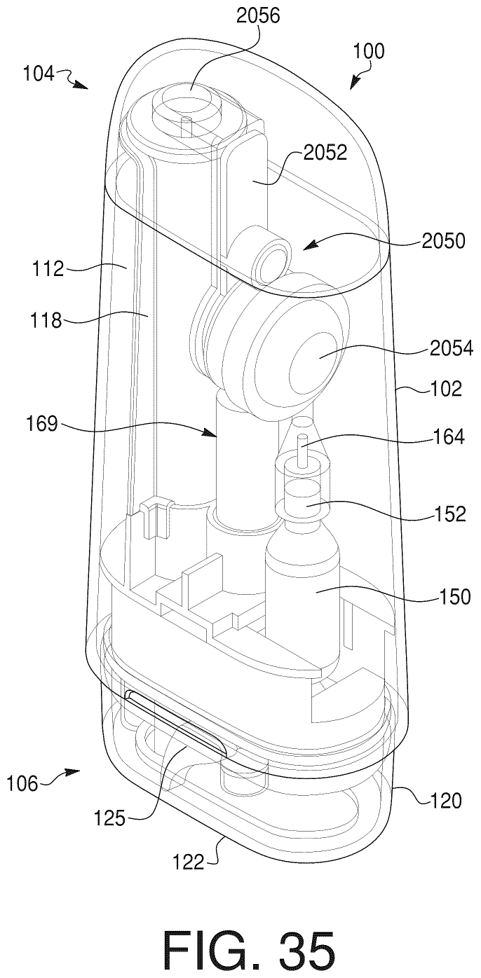

Referring to FIG. 35, another exemplary valve assembly 2050 of auto-injector 100 is depicted. It should be appreciated that valve assembly 2050 may be incorporated into auto-injector 100 in a substantially similar manner as valve assembly 160 shown and described above except for the differences explicitly noted herein. For example, valve assembly 2050 may include a valve body 2052 having a first (top) portion 2054 and a second (bottom) portion 2056 that are arranged within housing 102 in a compact configuration, thereby providing a reduced cross-sectional profile of housing 102 relative to valve assembly 160.

In the example, first (top) portion 2054 may be configured to receive at least a portion of canister 150, such as neck 152, to facilitate fluid communication with piercing mechanism 164. Valve assembly 2050 may include release outlet 169 with slidable piston 190 (not shown) movably disposed therein. Second (bottom) portion 2056 may be disposed horizontally and/or radially relative to first (top) portion 2054, such that an arrangement of valve body 2052 may be configured to form a reduced cross-sectional profile within housing 102 relative to valve assembly 160 shown and described above. In this instance, diaphragm 2012 of valve assembly 2050 (not shown) may be oriented within valve body 2052 in a vertical configuration such that diaphragm 2012 is arranged to move horizontally along its axis of action. Stated differently, a body of diaphragm 2012 may be arranged along a vertical plane that is parallel relative to a longitudinal axis of housing 102.

Still referring to FIG. 35, auto-injector 100 may omit carrier 140 entirely such that canister 150 may be moved into fluid communication with valve assembly 2050 directly by actuator 125. By positioning canister 150 within first (top) portion 2054 and slidable piston 190 within valve body 2052, valve assembly 2050 may be operable to provide a smaller sealed area and higher strength relative to valve assembly 160. Second (bottom) portion 2056 may be configured to interface with container 112, thereby reducing a height of housing 102.

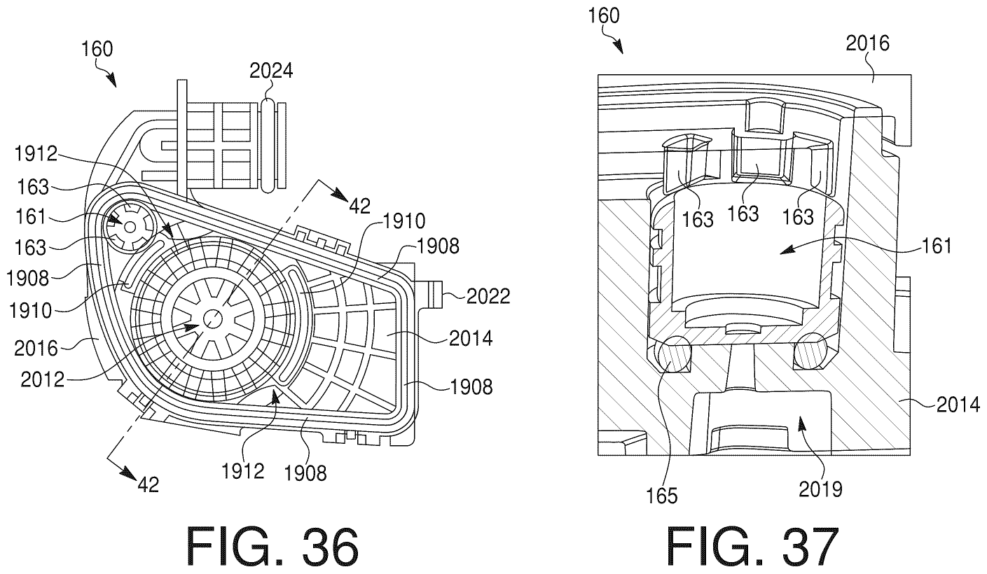

Referring now to FIG. 36, valve assembly 160 of auto-injector 100 is depicted. As described in detail herein, valve assembly 160 may include a high pressure (first) inlet 2022, a low pressure (second) inlet 2024, and diaphragm 2012 positioned between a low pressure body portion 2014 and a high pressure body portion 2016 (see FIGS. 44-46). In some embodiments, low pressure body portion 2014 may be attached to high pressure body portion 2016 via various suitable means and along one or more locations therebetween to securely couple diaphragm 2012 therein. For example, low pressure body portion 2014 may be attached to high pressure body portion 2016 via one or more weld paths to affix any one of diaphragm 2012, low pressure body portion 2014, and high pressure body portion 2016 to one another. Each of the one or more weld paths may extend along a predefined portions within valve assembly 160. In some embodiments, the one or more weld paths may be continuous without intermediate interruptions, while in other embodiments the one or more weld paths may include one or more interruptions forming stress relief gaps within the interior of valve assembly 160.

In the example, valve assembly 160 may include a first (outer) weld path 1908 disposed about an exterior perimeter of valve assembly 160, and particularly along an interface edge between low pressure body portion 2014 and high pressure body portion 2016. By continuously extending about the exterior perimeter, first weld path 1908 may be configured and operable to seal low pressure body portion 2014 to high pressure body portion 2016. As shown and described herein, first weld path 1908 may be further configured to securely couple diaphragm 2012 between low pressure body portion 2014 and high pressure body portion 2016 (see FIG. 41).

Still referring to FIG. 36, valve assembly 160 may include one or more second (inner) weld paths 1910 disposed along portions of valve assembly 160 located radially inwards from first (outer) weld path 1908. Second weld paths 1910 may be configured and operable to seal diaphragm 2012, low pressure body portion 2014, and high pressure body portion 2016 to each other within valve assembly 160. In the example, valve assembly 160 may include a pair of second weld paths 1910 disposed about diaphragm 2012. Each of the pair of second weld paths 1910 may be sized, shaped, and/or otherwise configured to form at least one stress relief gap 1912 about diaphragm 2012. Stress relief gaps 1912 may be configured and operable as a pressure relief mechanism for valve assembly 160 such that, during operation of auto-injector 100, stress relief gaps 1912 may be configured to release a portion of the pressurized gas received within valve assembly 160, such as when the pressure generated within valve assembly 160 reaches a predetermined threshold. In this instance, stress relief gaps 1912 may be operable to prevent an excessive accumulation of pressure within valve assembly 160, and particularly between a high pressure (first) cavity 2018 and a low pressure (second) cavity 2020 of valve assembly 160, as described in greater detail below (see FIGS. 44-46).

It should be appreciated that additional and/or fewer first weld paths 1908 and/or second weld paths 1910 may be included in valve assembly 160 without departing from a scope of this disclosure. Additionally and/or alternatively, first weld path 1908 and/or second weld paths 1910 may be positioned along various other portions of valve assembly 160, and may have various other suitable shapes and/or configurations than those shown and described herein.

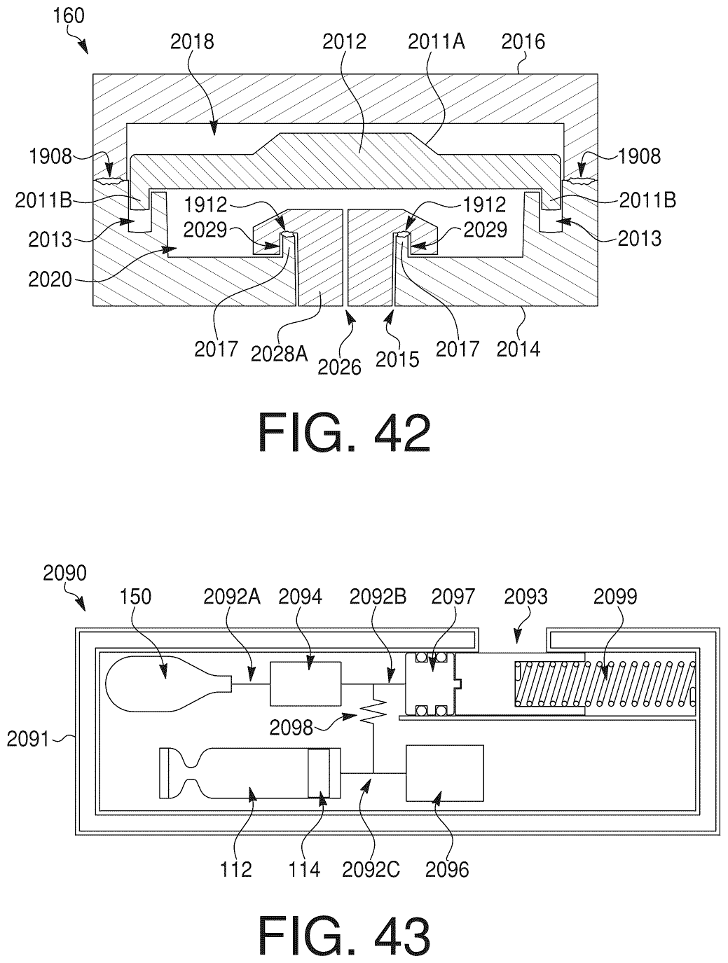

As best seen in FIG. 42, outer edges of low pressure body portion 2014 and high pressure body portion 2016 may be welded to one another via first (outer) weld path 1908 with diaphragm 2012 disposed therebetween. In some embodiments, diaphragm 2012 may include a central body 2011A with an outer rim 2011B extending outwardly from central body 2011A and about a periphery of diaphragm 2012, and particularly along a bottom surface of diaphragm 2012. In the example, low pressure body portion 2014 may include a corresponding cavity 2013 that is sized, shaped, and/or otherwise configured to receive outer rim 2011B, thereby coupling diaphragm 2012 to low pressure body portion 2014. Diaphragm 2012 may include a raised portion located at a radially center position of central body 2011A with a thickness protruding outward from central body 2011A at a distance that is greater than extension of outer rim 2011B.

As described in detail below, valve assembly 160 may include a valve seat 2028 formed along low pressure body portion 2014 (see FIGS. 44-46). Valve seat 2028 may be integral with low pressure body portion 2014, such that valve seat 2028 and low pressure body portion 2014 form a unitary body. In other embodiments, as seen in FIG. 42, the valve seat may include a vent insert 2028A coupled to low pressure body portion 2014. In this instance, vent insert 2028A may extend into an interior of valve assembly 160 between diaphragm 2012 and low pressure body portion 2014, such as within low pressure (second) cavity 2020. In the example, low pressure body portion 2014 may include a central opening 2015 that is sized, shaped, and/or otherwise configured to receive vent insert 2028A.

Still referring to FIG. 42, vent insert 2028A may include a cavity 2029 extending about a periphery of vent insert 2028A, and particularly along a bottom surface of vent insert 2028A. In the example, low pressure body portion 2014 may include a corresponding inner ledge 2017 that is sized, shaped, and/or otherwise configured to extend into cavity 2029, thereby coupling vent insert 2028A to low pressure body portion 2014. Vent insert 2028A may be coupled to low pressure body portion 2014 via a third weld path 1914 disposed between inner ledge 2017 and cavity 2029. Vent insert 2028A may further include a conduit 2026. Conduit 2026 may be formed within vent insert 2028A, and extends into the interior of valve assembly 160, and particularly low pressure cavity 2020. Vent insert 2028A may be sized and/or shaped such that conduit 2026 includes a reduced diameter relative to conduit 2026 of valve seat 2028, described further herein (see FIGS. 44-46). By providing conduit 2026 with the reduced diameter, vent insert 2028A may have a smaller cross-sectional profile relative to valve seat 2028, thereby facilitating diaphragm 2012 having a corresponding reduced diameter between low pressure body portion 2014 and high pressure body portion 2016.

Referring now to FIG. 37, valve assembly 160 may include an orifice assembly 161 (e.g., a pressure restrictor) disposed between low pressure body portion 2014 and high pressure body portion 2016. Orifice assembly 161 may be configured to restrict a flow of the pressurized fluid received in and subsequently released out of valve assembly 160, such as to container 112. High pressure body portion 2016 may include a plurality of retention teeth 163 abutting against a top surface of orifice assembly 161, thereby securely coupling orifice assembly 161 between low pressure body portion 2014 and high pressure body portion 2016. The plurality of retention teeth 163 may be configured to inhibit movement of orifice assembly 161 relative to low pressure body portion 2014 and high pressure body portion 2016 during use of auto-injector 100, and particularly during the release of pressurized fluid from canister 150 and through orifice assembly 161. Low pressure body portion 2014 may include a channel 2019 in fluid communication with orifice assembly 161 via an opening 166, such that pressurized fluid from low pressure body portion 2014 may travel into orifice assembly 161 via channel 2019 and through opening 166. Valve assembly 160 may further include a gasket 165 (e.g., O-ring) disposed beneath and in contact with orifice assembly 161, thereby inhibiting the pressurized fluid entering orifice assembly 161 at opening 166 from being redirected around an exterior of orifice assembly 161 and towards channel 2019.

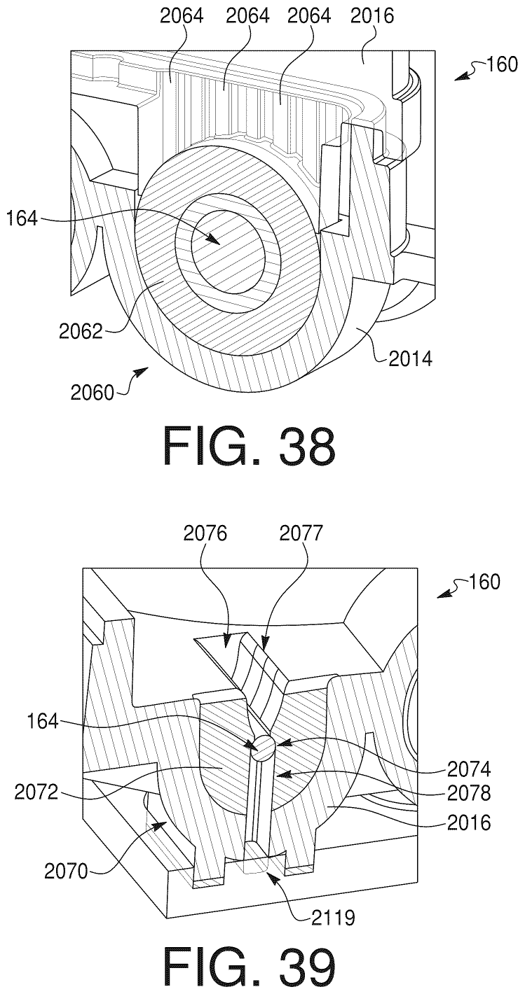

Referring now to FIGS. 38-40, exemplary retention mechanisms of valve assembly 160 are depicted. Specifically, the exemplary retention mechanisms of valve assembly 160 define a surrounding interface of piercing mechanism 164, which is configured and operable to establish fluid communication with canister 150 during use of auto-injector 100 as described above. The exemplary retention mechanisms may be configured and operable to retain, stabilize, and/or seal piercing mechanism 164 during use of auto-injector 100. It should be appreciated that any one of the exemplary retention mechanisms shown and described herein may be incorporated in auto-injector 100 without departing from a scope of this disclosure.

Referring specifically to FIG. 38, valve assembly 160 may include a first retention mechanism 2060 for retaining piercing mechanism 164. In the example, first retention mechanism 2060 may include a gasket 2062 (e.g., O-ring) disposed about an exterior of piercing mechanism 164, and high pressure body portion 2016 may include a plurality of retention teeth 2064 abutting against a top surface of gasket 2062, thereby securely coupling gasket 2062 about piercing mechanism 164. Gasket 2062 may be further configured to inhibit the pressurized fluid released from canister 150 and into valve assembly 160 via piercing mechanism 164 from being redirected about the exterior of piercing mechanism 164 and out of valve assembly 160. The plurality of retention teeth 2064 may be configured to inhibit movement of gasket 2062, and gasket 2062 may be configured to inhibit radial movement of piercing mechanism 164 relative to low pressure body portion 2014 and high pressure body portion 2016 during use of auto-injector 100, and particularly during connection with canister 150 and/or release of pressurized fluid from canister 150 into valve assembly 160.

In another example, as seen in FIG. 39, valve assembly 160 may include a second retention mechanism 2070 for retaining piercing mechanism 164. In the example, second retention mechanism 2070 may include a body 2072 disposed about an exterior of piercing mechanism 164. In the example, body 2072 may be integral with low pressure body portion 2014, such that body 2072 and low pressure body portion 2014 may form a unitary body. In other embodiments, body 2072 of second retention mechanism 2070 may be a separate from and selectively coupled to low pressure body portion 2014. Body 2072 may include a channel and/or lumen 2074 extending therethrough, and lumen 2074 may be sized, shaped, and/or otherwise configured to receive piercing mechanism 164 therethrough. In the example, a diameter of lumen 2074 may correspond to a diameter of piercing mechanism 164, thereby securely coupling piercing mechanism 164 to body 2072. Second retention mechanism 2070 may further include a cutout 2076 formed along a top surface of body 2072. Cutout 2076 may extend into body 2072 and towards lumen 2074. Cutout 2076 may include one or more grooves 2077 having an angled and/or tapered configuration or surface that extends radially inwards towards lumen 2074. In the example, cutout 2076 may be sized, shaped, and/or otherwise configured to receive and guide piercing mechanism 214 into lumen 2074. In some embodiments, the angled and/or tapered profile of grooves 2077 may be configured to inhibit movement of piercing mechanism 164 relative to body 2072, and particularly out of lumen 2074 upon receipt therein.

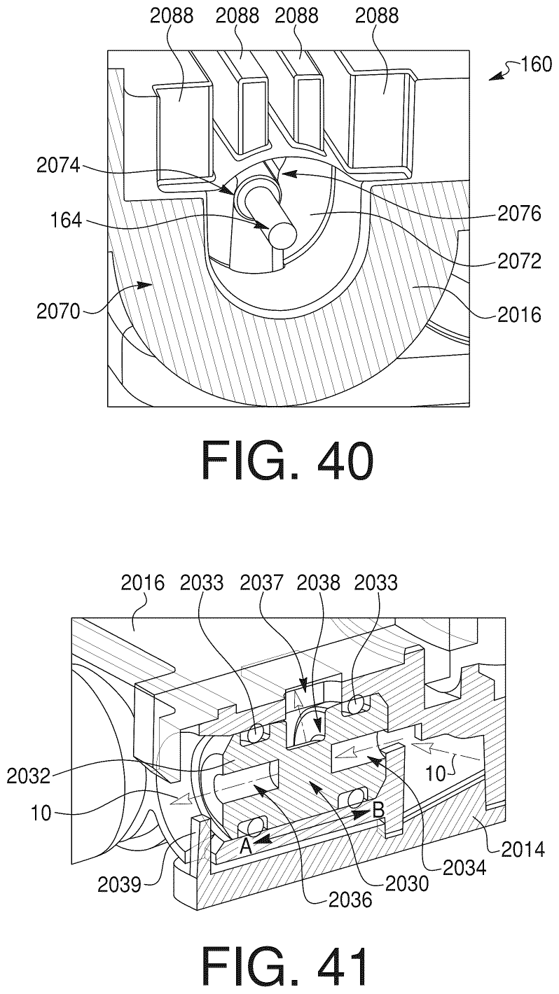

Retention mechanism 2070 may further include a first channel 2078 formed at least partially along body 2072, and a second channel 2119 formed at least partially along low pressure body portion 2014. First channel 2078 may extend from lumen 2074, and may define an undercut used to form the retention mechanism 2070. Second channel 2119 may extend below body 2072, and may define an undercut region within high pressure body portion 2016 and below body 2072 for forming a weld path between second retention mechanism 2070 and high pressure body portion 2016. As illustrated in FIG. 40, valve assembly 160 may also include one or more canister retention mechanisms 2088 (e.g., a retention boss) for retaining canister 150.

Referring to FIG. 41, and as described in further detail below, valve assembly 160 may be in fluid communication with a venting system 2030. In some embodiments, venting system 2030 may be disposed within valve assembly 160, while in other embodiments venting system 2030 may be separate from and fluidly coupled to valve assembly 160. In the example, venting system 2030 may be disposed within valve assembly 160 between low pressure body portion 2014 and high pressure body portion 2016. Venting system 2030 may be fluidly coupled to one or more of high pressure (first) cavity 2018 and/or low pressure (second) cavity 2020.

Venting system 2030 may be configured to release (e.g., vent) a pressure generated within valve assembly 160 upon completing delivery of a medicament from auto-injector 100. For example, venting system 2030 may be configured to move (e.g., translate) in a first direction A and/or a second direction B relative to low pressure body portion 2014 and high pressure body portion 2016 between one or more positions to vent the pressure in valve assembly 160. Venting system 2030 may be operable to selectively define one or more fluid paths 10 for a pressurized fluid to travel through valve assembly 160 and for release into an interior cavity of auto-injector 100 and/or a surrounding atmosphere when moving between the one or more positions. In the example, venting system 2030 may include a body 2032 and a pair of gaskets 2033 disposed about an exterior of body 2032. Body 2032 may include a first opening 2034 and a second opening 2036 that is positioned along an opposing end of body 2032 from first opening 2034.

Still referring to FIG. 41, first opening 2034 and second opening 2036 may be in fluid communication and aligned with one another along a longitudinal axis of body 2032. First opening 2034 may define an activation line of venting system 2030 for receiving the pressurized fluid along flow path 10 through body 2032. Body 2032 may have a third opening 2038 that is in fluid communication with first opening 2034, and aligned transversely relative to first opening 2034 and second opening 2036. Each of the openings of venting system 2030 may define a portion of fluid path 10 based on a relative position of body 2032 within valve assembly 160. For example, as seen in FIG. 41, body 2032 may be positioned relative to valve assembly 160 such that third opening 2038 is aligned with a vent slot 2037 of high pressure body portion 2016. In this instance, the pressurized fluid received in body 2032 along flow path 10 may exit third opening 2038 and enter vent slot 2037 to vent valve assembly 160. Venting system 2030 may include an impediment 2039 (e.g., a retention arm) extending from low pressure body portion 2014 to restrict movement (e.g., translation) of body 2032 in the first direction A, such as when venting system 2030 is in the position shown in FIG. 41 with third opening 2038 aligned with vent slot 2037. It should be appreciated that body 2032 may be configured to move (e.g., translate) in response to valve assembly 160 receiving the pressurized fluid from canister 150.

Referring now to FIG. 43, an exemplary pressure sensing system 2090 of auto-injector 100 is schematically depicted. Pressure sensing system 2090 may include a housing 2091 with container 112, canister 150, a first accumulator 2094, a second accumulator 2096, a pressure restrictor 2098, a piston 2097, and a biasing mechanism 2099 (e.g., a spring) disposed therein. In the example, canister 150 may be in fluid communication with first accumulator 2094 via a first conduit 2092A, and first accumulator 2094 may be in fluid communication with each of pressure restrictor 2098 and piston 2097 via a second conduit 2092B. Accordingly, the pressurized fluid released from canister 150 may be delivered to first accumulator 2094 via first conduit 2092A. First accumulator 2094 and second accumulator 2096 may each be operable as an energy storage device, such as a pressure storage reservoir in which a pressurized medium (e.g., gas) received therein may be held under a sufficient pressurization that is applied by an external source. For example, first accumulator 2094 may be configured to receive, store, and release the pressurized fluid from canister 150 to second conduit 2092B. In some embodiments, first accumulator 2094 may be configured to deliver the pressurized fluid to second conduit 2092B at a pressurization that is relatively higher than an original pressurization received from canister 150.

At least a first portion of the pressurized fluid may be released from first accumulator 2094 towards piston 2097 via second conduit 2092B, thereby causing piston 2097 to move (e.g., translate). In the example, piston 2097 may be urged by biasing mechanism 2099 towards a first position, as shown in FIG. 43. In response to receiving the pressurized fluid from first accumulator 2094, piston 2097 may be configured to compress biasing mechanism 2099 and move towards a second position. At least a second portion of the pressurized fluid may be released from first accumulator 2094 towards pressure restrictor 2098 via second conduit 2092B. Pressure restrictor 2098 may be configured to restrict the flow of pressurized fluid received from first accumulator 2094 via the second conduit 2092B prior to the pressurized fluid entering a third conduit 2092C of pressure sensing system 2090. Accordingly, pressure restrictor 2098 may define a high pressure flow area at second conduit 2092B and a low pressure flow area at third conduit 2092C. In some embodiments, pressure restrictor 2098 may include a porous material, a serpentine channel, and/or an orifice assembly.

Still referring to FIG. 43, at least a first portion of the pressurized fluid received in third conduit 2092C by pressure restrictor 2098 may be received by and stored in second accumulator 2096, and at least a second portion of the pressurized fluid received in third conduit 2092C by pressure restrictor 2098 may be received at container 112. In particular, the pressurized fluid received at container 112 may be operable to move (e.g., urge) piston 114 within container 112, thereby delivering the medicament stored therein out of container 112. In the example, biasing mechanism 2099 may be sized and/or configured to set a predetermined ending pressure such that biasing mechanism 2099 will return to an expanded configuration upon the pressurization within pressure sensing system 2090 dropping below a predetermined threshold (e.g., 150 psi). Stated differently, biasing mechanism 2099 may be configured to expand and move (e.g., urge) piston 2097 in an opposite direction as the flow of the pressurized fluid through pressure sensing system 2090 decreases. It should be appreciated that a pressurization within pressure sensing system 2090 may gradually decay as the medicament stored in container 112 is expelled in response to movement of piston 114 therein.

In the example, housing 2091 may include a window 2093 positioned such that as piston 2097 translates, window 2093 may be operable to provide visual feedback of the state of the auto-injector 100 from a visualization of a relative position of piston 2097 within auto-injector 100. In some examples, piston 2097 may include one or more markings and/or other indicia along an exterior surface of piston 2097 that may be visible through window 2093, with the one or more markings being indicative of various operating states of auto-injector 100 to a user. In further examples, pressure sensing system 2090 may be configured to generate an audible feedback indicative of the state of auto-injector 100, and particularly upon completing delivery of the medicament from container 112, upon piston 2097 moving (e.g., translating) to a position corresponding to the compressed configuration of biasing mechanism 2099.

Referring now to FIGS. 44-46, an exemplary drive system 2000 of auto-injector 100 is schematically depicted. Drive system 2000 may be configured to provide a drive force to deliver a medicament 20 from container 112 to a patient. Drive system 2000 may include canister 150 (i.e., a fluid source) fluidly coupled to one or more components of auto-injector 100, e.g., needle 116 and valve assembly 160. Drive system 2000 may further include a first high pressure line 2002, a second high pressure line 2004, a low pressure line 2006, a drive line 2008, and a third line 2010. Canister 150 may be operatively coupled to container 112 via first high pressure line 2002 and drive line 2008. Further, canister 150 may be fluidly coupled to valve assembly 160 via one or more of first high pressure line 2002, second high pressure line 2004, and low pressure line 2006. In some embodiments, drive system 2000 may include a pressure reducing mechanism (e.g. orifice) along first high pressure line 2002.

Valve assembly 160 may include diaphragm 2012 positioned between low pressure body portion 2014 and high pressure body portion 2016. Within valve assembly 160, diaphragm 2012 defines a high pressure (first) cavity 2018 and a low pressure (second) cavity 2020. Valve assembly 160 may further include a high pressure (first) inlet 2022, a low pressure (second) inlet 2024, and a conduit 2026. Conduit 2026 is formed within a valve seat 2028 that extends into the interior of valve assembly 160, and particularly low pressure cavity 2020. High pressure cavity 2018 may be in fluid communication with first high pressure line 2002 via second high pressure line 2004 and inlet 2022. Low pressure cavity 2020 may be in fluid communication with low pressure line 2006 via low pressure inlet 2024. Low pressure cavity 2020 may be in fluid communication with third line 2010 via conduit 2026. Drive system 2000 may include a venting system 2030 fluidly connected by a number of fluid lines or conduits. For example, venting system 2030 may be in fluid communication with valve assembly 160 via third line 2010. Venting system 2030 may be configured to vent drive system 2000 by releasing pressurized fluid into an interior cavity of auto-injector 100 and/or into the atmosphere. As described above, venting system 2030 may include slidable piston 190 of auto-injector 100.

Still referring to FIGS. 44-46, container 112 may include top end 112A and bottom end 112B. Container 112 may include a cavity 112C having an opening at top end 112A and extending towards bottom end 112B. Bottom end 112B may include a stopper 112D configured to assist with closing and/or sealing bottom end 112B, and allow for needle 116 to be fluidly coupled to container 112 at bottom end 112B. Stopper 112D may include a line seal, a protrusion disposed in a neck of container 112 (e.g., to reduce dead volume), and/or a septum formed of an uncoated bromobutyl material, or another suitable material. Cavity 112C may be closed at top end 112A by piston 114. Piston 114 may include a fluoropolymer coated bromobutyl material, and, in some embodiments, may include a conical nose to help reduce dead volume within container 112. Piston 114 may include one or more rubber materials such as, e.g., halobutyls (e.g., bromobutyl, chlorobutyl, florobutyl) and/or nitriles, among other materials. As described in detail above, container 112 may store a medicament 20 (e.g., drug, fluid substance, etc.) within cavity 112C.

Canister 150 may include a non-latching can or a latching can. Canister 150 may be configured to dispense liquid propellant for boiling outside of canister 150 so as to provide a pressurized gas (vapor pressure) that acts on piston 114 in container 112. In some embodiments, canister 150 may include a pressurized gas that is released from canister 150 and acts on piston 114 in container 112. In some embodiments, once opened, the latching can may be latched open so that the entire contents of propellant is dispensed therefrom. Alternatively, in some embodiments, canister 150 may be selectively controlled, including selectively activated and deactivated. For example, in an alternative embodiment, the flow of pressurized gas from canister 150 may be stopped after flow is initiated.

The fluid from canister 150 may be any suitable propellant for providing a vapor pressure to drive piston 114 relative to container 112. In some embodiments, canister 150 may be a high-pressure canister configured to hold a compressed gas. In certain embodiments, the propellant may be or contain a hydrofluoroalkane (“HFA”), for example HFA134a, HFA227, HFA422D, HFA507, or HFA410A. In certain embodiments, the propellant may be or contain a hydrofluoroolefin (“HFO”) such as HFO1234yf or HFO1234ze, an organic gas (e.g., carbon dioxide (CO₂), etc.), a cryogenic gas (e.g., Argon (Ar), Helium (He), etc.), a hydrocarbon gas (e.g., propane, butane, propylene, ethane, methane, etc.) or an inorganic gas (e.g., Ammonia, Nitrogen Dioxide (NO₂), Nitrous Oxide (N₂O), etc.).

FIG. 44 shows the pre-activated state of auto-injector 100. To move auto-injector 100 from the pre-activated state of FIG. 44, canister 150 may be activated to move piston 114 along a longitudinal axis of container 112 towards needle 116. Canister 150 may be actuated so as to move to an open configuration in which propellant may exit canister 150 as a pressurized gas. As described above, canister 150 may be actuated in response to one or more components of valve assembly 160 (e.g., piercing mechanism 164) contacting canister 150. In some embodiments, the actuation is irreversible such that the flow of pressurized gas from canister 150 is unable to be stopped upon releasing the pressurized gas from canister 150.

When canister 150 is actuated, pressurized gas may flow through first high pressure line 2002 and drive line 2008, and then to container 112. Some pressurized gas from first high pressure line 2002 may be diverted to high pressure cavity 2018 via second high pressure line 2004 and high pressure inlet 2022. As seen in FIG. 45, due to a change in pressurization, diaphragm 2012 may move into low pressure cavity 2020 and towards conduit 2026 (e.g., in a first direction), thereby sealing valve seat 2028. Reduced-pressure gas may be diverted to low pressure cavity 2020 via low pressure line 2006 and low pressure inlet 2024. The pressure difference between high pressure cavity 2018 and low pressure cavity 2020 may provide the force required to seal conduit 2026 by diaphragm 2012. Gas flowing through drive line 2008 may initiate movement of piston 114 within container 112 towards needle 116. In particular, with needle 116 in fluid communication with medicament 20 within container 112, activation of canister 150 may apply a pressure against piston 114 contained in cavity 112C, which may then be applied to medicament 20 itself by piston 114 driving towards bottom end 112B. In this instance, as seen in FIG. 45, needle 116 is in fluid communication with the contents of container 112 (e.g., medicament 20). As the gas from drive line 2008 urges piston 114 towards bottom end 112B, medicament 20 is expelled from container 112 until piston 114 reaches bottom end 112B (and bottoms out). As described above and below, the pressurized gas from canister 150 may also drive movement of one or more other components of auto-injector 100 (e.g., indicator assembly 170, slidable piston 190, etc.).

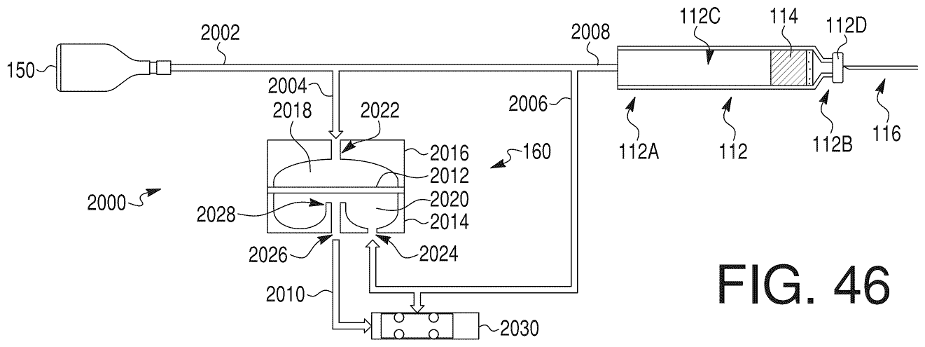

Referring now to FIG. 46, when piston 114 bottoms out at bottom end 112B, the pressure across high pressure cavity 2018 and low pressure cavity 2020 may equilibrate, thereby causing diaphragm 2012 to lift off of valve seat 2028 and open conduit 2026. In this instance, diaphragm 2012 may move in a second direction that is opposite of the first direction. This may allow the gas from low pressure line 2006 to travel to venting system 2030 via conduit 2026 and third line 2010 where the gas may vent out of venting system 2030 (see FIG. 41). It should be appreciated that canister 150 may be configured to contain enough pressurized fluid so that release of the pressurized gas may actuate both movement of piston 114 relative to cavity 112C and the one or more other components of auto-injector 100 (e.g., indicator assembly 170, slidable piston 190, etc.). In some instances, canister 150 may contain excess pressurized gas, i.e., more fluid than is necessary to complete delivery of medicament 20 from container 112. In this instance, release of the pressurized fluid from within drive system 2000 may allow for the movement of one or more components of auto-injector 100 (e.g., shroud 120, actuator 125, mandrel assembly 180, etc.) to initiate retraction of said components relative to housing 102 of auto-injector 100.

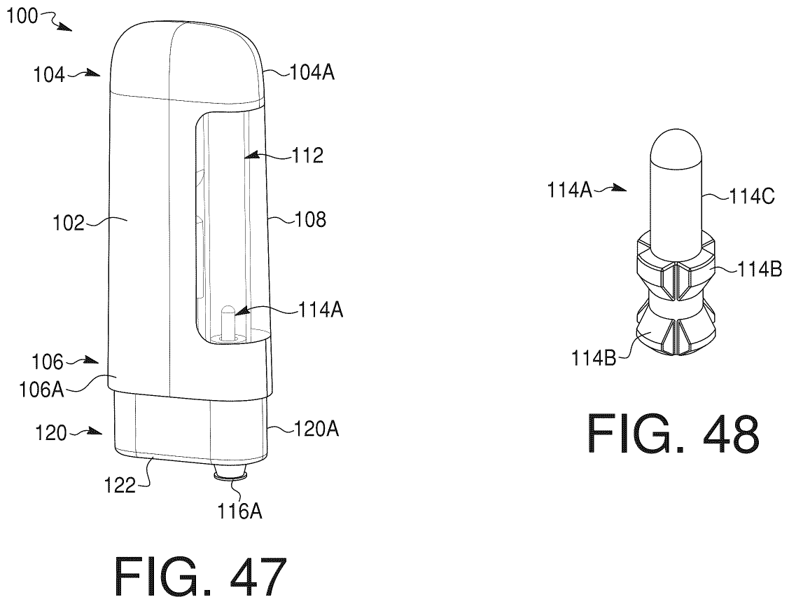

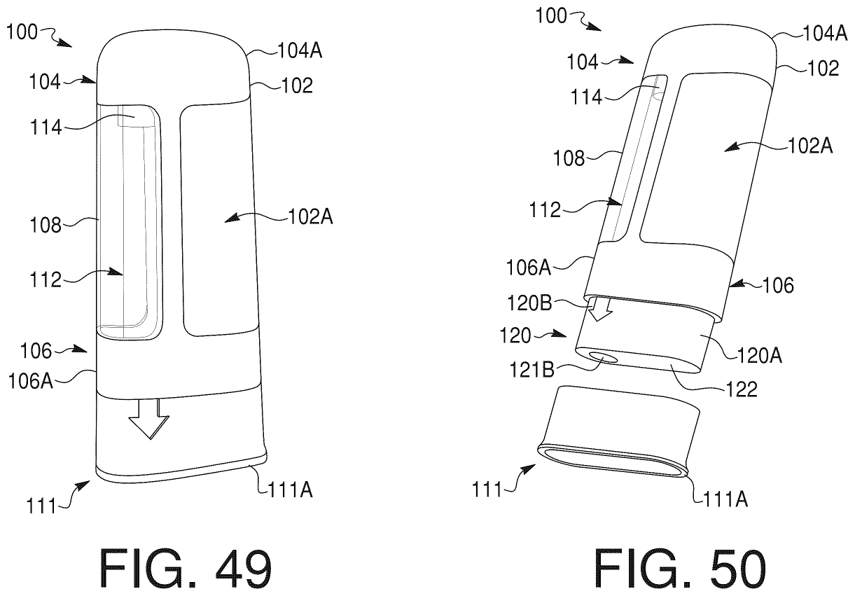

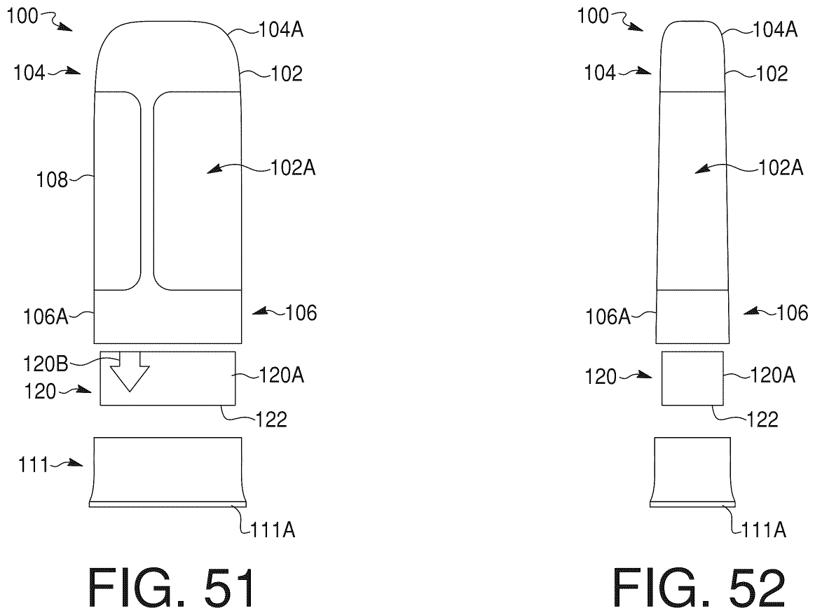

Referring now to FIGS. 47-52, auto-injector 100 is depicted in accordance with an example of the present disclosure. It should be appreciated that a size, a shape, and/or a layout of auto-injector 100, and particularly a vertical upright design of housing 102, may enhance various aspects of auto-injector 100 during use. For example, aspects such as a position and a visibility of first window 108; a size and a position of a secondary indicator (e.g., a piston flag 114A) in container 112; a shape and a size of shroud 120; and a controlled handling of housing 102 by a user may be improved by the vertical upright design of auto-injector 100. In some embodiments, housing 102 may be sized, shaped, and/or otherwise designed to provide an outer surface 102A between top end 104 and bottom end 106 (see FIGS. 49-52) for displaying a label and/or other marking(s) on auto-injector 100. For example, a label on outer surface 102A may provide information indicative of the contents of auto-injector 100, directions for using auto-injector 100, and more. It should be appreciated that outer surface 102A may define a front surface, a rear surface, and/or both of housing 102. In further embodiments, housing 102 may be sized, shaped, and/or otherwise designed to improve a visual dose indication (e.g., a primary indicator, a secondary indicator, etc.); minimize a perceived and/or an actual profile of auto-injector 100; and facilitate an intuitive configuration that is generally less intimidating for a user.

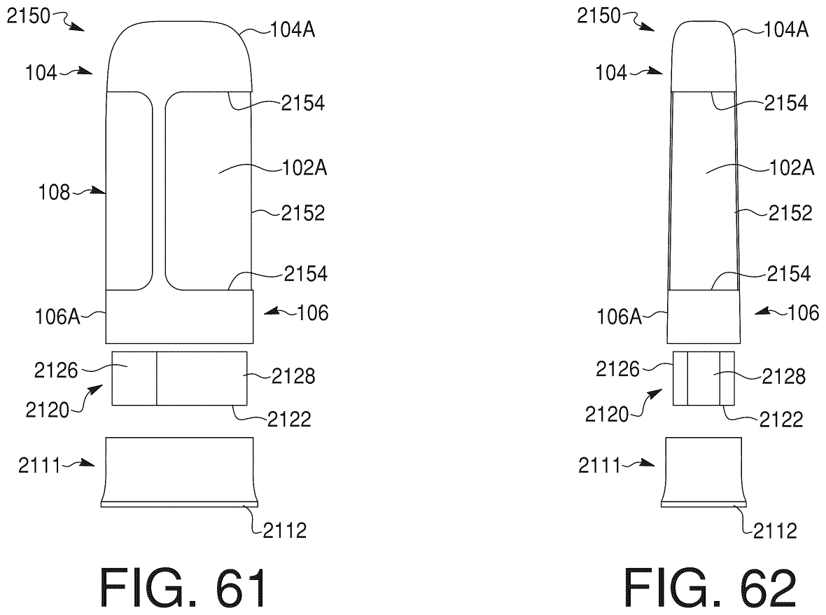

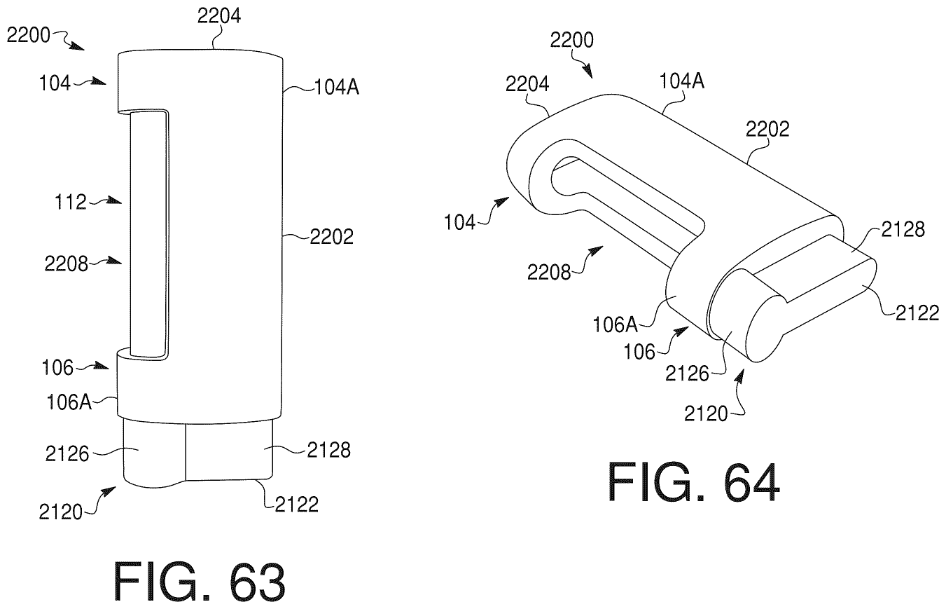

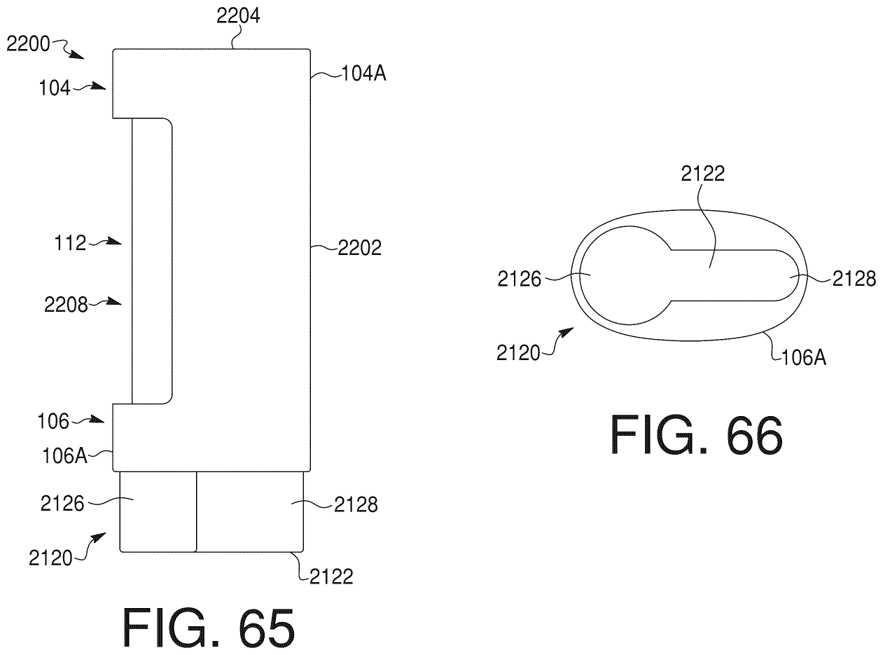

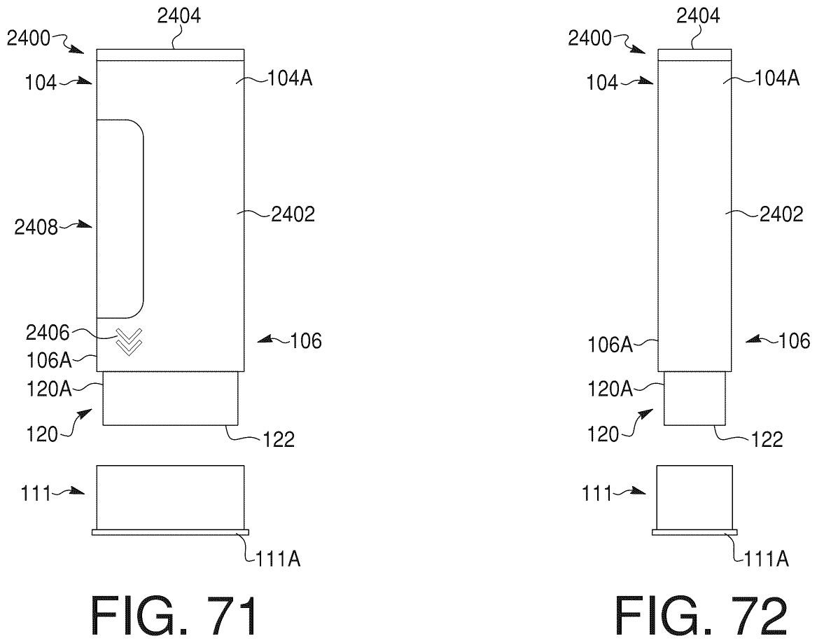

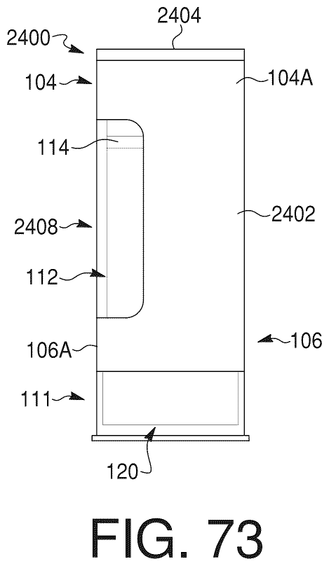

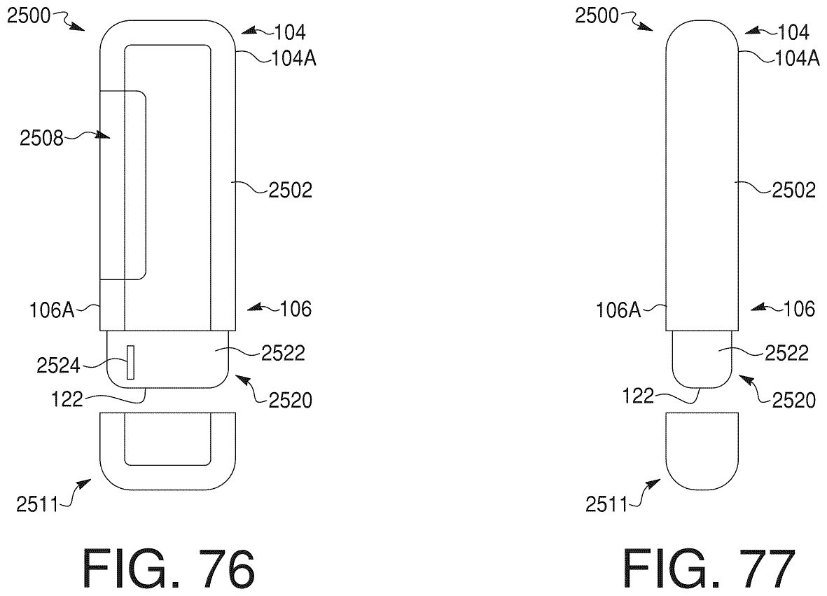

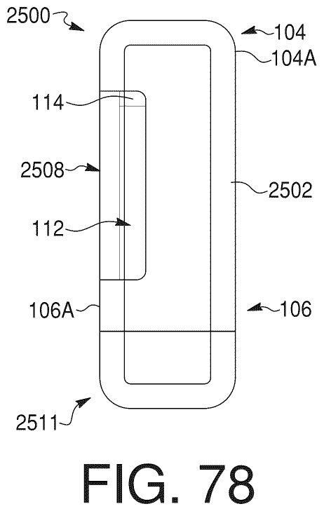

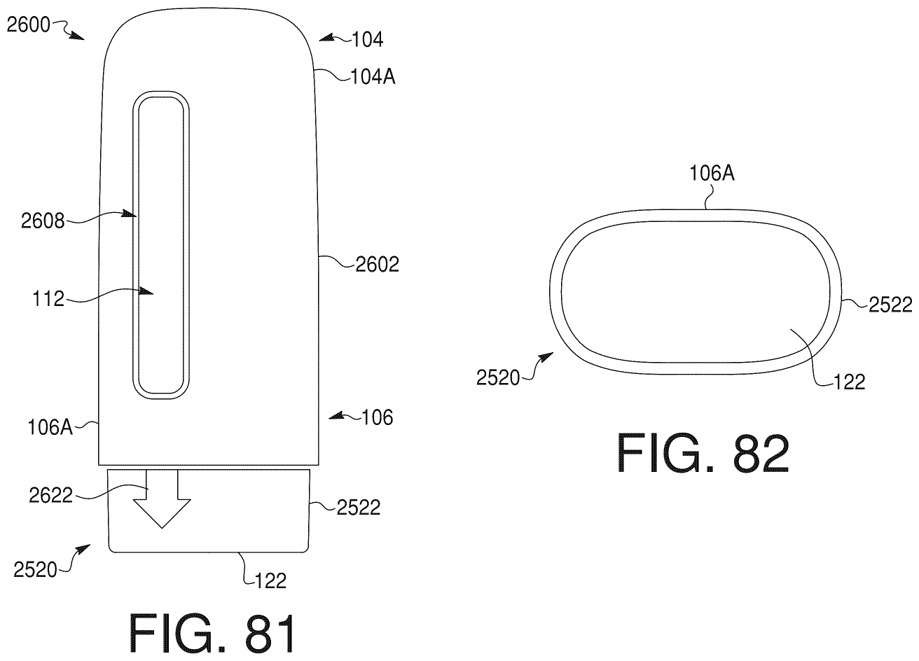

In the example, referring particularly to FIG. 47, a size, a shape, and/or a bulk design of shroud 120 may be relatively minimized. Additionally, a draft angle of housing 102, and particularly an angle of a pair of opposing sidewalls 104A, 106A of housing 102 between top end 104 and bottom end 106, may be minimized such that sidewalls 104A, 106A are generally parallel to one another. In some embodiments, top end 104 may have a domed and/or rounded configuration (see FIGS. 47-62 and 74-83), and in other embodiments top end 104 may have a flattened and/or planar configuration (see FIGS. 63-66 and 69-73).

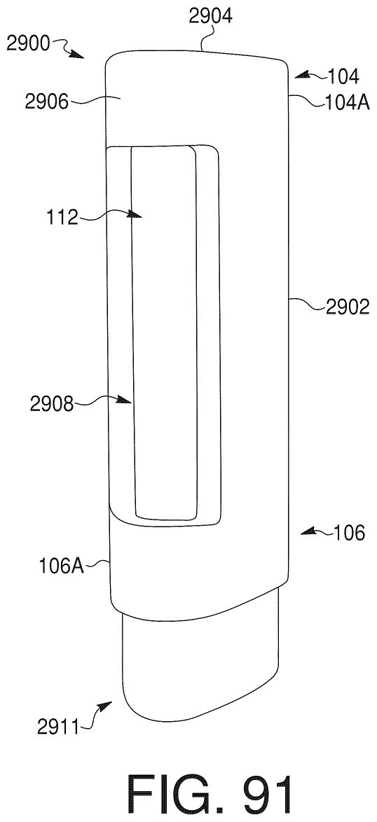

In further embodiments, the housing may generally have an irregular shape, such as a trilobe profile in which a cylindrical form of container 112 disposed within the housing may be emphasized (see FIGS. 91-93). In some embodiments, housing 102 may be formed of a clear-molding with one or more frosted elements, whereas in other embodiments housing 102 may be formed of a clear-molding with a back-sprayed finish. As described herein, one or more components of auto-injector 100 may include corresponding or distinguishing colors from one another to facilitate intuitive use and/or minimize a perceived intimidation in using auto-injector 100. As described in further detail below, auto-injector 100 may include one or more recesses, cavities, edges, ridges, and/or grip elements along an exterior of housing 102 that may be configured to enhance control and a frictional texture of housing 102.

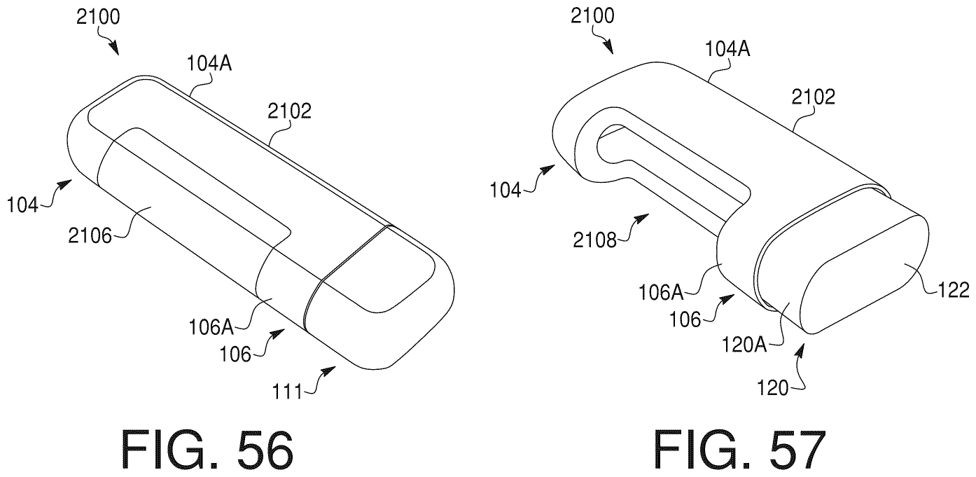

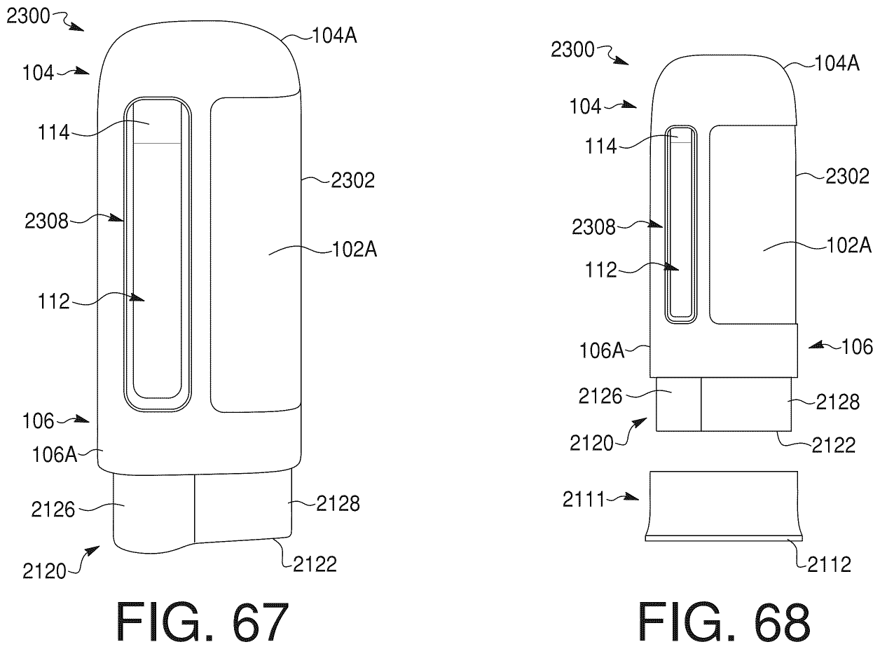

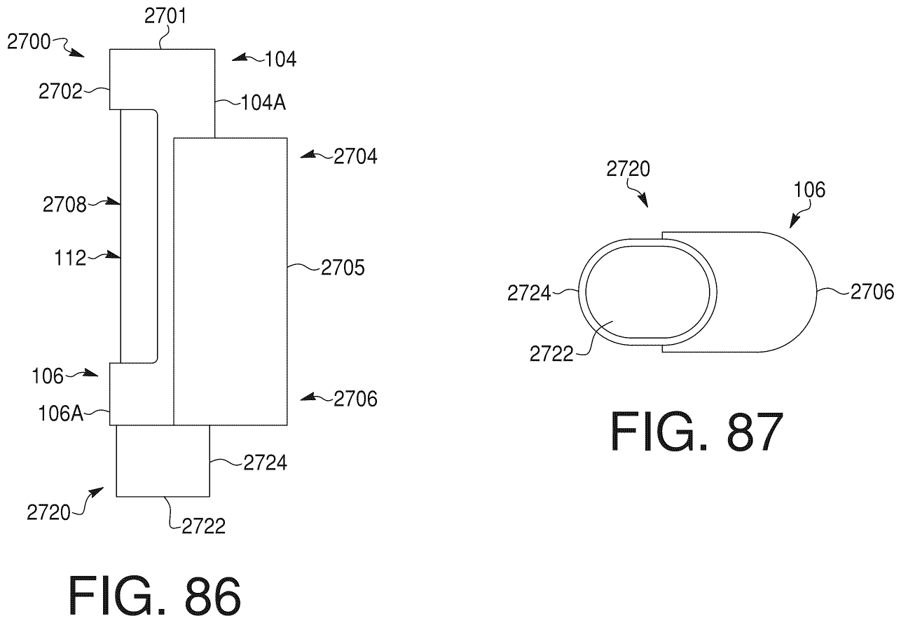

Auto-injector 100 may be configured to improve a visual dose indication with piston 114 having a vibrant coloring scheme (e.g. red, yellow, orange) that is visible through container 112 via first window 108. First window 108 may be disposed inboard along housing 102 (see FIGS. 67-68 and 79-83) in lieu of an outer side of housing 102 to further improve the visual dose indication. Still further, auto-injector 100 may omit an outer window lens disposed over first window 108 entirely (see FIGS. 56-57, 63-66, 79-93). In some embodiments, a secondary indicator may be provided within and/or along an exterior of housing 102 (see FIGS. 47-48, 53A-53C, 56 and 91-93). In additional embodiments, the pressurized fluid (e.g., gas) released into container 112 may be colored and easily visible via first window 108. Auto-injector 100 may be configured to facilitate an intuitive configuration that is generally less intimidating with an incorporation of softer, matte colors displayed along an exterior surface of housing 102, one or more markings (e.g., directional graphics) displayed along the exterior of housing 102 to indicate use directions or locations of a needle (see FIGS. 50-53C, 69-71, 79-83), and/or vibrant colors displayed along an exterior surface of housing 102.

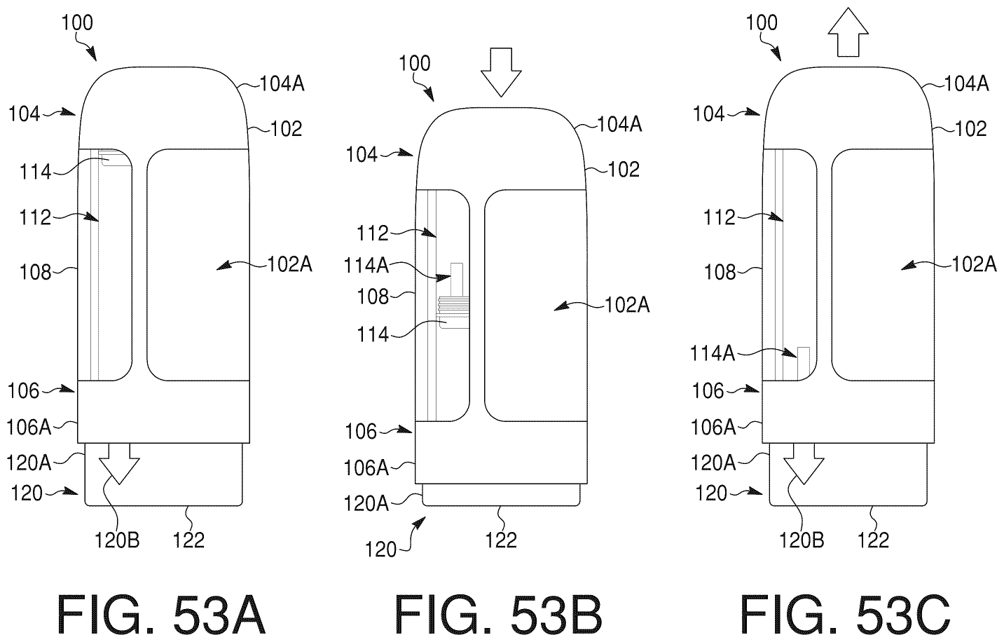

Still referring to FIG. 47, auto-injector 100 may include one or more secondary indicators. In the example, the secondary indicator may include a piston flag 114A (e.g., a peg) that is configured and operable to attach to piston 114 (see FIGS. 53A-53C). Piston flag 114A may be sized, shaped, and/or otherwise configured to provide a visual feedback of a dose indication during use of auto-injector 100. Stated differently, with piston flag 114A coupled to piston 114, piston flag 114A may be operable to visually indicate a current position of piston 114 relative to container 112, thereby indicating a dose delivery state of auto-injector 100. In some embodiments, piston flag 114A may include one or more colors, symbols, and/or other visual elements that may be operable for enhancing a visibility of piston flag 114A within container 112 via first window 108.

As shown in FIG. 48, piston flag 114A may include a plurality of threads 114B that may be configured to engage an interior lumen of piston 114, thereby threadably coupling piston flag 114A thereto. Piston flag 114A may further include a top end 114C that may be sized, shaped, and/or otherwise configured to extend outwardly from piston 114 when piston flag 114A is coupled thereto. In other words, top end 114C may be disposed outside of piston 114, and thereby easily visible from first window 108, when piston flag 114A is coupled to piston 114. In the example, top end 114C may have a cylindrical configuration, however, in other embodiments top end 114C may have various other suitable sizes, shapes, and/or configurations.

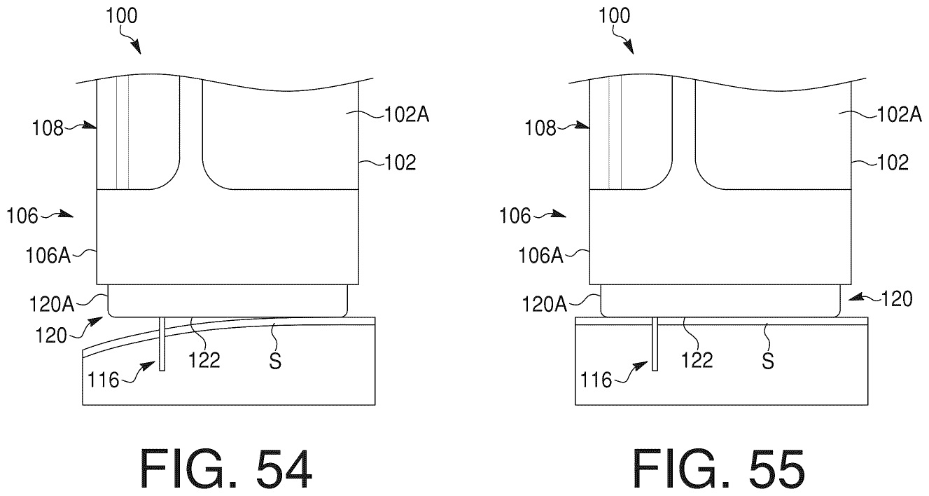

Referring to FIGS. 49-52, auto-injector 100 may include a rounded and/or domed configuration at top end 104, a window lens disposed over first window 108 for encapsulating container 112 within housing 102, and shroud 120 may include one or more markings 120B configured and operable to facilitate use of auto-injector 100. In the example, marking 120B may include a directional graphic (e.g., an arrow) indicating a direction of movement of housing 102 relative to shroud 120 for delivering a dose. Additionally and/or alternatively, marking 120B may be positioned along an exterior portion of shroud 120 to indicate a relative location of the needle (e.g., needle 116) disposed within shroud 120 to facilitate a placement of shroud 120 on a skin surface of the user (see FIGS. 54-55). In this instance, marking 120B may be configured to indicate an accurate alignment of needle 116 at the intended injection site as needle 116 remains disposed inside shroud 120.

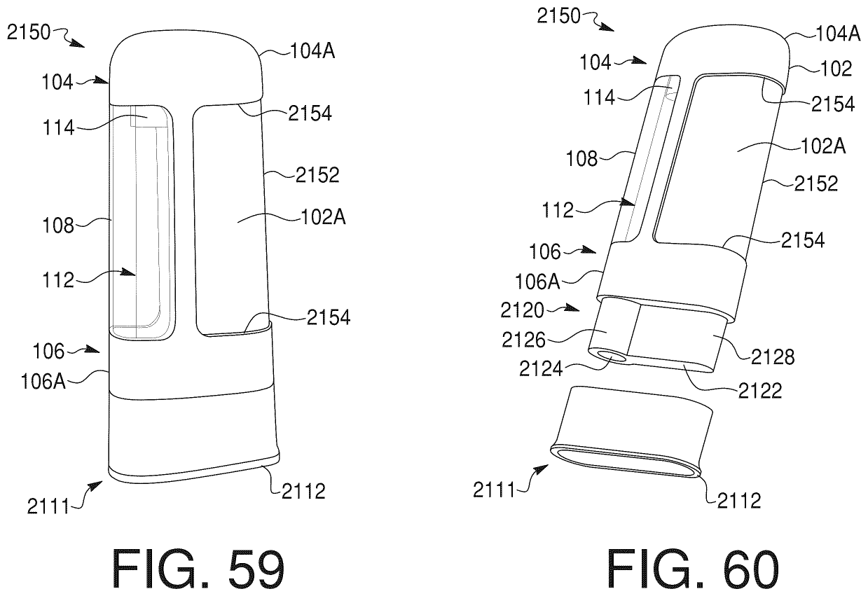

Shroud 120 may include a body 120A that is sized, shaped, and/or otherwise designed to have a cross-sectional dimension that substantially corresponds to a profile of housing 102 at bottom end 106. In other words, body 120A of shroud 120 may coincide with a cross-sectional profile of housing 102, and particularly bottom end 106. In other embodiments, as shown and described herein, a shroud may have a cross-sectional profile that varies relative to the housing for facilitating control of the shroud and/or providing an indication of a location of the needle disposed therein (see FIGS. 59-68 and 84-93).

Auto-injector 100 may further include one or more soft colors along an exterior of housing 102, and a side label disposed along outer surface 102A. In some embodiments, a draft angle of housing 102 may be approximately 1 degree between top end 104 and bottom end 106. In other word, the pair of opposing sidewalls 104A, 106A of housing 102 may be substantially parallel and/or minimally angled relative to one another. In the example, outer surface 102A may be flush with the adjacent exterior surfaces of housing 102. In other examples, as shown and described herein, outer surface 102A may be recessed and/or raised relative to the adjacent exterior surfaces of housing 102 for facilitating receipt of a label and/or enhanced grip of auto-injector 100. In some embodiments, housing 102 may have a soft pebble shape, surface, and/or texture along top end 104 for enhancing a gripping interface of auto-injector 100 by a user.

Cap 111 may be removably coupled to housing 102 at bottom end 106, with shroud 120 disposed therein. Cap 111 may include a bottom end 111A that is tapered and/or flared radially outward relative to the remaining portions of cap 111. Bottom end 111A may be configured to facilitate case in grasping cap 111, such as during removal of cap 111 from housing 102 to expose shroud 120 prior to dose delivery. In some embodiments, bottom end 111A may include a geometric flare that extends radially outward from cap 111, whereas in other embodiments bottom end 111A may include an extended edge or ridge.

In exemplary use, as seen in FIGS. 53A-53C, piston flag 114A may be configured to translate (vertically) through container 112 as piston 114 translates relative to container 112 to deliver a dose, in response to shroud 120 moving relative to housing 102. Prior to dose delivery, with shroud 120 in an extended position relative to housing 102, piston flag 114A may not be visible from first window 108 as piston 114 is maintained at a first position adjacent to top end 104, as seen in FIG. 53A. In this instance, a location of piston flag 114A may be indicative of auto-injector 100 being a new device that has not been previously activated. During dose delivery as shroud 120 is retracted into housing 102, as seen in FIG. 53B, piston flag 114A may be visible through first window 108 as piston 114 translates (vertically) downward through container 112. In this instance, a location of piston flag 114A may be indicative of auto-injector 100 being in use for delivering a portion of the dose stored in container 112. Upon delivering the dose and shroud 120 returning to the extended position relative to housing 102, as seen in FIG. 53C, piston flag 114A may be repositioned adjacent to bottom end 106, thereby indicating auto-injector 100 as being a used device.

FIGS. 54-55 depict auto-injector 100 positioned against an injection site, and more specifically a skin surface S of a user (e.g., a patient). In some instances, the injection site may be located along a region of the user's body at which skin surface S may be curved (FIG. 54), and in other instances the injection site may be located along a region where skin surface S may be substantially flat and/or planar (FIG. 55). Shroud 120 may be sized, shaped, and/or otherwise configured to minimize a cross-sectional profile of shroud 120 and maximize a distance at which needle 116 may extend outwardly from shroud 120, when shroud 120 is retracted into housing 102, to thereby maximize a depth at which needle 116 extends through skin surface S. Stated differently, shroud 120 may be sized to have a length, a width, and a depth that collectively define a minimal cross-sectional dimension of shroud 120, thereby allowing needle 116 to extend outwardly from shroud 120 to a maximum extent. As such, auto-injector 100 may be operable for use at injection sites that are either curved and/or flat while maintaining sufficient insertion depth of needle 116 through skin surface S. In other embodiments, as shown and described herein, shroud 120 may have various other suitable configurations for identifying an insertion point of needle 116 to allow the user to accurately position shroud 120 at a location along skin surface S for maximizing the insertion depth of needle 116.

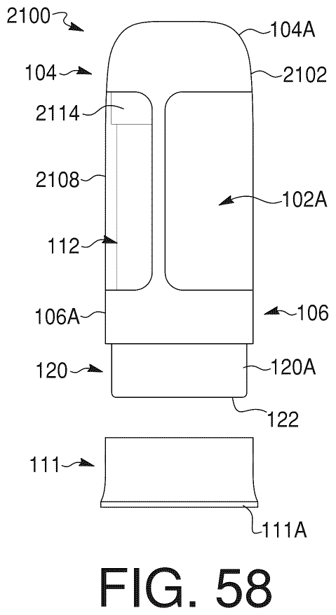

Referring to FIGS. 56-58, another exemplary auto-injector 2100 is depicted in accordance with an example of this disclosure. It should be appreciated that auto-injector 2100 may be configured, operable, and substantially similar to auto-injector 100 shown and described above except for the differences explicitly noted herein. As such, similar reference numerals of auto-injector 100 are used herein to identify similar components of auto-injector 2100. In the example, auto-injector 2100 may include a housing 2102 having a longitudinal length defined between top end 104 and bottom end 106, with shroud 120 movably coupled to bottom end 106 (FIG. 57). As best seen in FIG. 56, auto-injector 2100 may include cap 111 removably coupled to bottom end 106, thereby disposing shroud 120 therein, and a cover 2106 removably disposed over a window 2108 of housing 2102 (see FIG. 58). Cap 111 may be configured to inhibit inadvertent movement of shroud 120 prior to use of auto-injector 2100, when cap 111 is coupled to bottom end 106. Cover 2106 may be configured to inhibit inadvertent damage and/or contact with container 112 prior to use of auto-injector 2100, when cover 2106 is coupled to housing 2102. As described below, auto-injector 2100 may be configured such that window 2108 is not covered by an outer window lens, thereby exposing container 112, upon removal of cover 2106.

As best seen in FIG. 57, window 2108 may be positioned along at least one of the opposing sidewalls 106A of housing 2102. In other words, window 2108 may be formed along an outer edge of housing 2102. Window 2108 may define a cutout region in housing 2102 for receiving container 112, such that auto-injector 2100 may not include an outer window lens and/or barrier disposed over window 2108. In other embodiments, as seen in FIG. 58, window 2108 may include an outer window lens concealing container 112 within housing 2102. Additionally, auto-injector 2100 may include a secondary indicator in the form of a flag 2114 positioned within window 2108. Flag 2114 may be sized, shaped, and/or otherwise configured substantially similar to piston flag 114A shown and described above. Accordingly, flag 2114 may be configured to translate relative to container 112 as auto-injector 2100 is in use to provide a dose indication to the user. In the example, top end 104 of housing 102 may have a rounded and/or curved form, and housing 2102 may include a matte and/or a textured label disposed on outer surface 102A for enhancing a controlled grasp of auto-injector 2100 by a user. In some embodiments, housing 2102 may have a reduced draft configuration along its longitudinal length between top end 104 and bottom end 106 for enhancing an ergonomic profile of auto-injector 2100. In other words, the pair of opposing sidewalls 104A, 106B of housing 2102 may be minimally angled relative to one another.

Referring to FIGS. 59-62, another exemplary auto-injector 2150 is depicted in accordance with an example of this disclosure. It should be appreciated that auto-injector 2150 may be configured, operable, and substantially similar to auto-injector 100 shown and described above except for the differences explicitly noted herein. As such, similar reference numerals of auto-injector 100 are used herein to identify similar components of auto-injector 2150. In the example, auto-injector 2150 may include a housing 2152 having a longitudinal length defined between top end 104 and bottom end 106, first window 108 extending along at least one sidewall 106A, and outer surface 102A as described in detail above. In the example, top end 104 may include a sculpted pebble shape, surface, and/or texture for enhancing a gripping interface of housing 2152 by a user. Housing 2152 may have a side draft angle of approximately 1 degree between top end 104 and bottom end 106, and particularly along the pair of opposing sidewalls 104A, 106A of housing 2152. Housing 2152 may have a front draft angle of approximately 0 degrees between top end 104 and bottom end 106, and particularly along outer surface 102A of housing 2152.

In the example, outer surface 102A may be recessed relative to the adjacent exterior surfaces of housing 2152. As such, any labels and/or markings positioned on outer surface 102A may be positioned along a recessed plane relative to the remaining exterior surface of housing 2152. In some embodiments, a grip edge 2154 may be formed around a perimeter of outer surface 102A. Grip edge 2154 may be configured and operable to enhance a gripping interface of housing 2152 during use. In other words, grip edge 2154 may form a ridge for a user's fingers to grasp when manually controlling auto-injector 2150. Auto-injector 2150 may include a shroud 2120 movably coupled to housing 2152 at bottom end 106. Shroud 2120 may have a keyhole profile defined by a bottom surface 2122, a rounded end 2126, and a narrowed end 2128. Rounded end 2126 may have a generally circular and/or cylindrical shape, and may be positioned relative to housing 2152 in alignment with the needle (e.g., needle 116) of auto-injector 2150. Accordingly, rounded end 2126 may be configured to indicate a position of the needle disposed within housing 2152. In other words, rounded end 2126 may be positioned relative to housing 2152 at a location along shroud 2120 that is parallel to and/or coincides with the needle to facilitate case in positioning shroud 2120 at an injection site during use without extending the needle out of shroud 2120 via an opening 2124 on bottom surface 2122.

Still referring to FIGS. 59-62, auto-injector 2150 may include a cap 2111 removably coupled to housing 2152 at bottom end 106. Cap 2111 may include a bottom end 2112 that is tapered and/or flared radially outward relative to the remaining portions of cap 2111 to facilitate case in grasping cap 2111, such as during removal of cap 2111 from housing 2152 to expose shroud 2120. Auto-injector 2150 may further include one or more soft colors along an exterior of each of outer surface 102A and shroud 2120, relative to a color along an exterior of the remaining aspects of housing 2152, to aid a user in easily distinguishing the components of auto-injector 2150 from one another during use.

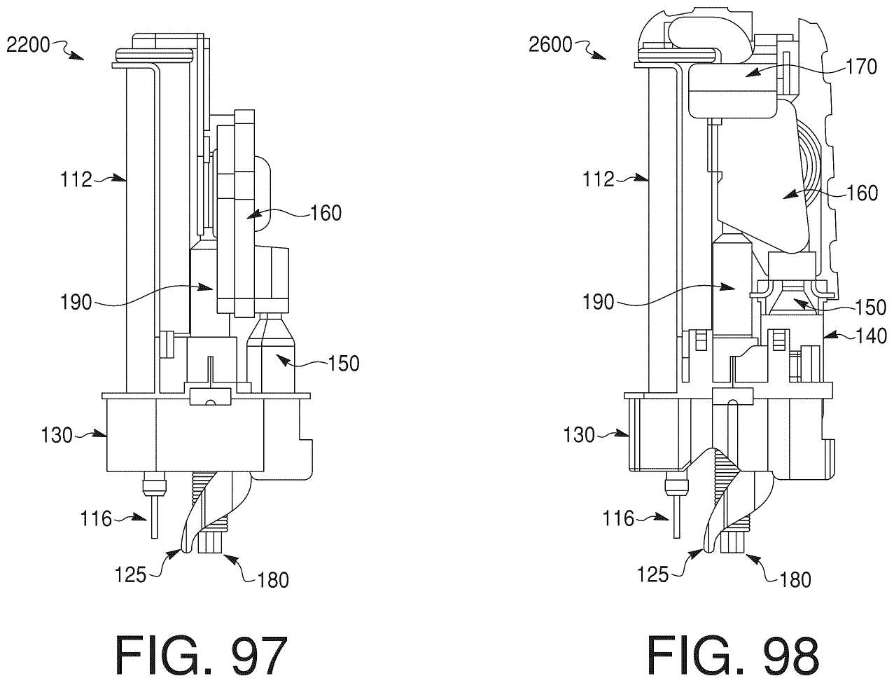

Referring now to FIGS. 63-66, another exemplary auto-injector 2200 is depicted in accordance with an example of this disclosure. It should be appreciated that auto-injector 2200 may be configured, operable, and substantially similar to auto-injectors 100, 2150 shown and described above except for the differences explicitly noted herein. As such, similar reference numerals of auto-injectors 100, 2150 are used herein to identify similar components of auto-injector 2200. For example, as best seen in FIG. 97, auto-injector 2200 may include one or more internal components that are similar to auto-injector 100 (e.g., container 112, needle 116, actuator 125, chassis 130, canister 150, valve assembly 160, mandrel assembly 180, slidable piston 190, and more), however, said components may be positioned and/or oriented in an alternative configuration within a housing 2202 of auto-injector 2200 based on a size and/or shape of auto-injector 2200 as described in detail herein.

In the example, auto-injector 2200 may include housing 2202 having a longitudinal length defined between top end 104 and bottom end 106, with shroud 2120 movably coupled to bottom end 106. Housing 2202 may generally have a rounded cross-sectional profile, thereby providing a curved configuration for auto-injector 2200. Housing 2202 may be defined by the pair of opposing sidewalls 104A, 106A, and may include a planar surface 2204 at top end 104. In some embodiments, a recess, a cavity, and/or an indentation may be formed along planar surface 2204 for facilitating receipt of a finger of a user during use of auto-injector 2200. In this instance, the recess along planar surface 2204 may be configured to enhance a gripping interface of housing 2202 during use of auto-injector 2200.

Auto-injector 2200 may include a window 2208 that is positioned along at least one of the sidewalls 106A of housing 2202. In other words, window 2208 may be formed along an outer edge of housing 2202. Window 2208 may define a cutout in housing 2202 for receiving container 112, such that auto-injector 2200 may not include an outer window lens and/or barrier disposed over window 2208 (see FIG. 64). Shroud 2120 may include a keyhole profile (see FIG. 66) defined by bottom surface 2122, rounded end 2126, and narrowed end 2128 as described in greater detail above. Shroud 2120, and particularly rounded 2126, may be configured to indicate a position of the needle disposed within housing 2202 for improving alignment of auto-injector 2200 at the injection site during use.

Referring to FIGS. 67-68, another exemplary auto-injector 2300 is depicted in accordance with an example of this disclosure. It should be appreciated that auto-injector 2300 may be configured, operable, and substantially similar to auto-injectors 100, 2150 shown and described above except for the differences explicitly noted herein. As such, similar reference numerals of auto-injectors 100, 2150 are used herein to identify similar components of auto-injector 2300. In the example, auto-injector 2300 may include a housing 2302 having a longitudinal length defined between top end 104 and bottom end 106, with shroud 2120 movably coupled to bottom end 106. In the example, top end 104 of housing 2302 may have a rounded and/or curved form, and a draft angle of housing 2302 may be relatively reduced such that the pair of opposing sidewalls 104A, 106A are substantially parallel to one another.

Auto-injector 2300 may include a window 2308 disposed along housing 2302 between top end 104 and bottom end 106. In particular, window 2308 may be positioned inboard within housing 2302. In other words, window 2308 may be formed between the pair of opposing sidewalls 104A, 106A of housing 2302. Positioning window 2308 at a radially offset location from the pair of opposing sidewalls 104A, 106A may enhance a visibility of container 112 and piston 114 disposed therein during use, such as when a user's hand is positioned along the pair of opposing sidewalls 104A, 106A to grasp housing 2302. With window 2308 inboard of housing 2302, an obstruction of window 2308 by a user's hand may be minimized. Window 2308 may define a cutout in housing 2302 for receiving container 112, such that auto-injector 2300 may not include an outer window lens and/or barrier disposed over window 2308.

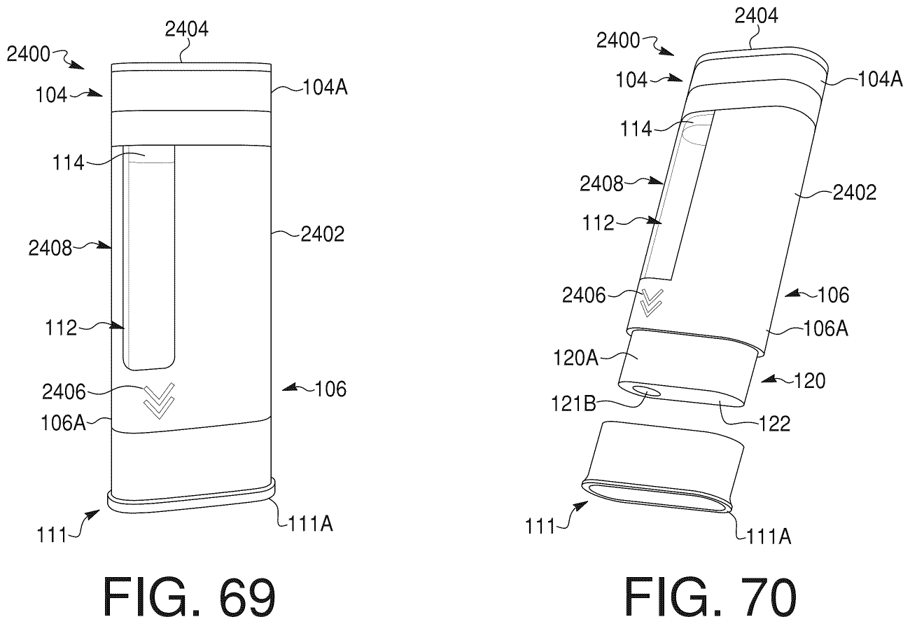

Referring to FIGS. 69-73, another exemplary auto-injector 2400 is depicted in accordance with an example of this disclosure. It should be appreciated that auto-injector 2400 may be configured, operable, and substantially similar to auto-injector 100 shown and described above except for the differences explicitly noted herein. As such, similar reference numerals of auto-injector 100 are used herein to identify similar components of auto-injector 2400. In the example, auto-injector 2400 may include a housing 2402 having a longitudinal length defined between top end 104 and bottom end 106, cap 111 removably coupled to housing 2402 at bottom end 106, and shroud 120 movably coupled to bottom end 106. As best seen in FIG. 69, cap 111 may be configured to conceal shroud 120 when coupled to bottom end 106. Shroud 120 may be exposed upon removing cap 111 from housing 2402, as shown in FIG. 70.

Auto-injector 2400 may have a flattened configuration along top end 104, and particularly a planar surface 2404. Top end 104 may define an elemental form, and the pair of opposing sidewalls 104A, 106A may be aligned substantially parallel to one another. In the example, housing 2402 may include a transparent outer body 2408 along a portion of housing 2402 that coincides with a location of container 112 disposed in housing 2402. Transparent outer body 2408 may be configured and operable to facilitate visual inspection of container 112, and particularly a position of piston 114 relative to container 112 during use of auto-injector 2400. In some embodiments, transparent outer body 2408 may include a window. In the example, auto-injector 2400 may include a marking 2406 on housing 2402, such as adjacent to bottom end 106. Marking 2406 may include a directional graphic (e.g., an arrow) indicating a direction of movement of housing 2402 relative to shroud 120 during use, and/or marking 2406 may be positioned along an exterior portion of housing 2402 to indicate a relative location of the needle (e.g., needle 116) within housing 2402 to facilitate a placement of auto-injector 2400 on a skin surface of the user for accurate alignment of the needle at the intended injection site. In some embodiments, auto-injector 2400 may be configured such that a label (not shown) may be wrapped about an exterior perimeter of housing 2402. In further embodiments, auto-injector 2400 may include a similar coloring scheme along an exterior of each of housing 2402 and shroud 2120.

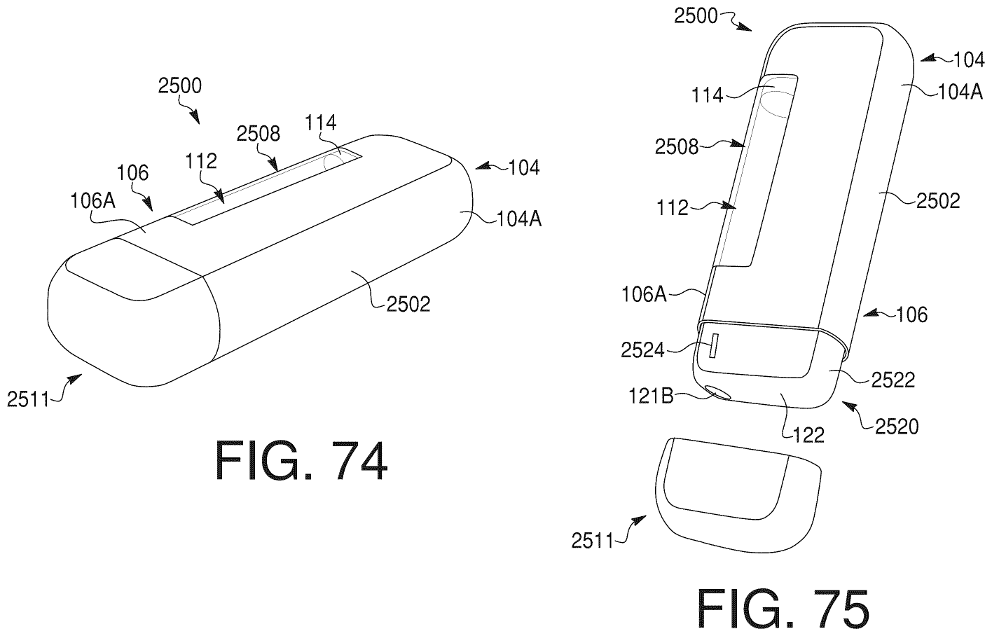

Referring to FIGS. 74-78, another exemplary auto-injector 2500 is depicted in accordance with an example of this disclosure. It should be appreciated that auto-injector 2500 may be configured, operable, and substantially similar to auto-injector 100 shown and described above except for the differences explicitly noted herein. As such, similar reference numerals of auto-injector 100 are used herein to identify similar components of auto-injector 2500. In the example, auto-injector 2500 may include a housing 2502 having a longitudinal length defined between top end 104 and bottom end 106, a cap 2511 removably coupled to housing 2502 at bottom end 106, and a shroud 2520 movably coupled to bottom end 106. As seen in FIG. 74, cap 2511 may be configured to conceal shroud 2520 when coupled to bottom end 106. Shroud 2520 may be exposed upon removing cap 2511 from housing 2502, as shown in FIG. 75.

Cap 2511 may include a body with a rounded and/or curved cross-sectional profile, and shroud 2520 may include a body 2522 having a corresponding rounded and/or curved cross-sectional profile. Auto-injector 2500 may include a rounded configuration along the longitudinal length of housing 2502, and particularly between top end 104 and bottom end 106. The rounded configuration of auto-injector 2500 may enhance a rounded tactility of housing 2502 during use. Auto-injector 2500 may include a window lens disposed over a window 2508 on housing 2502, and the pair of opposing sidewalls 104A, 106A may be substantially parallel to one another. Shroud 2520 may include one or more markings 2524 configured and operable to facilitate use of auto-injector 2500. In the example, marking 2524 may include a directional graphic (e.g., an arrow) indicating a direction of movement for shroud 2520. Additionally and/or alternatively, marking 2524 may be positioned along an exterior portion of shroud 2520 to indicate a relative location of the needle (e.g., needle 116) within shroud 2520 to facilitate a placement of shroud 2520 on a skin surface of the user for accurate alignment of the needle at the intended injection site. In some embodiments, top end 104 may be relatively flat to define a tactile form of housing 2502. Auto-injector 2500 may include a label disposed along one or more exterior surfaces of housing 2502, such as on front and rear surfaces between the pair of opposing sidewalls 104A, 104B.

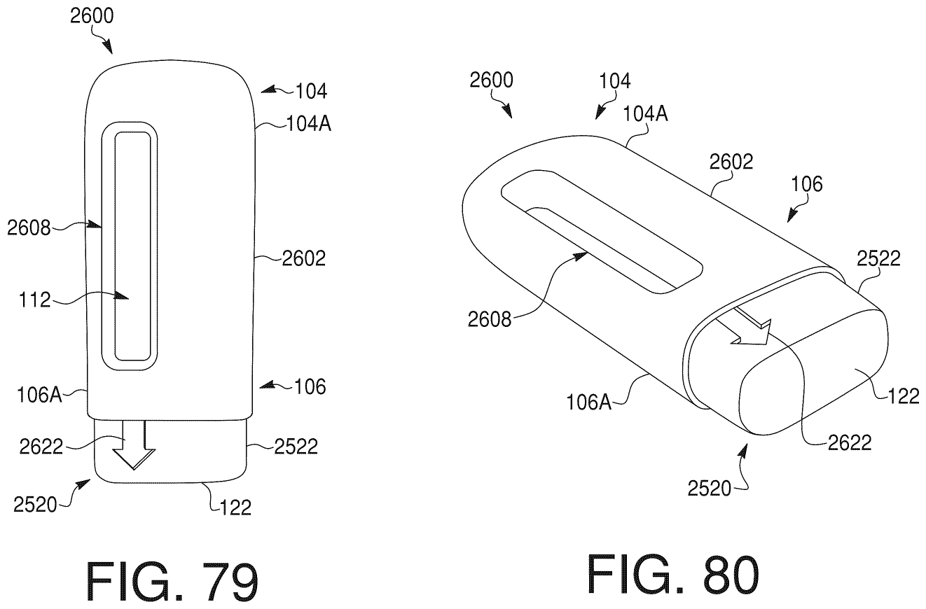

Referring to FIGS. 79-83, another exemplary auto-injector 2600 is depicted in accordance with an example of this disclosure. It should be appreciated that auto-injector 2600 may be configured, operable, and substantially similar to auto-injectors 100, 2500 shown and described above except for the differences explicitly noted herein. As such, similar reference numerals of auto-injectors 100, 2500 are used herein to identify similar components of auto-injector 2600. For example, as best seen in FIG. 98, auto-injector 2600 may include one or more internal components that are similar to auto-injector 100 (e.g., container 112, needle 116, actuator 125, chassis 130, carrier 140, canister 150, valve assembly 160, indicator assembly 170, mandrel assembly 180, slidable piston 190, and more), however, said components may be positioned and/or oriented in an alternative configuration within a housing 2602 of auto-injector 2600 based on a size and/or shape of auto-injector 2600 as described in detail herein.

In the example, auto-injector 2600 may include housing 2602 having a longitudinal length defined between top end 104 and bottom end 106, with shroud 2520 movably coupled to bottom end 106. Housing 2602 may generally have a reduced and/or narrowed cross-sectional profile, thereby providing a slimmed configuration for auto-injector 2600. Housing 2602 may be sized, shaped, and/or otherwise configured such that the pair of opposing sidewalls 104A, 106A are substantially parallel to one another. Each of top end 104 and bottom end 106 may be generally rounded and/or curved. Auto-injector 2600 may include a window 2608 that is inboard within housing 2602. In other words, window 2608 may be formed between the opposing sidewalls 104A, 106A of housing 2602. Window 2608 may define a cutout region in housing 2602 for receiving container 112, such that auto-injector 2600 may not include an outer window lens and/or barrier disposed over window 2608.

Shroud 2520 may include body 2522 as described in greater detail above. Shroud 2520 may include one or more markings 2622 along body 2522 that are configured and operable to facilitate use of auto-injector 2600. In the example, marking 2622 may include a directional graphic (e.g., an arrow) indicating a direction of movement of shroud 2520. Additionally and/or alternatively, marking 2622 may be positioned along an exterior portion of body 2522 to indicate a relative location of the needle (e.g., needle 116) within shroud 2520 to facilitate a placement of shroud 2520 on a skin surface of the user for accurate alignment of the needle at the intended injection site.

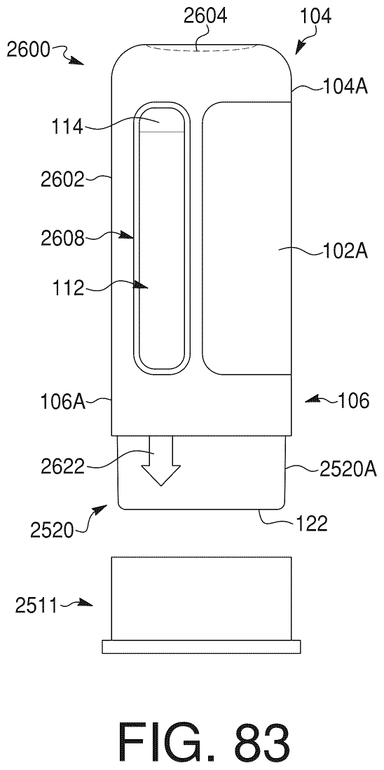

In other embodiments, as best seen in FIG. 83, top end 104 may be generally flat and bottom end 106 may be rounded. In this instance, auto-injector 2600 may have a flattened configuration along top end 104. Top end 104 may include a recess 2604 that is sized, shaped, and/or otherwise configured to enhance a gripping interface of housing 2602 during use of auto-injector 2600. For example, recess 2604 may be sized and/or shaped to receive a finger of a user when grasping top end 104, thereby providing increased control of housing 2602.

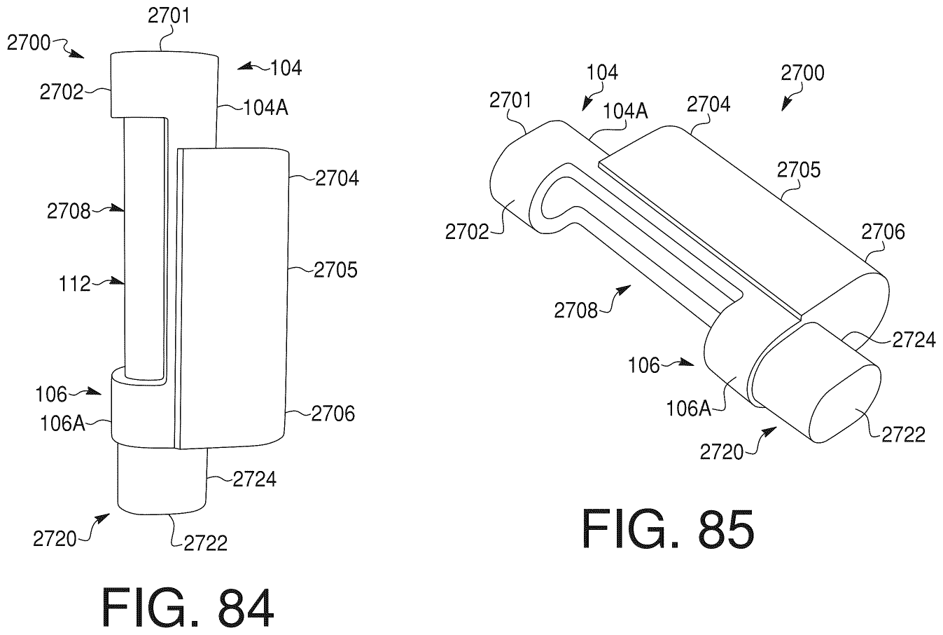

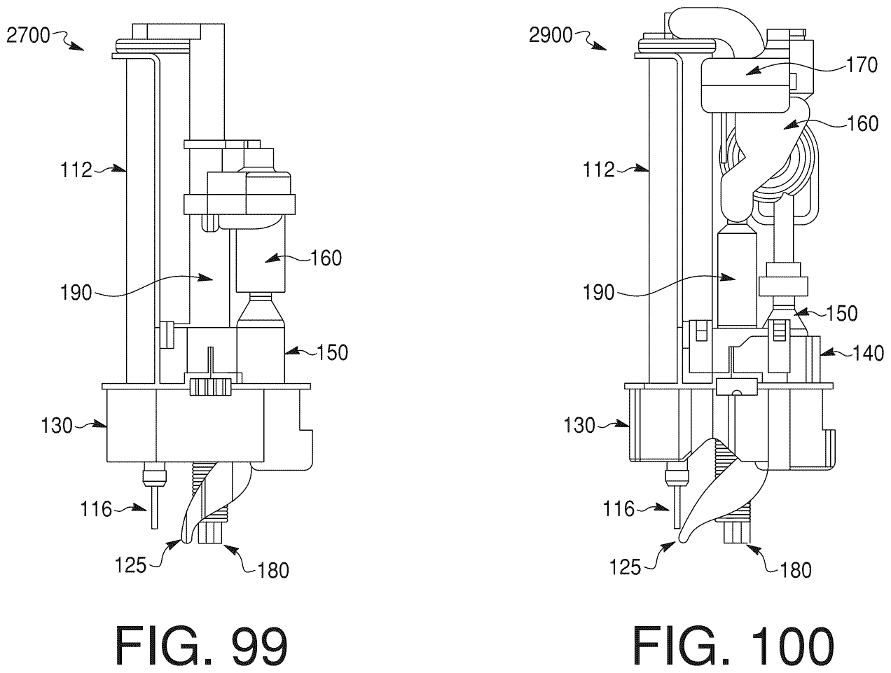

Referring now to FIGS. 84-87, another exemplary auto-injector 2700 is depicted in accordance with an example of this disclosure. It should be appreciated that auto-injector 2700 may be configured, operable, and substantially similar to auto-injector 100 shown and described above except for the differences explicitly noted herein. As such, similar reference numerals of auto-injector 100 are used herein to identify similar components of auto-injector 2700. For example, as best seen in FIG. 99, auto-injector 2700 may include one or more internal components that are similar to auto-injector 100 (e.g., container 112, needle 116, actuator 125, chassis 130, canister 150, valve assembly 160, mandrel assembly 180, slidable piston 190, and more), however, said components may be positioned and/or oriented in an alternative configuration within a housing 2702 of auto-injector 2700 based on a size and/or shape of auto-injector 2700 as described in detail herein.

In the example, auto-injector 2700 may include housing 2702 having a longitudinal length defined between top end 104 and bottom end 106, with a shroud 2720 movably coupled to bottom end 106. Shroud 2720 may include a bottom surface 2722 (e.g., a user-engaging interface) and a body 2724 defining a cross-sectional profile of shroud 2720 that is relatively smaller than shroud 120 described above. The cross-sectional profile of shroud 2720 may correspond with a location of the needle (e.g., needle 116) disposed within housing 2702. Accordingly, a position of shroud 2720 may be indicative of an injection location of the needle during use of auto-injector 2700. Housing 2702 may include a planar surface 2701 at top end 104, such that housing 2702 may have a flattened configuration along top end 104.

In the example, auto-injector 2700 may include an extended housing 2705 that is radially offset from housing 2702. Extended housing 2705 may have a longitudinal length defined between a top end 2704 and a bottom end 2706, with top end 2704 being positioned relatively below top end 104 and bottom end 2706 being positioned along a plane substantially parallel to bottom end 106. Extended housing 2705 may extend radially outwards from at least one of the opposing sidewalls 104A of housing 2702, thereby breaking up a form of auto-injector 2700 into a pair of housings 2702, 2705. Top end 2704 may define a user interface along which a user may control auto-injector 2700 during use, such as manually grasping extended housing 2705. It should be appreciated that one or more internal components of auto-injector 2700 may be disposed inside each of housing 2702 and extended housing 2705. In other words, extended housing 2705 may provide a shrink-wrap enclosure for containing one or more components of auto-injector 2700, as seen in FIG. 99.

Auto-injector 2700 may include a window 2708 that is positioned along at least one of the opposing sidewalls 106A of housing 2702. In other words, window 2708 may be formed along an outer side of housing 2702. Window 2708 may define a cutout region in housing 2702 for receiving container 112, such that auto-injector 2700 may not include an outer window lens and/or barrier disposed over window 2708 (see FIG. 85).

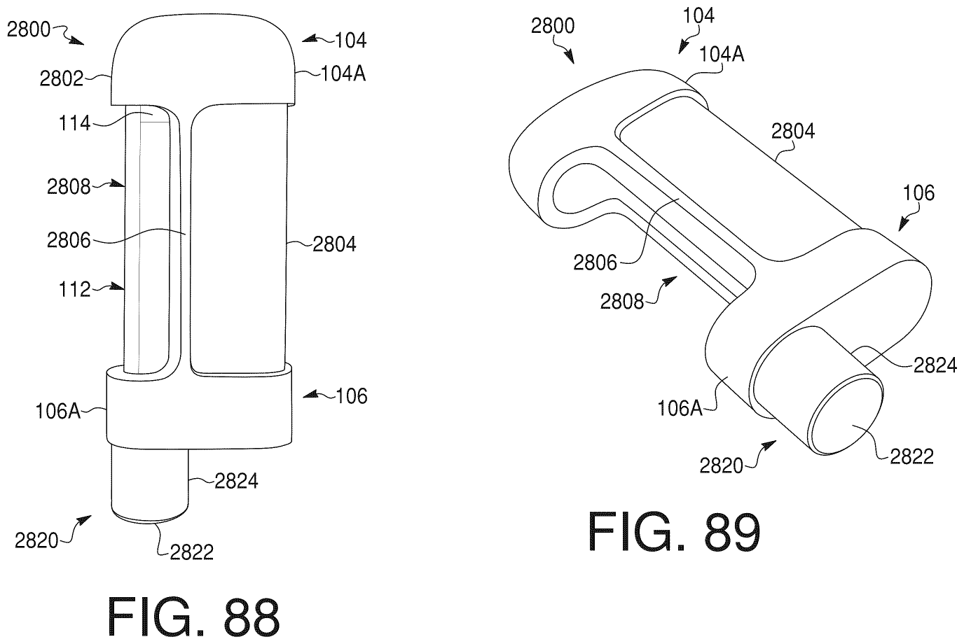

Referring to FIGS. 88-90, another exemplary auto-injector 2800 is depicted in accordance with an example of this disclosure. It should be appreciated that auto-injector 2800 may be configured, operable, and substantially similar to auto-injector 100 shown and described above except for the differences explicitly noted herein. As such, similar reference numerals of auto-injector 100 are used herein to identify similar components of auto-injector 2800. In the example, auto-injector 2800 may include a housing 2802 having a longitudinal length defined between top end 104 and bottom end 106, with a shroud 2820 movably coupled to bottom end 106. Shroud 2820 may include a bottom surface 2822 (e.g., a user-engaging interface) and a body 2824 defining a narrowed cylindrical profile of shroud 2820. In other words, body 2824 may be sized, shaped, and/or otherwise designed as a single barrel configuration. The narrowed cylindrical profile of body 2824 may correspond with a location of the needle (e.g., needle 116) disposed within housing 2802. Accordingly, a position of shroud 2820 may be indicative of an injection location of the needle during use of auto-injector 2800.

In the example, top end 104 of housing 2802 may have a rounded and/or curved form, and auto-injector 2100 may include a window 2808 that is positioned along at least one of the opposing sidewalls 106A of housing 2802. In other words, window 2808 may be formed along an outer edge of housing 2802. Window 2808 may define a cutout region in housing 2802 for receiving container 112, such that auto-injector 2800 may not include an outer window lens and/or barrier disposed over window 2808. In the example, housing 2802 may include an outer surface 2804 that is recessed relative to the adjacent exterior surfaces of housing 2802. As such, any labels and/or markings positioned on outer surface 2804 may be recessed relative to the adjacent exterior of housing 2802. In some embodiments, outer surface 2804 may define a grip region of housing 2802 for manually controlling auto-injector 2800 during use. Auto-injector 2800 may include a raised edge 2806 formed along an exterior of housing 2802 between outer surface 2804 and window 2808. Raised edge 2806 may be sized, shaped, and/or otherwise configured to enhance a griping interface of housing 2802 by a user of auto-injector 2800.

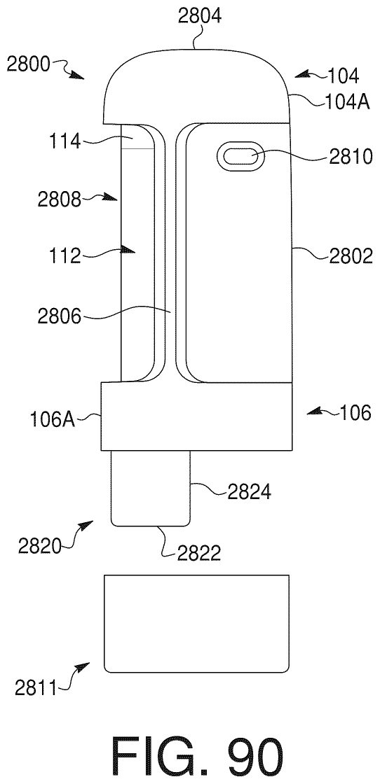

Referring specifically to FIG. 90, auto-injector 2800 may include a secondary indicator 2810 positioned along housing 2802, and particularly on outer surface 2804. Secondary indicator 2810 may be configured and operable to facilitate use of auto-injector 2800, such as by providing a dose indication to the user. In some embodiments, secondary indicator 2810 may be configured to display one or more colors and/or other visual elements indicative of an operating state of auto-injector 2800. For example, secondary indicator 2810 may coincide with an indicator assembly (e.g., indicator assembly 170) disposed within housing 2802, such that the indicator assembly may be visible from an exterior of housing 2802 via secondary indicator 2810. In this instance, secondary indicator 2810 may include a window or cutout opening in housing 2802. In the example, auto-injector 2800 may include a cap 2811 removably coupled to housing 2802 at bottom end 106. Cap 2811 may be sized and/or shaped to have a cross-sectional dimension that corresponds to a cross-sectional dimension of housing 2802, such that cap 2811 may have a greater profile than the narrowed cylindrical profile of shroud 2820.

Referring to FIGS. 91-93, another exemplary auto-injector 2900 is depicted in accordance with an example of this disclosure. It should be appreciated that auto-injector 2900 may be configured, operable, and substantially similar to auto-injector 100 shown and described above except for the differences explicitly noted herein. As such, similar reference numerals of auto-injector 100 are used herein to identify similar components of auto-injector 2900. For example, as best seen in FIG. 100, auto-injector 2900 may include one or more internal components that are similar to auto-injector 100 (e.g., container 112, needle 116, actuator 125, chassis 130, carrier 140, canister 150, valve assembly 160, indicator assembly 170, mandrel assembly 180, slidable piston 190, and more), however, said components may be positioned and/or oriented in an alternative configuration within a housing 2902 of auto-injector 2900 based on a size and/or shape of auto-injector 2900 as described in detail herein.

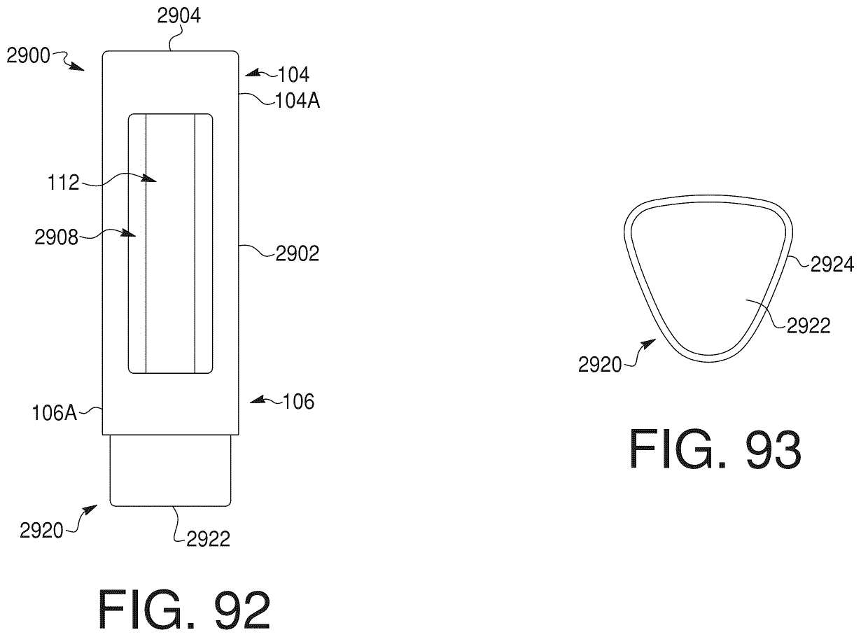

In the example, auto-injector 2900 may include housing 2902 having a longitudinal length defined between top end 104 and bottom end 106, with a shroud 2920 movably coupled to bottom end 106 (see FIG. 92). Auto-injector 2900 may include a cap 2911 removably coupled to bottom end 106, thereby disposing shroud 2920 within cap 2911 when cap 2911 is coupled onto housing 2902. Housing 2902 may include a planar surface 2904 at top end 104, such that housing 2902 may have a flattened configuration along top end 104. Housing 2902 may be sized, shaped, and/or otherwise designed to have a generally trilobe profile in which the pair of opposing sidewalls 104A, 106A converge at a central corner 2906. In other words, housing 2902 may form a rounded triangular configuration (e.g., a rouleaux triangle). It should be appreciated that auto-injector 2900 may include alternative configurations and/or orientations of internal components than that of auto-injector 100 shown and described above in view of the trilobe profile of housing 2902.

Housing 2902 may include a window 2908 that is positioned along central corner 2906, such that window 2908 is positioned between the pair of opposing sidewalls 104A, 106A. Window 2908 may define a cutout region in housing 2902 for receiving container 112, such that auto-injector 2900 may not include an outer window lens and/or barrier disposed over window 2908. Shroud 2920 may include a bottom surface 2922 (e.g., a user-engaging interface) and a body 2724 defining a cross-sectional dimension of shroud 2920 that corresponds to the trilobe profile of housing 2902 described above.

It should be appreciated that the embodiments shown and described herein may generally have a cross-sectional dimension (e.g., a height, a width, and a depth) that is relatively reduced to minimize a user perception of the auto-injector having a bulky appearance. The reduced profile of the auto-injectors of this disclosure may facilitate an intuitive configuration that is easier to control and generally less intimidating for a user. The cross-sectional profile of the embodiments shown and described herein may further facilitate grasping the auto-injector with a variety of suitable hand postures and grips. By incorporating a reduced profile, a need for users to stabilize the auto-injectors of this disclosure with multiple hands may be minimized.

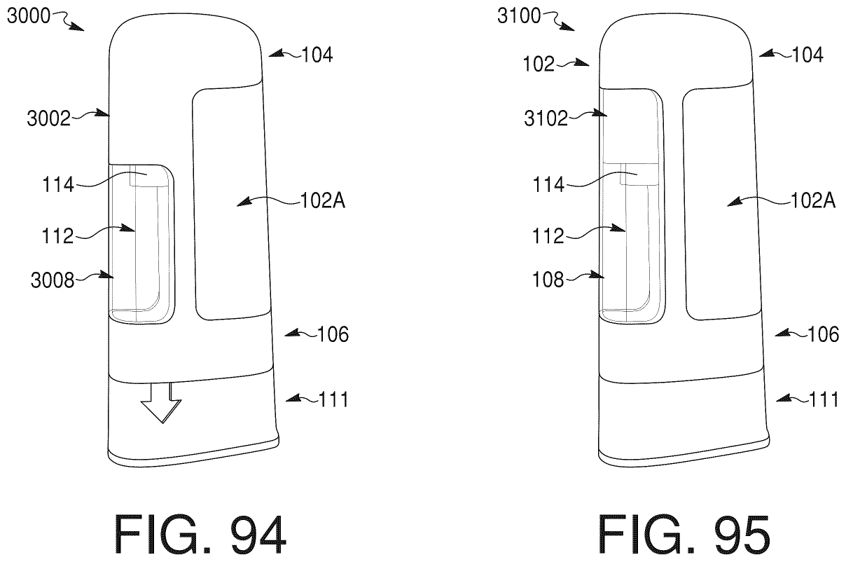

In the embodiments shown and described herein, container 112 may include a 3 milliliter prefilled syringe. In other embodiments, the auto-injector may be configured and operable to deliver smaller fill volumes from container 112. In this instance, one or more components of the auto-injector may be designed to accommodate a relative position of piston 114 within container 112 for delivering the intended dose volume. For example, as seen in FIG. 94, an exemplary auto-injector 3000 may include a housing 3002 having a window 3008 with a longitudinal length that is relatively shorter to accommodate the smaller fill volume of container 112. In other words, window 3008 may be formed on housing 3002 with a length that coincides with the fill volume of container 112, such that the length of window 3008 is substantially shorter than a length of container 112 disposed inside housing 3002. In this instance, an initial position of piston 114 in container 112 may be visible from window 3008.

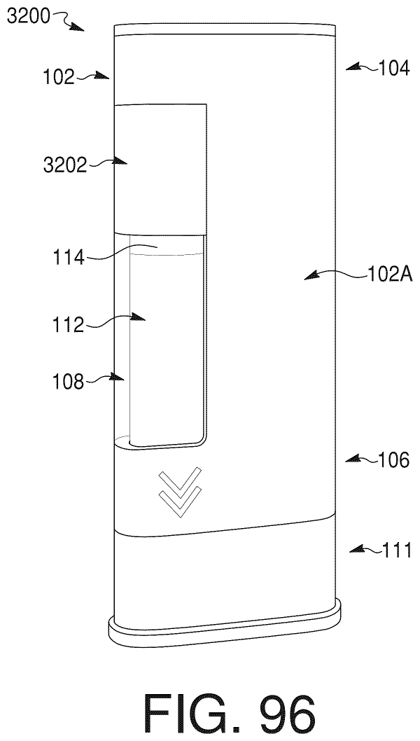

By way of further example, as seen in FIG. 95, an exemplary auto-injector 3100 may include a secondary label 3102 disposed over at least a portion of first window 108, thereby covering the portion of first window 108 coinciding with an unfilled volume of container 112. In this instance, secondary label 3102 may cover and/or conceal the portion of container 112 devoid of medicament, such that an initial position of piston 114 in container 112 may be visible from first window 108. It should be appreciated that secondary label 3102 may be separate from the one or more labels disposed along outer surface 102A of housing 102, and secondary label 3102 may be coupled or otherwise attached directly to the outer window lens of first window 108. As another example, as seen in FIG. 96, an exemplary auto-injector 3200 may include a label 3202 that is integral with the label disposed over outer surface 102A of housing 102. In this instance, label 3202 may be disposed over at least a portion of first window 108, thereby covering the portion of first window 108 coinciding with an unfilled volume of container 112. In this instance, label 3202 may cover and/or conceal the portion of container 112 devoid of medicament, such that an initial position of piston 114 in container 112 may be visible from first window 108.

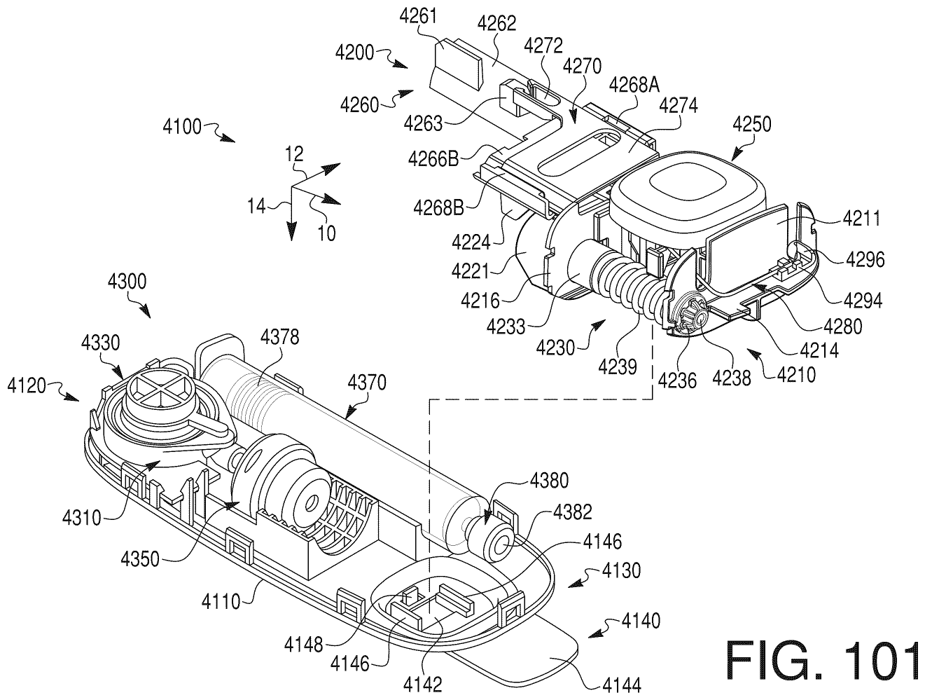

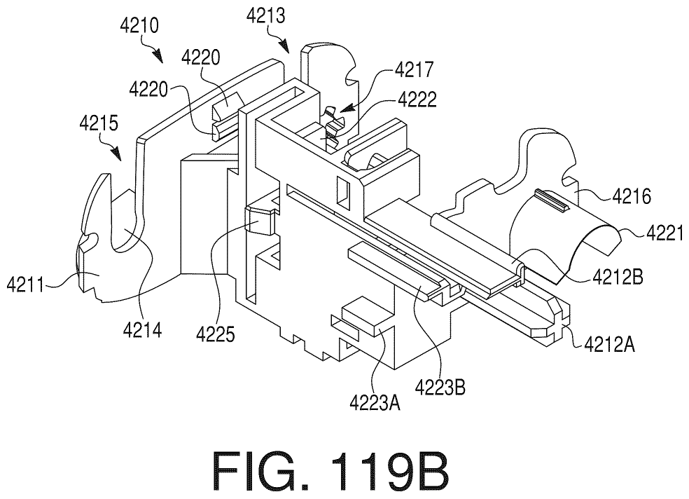

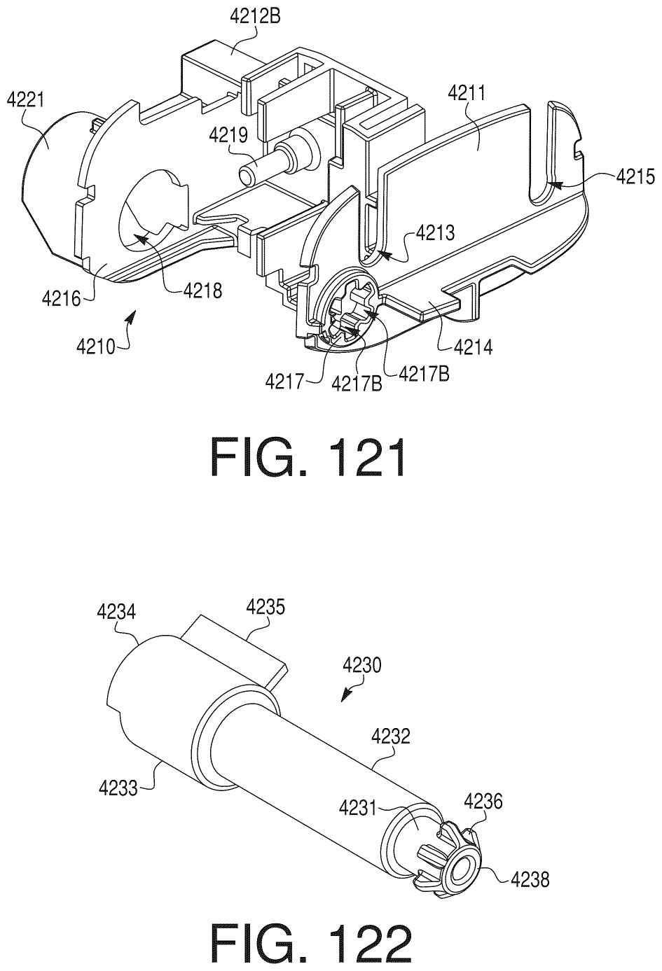

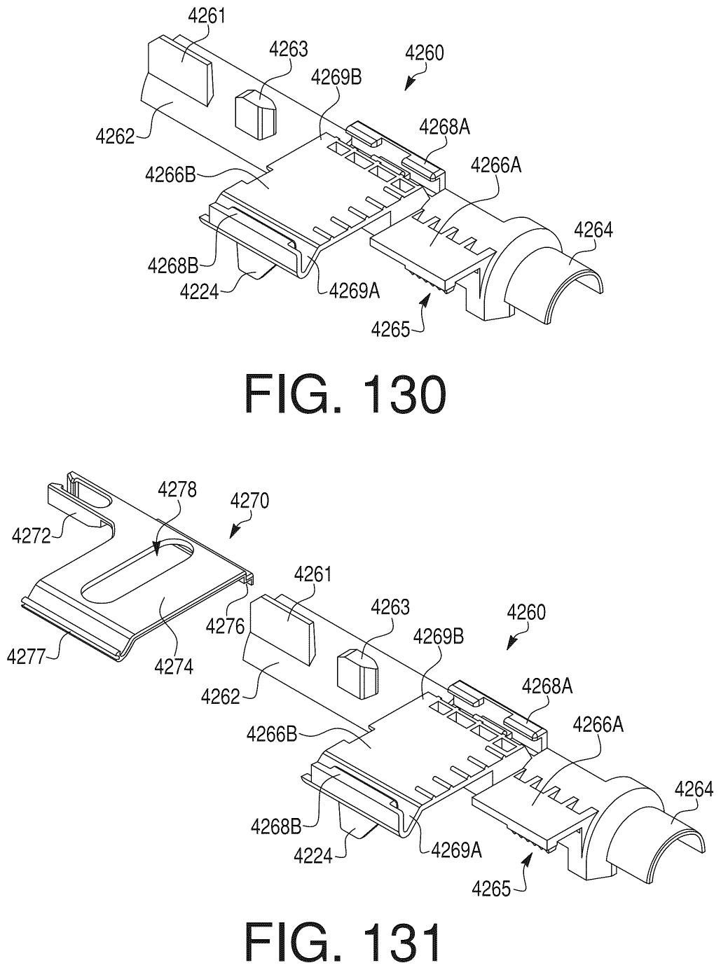

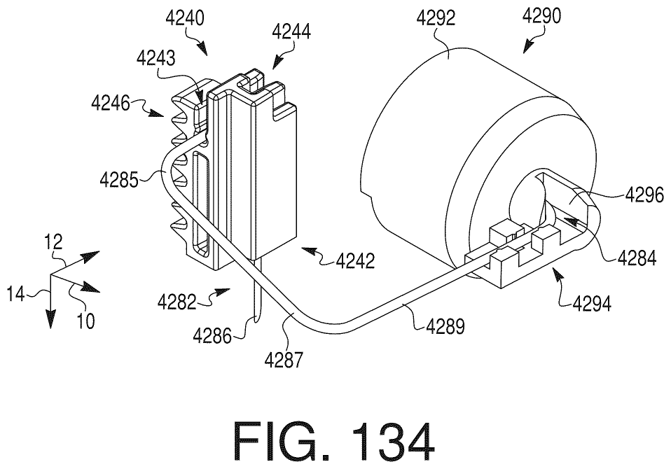

Referring now to FIG. 101, another exemplary auto-injector 4100 is depicted in accordance with an example of the present disclosure. Auto-injector 4100 may include a housing having a bottom cover 4110 and a top cover (not shown). Bottom cover 4110 may be defined by a first end 4120 and a second end 4130. Bottom cover 4110 may define a tissue-engaging surface along an exterior interface of bottom cover 4110, through which a fluid conduit (e.g., a needle) may be deployed and retracted (see FIG. 133). The top cover may define a user interface surface from which a user may control auto-injector 4100, such as through a window providing visualization of one or more internal components of auto-injector 4100. Auto-injector 4100 may include a needle mechanism 4200 and a valve assembly 4300 housed between the bottom cover 4110 and the top cover of the housing. Needle mechanism 4200 may include a carrier 4210 (FIGS. 119A-119B and 121), an activator 4230 (FIG. 122), an actuator or button 4250 (FIGS. 127-129), a shuttle actuator 4260 (FIGS. 130-132), an indicator slide 4270 (FIGS. 130-132), a fluid conduit 4280 (FIG. 133), and a needle retainer or sterile connector 4290 (FIG. 134).

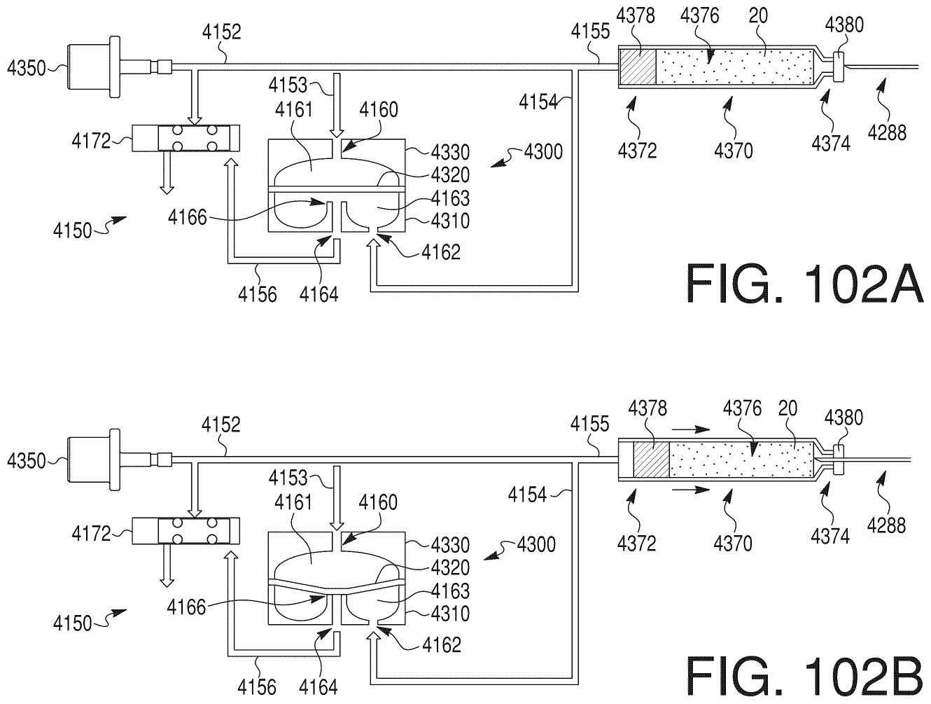

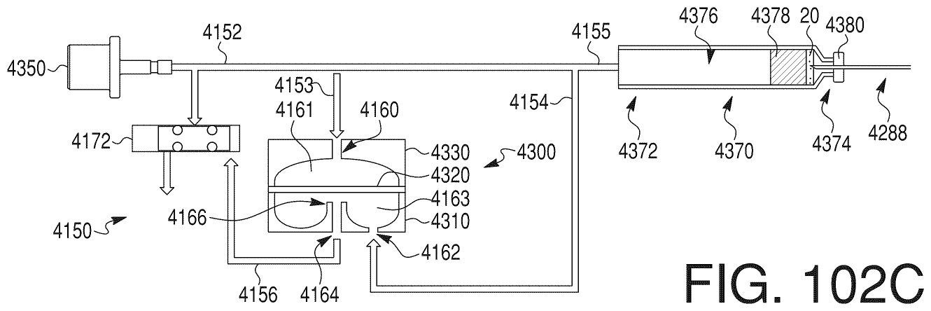

Valve assembly 4300 may be configured and operable substantially similar to valve assembly 160 shown and described above except for the differences explicitly noted herein. For example, valve assembly 4300 may include a low pressure body portion 4310, a high pressure body portion 4330, and a fluid source 4350. It should be appreciated that fluid source 4350 may be configured and operable similar to canister 150 shown and described above. Valve assembly 4300 may be operatively coupled to fluid conduit 4280 via a container 4370 having a stopper 4380. Stated differently, fluid conduit 4280 may establish fluid communication with container 4370, and container 4370 may be operatively coupled to valve assembly 4300, thereby fluidly coupling valve assembly 4300 to fluid conduit 4280 through container 4370. Container 4370 includes a piston 4378 that inhibits receipt of a fluid (e.g., a pressurized gas) from valve assembly 4300 into container 4370 and/or fluid conduit 4280. As described in detail herein, needle mechanism 4200 and valve assembly 4300 may be coupled to one another, and collectively configured to deploy fluid conduit (FIG. 133) through bottom cover 4110, deliver a dosage of a medicament from container 4370 (FIGS. 102A-102C), and retract the needle back into the housing of auto-injector 4100 in response to a single actuation of auto-injector 4100 by a user.

Auto-injector 4100 may have any suitable dimensions to enable portability and self-attachment by a user. Auto-injector 4100, for example, may have a length from about 0.5 inches to about 5.0 inches, a width of about 0.5 inches to about 3.0 inches, and a height from 0.5 inches to about 2.0 inches. Auto-injector 4100 also may include a grip or frictional coating such that the outer surface of auto-injector 4100 is a non-slip surface. Auto-injector 4100 may be oriented about a longitudinal axis 10 (e.g., an X axis), a lateral axis 12 (e.g., a Y axis) that is substantially perpendicular to longitudinal axis 10, and a transverse axis 14 (e.g., a Z axis) that is substantially perpendicular to both longitudinal axis 10 and lateral axis 12. Transverse auto-injectors of the present disclosure, in some embodiments, may have a longer dimension along longitudinal axis 10 than along lateral axis 12 and/or transverse axis 14. It should be appreciated that auto-injector 4100 may be sized, shaped, and/or otherwise configured as a transverse and/or non-transverse device without departing from a scope of this disclosure.

In certain embodiments of auto-injector 4100, such as when auto-injector 4100 is a wearable auto-injector, auto-injector 4100 may include one or more features, such as, e.g., an adhesive patch, straps, or the like, for securing to the user. For example, the wearable auto-injector may include an adhesive patch positioned along an exterior of bottom cover 4110. The adhesive patch may be coupled to the exterior of bottom cover 4110 (e.g., the tissue-engaging surface) to help secure auto-injector 4100 to a user's body (e.g., skin). The adhesive patch may be formed from fabric or any other suitable material, and may include an adhesive. The adhesive may be an aqueous or solvent-based adhesive, or may be a hot melt adhesive, for example. Suitable adhesives also include acrylic based, dextrin based, and urethane based adhesives as well as natural and synthetic elastomers. In some examples, the adhesive provided on the patch may be activated upon contact with a user's skin. In yet another example, the adhesive patch may include a non-woven polyester substrate and an acrylic or silicone adhesive. The adhesive patch may be joined to bottom cover 4110 by, e.g., a double-sided adhesive, or by other mechanisms like ultrasonic welding. The adhesive patch may have a longitudinal dimension (e.g., a dimension parallel to longitudinal axis 10) that is greater than a width (e.g., a dimension parallel to lateral axis 12) of auto-injector 4100. In other embodiments of the disclosure, auto-injector 4100 does not include an adhesive patch. For example, auto-injector 4100 may be a handheld auto-injector, as opposed to a wearable auto-injector. In at least some embodiments, a handheld auto-injector may require a user to hold auto-injector 4100 against the user's skin for the entirety of an injection procedure, whereas, a wearable injector may include features for securing auto-injector 4100 to the skin.

Still referring to FIG. 101, container 4370 may be sized and shaped to store a nominal value of a medicament. The “nominal volume” (also called the “specified volume,” or “specified capacity”) of a container refers to the container's maximum capacity, as identified by the container's manufacturer or a safety standards organization. A manufacturer or a safety standards organization may specify a container's nominal volume to indicate that the container can be filled with that volume of fluid (either aseptically or not) and be closed, stoppered, sterilized, packaged, transported, and/or used while maintaining container closure integrity, and while maintaining the safety, sterility, and/or aseptic nature of the fluid contained inside. In determining the nominal volume of a container, a manufacturer or a safety standards organization may also take into account variability that occurs during normal filling, closing, stoppering, packaging, transportation, and administration procedures. As an example, a prefillable syringe may be either hand- or machine-filled with up to its nominal volume of fluid, and may then be either vent tube- or vacuum-stoppered, without the filling and stoppering machinery and tools touching and potentially contaminating the contents of the syringe. Alternatively, the stopping machinery and tools may be sterile or aseptic, and are able to contact the contents of the syringe and/or the syringe itself without resulting in any contamination.

Container 4370 may have about a 5.0 mL nominal volume in some examples, although any other suitable nominal volume (e.g., from about 0.5 mL to about 50.0 mL, or from about 2.0 mL to about 10.0 mL, or from about 3.0 mL to about 6.0 mL, or from about 1.0 mL to about 3.0 mL, or from about 2.0 mL to about 5.0 mL, or another suitable range) also may be utilized depending on the drug to be delivered. In other examples, container 4370 may have a nominal volume greater than or equal to about 0.5 mL, or greater than or equal to about 2.0 mL, or greater than or equal to about 3.0 mL, or greater than or equal to about 4.0 mL, or greater than or equal to about 5.0 mL. Container 4370 may contain and preserve a drug for injection into a user, and may help maintain sterility of the drug. In one embodiment, container 4370 may be configured to deliver a delivered quantity of medicament (e.g., from about 0.5 mL to about 4.0 mL, about 1.0 mL to about 3.5 mL, about 3.0 mL, about 3.1 mL, about 3.2 mL, about 3.3 mL, about 3.4 mL, about 3.5 mL, greater than about 1.0 mL, greater than about 2.0 mL, greater than about 3.0 mL, greater than about 4.0 mL, greater than about 5.0 mL, greater than about 10.0 mL, greater than about 20.0 mL or another delivered quantity).

The delivered quantity may be less than the nominal volume of container 4370. Furthermore, in order to deliver the delivered quantity of medicament to a user, container 4370 itself may be filled with a different quantity of medicament than the delivered quantity (i.e., a filled quantity). The filled quantity may be an amount of medicament greater than the delivered quantity to account for medicament that cannot be transferred from container 4370 to the user due to, e.g., dead space in container 4370 or fluid conduit 4280. Thus, while container 4370 may have a nominal volume of 5 mL, the filled quantity and delivered quantity of medicament may be less than 5 mL.

In one embodiment, when container 4370 is used in a handheld auto-injector, the delivered quantity of medicament from container 4370 may be from about 0.5 mL to about 4.0 mL, about 1.0 mL to about 3.5 mL, about 3.0 mL, about 3.1 mL, about 3.2 mL, about 3.3 mL, about 3.4 mL, about 3.5 mL. The delivered quantity of medicament may be related to the viscosity of the medicament and the hand-held nature of auto-injector 4100. That is, in at least some embodiments, at certain viscosities, higher volumes of medicament may prohibit the ability of auto-injector 4100 to complete an injection procedure in less than an acceptable amount of time, e.g., less than about 30 seconds. Thus, the delivered quantity of medicament from auto-injector 4100 may be set such that an injection procedure, measured from 1) the point in time at which auto-injector 4100 is placed onto a user's skin, to 2) the point in time at which auto-injector 4100 is removed from the skin, is less than about 30 seconds or less than about another time period (e.g., less than about 25 seconds, less than about 20 seconds, less than about 15 seconds, or less than about 10 seconds).

When the delivered quantity and/or viscosity of the medicament is too high, auto-injector 4100 may not be able to function as a handheld auto-injector, since the time required to complete the injection procedure may be higher than commercially or clinically acceptable for handheld devices. Again, as stated above, in embodiments where container 4370 is used in a hand-held auto-injector, regardless of the nominal volume of container 4370, the delivered quantity of medicament from container 4370 may be set such that the injection procedure as defined above is completed in a relatively short period of time (so as to avoid the need for additional features to attach auto-injector 4100 to the user so that auto-injector 4100 is a wearable auto-injector). However, it is contemplated that various embodiments of the present disclosure relate to wearable auto-injectors that deliver relatively large quantities of medicament (e.g., greater than about 3.5 mL) and/or have relatively longer injection procedure times as opposed to handheld auto-injectors (e.g., longer than about 30 seconds, longer than about 1 minute, longer than about 2 minutes, longer than about 5 minutes, or longer than about 1 hour) to complete an injection procedure as measured from 1) the point in time at which the auto-injector is placed onto a user's skin, to 2) the point in time at which the auto-injector is removed from the skin.

Container 4370 may have about a 13 mm diameter neck, about a 45 mm length, and an internal diameter of about 19.05 mm. In another embodiment, container 4370 may be a standard 3 mL container having an 8 mm crimp top, a 9.7 mm inner diameter, and a 64 mm length. In further embodiments, container 4370 may have a length of about 64 mm to 74 mm, such as, for example, about 69.3 mm+0.15 mm (excluding a length of the neck of container 4370 at a second end 4374 of container 4370, as seen in FIGS. 102A-102C). In embodiments including the neck, container 4370 may have a length ranging from about 65 mm to 75 mm, such as, for example, about 70.8 mm+0.4 mm. These values are merely exemplary, and other suitable dimensions may be utilized as appropriate. In some examples, container 4370 may be formed using conventional materials, and may be shorter than existing devices, which can help auto-injector 4100 remain cost-effective and small. In some embodiments, container 4370 may be a shortened ISO 10 mL cartridge. Auto-injectors of the present disclosure may be configured to deliver highly viscous liquid to a patient. For example, auto-injector 4100 of the present disclosure may be configured to deliver liquid having a viscosity from about 0 cP to about 100 cP, from about 5 cP to about 45 cP, from about 10 cP to about 40 cP, from about 15 cP to about 35 cP, from about 20 cP to about 30 cP, or about 25 cP.

Still referring to FIG. 101, container 4370 may include a piston 4378 movably disposed within a cavity of container 4370. Piston 4378 may be movable by a pressurized fluid expelled from a fluid source, such as, e.g., fluid source 4350 (FIGS. 102A-102C). As described further herein, pressurized fluid (e.g., gas) expelled from fluid source 4350 may translate piston 4378 within container 4370, and translate container 4370 horizontally along longitudinal axis 10 toward second end 4130. The movement of piston 4378 towards second end 4130 may cause piston 4378 to act against the contents within container 4370 (e.g., drugs, medications, medicaments, etc.), which ultimately transfers force against container 4370, thereby causing container 4370 to move along longitudinal axis 10. In some embodiments, transverse auto-injectors may be oriented such that fluid source 4350 and piston 4378 are offset, or are otherwise not longitudinally aligned with one another.

Referring to FIGS. 102A-102C, a drive system 4150 of auto-injector 4100 is schematically depicted. It should be appreciated that drive system 4150 may be substantially similar to drive system 2000 shown and described above except for the differences explicitly described herein. Drive system 4150 may be configured to provide a drive force to deliver medicament 20 from container 4370 to a patient. Drive system 4150 may include fluid source 4350 fluidly coupled to one or more components of auto-injector 4100, e.g., needle mechanism 4200 and valve assembly 4300. Drive system 4150 may further include a first high pressure line 4152, a second high pressure line 4153, a low pressure line 4154, a drive line 4155, and a third line 4156. Fluid source 4350 may be operatively coupled to container 4370 via first high pressure line 4152 and drive line 4155. Further, fluid source 4350 may be fluidly coupled to valve assembly 4300 via one or more of first high pressure line 4152, second high pressure line 4153, and low pressure line 4154.

Still referring to FIGS. 102A-102C, valve assembly 4300 may include a diaphragm 4320 positioned between low pressure body portion 4310 and high pressure body portion 4330. It should be appreciated that diaphragm 4320 may be configured and operable substantially similar to diaphragm 2012 shown and described above except for the differences explicitly described herein. Within valve assembly 4300, diaphragm 4320 defines a high pressure (first) cavity 4161 and a low pressure (second) cavity 4163. Valve assembly 4300 may further include a high pressure (first) inlet 4160, a low pressure (second) inlet 4162, and a conduit 4164. Conduit 4164 is formed within a valve seat 4166 that extends into the interior of valve assembly 4300, and particularly low pressure cavity 4163. High pressure cavity 4161 may be in fluid communication with high pressure line 4152 via second high pressure line 4153 and inlet 4160. Low pressure cavity 4163 may be in fluid communication with low pressure line 4154 via low pressure inlet 4162. Low pressure cavity 4163 may be in fluid communication with third line 4156 via conduit 4164.

Drive system 4150 may include a venting system 4172 fluidly connected by a number of fluid lines or conduits. For example, venting system 4172 may be in fluid communication with fluid source 4350 via high pressure line 4152, and with valve assembly 4300 via third line 4156. Venting system 4172 may be configured to vent drive system 4150 by releasing pressurized fluid into an interior cavity of auto-injector 4100 (e.g., defined between bottom cover 4110 and the top cover) and/or into the atmosphere. In some embodiments, venting system 4172 may be configured and operable similar to venting system 2030 shown and described above.

Still referring to FIGS. 102A-102C, container 4370 may include a first end 4372 and a second end 4374. Container 4370 also may include a cavity 4376 having an opening at first end 4372 and extending toward second end 4374. Second end 4374 may include a stopper 4380 configured to assist with closing and/or sealing second end 4374, and allow for a needle 4288 (e.g., a staked needle) of fluid conduit 4280 (FIG. 133) to be inserted into container 4370. As seen in FIG. 101, stopper 4380 may include a line seal, a protrusion disposed in a neck of container 4370 (e.g., to reduce dead volume), and/or a septum 4382. Septum 4382 may be formed of an uncoated bromobutyl material, or another suitable material. Cavity 4376 may be closed at first end 4372 by piston 4378. Piston 4378 may include a fluoropolymer coated bromobutyl material, and, in some embodiments, may include a conical nose to help reduce dead volume within container 4370. Piston 4378 may include one or more rubber materials such as, e.g., halobutyls (e.g., bromobutyl, chlorobutyl, florobutyl) and/or nitriles, among other materials. As described in detail above, container 4370 may store a medicament 20 (e.g., drug, fluid substance, etc.) within cavity 4376.

Fluid source 4350 may include a non-latching can or a latching can. Fluid source 4350 may be configured to dispense liquid propellant for boiling outside of fluid source 4350 so as to provide a pressurized gas (vapor pressure) that acts on piston 4378 in container 4370. In some embodiments, fluid source 4350 may include a pressurized gas that is released from fluid source 4350 and acts on piston 4378 in container 4370. In some embodiments, once opened, the latching can may be latched open so that the entire contents of propellant is dispensed therefrom. Alternatively, in some embodiments, fluid source 4350 may be selectively controlled, including selectively activated and deactivated. For example, in an alternative embodiment, the flow of pressurized gas from fluid source 4350 may be stopped after flow is initiated.

The fluid from fluid source 4350 may be any suitable propellant for providing a vapor pressure to drive piston 4378 relative to container 4370, and container 4370 relative to the housing of auto-injector 4100. In some embodiments, fluid source 4350 may be a high-pressure canister configured to hold a compressed gas, similar to canister 150 shown and described above. In certain embodiments, the propellant may be or contain a hydrofluoroalkane (“HFA”), for example HFA134a, HFA227, HFA422D, HFA507, or HFA410A. In certain embodiments, the propellant may be or contain a hydrofluoroolefin (“HFO”) such as HFO1234yf or HFO1234ze, an organic gas (e.g., carbon dioxide (CO₂), etc.), a cryogenic gas (e.g., Argon (Ar), Helium (He), etc.), a hydrocarbon gas (e.g., propane, butane, propylene, ethane, methane, etc.) or an inorganic gas (e.g., Ammonia, Nitrogen Dioxide (NO₂), Nitrous Oxide (N₂O), etc.).

In the pre-activated state of auto-injector 4100 shown in FIG. 102A, needle 4288 may be in contact with second end 4374 of container 4370. In some embodiments, needle 4288 may be spaced apart from second end 4374 of container 4370 in the pre-activated state of auto-injector 4100. To move auto-injector 4100 from the pre-activated state of FIG. 102A, fluid source 4350 may be activated to move container 4370 along longitudinal axis 10 toward needle 4288. Fluid source 4350 may be actuated so as to move to an open configuration in which propellant may exit the fluid source 4350 as a pressurized gas. As described in further detail below, fluid source 4350 may be actuated in response to one or more components of needle mechanism 4200 (e.g., activator 4230) contacting fluid source 4350. In some embodiments, the actuation is irreversible such that the flow of pressurized gas from fluid source 4350 is not able to be stopped.

When fluid source 4350 is actuated, pressurized gas may flow through high pressure line 4152 and drive line 4155, and then to container 4370. Some pressurized gas from high pressure line 4152 may be diverted to high pressure cavity 4161 via second high pressure line 4153 and high pressure inlet 4160. As seen in FIG. 102B, this may cause diaphragm 4320 to move into low pressure cavity 4163 and toward conduit 4164, thereby sealing valve seat 4166. Reduced-pressure gas may be diverted to low pressure cavity 4163 via low pressure line 4154 and low pressure inlet 4162. The pressure difference between high pressure cavity 4161 and low pressure cavity 4163 may provide the force required to seal conduit 4164 by diaphragm 4320. Gas flowing through drive line 4155 to drive piston may initiate movement of container 4370 toward needle 4288.

When needle 4288 is not yet in fluid communication with medicament 20 within container 4370, activation of fluid source 4350 may apply a pressure against medicament 20 contained in cavity 4376, which may then be applied to container 4370 itself. This pressure may cause container 4370 to move toward the needle 4288, thereby forcing needle 4288 through septum 4382 of stopper 4380. In this instance, as seen in FIG. 102B, needle 4288 is in fluid communication with the contents of container 4370 (e.g., medicament 20). The gas from drive line 4155 may subsequently urge piston 4378 toward second end 4374 to expel medicament 20 through container 4370 until piston 4378 reaches second end 4374 (and bottoms out). Stated differently, once needle 4288 is in fluid communication with medicament 20 of container 4370, further movement of piston 4378 toward second end 4374 may urge medicament 20 through needle 4288. As described further herein, pressurized gas from fluid source 4350 may also drive movement of one or more components of needle mechanism 4200 (e.g., activator 4230, shuttle actuator 4260, indicator slide 4270, etc.) to drive injection of a needle 4286 of fluid conduit 4280 (FIG. 133) into a user.

Referring now to FIG. 102C, when piston 4378 bottoms out at second end 4374, the pressure across high pressure cavity 4161 and low pressure cavity 4163 may equilibrate, thereby causing diaphragm 4320 to lift off of valve seat 4166 and open conduit 4164. This may allow the gas from low pressure line 4154 to travel to venting system 4172 via conduit 4164 and third line 4156 where the gas may vent out of drive system 4150. It should be appreciated that fluid source 4350 may be configured to contain enough pressurized fluid so that release of the pressurized gas may actuate both movement of container 4370 relative to the housing of auto-injector 4100 and movement of piston 4378 relative to cavity 4376. In some instances, fluid source 4350 may contain excess pressurized gas, i.e., more fluid than is necessary to complete delivery of medicament 20 from container 4370. Release of the pressurized fluid from within drive system 4150 may allow for the movement of one or more components of needle mechanism 4200 (e.g., activator 4230, shuttle actuator 4260, indicator slide 4270, etc.) to initiate retraction of needle 4286 (FIG. 133) back into auto-injector 4100.

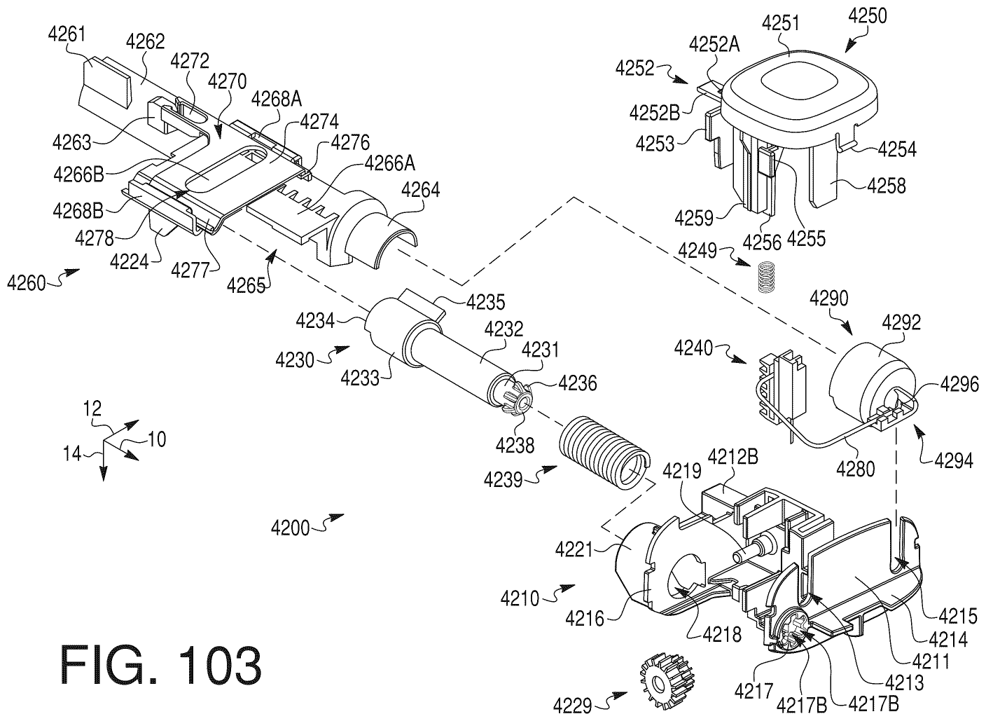

Referring now to FIG. 103, and as seen in further detail in FIGS. 119A-119B, carrier 4210 may include a first flange 4211 with a ledge 4214 extending laterally outwards from first flange 4211 at a first end of carrier 4210. Carrier 4210 may include a second flange 4212 with a pair of legs 4212A, 4212B extending laterally outwards from second flange 4216 at a second end of carrier 4210 in a direction opposite of the first end of carrier 4210. First flange 4211 may extend parallel to transverse axis 14, and ledge 4214 may extend parallel to longitudinal axis 10, such that first flange 4211 and ledge 4214 may be transverse relative to one another. In the embodiment, first flange 4211 and ledge 4214 may form a 90 degree junction relative to one another. First flange 4211 may include a first slot opening 4213 that is sized, shaped, and/or otherwise to receive at least a portion of fluid conduit 4280, and a second slot opening 4215 that is sized, shaped, and/or otherwise configured to receive at least a portion of sterile connector 4290. Slot openings 4213, 4215 may allow carrier 4210 to receive fluid conduit 4280 and sterile connector 4290 in a preassembled state of auto-injector 4100.

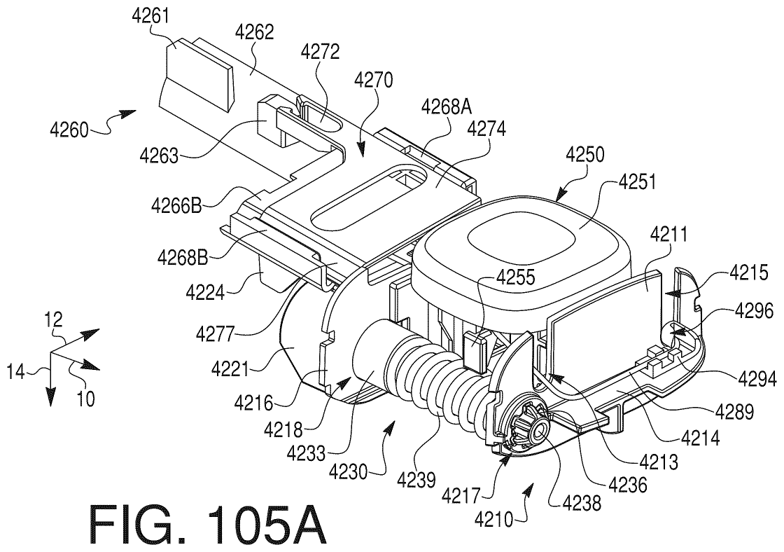

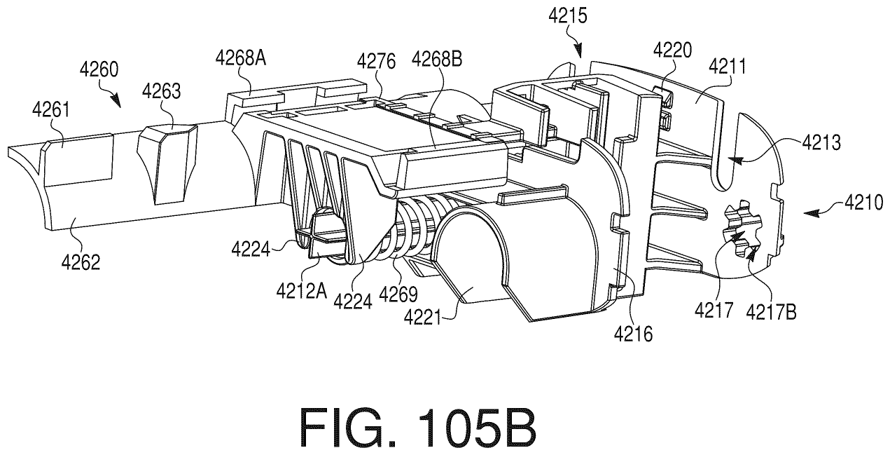

First flange 4211 may include a first opening 4217 positioned adjacent to first slot opening 4213, and second flange 4216 may include a second opening 4218 positioned in longitudinal alignment with first opening 4217 along the longitudinal axis 10. First flange 4211 may be configured to receive at least a portion of activator 4230 through first opening 4217, and second flange 4216 may be configured to receive at least another portion of activator 4230 through second opening 4218, thereby coupling activator 4230 to carrier 4210 when needle mechanism 4200 is in an assembled state, as seen in FIG. 105A. As described herein, activator 4230 may be configured to move relative to carrier 4210 upon activation of auto-injector 4100, such as in response to movement of button 4250 into engagement with activator 4230.

As best seen in FIGS. 119A-119B, carrier 210 may further include the pair of legs 4212A, 4212B extending parallel to longitudinal axis 10. A first leg 4212A may be sized, shaped, and/or otherwise configured to receive a resilient member 4269 (e.g., a spring), such as, for example, about an exterior of first leg 4212A (see FIG. 105B). In the example, first leg 4212A may be sized, shaped, and/or otherwise configured as a mandrel assembly that extends through and supports resilient member 4269. As described further herein, first leg 4212A may be configured to extend into at least a portion of shuttle actuator 4260, such as between a pair of guide flanges 4224 of shuttle actuator 4260, with resilient member 4269 disposed about first leg 4212A. Resilient member 4269 may be configured to move shuttle actuator 4260 by pushing against the pair of guide flanges 4224 upon expansion. Second leg 4212B may be sized, shaped, and/or otherwise configured to at least partially define a travel path for shuttle actuator 4260, along which shuttle actuator 4260 may be configured to move (e.g., translate) within the housing of auto-injector 4100. Second leg 4212B may be configured to align shuttle actuator 4260 with carrier 4210, thereby coupling shuttle actuator 4260 to carrier 4210 when needle mechanism 4200 is in the assembled state.

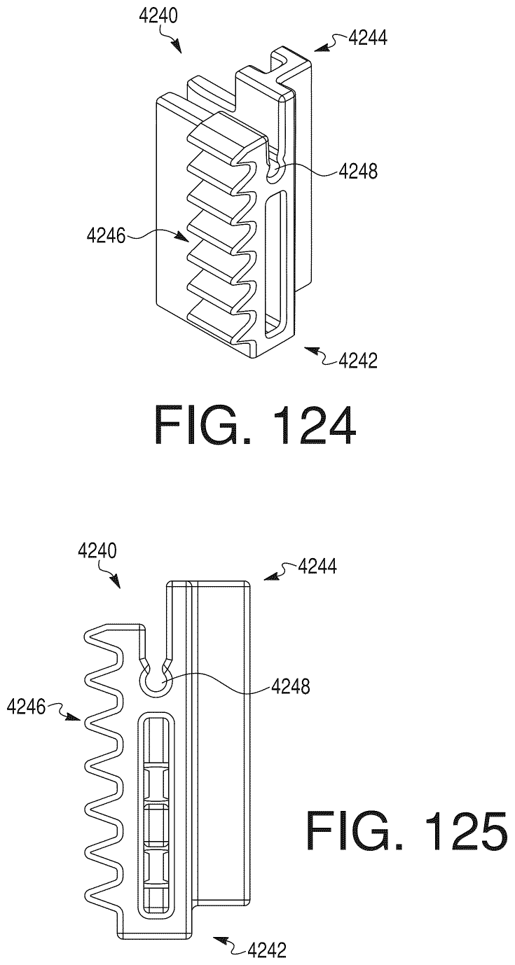

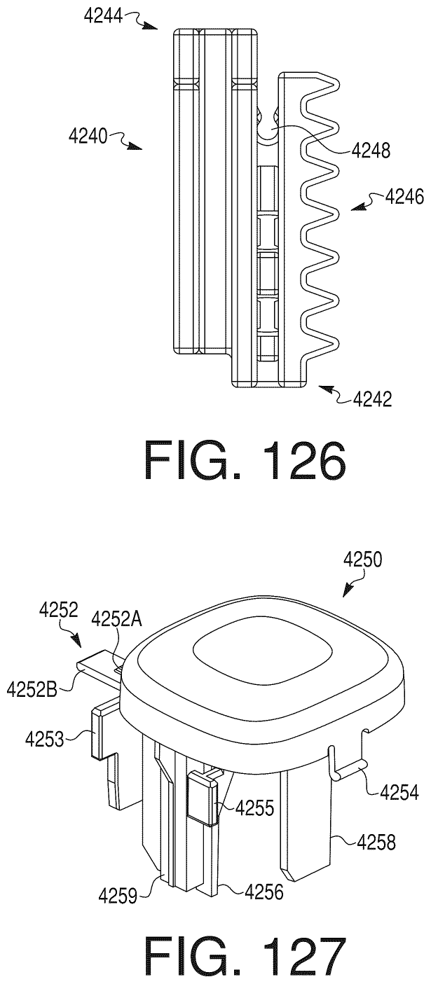

Carrier 4210 may include one or more features (e.g., walls, slots, cavities, platforms, etc.) positioned between first flange 4211 and second flange 4216 for engaging one or more of components of needle mechanism 4200, such as driver 4240 (FIGS. 124-126) and button 4250 (FIGS. 127-129). For example, carrier 4210 may include a rail 4222 that is sized, shaped, and/or otherwise configured to mate with a slot of driver 4240. Rail 4222 may be configured to guide, stabilize, and control movement of driver 4240 relative to carrier 4210. It is noted that carrier 4210 and driver 4240 of the present disclosure are not limited to any specific combination of features, such that, for example, driver 4240 may include a rail configured to be received within a corresponding slot of carrier 4210. Carrier 4210 may include an impediment defined by a pair of protrusions 4220 with an intermediate gap formed in between the pair of protrusions 4220. Protrusions 4220 may be positioned along an interior surface of first flange 4211, and may be configured to securely couple carrier 4210 to button 4250 by engaging a fastening mechanism 4254 of button 4250 (FIG. 127).

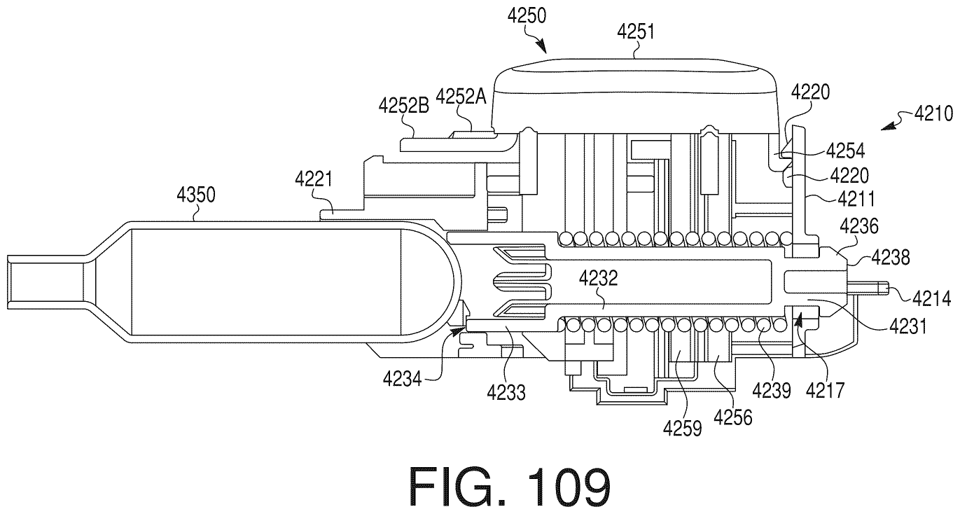

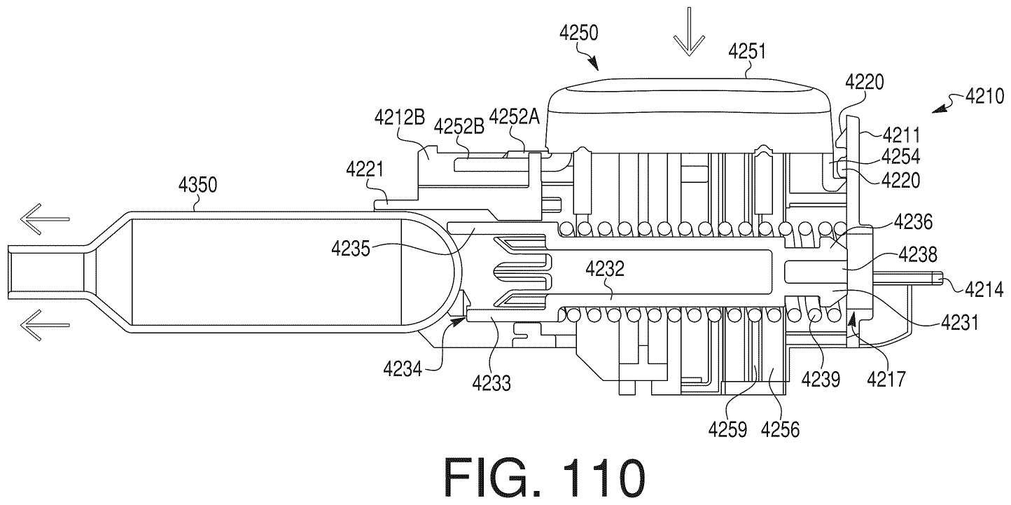

For example, fastening mechanism 4254 may include a tab and/or a hook that is received between the pair of protrusions 4220 (e.g., within the gap defined by protrusions 4220) when button 4250 is in a first (unactuated) position (see FIG. 109). In other words, fastening mechanism 4254 may be locked between the pair of protrusions 4220 when button 4250 is in the first (unactuated) position and/or extended state relative to carrier 4210. Fastening mechanism 4254 may be configured to exit the gap between the pair of protrusions 4220 when button 4250 is moved towards a second (actuated) position (see FIG. 110). In this instance, fastening mechanism 4254 may remain engaged by at least a bottommost protrusion 4220 of the pair of protrusions 4220, thereby maintaining button 4250 in the second (actuated) position. In this instance, fastening mechanism 4254 and the bottommost protrusion 4220 may be collectively configured to lockout and inhibit further movement of button 4250, such as back towards the first (unactuated) position, thereby maintaining button 4250 in a depressed state relative to carrier 4210. In some embodiments, fastening mechanism 4254 may be configured to balance button 4250 relative to carrier 4210.

As best seen in FIG. 119B, carrier 4210 may include a pair of ledges 4223A, 4223B along a sidewall of carrier 4210 that are offset from one another by a distance. The pair of ledges 4223A, 4223B may be positioned along the sidewall of carrier 4210 that includes an impediment 4225. The pair of ledges 4223A, 4223B may be configured to guide the translation of activator 4230 in response to the resilient member 4239 expanding or being compressed, as described below.

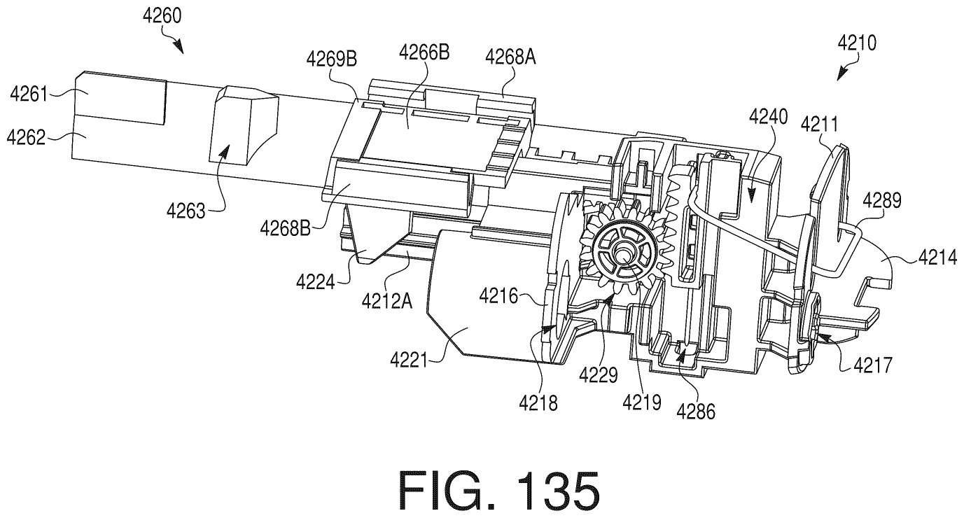

Referring to FIGS. 103 and 121, carrier 210 may include a post 4219 positioned between first flange 4211 and second flange 4216. In the example, post 4219 may extend in a direction parallel to each of first flange 4211 and second flange 4216, such as parallel to the lateral axis 12. Post 4219 may be sized, shaped, and/or otherwise configured to receive a gear 4229 of needle mechanism 4200 (see FIG. 135). Gear 4229 may be configured to rotate relative to post 4219 in response to movement of one or more features of needle mechanism 4200 (e.g., driver 4240, button 4250, shuttle actuator 4260, and more). As described in further detail herein, gear 4229 may provide selective deployment and retraction of fluid conduit 4280 (FIG. 133) from the housing of auto-injector 4100.

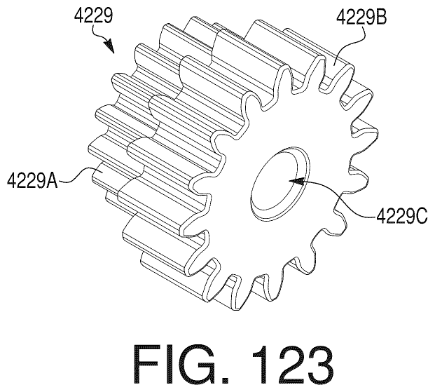

As seen in FIG. 123, gear 4229 may include a double-spur gear having a first gear portion 4229A and a second gear portion 4229B, each of which include a plurality of teeth disposed about an outer circumference of the respective gear portion. It should be appreciated that a pitch ratio of the plurality of teeth on each of first gear portion 4229A and second gear portion 4229B may vary relative to one another. In the embodiment, first gear portion 4229A and second gear portion 4229B may have a generally circular shape, with first gear portion 4229A having a smaller outer diameter than second gear portion 4229B, or alternatively first gear portion 4229A may have a larger outer diameter than the second gear portion 4229B. In this instance, first gear portion 4229A may have a first gear ratio that is different (e.g., smaller) than a second gear ratio of second gear portion 4229B.

First gear portion 4229A may abut second gear portion 4229B, and the two gear portions 4229A, 4229B may rotate about a same axis. It should be appreciated that gear 4229 may be configured to mesh and interact with multiple components of needle mechanism 4200. For example, first gear portion 4229A may be configured and operable to interact with a driver 4240 (see FIGS. 124-126) of needle mechanism 4200, and second gear portion 4229B may be configured and operable to interact with shuttle actuator 4260 (see FIGS. 130-132). In the example, shuttle actuator 4260 and driver 4240 may be configured to move different distances relative to one another, during the same rotation of gear 4229, due to a difference in size and/or pitch ratio of the two gear portions 4229A, 4229B. Gear 4229 may further include an opening 4229C extending through each of the gear portions 4229A, 4229B for receiving post 4219 of carrier 4210 therethrough.

Referring to FIGS. 103 and 122, activator 4230 may include a central body 4232 disposed between a between a first end 4231 and a second end 4233 that is positioned opposite of first end 4231. In the example, second end 4233 may have a cross-sectional profile that is relatively greater than central body 4232 and first end 4231, and central body 4232 may have a cross-sectional profile that is relatively greater than first end 4231. In the example, each of central body 4232, first end 4231, and second end 4233 may generally have a cylindrical and/or elongated profile. Activator 4230 may have a longitudinal length defined between opposing terminal ends 4234, 4238 of first end 4231 and second end 4233. Activator 4230 may include a flange 4235 extending radially outwards from second end 4233, and one or more splines 4236 extending radially outwards from first end 4231. In the example, activator 4230 may include a plurality of splines 4236 disposed about an exterior surface of first end 4231. As described herein, activator 4230 may include a keyed arrangement with carrier 4210, with the keyed arrangement configured and operable to control a longitudinal movement of one or more components of auto-injector 4100. The keyed arrangement between activator 4230 and carrier 4210 may be at least partially defined by the plurality of splines 4236.

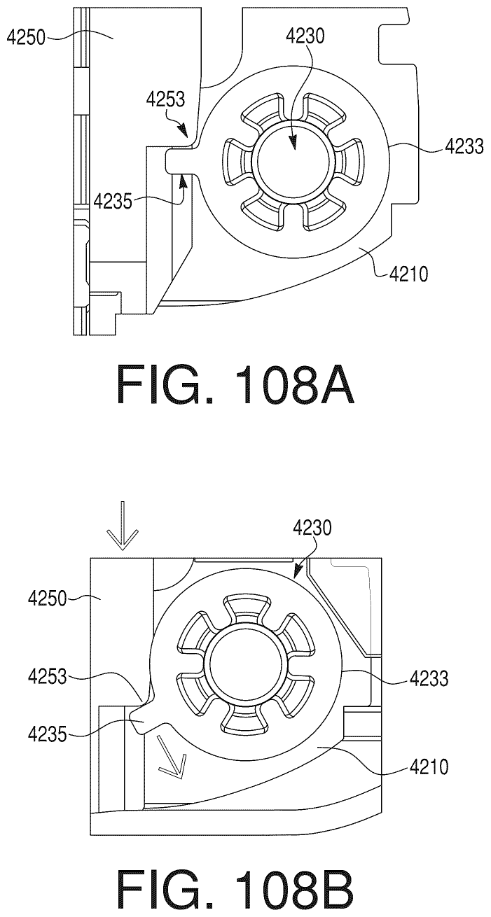

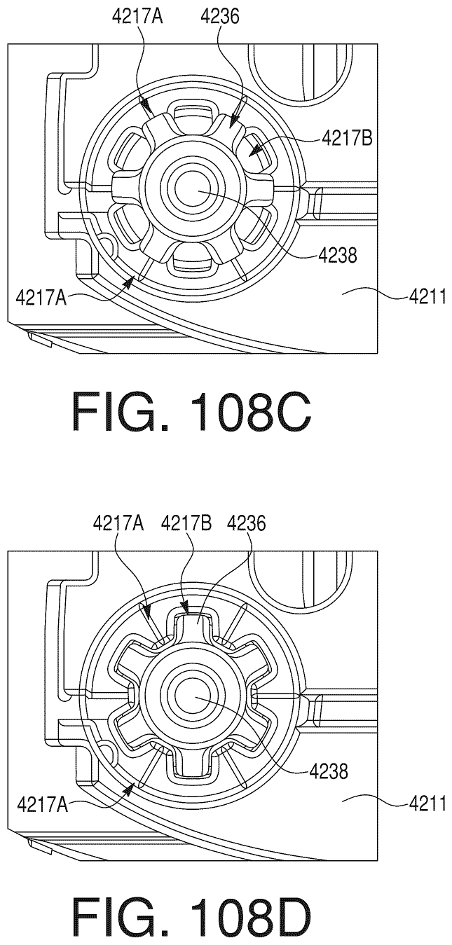

In some embodiments, splines 4236 may include, but are not limited to, tabs, protrusions, grooves, projections, raised surfaces, and/or various other suitable features disposed along the exterior surface of first end 4231 for defining a keyed configuration for activator 4230 interfacing with carrier 4210. The plurality of splines 4236 may be configured to couple with one or more features on first flange 4211 for securely coupling activator 4230 to carrier 4210. For example, the plurality of splines 4236 may be sized, shaped, and/or otherwise configured to mate with a plurality of corresponding detents 4217A disposed about first opening 4217. In instances where splines 4236 may be aligned with detents 4217A, activator 4230 may be in a fixed and/or locked state relative to carrier 4210. The plurality of splines 4236 may be further sized, shaped, and/or otherwise configured to extend through a plurality of corresponding slots 4217B formed on first opening 4217 (see FIGS. 108B-108C) for decoupling activator 4230 from first flange 4211. Stated differently, first opening 4217 may be at least partially defined by the plurality of slots 4217B, which are configured to receive a corresponding spline 4236 of the plurality of splines 4236 when aligned therewith. In instances where splines 4236 may be misaligned with detents 4217A and aligned with slots 4217B, activator 4230 may be in a movable and/or unlocked state relative to carrier 4210. In conjunction with the plurality of splines 4236, the keyed arrangement between activator 4230 and carrier 4210 may be at least partially defined by the plurality of detents 4217A and the plurality of slots 4217B.

As seen in FIGS. 108C-108D, at least one detent 4217A of the plurality of detents 4217A may be disposed between an adjacent pair of slots 4217B of the plurality of slots 4217B. Each of the detents 4217A may be configured to receive a corresponding spline 4236 therein for securely coupling central body 4232 of activator 4230 to first flange 4211 at a fixed configuration (e.g., orientation), as seen in FIG. 108C. In this instance, first end 4231 may extend through first opening 4217 and carrier 4210 may be configured to maintain activator 4230 in the fixed configuration absent an application of force applied thereto, such as by button 4250. In other words, the plurality of detents 4217A may be configured to inhibit movement (e.g. rotation, translation, etc.) of the plurality of splines 4236 upon receipt therein. As described herein, button 4250 may be configured to move (e.g., rotate) central body 4232, thereby causing splines 4236 to move (e.g. rotate) out of alignment with detents 4217A and into alignment with slots 4217B, as seen in FIG. 108D, thereby allowing first opening 4217 to receive splines 4236 and terminal end 4238 therethrough for decoupling activator 4230 from first flange 4211. Stated differently, upon aligning splines 4236 with slots 4217B, activator 4230 is no longer maintained at the fixed configuration by carrier 4210 and/or inhibited from moving relative to carrier 4210. Activator 4230 may be configured to move in a direction away from first flange 4211, thereby causing first end 4231, splines 4236, and terminal end 4238 to move out of first opening 4217.

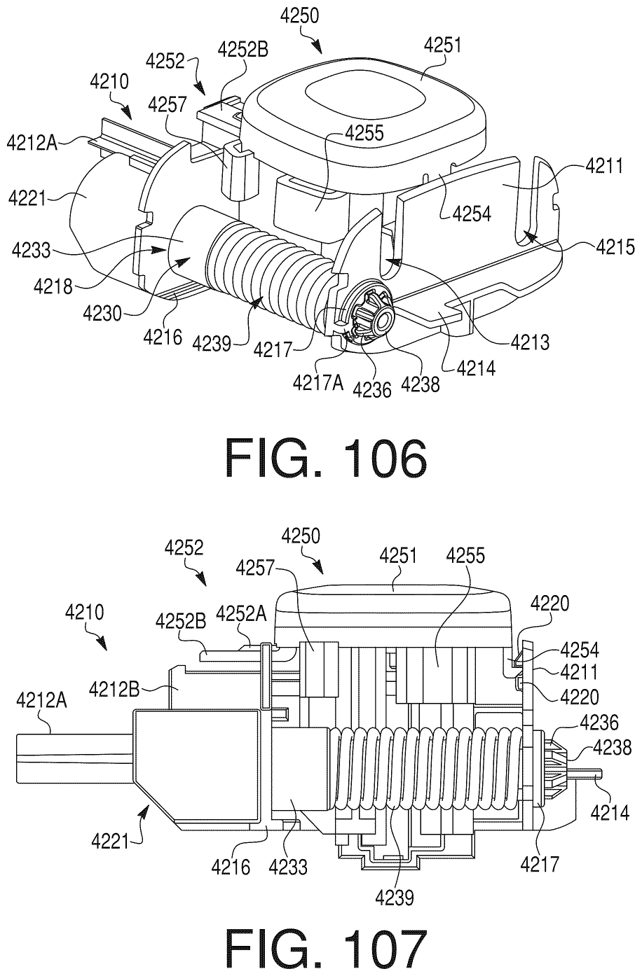

Referring to FIGS. 105A and 106-107, activator 4230 may include a resilient member 4239 (e.g., a spring) that is sized, shaped, and/or otherwise configured to extend about an exterior of central body 4232. For example, resilient member 4239 may be disposed over central body 4232 and positioned against second end 4233 at one end of resilient member 4239 and against an interior surface of first flange 4211 at an opposing end of resilient member 4239. Resilient member 4239 may be configured to transition between a compressed configuration and an expanded configuration in response to a rotation of activator 4230, and particularly the plurality of splines 4236 relative to first opening 4217, as caused by an actuation of button 4250. In the example, resilient member 4239 may be compressed in a longitudinal direction parallel to longitudinal axis 10 (see FIG. 103) between second end 4233 and first flange 4211 when in an energy-storing state, as seen in FIG. 109. With resilient member 4239 in the energy-storing state (e.g., a compressed configuration), central body 4232 may be positioned in a first location in which first end 4231 is received through first opening 4217 and splines 4236 are engaged with detents 4217A and misaligned with slots 4217B.

Resilient member 4239 may be movable to an energy-released state (e.g., an expanded configuration), in which central body 4232 is moved towards a second location that is different than the first location (see FIG. 110), upon movement (e.g., rotation) of activator 4230, and particularly the plurality of splines 4236 relative to first opening 4217. In this instance, the plurality of splines 4236 may disengage the plurality of detents 4217A and may become aligned with the plurality of slots 4217B, such that the plurality of splines 4236 may be received through slots 4217B upon the explanation of resilient member 4239. With the plurality of splines 4236 and terminal end 4238 no longer inhibited from moving relative to first flange 4211 due to an engagement between the plurality of splines 4236 and the plurality of detents 4217A, resilient member 4239 may be configured to apply a force onto central body 4232 and against first flange 4211, thereby causing activator 4230 to move relative to carrier 4210 and away from first flange 4211. As resilient member 4239 pushes central body 4232 away from first flange 4211, the plurality of splines 4236 may extend through the plurality of slots 4217B and terminal end 4238 may extend through first opening 4217 until the plurality of splines 4236 and terminal end 4238 are positioned on an opposite (interior) side of first flange 4211, as seen in FIG. 110.

As seen in FIGS. 109-110, activator 4230 may be in contact with fluid source 4350, and particularly second end 4233 may abut against fluid source 4350 along terminal end 4234. Accordingly, activator 4230 may be configured to move fluid source 4350 in response to resilient member 4239 moving from the energy-storing state (e.g., the compressed configuration) to the energy-released state (e.g., the expanded configuration). Stated differently, terminal end 4234 may push against fluid source 4350, thereby causing fluid source 4350 to move relatively away from second flange 4216 of carrier 4210 and towards valve assembly 4300, in response to button 4250 moving from the first (unactuated) position towards the second (actuated) position to release resilient member 4239 from the expanded configuration towards the expanded configuration. Fluid source 4350 may be activated, pierced, and/or opened in response to interacting with valve assembly 4300, thereby releasing the pressurized medium stored in fluid source 4350 into valve assembly 4300. It should be appreciated that activator 4230 may be held in the first position by carrier 4210, prior to button 4250 moving from the first position towards the second position, due to the interaction of the plurality of splines 4236 on first end 4231 with the corresponding plurality of detents 4217A on first flange 4211. As such, carrier 4210 may be configured to prevent horizontal movement of activator 4230 from the first position towards the second position prior to activation of auto-injector 4100 by button 4250.

Referring to FIGS. 103 and 127-129, button 4250 may be a depressible actuator having a body 4251, a stop tab 4252 (e.g., an impediment), a boss 4253, a fastening mechanism 4254, a first leg 4256, and a second leg 4258. Body 4251 may include a top surface defining a contact interface for actuating button 4250. Stop tab 4252 may extend outwardly from a first side of body 4251, and fastening mechanism 4254 may extend outwardly from a second side of body 4251 that is opposite of stop tab 4252. Stop tab 4252 may include one or more ridges and/or protrusions extending outwardly therefrom. The one or more ridges of stop tab 4252 may abut against at least a portion of shuttle actuator 4260 to inhibit movement of shuttle actuator 4260 relative to carrier 4210 prior to actuation of button 4250. The one or more protrusions of stop tab 4252 may block or otherwise impede movement of indicator slide 4270.

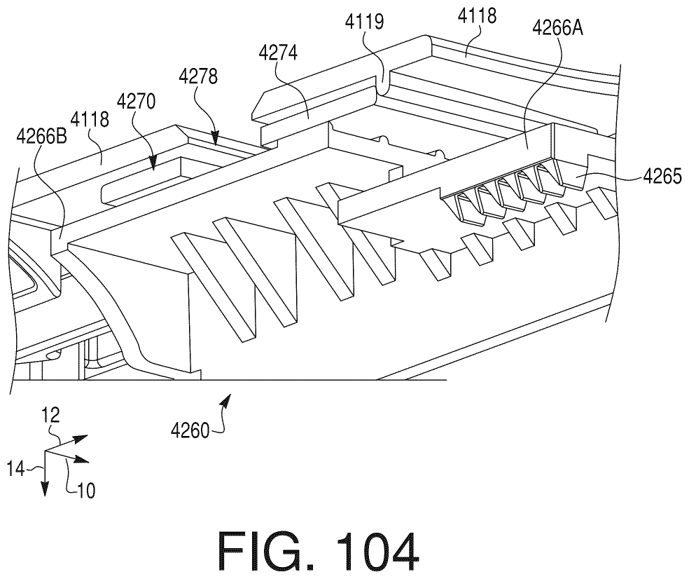

For example, as best seen in FIG. 107, stop tab 4252 may include at least one protrusion 4252A extending outwardly from body 4251 and at least one ridge 4252B defining a terminal end of stop tab 4252 opposite of body 4251. In other embodiments, stop tab 4252 may include additional and/or fewer ridges and/or protrusions along various other sides of stop tab 4252 without departing from a scope of this disclosure. In some embodiments, and as described in further detail herein, protrusion 4252A may be omitted from stop tab 4252 and top cover 4118 may include a stop tab 4119, as seen in FIG. 104. Fastening mechanism 4254 may include a flexible hook or clasp for engaging the pair of protrusions 4220 of carrier 4210. As described in detail herein, fastening mechanism 4254 may be configured to position button 4250 at the first (unactuated) position relative to carrier 4210 prior to actuation, and maintain button 4250 at the second (actuated) position after actuation, in response to interacting with one or more of the pair of protrusions 4220.

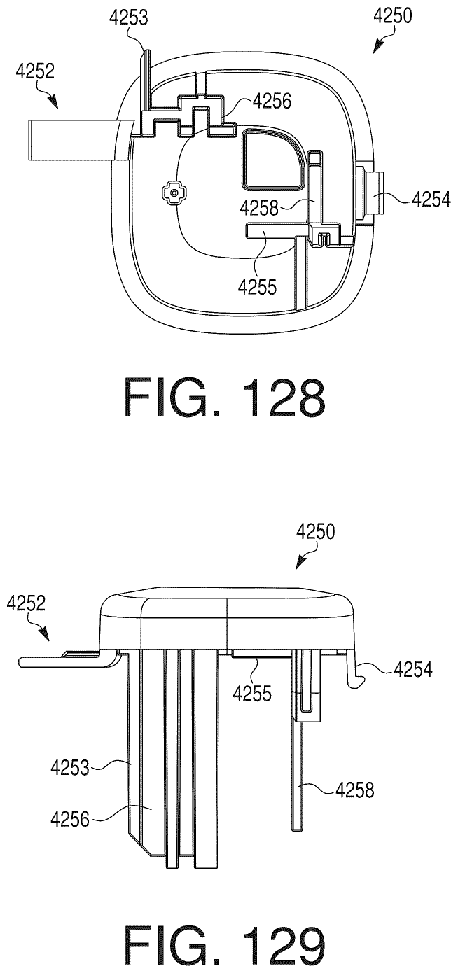

As best seen in FIGS. 128-129, button 4250 may include a protrusion 4255 along a bottom surface of body 4251. Protrusion 4255 may be configured to engage a resilient member 4249 (see FIG. 103) disposed within carrier 4210. Resilient member 4249 may include a spring that is configured to apply a resistive force against button 4250 to generate a tactile feedback against body 4251 when a user actuates button 4250. In other embodiments, resilient member 4249 may be omitted entirely such that button 4250 may exclude protrusion 4255. First leg 4256 and second leg 2458 may extend outwardly (e.g., downwardly) from body 4251, and first leg 4256 may have a longitudinal length that is greater than second leg 4258. First leg 4256 may include a ramp 4259 positioned along an exterior surface of first leg 4256, which may be configured to engage at least a portion of carrier 4210 upon actuation of button 4250. Second leg 4258 may extend outwardly (e.g., downward) from body 4251 along a side of button 4250 that is adjacent to fastening mechanism 4254. Second leg 4258 may be configured to engage at least a portion of carrier 4210 upon actuation of button 4250. Boss 4253 of button 4250 may extend outwardly (e.g., downward) from body 4251 along a side of button 4250 that is opposite of second leg 4258. Boss 4253 may be sized, shaped, and/or otherwise configured to interact with activator 4230 upon actuation of button 4250, such as to cause movement (e.g., rotation) of activator 4230.

As seen in FIG. 108A, boss 4253 may be positioned adjacent to and/or against flange 4235 of activator 4230 when button 4250 is in the first (unactuated) position. In this instance, movement of button 4250 towards the second (actuated) position may cause boss 4253 to abut against flange 4235, thereby causing a corresponding movement of activator 4230. For example, referring now to FIG. 108B, button 4250 may be configured to move activator 4230 in response to boss 4253 pushing against flange 4235, thereby causing second end 4233 to rotate relative to carrier 4210. With second end 4233 forming an integral body with central body 4232 and first end 4231, rotation of second end 4233 causes a simultaneous rotation of central body 4232 and first end 4231 relative to carrier 4210. As described above, movement (e.g., rotation) of first end 4231 relative to first flange 4211 may cause the plurality of splines 4236 to move out of alignment with the plurality of detents 4217A and into alignment with the plurality of slots 4217B. In this instance, with splines 4236 no longer in engagement with detents 4217A, terminal end 4238 may no longer be constrained to the fixed position relative to first flange 4211. In response to splines 4236 disengaging detents 4217A, a force applied to central body 4232 by resilient member 4239, in a direction away from first flange 4211, may move splines 4236 and terminal end 4238 of first end 4231 through first opening 4217, as seen in FIG. 110. As such, activator 4230 may be configured to move towards second flange 4216 and through second opening 4218 to push terminal end 4234 against fluid source 4350 to initiate delivery of the pressurized fluid stored in fluid source 4350 to valve assembly 4300.

Referring back to FIG. 103, container 4370 may be prevented from establishing fluid connection with fluid conduit 4280 by stop tab 4252 extending from button 4250 that may be configured to engage shuttle actuator 4260, which is coupled to container 4370. For example, ridge 4252B may be positioned in a movement path of shuttle actuator 4260 when button 4250 is in the first (unactuated) position, thereby preventing horizontal movement of shuttle actuator 4260, and container 4370 coupled to shuttle actuator 4260, in a direction towards sterile connector 4290 and fluid conduit 4280. Protrusion 4252A may be configured to engage indicator slide 4270 to obstruct movement of indicator slide 4270 in the same direction.

In other embodiments, as seen in FIG. 104, stop tab 4252 may omit protrusion 4252A entirely, and top (outer) cover 4118 of auto-injector 4100 may include stop tab 4119 (e.g., an impediment) for obstructing movement of indicator slide 4270 in the direction towards sterile connector 4290. Stop tab 4119 may extend radially inwards from an interior surface of top cover 4118 in a direction parallel to transverse axis 14, and may be positioned along top cover 4118 at a location that aligns with a first end of indicator slide 4270. In other words, stop tab 4119 may extend vertically within auto-injector 4100. Accordingly, stop tab 4119 may be configured to engage and/or abut indicator slide 4270 to obstruct movement of indicator slide 4270 in a direction parallel to longitudinal axis 10 (e.g., towards sterile connector 4290). Stop tab 4119 may be sized and/or shaped to abut against body 4274 when button 4250 is in the first (unactuated) position and in the second (actuated) position. Stop tab 4119 may be configured to hold indicator slide 4270 in a fixed position such that indicator slide 4270 may be mated and/or coupled to shuttle actuator 4260 after fluid released from fluid source 4350 drives container 4370 (and shuttle actuator 4260) in the horizontal direction towards sterile connector 4290. It should be appreciated that, in further embodiments, auto-injector 4100 may include each of protrusion 4252A on stop tab 4252 and stop tab 4119 on top cover 4118.

In this state, prior to actuation of auto-injector 4100, indicator slide 4270 may be in a first position relative to shuttle actuator 4260. In embodiments where a housing of auto-injector 4100 includes a window for visualizing needle mechanism 4200, a user may identify a relative state of auto-injector 4100 through the window based on visualizing the first position of indicator slide 4270. As described in detail herein, in some embodiments, indicator slide 4270 may include a graphical interface on a body 4274 of indicator slide 4270 that may be configured to indicate the relative position of indicator slide 4270 and/or a state of auto-injector 4100. Furthermore, the first position of indicator slide 4270 may be visually indicated to a user based on a graphical display and/or interface (e.g., a sticker, a color, etc.) positioned on shuttle actuator 4260, which may be visible from outside of auto-injector 4100 through both the window on the outer surface of auto-injector 4100 and a window 4278 of indicator slide 4270.

Prior to actuation of auto-injector 4100, as seen in FIG. 105A, a leg 4272 of indicator slide 4270 may be positioned adjacent to, but disengaged from, fastening mechanism 4263 of shuttle actuator 4260. As described herein, shuttle actuator 4260 may initially be uncoupled from indicator slide 4270, and may become coupled to indicator slide 4270 such that shuttle actuator 4260 and indicator slide 4270 may be configured to move together within the housing of auto-injector 4100 upon connection with one another. Auto-injector 4100 may include a peel tab 4140 that is removably coupled to bottom cover 4110 (see FIG. 101) to expose one or more openings along a (bottom) tissue-engaging surface of bottom cover 4110, and to allow button 4250 to be depressed. Button 4250 may be actuated by applying a downward force onto body 4251, thereby moving button 4250 from the first (unactuated) position towards the second (actuated) position. Prior to activation of auto-injector 4100, movement of shuttle actuator 4260 relative to carrier 4210 may be inhibited due to stop tab 4252 abutting against a portion of shuttle actuator 4260. Accordingly, moving button 4250 towards the second position may disengage stop tab 4252 from shuttle actuator 4260, thereby permitting movement of shuttle actuator 4260 relative to (e.g., horizontally towards) carrier 4210. In embodiments in which top cover 4118 includes stop tab 4119 in engagement with indicator slide 4270 (see FIG. 104), movement of indicator slide 4270 towards sterile connector 4290 may be initially inhibited as shuttle actuator 4260 moves away from carrier 4210 upon initial actuation of button 4250.

Referring to FIGS. 109-110, upon moving button 4250 towards the second position to rotate activator 4230, resilient member 4239 may be configured to automatically move from the first energy-storing state (FIG. 109) towards the first energy-released state (FIG. 110) in which resilient member 4239 expands. In this instance, resilient member 4239 may be configured to push activator 4230 away from first flange 4211 and towards fluid source 4350. Carrier 4210 may include a guide face 4221 that is sized, shaped, and/or otherwise configured to at least partially receive fluid source 4350 therein. Guide face 4221 may be further configured to direct and/or guide movement of fluid source 4350 towards valve assembly 4300 in response to resilient member 4239 pushing activator 4230 towards fluid source 4350. In other words, a release of energy from resilient member 4239 may be configured to move activator 4230, and particularly second end 4233, horizontally from a first lateral position (FIG. 109) towards a second lateral position (FIG. 110) to cause movement of fluid source 4350 within auto-injector 4100 and into fluid communication with valve assembly 4300.

Additionally, activator 4230 may be configured to activate fluid source 4350 by, for example, directly contacting a portion of fluid source 4350 with terminal end 4234 for releasing the pressurized fluid stored therein. For example, activator 4230 may contact, abut, and/or and move a valve stem of fluid source 4350 into an open configuration, to enable the flow of fluid (e.g., gas) from fluid source 4350. In other examples, the valve stem of fluid source 4350 may be positioned within or adjacent to valve assembly 4300 and in a stationary state, such that activator 4230 may be configured to push against the valve stem to thereby move fluid source 4350 to the open configuration. In some embodiments, a feedback (e.g., tactile, audible, etc.) may be generated by needle mechanism 4200 in response to activator 4230 contacting fluid source 4350, to indicate initiation of a dose delivery sequence of auto-injector 4100 to a user.

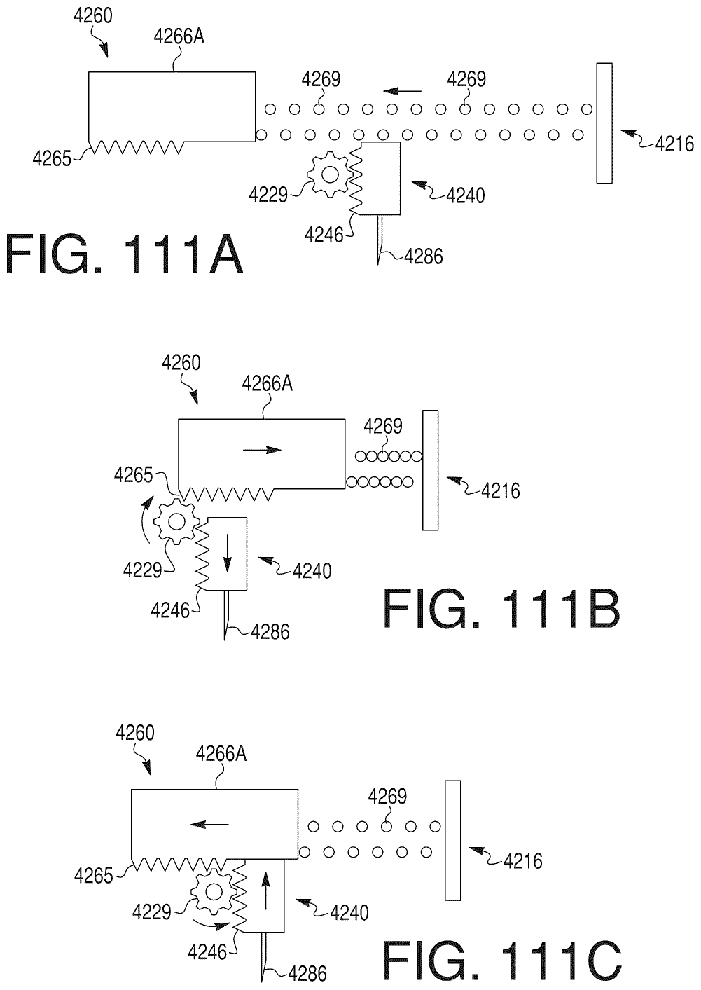

Referring to FIGS. 105A-105B, resilient member 4269 of needle mechanism 4200 may be disposed about first leg 4212A of carrier 4210 and compressed between second flange 4216 and shuttle actuator 4260, and particularly against the pair of guide flanges 4224. Resilient member 4269 may be configured to move shuttle actuator 4260 horizontally from a first position towards a second position upon expansion in response to the release of pressurized fluid by fluid source 4350. As seen in FIG. 111A, shuttle actuator 4260 may be configured to translate horizontally as resilient member 4269 expands due to the contact between resilient member 4269 and guide flanges 4224. Shuttle actuator 4260 may be configured to move from the second position towards a third position, causing resilient member 4269 to compress, in response to the pressurized fluid released by fluid source 4350. Thus, when shuttle actuator 4260 moves in the horizontal direction from a first state towards a second state, shuttle actuator 4260 may be configured to compress resilient member 4269 in the same horizontal direction as seen in FIG. 111B. Stated differently, once the pressurized fluid is release from fluid source 4350, the released energy from the pressurized fluid may be operable to act on container 4370 (see FIG. 101) in a horizontal direction that is opposite of resilient member 4269 during its initial expansion to the energy-released state.

The released energy may be configured to push container 4370, and shuttle actuator 4260 coupled to container 4370, in the opposite horizontal direction, thereby compressing resilient member 4269. It should be understood that this movement of the components of needle mechanism 4200 in the opposite horizontal direction may be operable to establish fluid communication between container 4370 and fluid conduit 4280. For example, fluid communication may be established when container 4370 moves onto a stationary, second end (needle) 4288 of fluid conduit 4280 (see FIG. 102B), thereby forcing needle 4288 through stopper 4380 of container 4370. Still further, this same opposite horizontal movement of the components of needle mechanism 4200 may be configured to drive injection of a first end (needle) 4286 of fluid conduit 4280 (see FIG. 133) out of auto-injector 4100 and into a patient.

Referring to FIG. 111B, movement of shuttle actuator 4260 in the second horizontal direction causes a rack 4265 of shuttle actuator 4260 to rotate gear 4229 in a first rotational direction (e.g., clockwise), which forces driver 4240 in the downward vertical direction (needle injection) via contact between gear 4229 and a rack 4246 of driver 4240. As described above, gear 4229 may include two gear portions 4229A, 4229B of varying sizes such that each body portion may have a different pitch ratio (see FIG. 123). For example, gear 4229 may have a 4:3 pitch ratio difference between the two gear portions 4229A, 4229B, and the gear portions 4229A, 4229B may have a different diameter relative to one another, such that gear 4229 may move shuttle actuator 4260 along a first distance (e.g., ranging from about 1 mm to 6 mm) while simultaneously moving driver 4240 along a second distance (e.g., ranging from about 6 mm to 10 mm) that is different (e.g., greater) than the first distance. For example, 6 mm of travel of shuttle actuator 4260 may correspond to 8 mm of travel for driver 4240.

It is further noted that the one or more gears and/or gear portions of the present disclosure are not limited to a specific pitch, diameter, length, and/or combination thereof. The gears and gear portions can be adjustable and may be modified to accommodate any patient needle insertion depth. As described above, once container 4370 is in fluid communication with fluid conduit 4280, and first end 4282 of fluid conduit 4280 is deployed into a patient (FIG. 111B), further release of energy (e.g., pressurized fluid) from fluid source 4350 may be operable to move piston 4378 through container 4370, thereby expelling medicament 20 into the patient (see FIG. 102C).

In some embodiments, a feedback (e.g., tactile, audible, etc.) may be generated by needle mechanism 4200 in response to shuttle actuator 4260 contacting one or more components of needle mechanism 4200 (e.g., gear 4229, resilient member 4269, etc.) to indicate delivery of a dose to the user. With button 4250 having been moved towards the second position (FIG. 110) to initiate the dose delivery sequence described above, stop tab 4252 may be positioned along a new plane that is relatively lower than when button 4250 was in the first position (FIG. 109). Accordingly, stop tab 4252 may now engage one or more of shuttle actuator 4260 and/or indicator slide 4270 as they move horizontally toward button 4250. In this state, leg 4272 may move toward, and engage, fastening mechanism 4263 (see FIG. 105A) such that shuttle actuator 4260 may become coupled to indicator slide 4270. In this instance, shuttle actuator 4260 and indicator slide 4270 may be configured to move together in response to shuttle actuator 4260 and indicator slide 270 being coupled to one another. In particular, stop tab 4252 (and/or stop tab 4119) may maintain the horizontal position of indicator slide 4270 (preventing indicator slide 4270 from moving toward sterile connector 4290) while shuttle actuator 4260 moves from its first state to its second state, allowing shuttle actuator 4260 to interlock with indicator slide 4270.

As seen in FIG. 103 and described above, protrusion 4252A on stop tab 4252 may abut against indicator slide 4270 while ridge 4252B of stop tab 4252 contacts shuttle actuator 4260. Protrusion 4252A may extend parallel to transverse axis 14 and ridge 4252B may extend parallel to longitudinal axis 10, such that protrusion 4252A is arranged perpendicular relative to ridge 252B. Upon actuation of button 4250, protrusion 4252A may maintain contact with indicator slide 4270, preventing movement of indicator slide 4270 in the horizontal direction towards sterile connector 4290, even after shuttle actuator 4260 has disengaged ridge 4262B. It should be appreciated that protrusion 4252A may have a length sufficient to maintain contact with indicator slide 4270 after movement of button 4250 from the first (unactuated) position towards the second (actuated) position. In other embodiments, as seen in FIG. 104 and described above, stop tab 4119 may maintain contact with indicator slide 4270 in lieu of and/or in addition to protrusion 4252A.

Therefore, upon actuation of auto-injector 4100, movement of shuttle actuator 4260 from its first state to its second state causes a second indicator (positioned on the surface of shuttle actuator 4260), to be visible through the window of the housing of auto-injector 4100 and through window 4278. For example, shuttle actuator 4260 may include a second platform 4266B including the second indicator thereon. In this instance, the window may align with a portion of body 4274 and/or window 4278 of indicator slide 4270, which may thereby reveal a portion of second platform 4266B (containing the second indicator) disposed underneath body 4274. As described herein, second platform 4266B may include one or more graphical interfaces (e.g., color, text, marking, etc.) indicative of a state of auto-injector 4100 when viewable by a user through window 4278. The first and second indicators on second platform 4266B may have different characteristics and/or attributes, such as but not limited to, color and/or patterned pieces of material, stickers, etching, or other suitable mechanism for providing different visual cues to the user.

Upon completing delivery of the medicament 20 when piston 4378 bottoms out at second end 4374 of container 4370, the pressure acting on container 4370 may be reduced and/or eliminated. For example, venting system 4172 (FIG. 102C) may release a pressure generated within valve assembly 4300 upon delivering the medicament 20 through fluid conduit 4280. The absence of a fluid force acting on container 4370 allows resilient member 4269 to move from its second energy-storing state and/or compressed configuration (FIG. 111B) to its second energy-released state and/or expanded state (FIG. 111C). This expansion or movement of resilient member 4269 may drive shuttle actuator 4260 in a second horizontal direction. The movement of shuttle actuator 4260 horizontally causes rack 4265 to drive gear 4229 in a second rotational direction (e.g., counter-clockwise) that is opposite of the first rotational direction. This opposing rotation retracts needle 4286 out of the patient and into auto-injector 4100 by forcing driver 4240 in the upward vertical direction, as seen in FIG. 111C.

With shuttle actuator 4260 moved horizontally by the expansion of resilient member 4269, indicator slide 4270 may similarly move with shuttle actuator 4260. Accordingly, indicator slide 4270 may move towards a third position, viewable through the window of the housing of auto-injector 4100 to indicate a final state of auto-injector 4100. In some embodiments, a feedback (e.g., tactile, audible, etc.) may be generated by needle mechanism 4200 in response to resilient member 4269 expanding and/or driver 4240 moving vertically to retract fluid conduit 4280 into the housing of auto-injector 4100, thereby indicating completion of the dose delivery to the user. In some embodiments, driver 4240 may contact a surface of carrier 4210 or a top interior surface of the housing of auto-injector 4100 (e.g., top cover 4118), thereby generating a feedback (e.g. tactile, audible) indicating completion of the delivery. In other embodiments, activator 4230 and/or shuttle actuator 4260 may contact one or more components and/or surfaces within auto-injector 4100 to generate a corresponding feedback when the dose is delivered to the user, such as with the force being provided by the second or subsequent expansion of resilient member 4269 after dose delivery. This movement of the indicator slide 4270 with shuttle actuator 4260 may be configured to move window 4278 out of alignment with the window on the exterior of the auto-injector 4100. Instead, a surface of indicator slide 4270 containing a third indicator, that is different than the first and second indicators, may now be visible through the exterior window of auto-injector 4100.

Referring back to FIG. 101 and as described above, auto-injector 4100 may include peel tab 4140 coupled to bottom cover 4110. Peel tab 4140 may include a first end 4142 positioned at least partially beneath, and extending through one or more openings in bottom cover 4110. Peel tab 4140 may include a second end 4144 extending outwardly from an end of bottom cover 4110 at various suitable lengths, shapes, sizes, and/or configurations. Second end 4144 may be selectively graspable by a user to remove peel tab 4140 from bottom cover 4110. It should be appreciated that peel tab 4140 may be configured to enclose the one or more openings along bottom cover 4110 when coupled thereto. Peel tab 4140 may be configured to prevent the depression of button 4250 when peel tab 4140 is coupled to auto-injector 4100.

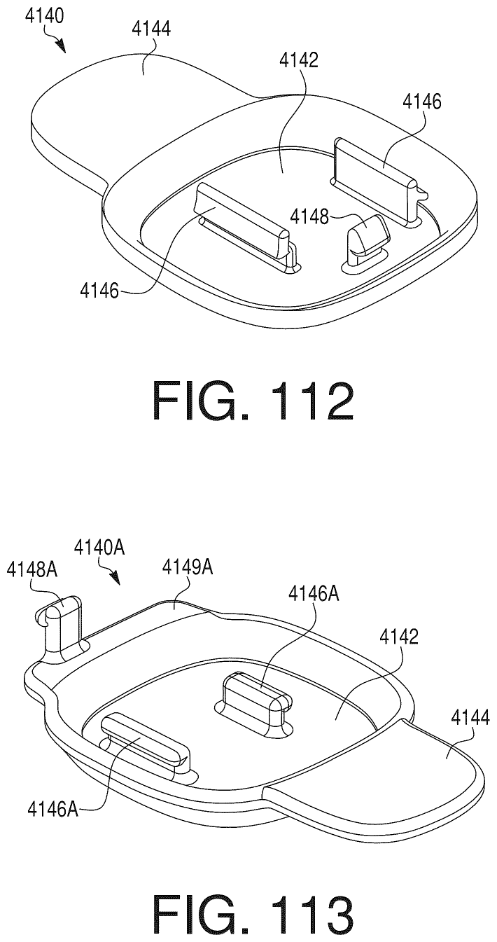

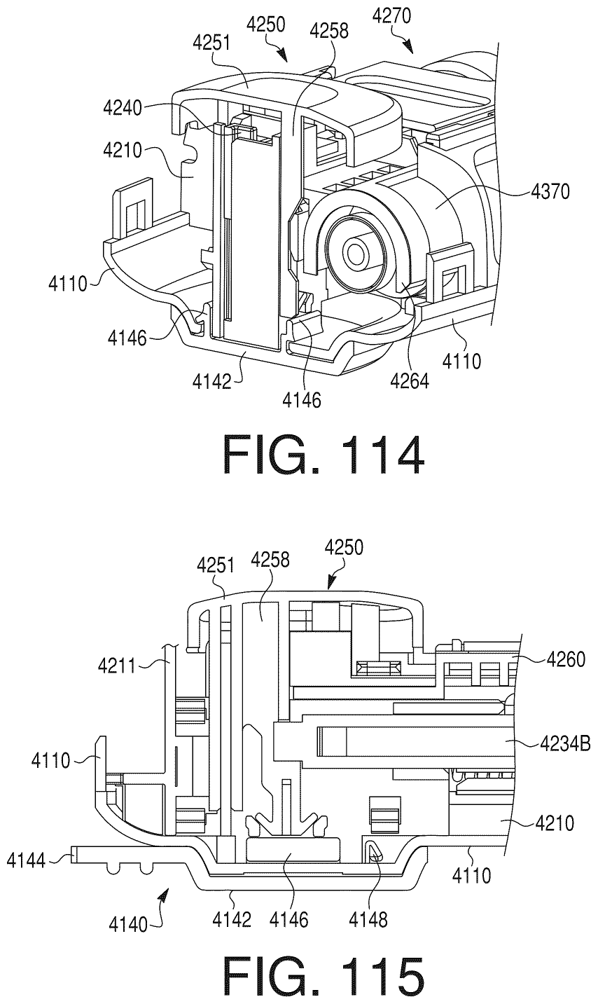

First end 4142 may include a pair of tabs 4146 and a clasp 4148 extending through bottom cover 4110 and into the interior of auto-injector 4100 when peel tab 4140 is coupled to bottom cover 4110. Peel tab 4140 may be disposed on at least a portion of the exterior (tissue-engaging) surface of bottom cover 4110. As best seen in FIG. 112, the pair of tabs 4146 may include vertical stops and/or projections extending outwardly (e.g., upward) from an interior surface of first end 4142. As described in detail herein, the pair of tabs 4146 may be configured to form an impediment that obstructs actuation (e.g., vertical translation) of button 4250 until removal of peel tab 4140 from bottom cover 4110 (see FIGS. 114-118). In the embodiment, the pair of tabs 4146 may have varying sizes (e.g., length, height, width) relative to one another, corresponding to a height of a portion of button 4250 (e.g., first leg 4256, second leg 4258) that the tab 4146 will be aligned with when peel tab 4140 is coupled to bottom cover 4110. Clasp 4148 may include a hook or other suitable fastening mechanism capable of securing peel tab 4140 to bottom cover 4110.

FIG. 113 shows another implementation of a peel tab 4140A, which may be compatible with bottom cover 4110 in a manner substantially similar to that shown and described above. Peel tab 4140A may include a pair of tabs 4146A extending outward from first end 4142, and a clasp 4148A extending outward from a ledge 4149A. In the embodiment, ledge 4149A may be positioned along a plane that is elevated relative to first end 4142, such that clasp 4148A may located along a different surface than the pair of tabs 4146A. It should be appreciated that a position, size, and arrangement of the tabs 4146, 4146A and clasps 4148, 4148A of peel tabs 4140, 4140A shown and described herein are merely exemplary, such that various other suitable configurations may be possible without departing from a scope of this disclosure.

In the embodiment, as seen in FIGS. 114-115, peel tab 4140 may be coupled to bottom cover 4110 and the pair of tabs 4146 may be received through bottom cover 4110 and into a cavity of the housing. At least one of the pair of tabs 4146 may abut a portion of button 4250, such as legs 4256, 4258. Particularly, tabs 4146 may abut against a bottom end of the one or more of legs 4256, 4258 such that the depression of button 4250 may be prevented until peel tab 4140 is removed by a user. Accordingly, peel tab 4140 may be configured to inhibit movement of button 4250 due to tabs 4146 forming an impediment obstructing a (downward) vertical travel path of button 4250 until removal of peel tab 4140. As seen in FIG. 115, clasp 4148 may extend into bottom cover 4110 and engage at least an interior surface (and/or feature) of the housing to maintain an attachment between peel tab 4140 and bottom cover 4110 until a user manually removes peel tab 4140.

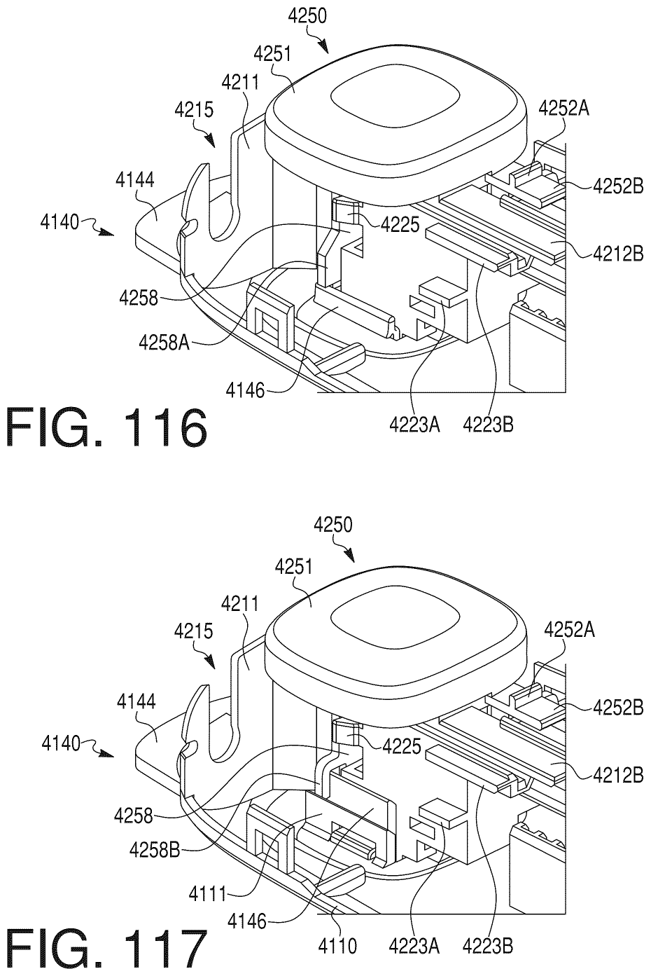

In some embodiments, as seen in FIG. 116, button 4250 may include an extension 4258A at a bottom end of second leg 4258. Extension 4258A may be sized, shaped, and configured to abut against at least one of the pair of tabs 4146. In this instance, peel tab 4140 may be configured to inhibit movement of button 4250 due to tab 4146 forming an impediment obstructing a (downward) travel path of extension 4258A. A user may remove peel tab 4140 (e.g., by applying a downward force onto second end 4144) to separate peel tab 4140 from bottom cover 4110, thereby removing the impediment against extension 4258A and permitting actuation of button 4250.

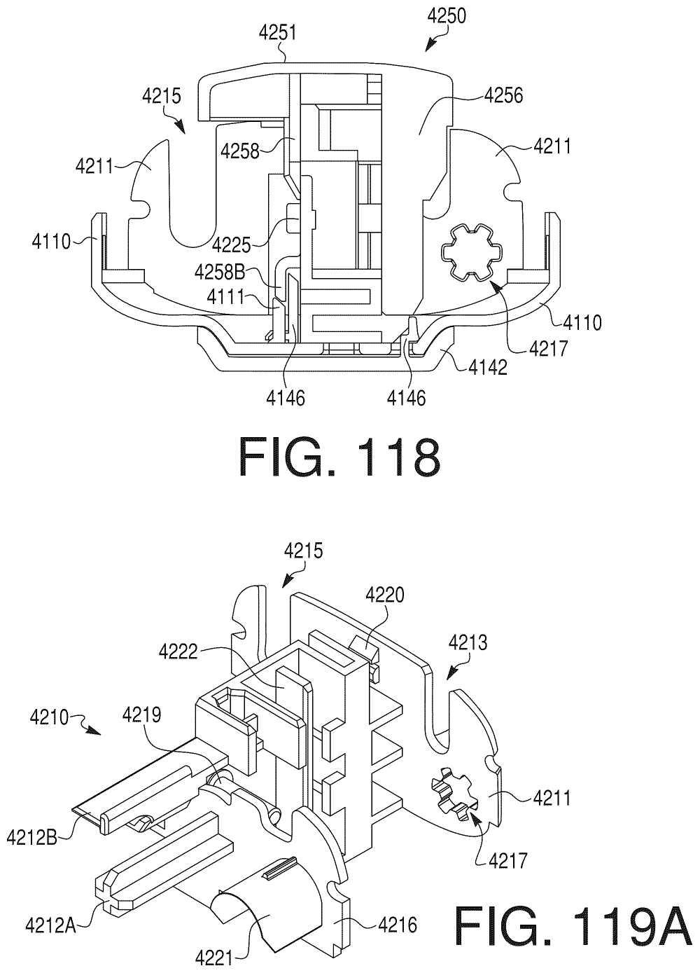

In other embodiments, as seen in FIGS. 117-118, actuator may include a hook 4258B at a bottom end of second leg 4258. Hook 4258B may be sized, shaped, and configured to engage at least one of the pair of tabs 4146. Bottom cover 4110 may include a rib 4111 extending outwardly (upward) from an inner surface of bottom cover 4110. Rib 4111 may be positioned in vertical alignment with hook 4258B. Peel tab 4140 may be configured to inhibit movement of button 4250 due to tab 4146 forming an impediment obstructing a (downward) travel path of second leg 4258.

As seen in FIG. 118, hook 4258B may abut against rib 4111 in response to applying a downward force onto button 4250. A user may remove peel tab 4140 from bottom cover 4110, thereby removing the impediment against extension 4258A by tab 4146 and permitting actuation of button 4250. Upon removal of peel tab 4140, an inclined surface of rib 4111 may be configured to flexibly deform hook 4258B in a radially inward direction (e.g., toward where tab 4146 was previously positioned prior to removal) to facilitate the downward vertical movement of button 4250.

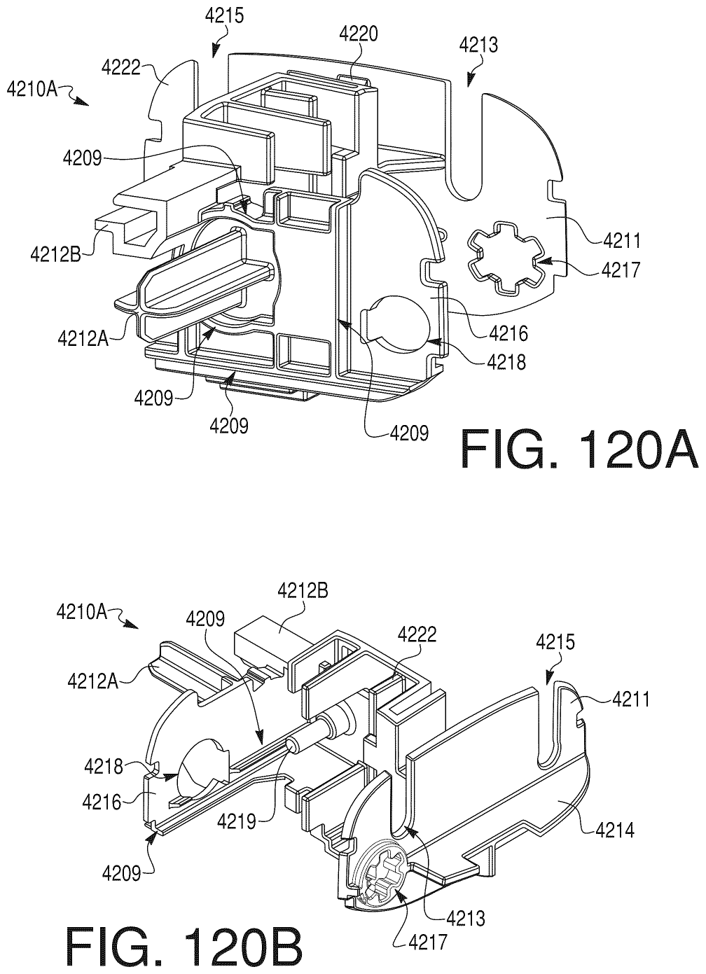

FIGS. 120A-120B depict another exemplary carrier 4210A that may be substantially similar to carrier 4210, such that carrier 4210A may be configured and operable similar to carrier 4210 shown and described above in FIGS. 119A-119B except for the differences explicitly noted herein. As such, carrier 4210A may be incorporated in auto-injector 4100 without departing from a scope of this disclosure. In the example, carrier 4210A may include one or more reinforcing ribs 4209 along various surfaces and/or features of carrier 4210A. For example, carrier 4210A may include at least one reinforcing rib 4209 positioned along and/or adjacent to first leg 4212A, second flange 4216, post 4219, etc. In other examples, additional and/or fewer reinforcing ribs 4209 may be positioned along other various surfaces and/or features of carrier 4210A without departing from a scope of this disclosure.

Each of the one or more reinforcing ribs 4209 may be configured to increase a load strength of carrier 4210A, and particularly of the surface and/or feature of carrier 4210A that the reinforcing rib(s) 4209 is positioned on. Accordingly, reinforcing rib 4209 may allow the corresponding surface and/or feature to carry a greater structural load (e.g., a spring force, an insertion force, etc.) during use of auto-injector 4100. In some embodiments, the one or more reinforcing ribs 4209 and/or carrier 4210A as a whole may be formed of a partial-glass fill material to further enhance a structural strength of carrier 4210A. In further embodiments, the one or more reinforcing ribs 4209 may be configured to allow for the case of moldability of carrier 4210A.

Referring now to FIGS. 124-126, driver 4240 may include a first (bottom) end 4242 and a second (top) end 4244 defining a longitudinal length of driver 4240. Driver 4240 may include a rack 4246 positioned along at least a portion of one of the sidewalls of driver 4240. Rack 4246 may include a plurality of teeth, and may extend along a substantial portion of the longitudinal length of driver 4240 between first end 4242 and second end 4244. Rack 4246 may be configured to engage and mesh with corresponding teeth of gear 4229 (FIG. 123). Driver 4240 may include one or more slots disposed through a body of driver 4240, such as slot 4248 that is sized and shaped to receive a portion of fluid conduit 4280. Accordingly, fluid conduit 4280 may be coupled to driver 4240 at slot 4248 such that movement of driver 4240 relative to auto-injector 4100 (e.g., vertical translation) may provide for a corresponding movement of fluid conduit 4280 (e.g., vertical translation).

Referring to FIGS. 103 and 130-132, shuttle actuator 4260 may include a longitudinal body defined between a first end 4262 and a second end 4264. The longitudinal body of shuttle actuator 4260 may be generally cylindrical, and sized to at least partially receive container 4370. In some embodiments, shuttle actuator 4260 may have an irregularly-shaped body, such as semi-circular (e.g., C-shaped). In this instance, at least a portion of container 4370 may be exposed from underneath shuttle actuator 4260 when received between first end 4262 and second end 4264 (e.g., to facilitate visualization of container 4370 from underneath shuttle actuator 4260 through a housing of auto-injector 4100). As described further herein, shuttle actuator 4260 may be coupled to container 4370, with first end 4262 sized and shaped to receive first end 4372, and second end 4264 sized and shaped to receive second end 4374. Shuttle actuator 4260 may have a longitudinal length that is shorter, equal to, or longer than container 4370. With shuttle actuator 4260 coupled to container 4370, it should be appreciated that shuttle actuator 4260 and container 4370 may be configured to move together and/or simultaneously within the housing of auto-injector 4100 during use.

As best seen in FIG. 130, shuttle actuator 4260 may include a first platform 4266A and a second platform 4266B. First platform 4266A may be positioned adjacent to second end 4264 relative to second platform 4266B, and may include a rack 4265 disposed along a bottom surface of first platform 4266A. As described herein, movement of shuttle actuator 4260 may be at least partially driven by movement of activator 4230, such as from the expansion of resilient member 4239, and pressurized fluid released from fluid source 4350. Further, first platform 4266A may at least partially define a travel path of indicator slide 4270 along shuttle actuator 4260. Second platform 4266B may be positioned adjacent to first end 4262 relative to first platform 4266A, and may include one or more rails for maintaining indicator slide 4270 on shuttle actuator 4260. Second platform 4266B may be operable to serve as an indicator by providing a graphical interface to facilitate visualization of a relative position of shuttle actuator 4260 to a user, which may be indicative of a state of auto-injector 4100 during use.

Second platform 4266B may include a first rail 4268A and a second rail 4268B extending along or parallel to longitudinal axis 10 (see FIG. 103). First rail 4268A and second rail 4268B may be positioned along opposing sides of second platform 4266B, such that rails 4268A, 4268B may define a width of platform 4266B. It should be appreciated that the width of platform 4266B may correspond to a size of indicator slide 4270. Each of rails 4268A, 4268B may include a protrusion and/or overhang portion that is configured to at least partially extend over indicator slide 4270 for maintaining indicator slide 4270 on second platform 4266B during use of auto-injector 4100.

Still referring to FIG. 130, shuttle actuator 260 may include a recessed channel 4269A along second platform 4266B. Recessed channel 4269A may be positioned adjacent to second rail 4268B, and may extend along or parallel to longitudinal axis 10 (see FIG. 103). Recessed channel 4269A may be sized and shaped to receive at least a portion of indicator slide 4270, such as a first edge 4276 of indicator slide 4270. Recessed channel 4269A may be configured to maintain indicator slide 4270 on second platform 4266B during use of auto-injector 4100. Indicator slide 4270 may include the pair of guide flanges 4224 extending outwardly (e.g., downwardly) from a bottom surface of platform 4266B. As described in detail above, the pair of guide flanges 4224 may be configured to couple indicator slide 4270 to carrier 4210 by extending about first leg 4212A. The pair of guide flanges 4224 may be configured to facilitate moment of indicator slide 4270 relative to carrier 4210 in response to resilient member 4269 pushing against the pair of guide flanges 4224 upon expansion (see FIG. 105B).

Referring now to FIG. 131, shuttle actuator 4260 may include a projection 4261 and a fastening mechanism 4263 positioned adjacent to first end 4262. Projection 4261 may be sized and shaped to extend outwardly (e.g., upward) from the longitudinal body of shuttle actuator 4260, and positioned in alignment with fastening mechanism 4263. Projection 4261 may be configured to facilitate receipt of indicator slide 4270 onto shuttle actuator 4260, and alignment of indicator slide 4270 with second platform 4266B. In the embodiment, indicator slide 4270 may include a body 4274 with a first edge 4276 along a sidewall of body 4274. First edge 4276 may be sized and shaped to be received through first rail 4268A, and projection 4261 may align first edge 4276 with first rail 4268A upon receipt of indicator slide 4270 onto shuttle actuator 4260. Indicator slide 4270 may include a second edge 4277 positioned along an opposing sidewall of body 4274 from first edge 4266. Second edge 4277 may be sized and shaped to slide beneath second rail 4268B. In the embodiment, second edge 4277 may be angled relative to body 4274 such that second edge 4277 extends along a different plane than first edge 4276.

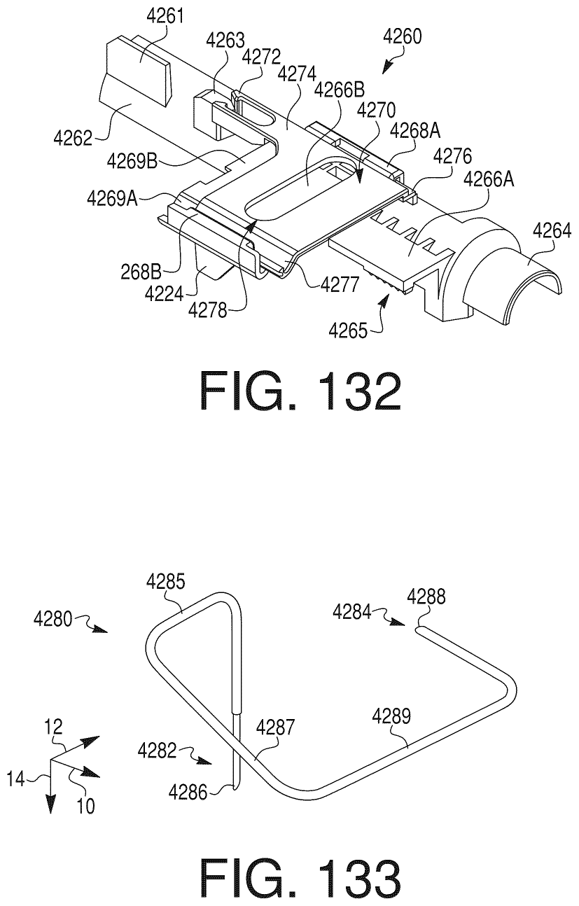

As best seen in FIG. 132, second edge 4277 may be received within recessed channel 4269A when indicator slide 4270 is coupled to shuttle actuator 4260. Fastening mechanism 4263 may be configured to engage a leg 4272 of indicator slide 4270 to couple body 4274 to shuttle actuator 4260. Accordingly, the pair of rails 4268A, 4268B and fastening mechanism 4263 may collectively maintain indicator slide 4270 on shuttle actuator 4260 during use of auto-injector 4100. In some embodiments, indicator slide 4270 may further include a shelf 4269B positioned adjacent to rail 4268A and defining a first edge of second platform 4266B. Shelf 4269B may be configured to engage at least a portion of indicator slide 4270 (e.g., a bottom surface of body 4274) to further couple indicator slide 4270 to shuttle actuator 4260, particularly during assembly of auto-injector 4100. Shelf 4269B, in conjunction with fastening mechanism 4263, may be collectively configured to fix indicator slide 4270 relative to shuttle actuator 4260 upon receipt. Indicator slide 4270 may further include a window 4278 positioned within body 4274. Window 4278 may facilitate visualization of a secondary indicator positioned beneath body 4274, such as, for example, a surface of second platform 4266B. As described in detail above, second platform 4266B may serve as an indicator by providing a graphical interface. Accordingly, window 4278 may facilitate visualization of second platform 4266B through indicator slide 4270.

Referring to FIG. 133, fluid conduit 4280 may be substantially similar to the fluid conduit described in International PCT Application No. PCT/US2020/040729, published as WO 2021/003409, which is incorporated by reference. In the embodiment, fluid conduit 4280 may extend from a first end 4282 towards a second end 4284. First end 4282 may include needle 4286 that is configured to be injected into a user. Needle 4286 may include a sharp and/or beveled tip, and may extend generally along or parallel to transverse axis 14. Second end 4284 may include needle 288 (described previously with respect to FIGS. 102A-102C) that is substantially similar to needle 286, but may be positioned within auto-injector 4100 to penetrate container 4370 to access medicament 420 to be injected into the user. Fluid conduit 4280 may include an intermediate section with a first segment 4285 extending along or parallel to lateral axis 12, a second segment 4287 extending along or parallel to longitudinal axis 10, and a third segment 4289 extending along or parallel to lateral axis 12. First segment 4285 and third segment 4289 may be joined to one another by second segment 4287.

Fluid conduit 4280 may be flexibly deformable along one or more of the ends 4282, 4284 and/or segments 4285, 4287, 4289 of the intermediate section. In this instance, fluid conduit 4280 may flex and/or move, such as, for example, when deploying or retracting needle 4286 relative to a user and/or inserting needle 4288 into container 4370. In some embodiments, fluid conduit 4280 may include a coil (not shown) that may facilitate flexion of fluid conduit 4280 and movement of needle 4286 along transverse axis 14 during deployment and retraction from auto-injector 4100 relative to the user. It should be appreciated that any suitable shape for the coil may be included, e.g., a serpentine, curved, or other shape that enables flexion of fluid conduit 4280. The coil, or similar structure, may act as a cantilever when needle 4286 is deployed and/or retracted.

In some examples, fluid conduit 4280 may include only metal or a metal alloy. In other examples, fluid conduit 4280 may be include any other suitable material, such as, e.g., polymers or the like. Needle 4288 and the intermediate sections may define a 22 or 23 Gauge, thin-walled needle, while needle 4286 may be a 27 Gauge needle. In other words, fluid conduit 4280 may have a varying needle gauge across its length, and in particular, needle 4286 and needle 4288 may have different needle gauges. Other needle sizes ranging from, e.g., 6 Gauge to 34 Gauge, also may be utilized as appropriate. Fluid conduit 4280 may reduce the amount of material that contacts the medicament 20, reduce the number of joints, reduce the number of assembly steps, and/or require less sterilization than conventional devices.

Referring now to FIGS. 134-135, fluid conduit 4280 may be coupled to driver 4240 along first segment 4285. In the embodiment, first segment 4285 may be received within slot 4248 and coupled to driver 4240. Accordingly, first end 4282, needle 4286, and first segment 4285 may be configured to move in response to movement of driver 4240 relative to auto-injector 4100. It should be appreciated that one or more other intermediate segments of fluid conduit 4280 (e.g., second segment 4287, third segment 4289) may be configured to flex, deform, and/or more to accommodate movement of first end 4282, needle 4286, and first segment 4285. Needle 4286 may extend outward from driver 4240, and positioned adjacent to first (bottom) end 4242.

Fluid conduit 4280 may be further coupled to sterile connector 4290 along second end 4284. In the embodiment, second end 4284 may extend through an opening 4296 of sterile connector 4290 and needle 4288 may be disposed within body 4292. At least a portion of third segment 4289 may be received along seat 4294, thereby fixing fluid conduit 4280 to sterile connector 4290. Once needle 4288 penetrates and establishes fluid communication with container 4370, in response to container 4370 moving relative to (e.g., toward) needle 4288 and sterile connector 4290 (see FIG. 102B), a medicament may travel from container 4370, through needle 4288, the intermediate segments 4285, 4287, 4289, and needle 4286 (pierced through the user's skin), and into the user. Sterile connector 4290 may include a rail disposed within body 292 to facilitate an alignment and receipt of container 4370. In some embodiments, sterile connector 4290 may be configured to move (e.g., rotate) to facilitate movement of fluid conduit 4280 within auto-injector 4100. In this instance, deformation and/or deflection of fluid conduit 4280 may be minimized.

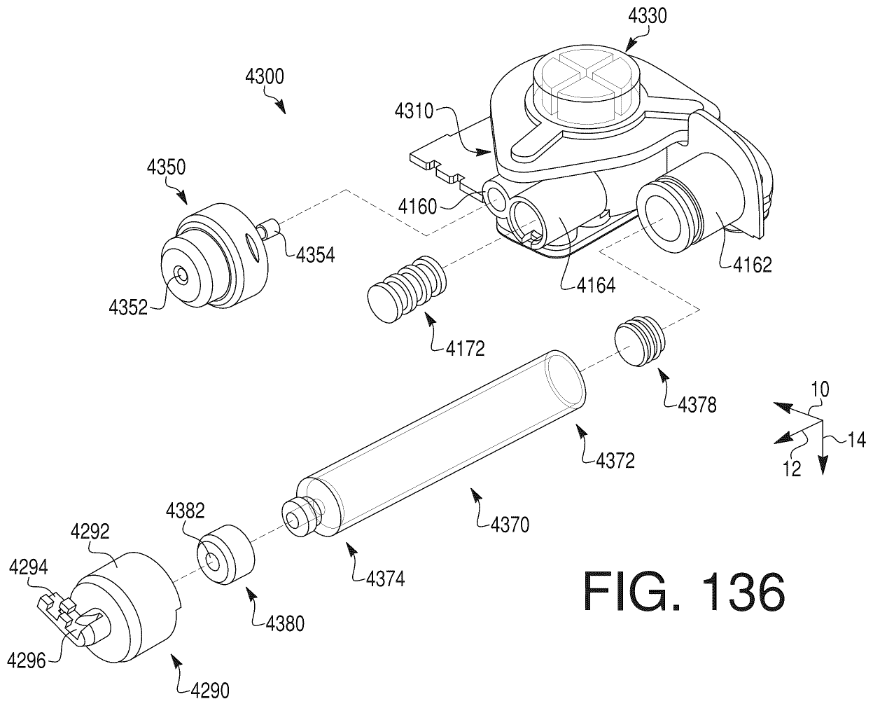

Referring now to FIG. 136 an implementation of valve assembly 4300 is depicted. Valve assembly 4300 may be compatible with container 4370 whose longitudinal axis may be parallel to the surface of the skin of a patient. Valve assembly 4300 may be designed to operate at a specific pressure, based on a balancing of one or more parameters including a thickness and/or durometer of diaphragm 4320, a height of valve seat 4166, and/or a diameter of high pressure cavity 4161 (see FIGS. 102A-102C). In some embodiments, low pressure inlet 4162 may include a seal to facilitate movement of container 4370 relative to valve assembly 4300 (e.g., ranging from between 50 durometer to 70 durometer). During pressure equalization between high pressure cavity 4161 and low pressure cavity 4163, the low pressure in conduit 4164 may create a retention force that may prevent diaphragm 4320 from returning to the neutral stage (FIG. 102A). This may be avoided by reducing the diameter of conduit 4164 and/or increasing the return force of diaphragm 4320 by adjusting one or more of pre-tension, a thickness and/or diameter of diaphragm 4320, or a height of valve seat 4166. For example, a flat, stamped diaphragm 4320 may shift in relation to the rest of the valve due to forces acting on it during deflection such that it may lose its return force.

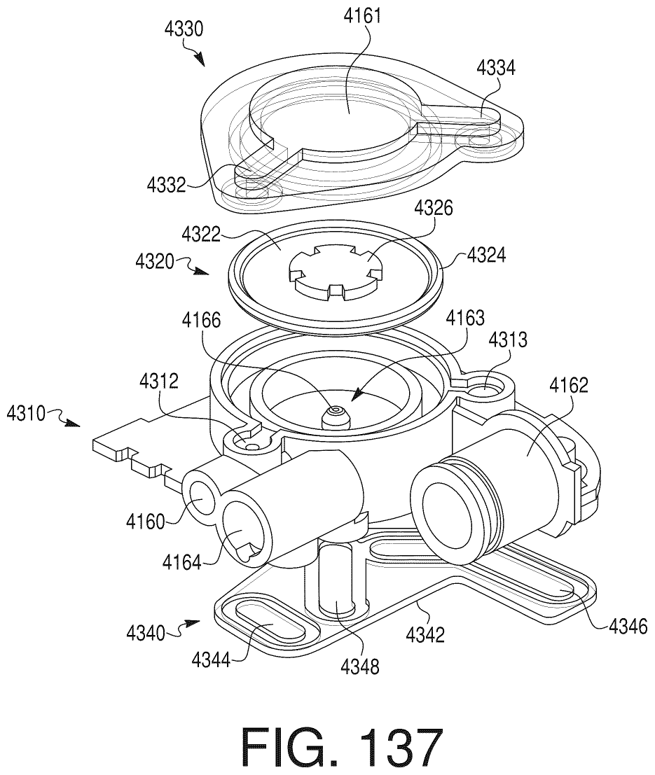

FIG. 137 illustrates a four-piece construction valve assembly 4300 (including diaphragm 4320). The embodiment of FIG. 137 may exhibit improved moldability and simplicity of construction, requiring only one type of weld. In some embodiments, the push rod port may be omitted. Valve assembly 4300 may include low pressure body portion 4310 and high pressure body portion 4330. Low pressure body portion 4310 may include low pressure cavity 4163, and high pressure body portion 4330 may include high pressure cavity 4161 (FIGS. 102A-102C). In some embodiments, high pressure body portion 4330 may include a tenting boss surrounding low pressure cavity 4163, and that stretches diaphragm 4320 (in a manner similar to a drum head) when low pressure body portion 4310 and high pressure body portion 4330 are mated to one another.

Valve seat 4166 may be positioned within low pressure cavity 4163, as shown and described above in FIGS. 102A-102C. Pressurized fluid may flow from the energy source (e.g., fluid source 4350) through high pressure (first) inlet 4160 on low pressure body portion 4310, and flow into high pressure cavity 4161 via a port 4312 of low pressure body portion 4310 and a channel 4332 of high pressure body portion 4330. Low pressure body portion 4310 may further include a port 4313 that contains flow restrictor 4170. Low pressure body portion 4310 may include low pressure inlet 4162 for interfacing with container 4370, and conduit 4164.

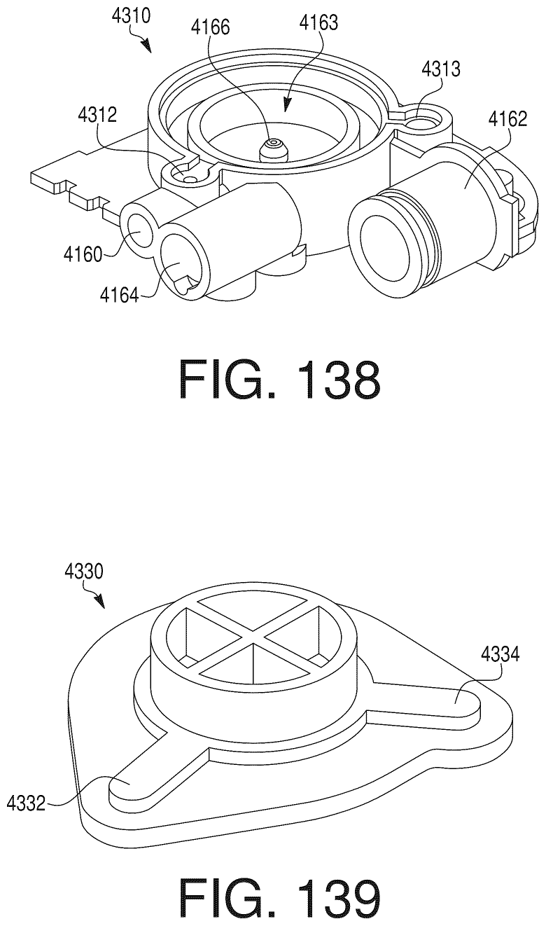

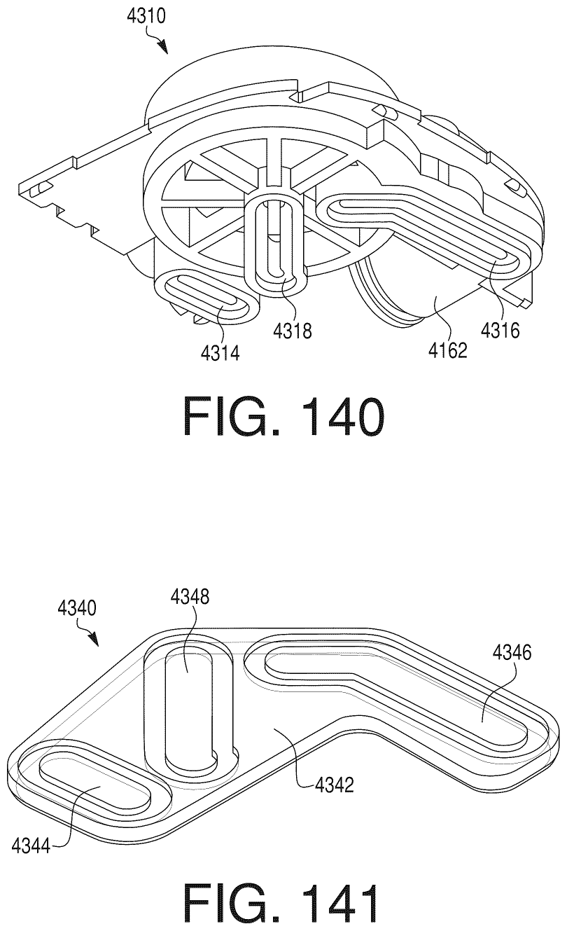

FIGS. 138-141 depict the four-piece construction valve assembly 4300 disassembled into separate components, including low pressure body portion 4310 (FIGS. 138 and 140), high pressure body portion 4330 (FIG. 139), and a bottom cover 4340 (FIG. 141). FIG. 140 illustrates one or more pressure lines 4314, 4316, 4318 positioned along a bottom surface of low pressure body portion 4310. FIG. 141 illustrates one or more pressure cavities 4344, 4346, 4348 formed (e.g., etched) along a body 4342 of bottom cover 4340. Bottom cover 4340 may be coupled to the bottom surface of low pressure body portion 310 such that pressure lines 4314, 4316, 4318 may be aligned with pressure cavities 4344, 4346, 4348. It should be appreciated that bottom cover 4340 may allow gas from low pressure body portion 4310 to travel to one or more locations in valve assembly 4300 via the one or more pressure lines 4314, 4316, 4318 and pressure cavities 4344, 4346, 4348.

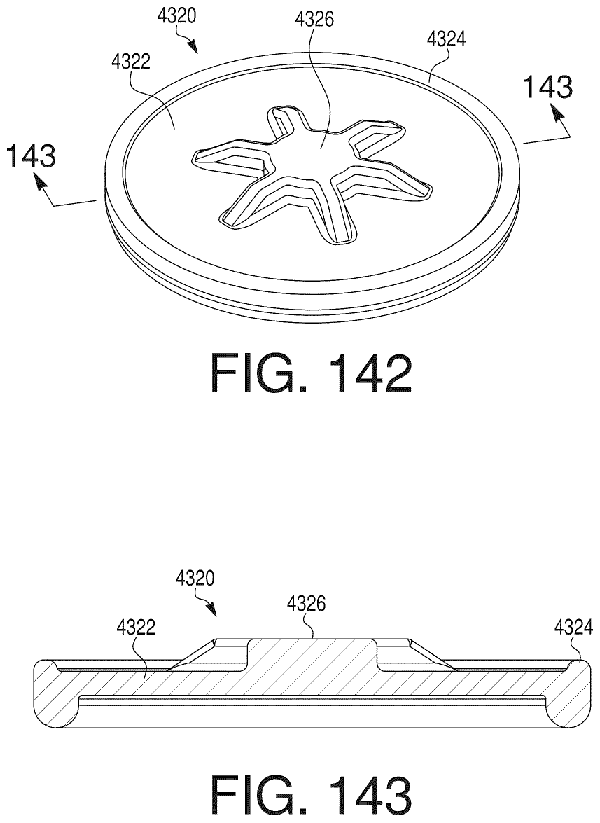

FIGS. 142-143 illustrate different views of an exemplary diaphragm 4320, which may be incorporated in valve assembly 4300 or any other valve assemblies discussed herein (e.g., valve assembly 160). FIG. 142 is a perspective view of a first side of diaphragm 4320. FIG. 143 is a cross-sectional view of a portion of diaphragm 4320. Diaphragm 4320 may have a unitary central body 4322 that is generally circular. Diaphragm 4320 may include an outer rim or gland 4324 that extends around the periphery of body 4322. Gland 4324 may extend away from body 4322 in opposite directions, and may include an increased thickness relative to body 4322 of diaphragm 4320. Gland 4324 may also include a round face, for example, along an entire face of gland 4324 (e.g., the surface extending perpendicularly from the radial direction of diaphragm 4320). Additionally, diaphragm 4320 may include a raised portion 4326 positioned on and/or coupled to body 4322, for example, in a radially centered position on diaphragm 4320. Raised portion 4326 may have a circular center, and may include a thickness (e.g., extending away from body 4322). The thickness of raised portion 4326, may be about 1 mm, about 2 mm, from about 0.5 mm to about 10 mm, from about 1 mm to about 9 mm, from about 3 mm to about 8 mm, from about 4 mm to about 6 mm, or about 5 mm. In some embodiments, the thickness of raised portion 4326 may be at least 1 mm to assist with manufacturability. As shown, raised portion 4326 may include a plurality of fingers that protrude radially outward from the center portion.

Diaphragm 4320 may be formed of a unitary, single, or composite material, or any other suitable material. Diaphragm 4320 with raised portion 4326 may be able to receive a greater force and/or pressure, for example, such that diaphragm 4320 deflects and/or changes shape more uniformly, which may help during lift-off from a valve seat at higher pressures. For example, diaphragm 4320 may have a material composition having a hardness ranging from about 30 durometer to 80 durometer, such as 40 durometer.

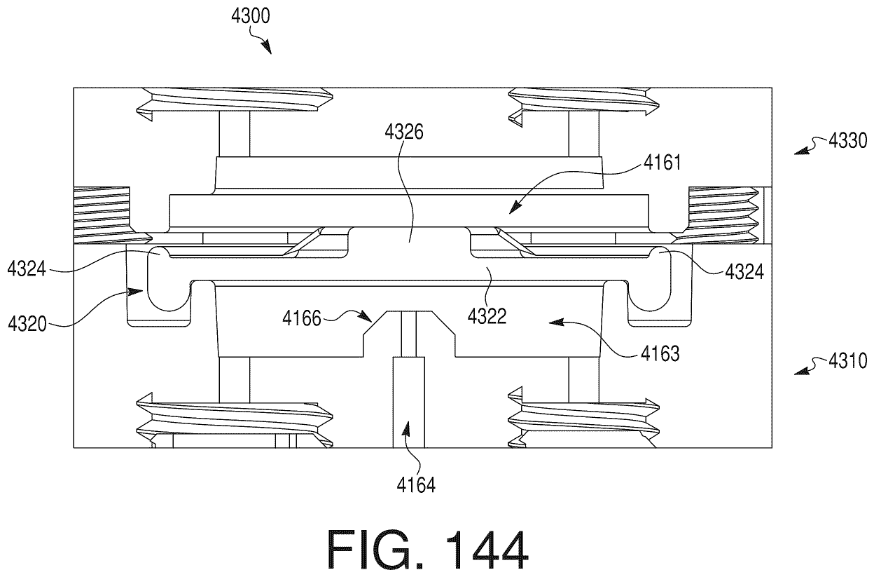

FIG. 144 shows diaphragm 4320 assembled between low pressure body portion 4310 and high pressure body portion 4330. In an initial configuration, diaphragm 4320 may be positioned in a stretched and/or tensioned state with gland 4324 in contact with an upper tooth of high pressure body portion 4330 and offset from a bottom surface of low pressure body portion 4310 by a distance. In the embodiment, a size and/or shape of gland 4324 (in both directions relative to body 4322) may facilitate a deflection and lift off of diaphragm 4320 at specified pressure differentials.

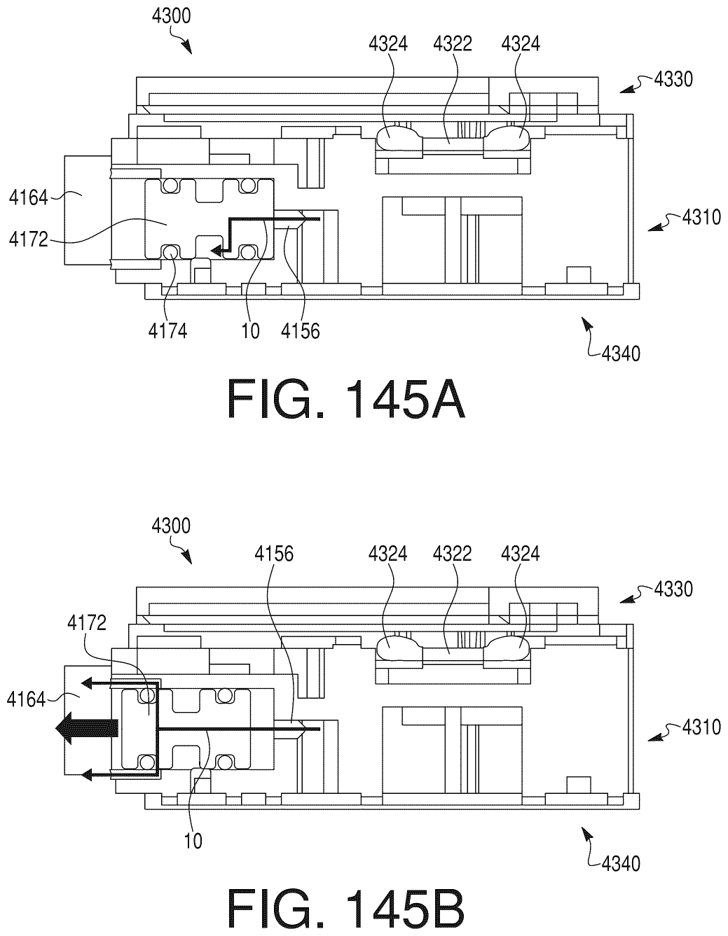

FIGS. 145A-145B illustrate valve assembly 4300 in fluid communication with venting system 4172, and particularly a fluid path 10 that travels through valve assembly 4300 when venting system 4172 is in a first position (FIG. 145A) and a second position (FIG. 145B).

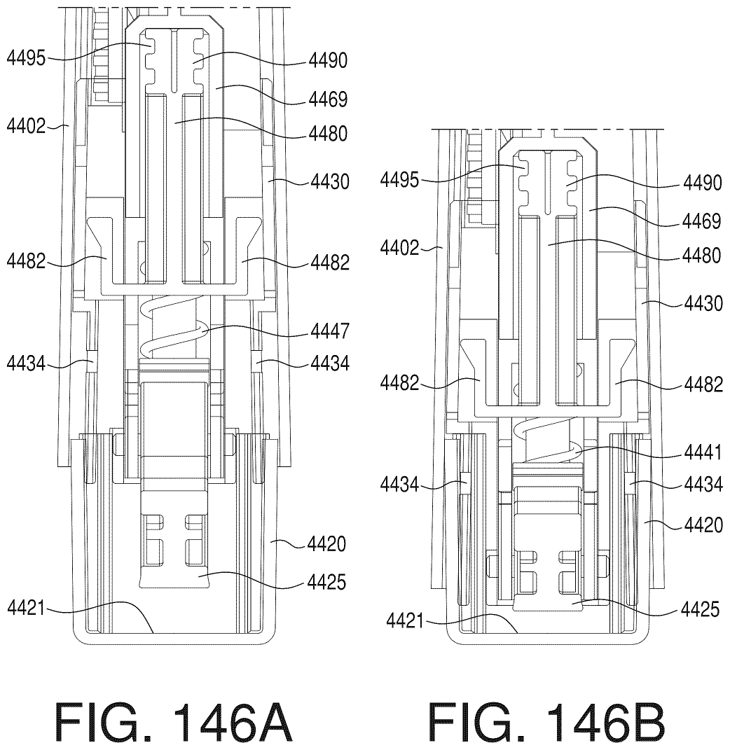

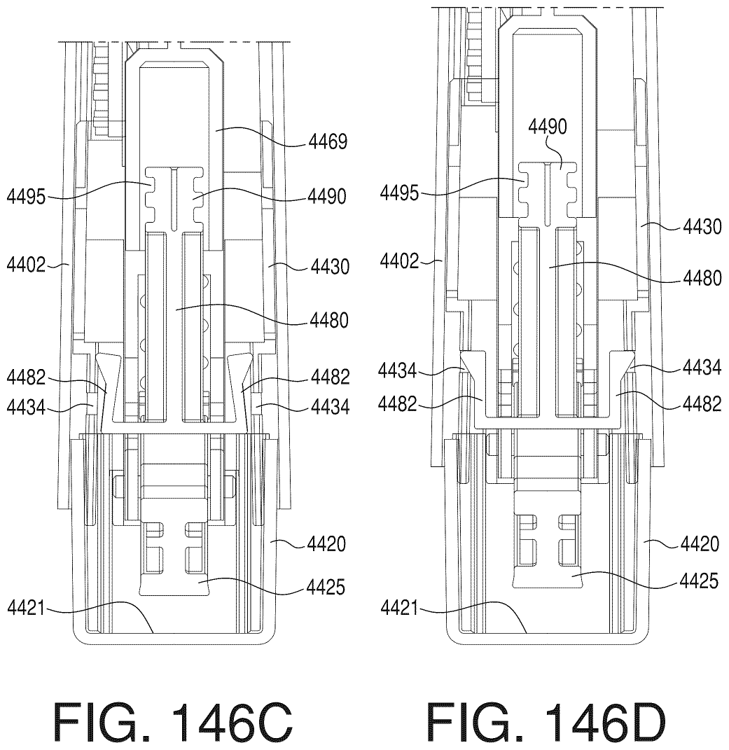

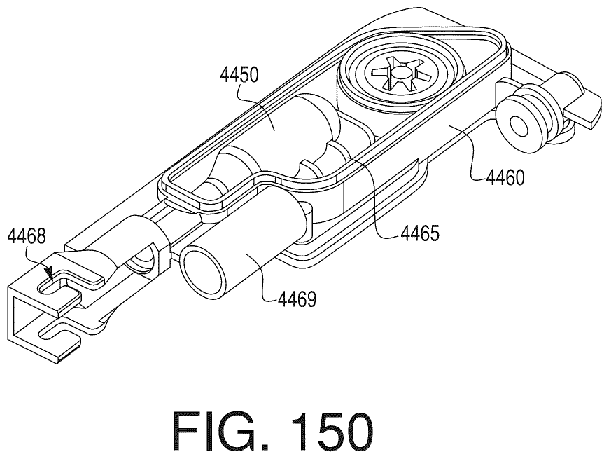

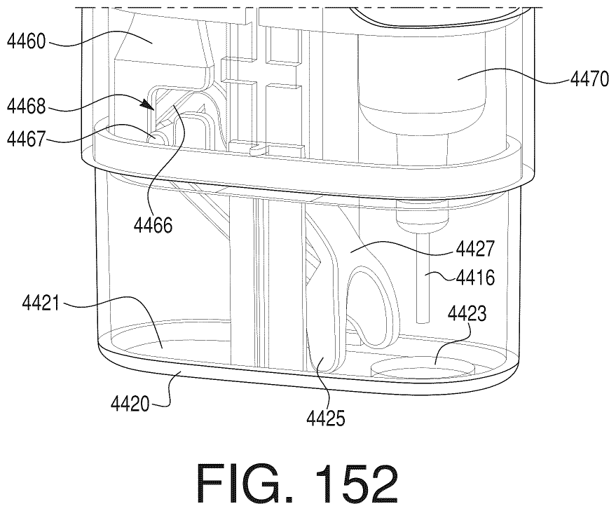

Referring now to FIGS. 146A-146D, another exemplary shroud 4420, chassis 4430, and mandrel assembly 4480 are depicted. It should be appreciated that shroud 4420, chassis 4430, and mandrel assembly 4480 may be incorporated into auto-injector 100 in a substantially similar manner as shroud 120, chassis 130, and mandrel assembly 180 shown and described above, respectively, except for the differences explicitly noted herein. For example, mandrel assembly 4480 may include a slidable piston 4490. The slidable piston 4490 may be coupled to or integrally formed with mandrel assembly 4480, such that mandrel assembly 4480 and slidable piston 4490 may move together as one unit, e.g., in response to a pressurized medium being released by a canister 4450 (FIG. 150) through a valve assembly 4460 (FIG. 150) and into a release outlet 4469 after auto-injector 100 transitions from an activated state to a post-injection state, as described above and below. After auto-injector 100 transitions from the activated state to the post-injection state, mandrel assembly 4480 may be configured to engage with shroud 4420, pushing shroud 4420 downward and/or outward relative to the housing 4402 of auto-injector 100. Mandrel assembly 4480 may further include one or more retention mechanisms 4482. Retention mechanisms 4482 may include, but are not limited to, a clip, a hand, a finger, a tab, a protrusion, a detent, etc. Retention mechanisms 4482 may be configured to engage with one or more lockout windows 4434 included in chassis 4430 as mandrel assembly 4480 pushes downward and/or outward on shroud 4420, allowing mandrel assembly 4480 to lockout shroud 4420 relative to housing 4402, e.g., on or after completing delivery of a dose from auto-injector 100, to inhibit reactivation or prevent re-exposure of a needle (e.g., needle 4416; FIG. 152).

In the example illustrated in FIGS. 146A-146D, mandrel assembly 4480 may be integrally attached to slidable piston 4490. Mandrel assembly 4480 and slidable piston 4490 may be at least partially disposed within release outlet 4469. Slidable piston 4490 may include one or more recesses 4495 that are sized, shaped, and/or otherwise configured to receive a seal (e.g., an O-ring) therein to inhibit a pressurized medium received by release outlet 4469 from passing along an exterior surface of slidable piston 4490. Accordingly, the pressurized medium received by release outlet 4469 may be operable to exert a downward and/or outward force on slidable piston 4490, thereby causing slidable piston 4490 to translate within release outlet 4469, such as in a downward and/or outward direction, from a first (upper) position, as shown in FIGS. 146A-146B, toward a second (lower) position, as shown in FIG. 146D.

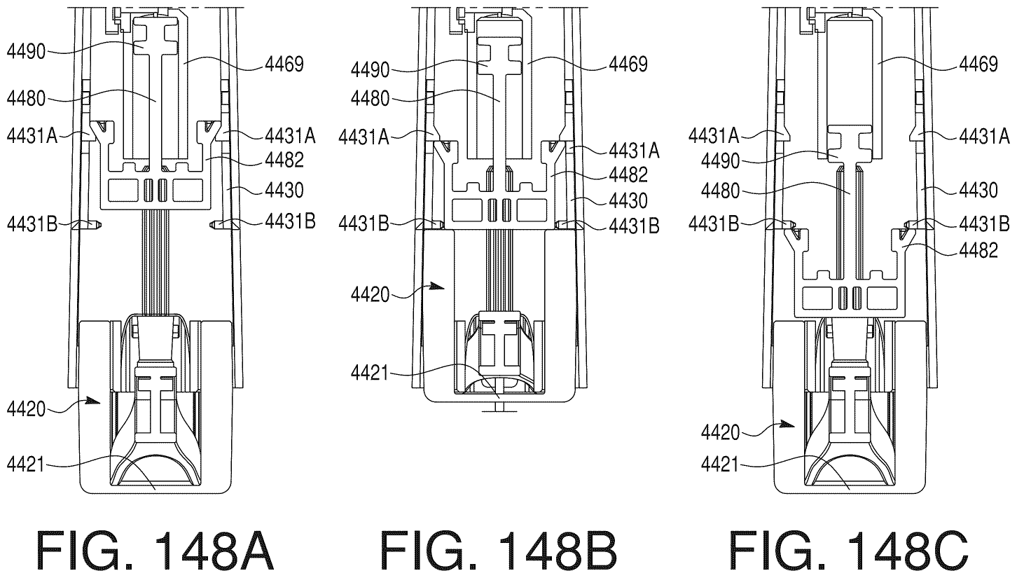

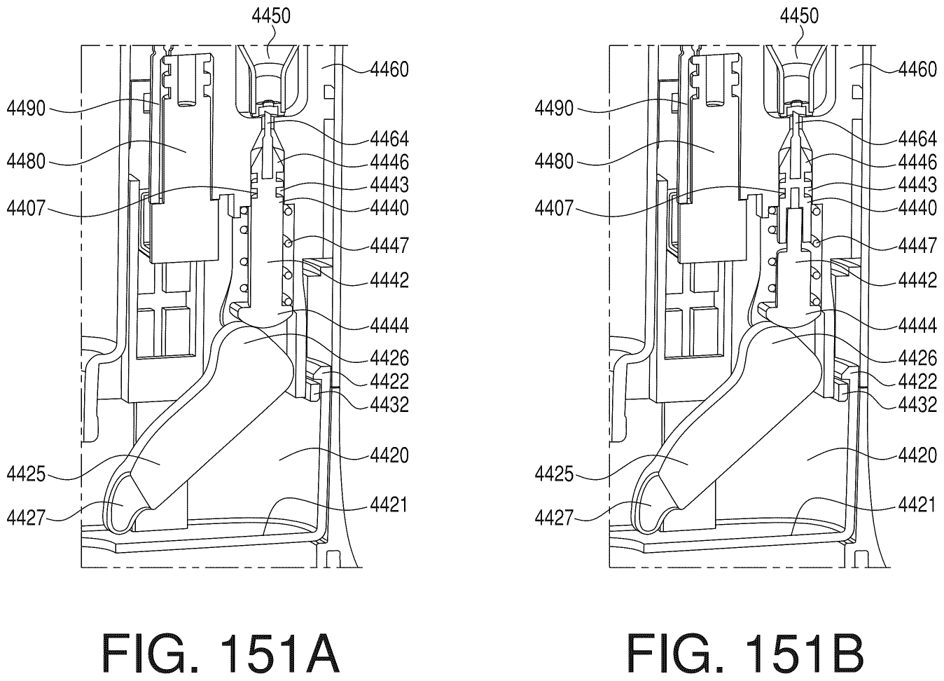

FIG. 146A illustrates auto-injector 100 in a pre-activation state, e.g., before shroud 4420 has been depressed inward and/or upward relative to chassis 4430 to activate auto-injector 100. When auto-injector 100 is in the pre-activation state, shroud 4420 and mandrel assembly 4480 may be in respective first positions. In its respective first position, shroud 4420 may be fully extended downward and/or outward relative to chassis 4430. Referring now to FIGS. 151A and 151B), when auto-injector 100 is at rest in the pre-activation state, shroud 4420 may be held in its first position by a downward and/or outward force exerted on shroud 4420 by an actuator 4425 engaged with an interior bottom surface 4421 of shroud 4420. For example, the downward and/or outward force exerted on shroud 4420 by actuator 4425 may be a translation of a downward and/or outward force exerted on actuator 4425 by an activator 4440. The downward and/or outward force exerted on actuator 4425 by activator 4440 may be a translation of a downward and/or outward force exerted on activator 4440 by a biasing member 4447 (e.g., a spring) disposed about and configured to engage with activator 4440. In other words, when auto-injector 100 is in the pre-activation state, biasing member 4447 may be in a compressed configuration, thereby exerting a downward and/or outward force on activator 4440, which may be translated via activator 4440 and/or actuator 4425 onto the interior bottom surface 4421 of shroud 4420, thereby holding shroud 4420 in its respective first position. Additionally or alternatively, when auto-injector 100 is at rest in the pre-activation state, shroud 4420 may be held in its first position by one or more retaining elements 4422 of shroud 4420 (e.g., one or more lips that protrude from a surface of shroud 4420) configured to engage with one or more corresponding retaining elements 4432 of chassis 4430 (e.g., one or more lips that protrude from a surface of chassis 4430), such that the one or more retaining elements 4422 of shroud 4420 and the one or more corresponding retaining elements 4432 of chassis 4430 prohibit shroud 4420 from being pushed down and/or out of housing 4402. For example, a downward-facing surface of the one or more retaining elements 4422 may be configured to engage an upward-facing surface of the one or more retaining elements 4432. Referring again to FIG. 146A, when mandrel assembly 4480 is in its respective first position, the one or more retention mechanisms 4482 are positioned before the one or more lockout windows 4434 of chassis 4430 along a direction extending from the slidable piston 4490 to the interior bottom surface 4421 of shroud 4420. Mandrel assembly 4480 may be held in its respective first position by friction between one or more seals disposed within the one or more recesses 4495 of slidable piston 4490 and an interior surface of release outlet 4469. Additionally or alternatively, mandrel assembly 4480 and/or slidable piston 4490 may be held in its respective first position at least partially by the engagement between the one or more retention mechanisms 4482 of mandrel assembly 4480 and one or more features of chassis 4430, such as one or more audible feedback elements 4431 (FIG. 148A).

FIG. 146B illustrates auto-injector 100 in an activated state, e.g., after shroud 4420 has been depressed toward chassis 4430 to activate auto-injector 100, thereby causing canister 4450 to release a pressurized medium. For example, referring again to FIGS. 151A and 151B, depressing shroud 4420 may cause shroud 4420 to exert an upward and/or inward force on actuator 4425. Actuator 4425 may translate the upward and/or inward force to activator 4440, which may cause a piercing mechanism 4464 (e.g., a needle, an activator pin, etc.) to puncture a seal and/or a valve of canister 4450. When shroud 4420 is depressed, biasing member 4447 may be in a contracted configuration. Referring back to FIG. 146B, shroud 4420 is depicted in a second, fully depressed position. For example, when shroud 4420 is pressed against an exterior surface of a subject's (e.g., a patient) skin, shroud 4420 may be depressed (e.g., translated) upward and/or inward relative to chassis 4430. The upward and/or inward translation of shroud 4420 may be limited by one or more restricting elements (not shown) of housing 4402 or chassis 4430, such that shroud 4420 may be depressed no further than its second, fully depressed position. Shroud 4420 may remain in its second, fully depressed position until shroud 4420 is released, such as by being removed from the exterior surface of the subject's skin. When shroud 4420 is depressed, a needle 4416 (FIG. 152) of auto-injector 100 may be exposed outside of housing 4402, such as through an opening 4423 (FIG. 152) of shroud 4420, as described above.

FIG. 146C illustrates auto-injector 100 in a post-activation, pre-lockout state, e.g., after shroud 4420 has been depressed upward and/or inward relative to chassis 4430 to activate auto-injector 100 and after shroud 4420 has been released, such as by being removed from the exterior surface of a subject's skin. In the post-activation, pre-lockout state, shroud 4420 may be allowed to move from a depressed position (e.g., its second, fully depressed position) back toward to its first, resting position. In the post-activation, pre-lockout state, shroud 4420 may be alternatively or additionally urged back toward its first, resting position by one or more forces generated by one or more elements of auto-injector 100. For example, upon release of shroud 4420 (e.g., the removal of shroud 4420 from the exterior surface of a subject's skin), biasing member 4447, which may have been put into a further compressed configuration in response to the depression of shroud 4420, may expand back toward its original, less compressed configuration, thereby generating a downward and/or outward force that may be translated and/or applied to shroud 4420 by activator 4440 (FIGS. 151A and 151B) and/or actuator 4425. Or for example, a release of a pressurized medium from canister 4450 (FIG. 150) through valve assembly 4460 (FIG. 150) and into release outlet 4469 may cause the pressurized medium to exert a downward and/or outward force on slidable piston 4490, e.g., in a post-injection state of auto-injector 100, as described in further detail below. In turn, slidable piston 4490 and mandrel assembly 4480 may translate down and/or out (e.g., within release outlet 4469), eventually engaging with and urging shroud 4420 downward toward its first, resting position.

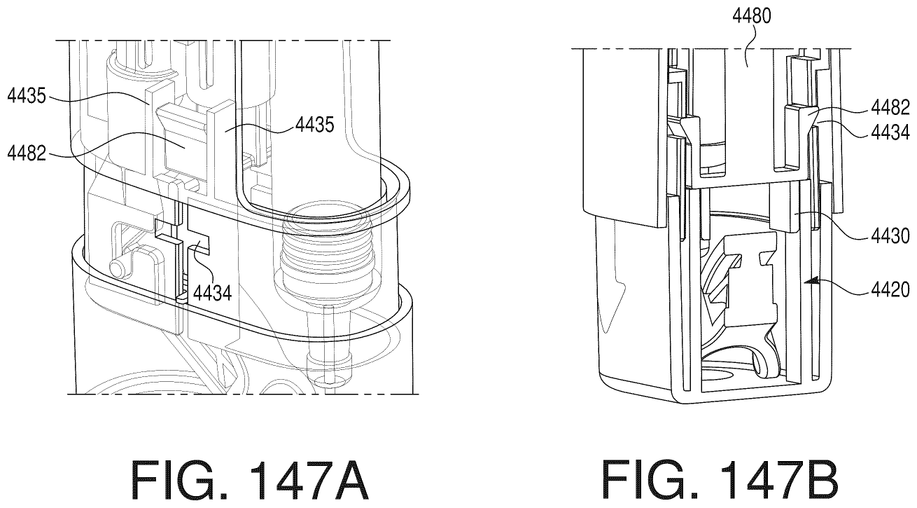

In the example illustrated in FIG. 146A-146D, a width of the interior space of chassis 4430 decreases from an upper portion of chassis 4430 to a lower portion of chassis 4430. Thus, as slidable piston 4490 and mandrel assembly 4480 translate down and/or out in response to the downward and/or outward force exerted by the pressurized medium within release outlet 4469, the one or more retention mechanisms 4482, which are flexible and in contract with the interior wall of chassis 4430, are pressed inward by the decreasing width of the interior space of chassis 4430 (as illustrated in FIG. 146C), until they eventually move into an opening of the one or more lockout windows 4434 of chassis 4430. For example, the one or more lockout windows 4434 may include holes, cutouts, recesses, etc. disposed in the inner and/or outer surface of chassis 4430 and having an opening configured to engage with the one or more retention mechanisms 4482. When the one or more retention mechanisms 4482 move into an opening of the one or more lockout windows 4434, the inwardly pressed retention mechanisms 4482 expand back radially outward and engage with the one or more lockout windows 4434, as illustrated in FIG. 146D. In this example, after the one or more retention mechanisms 4482 engage with the one or more lockout windows 4434, auto-injector 100 is in a lockout state. As best depicted in FIG. 147B, in the lockout state, the one or more retention mechanisms 4482 and the one or more lockout windows 4434 function cooperatively to prevent shroud 4420 from being depressed, thereby preventing a needle 4416 (FIG. 152) of auto-injector 100 from being exposed outside of housing 4402 and improving patient safety. For example, the one or more lockout windows 4434 may include a rectangular opening having a flat, downward-facing edge, and the one or more retention mechanisms 4482 may include a flat, upward-facing edge configured to engage with the downward-facing edge of the one or more lockout windows 4434, such that the one or more retention mechanisms 4482, after engaging with the one or more lockout windows 4434, may no longer be translated upward and/or inward.

As depicted in FIG. 147A, chassis 4430 may include one or more guide rails 4435 configured to guide the movement of the one or more retention mechanisms 4482 of mandrel assembly 4480. For example, an interior surface of chassis 4430 may include a pair of guide rails 4435 that are vertical with respect to a longitudinal axis of auto-injector 100, or otherwise parallel with respect to a longitudinal axis of mandrel assembly 4480, and disposed on either side of a retention mechanism 4482. The pair of guide rails 4435 may be spaced apart at a distance that is slightly greater than a width of the retention mechanism 4482, such that the pair of guide rails 4435 form a slot or track configured to receive and guide the retention mechanism 4482, e.g., as the retention mechanism 4482 moves down and/or out toward the a lockout windows 4434 in a post-activation, pre-lockout state of auto-injector 100.

As depicted in FIGS. 148A-148C, chassis 4430 may include one or more features or mechanisms configured to generate and provide audible feedback to a user of auto-injector 100. For example, chassis 4430 may include one or more audible feedback elements 4431 configured to engage with one or more retention mechanisms 4482 of mandrel assembly 4480 to produce an audible sound, e.g., a click. Audible feedback elements 4431 may include bumps, ramps, grooves, divots, holes, or any other element suitable for engaging with retention mechanisms 4482 to produce an audible sound.

In the example depicted in FIGS. 148A-148C, audible feedback elements 4431 include protrusions extending in a radially inward direction away from an interior surface of chassis 4430 and toward mandrel assembly 4480. In this example, mandrel assembly 4480 includes two retention mechanisms 4482, e.g., two arms, in contact with chassis 4430 on opposite sides of mandrel assembly 4480, and chassis 4430 includes two pairs of audible feedback elements 4431, a first pair of audible feedback elements 4431A disposed on opposite interior walls of chassis 4430 along a first horizontal axis 4417 perpendicular to a longitudinal axis 4419 of mandrel assembly 4480, and a second pair of audible feedback elements 4431B disposed on the opposite interior walls of chassis 4430 along a second horizontal axis 4418 perpendicular to the longitudinal axis 4419 of mandrel assembly 4480 and below the first horizontal axis 4417. In a downward direction extending toward the bottom surface 4421 of shroud 4420 along a longitudinal axis of auto-injector 100, the protrusions of the audible feedback elements 4431 may extend away from the interior surfaces of chassis 4430 to their apexes gradually, such as in the ramped configuration of the first pair of audible feedback elements 4431A, or may extend away from the interior surfaces of chassis 4430 to their apexes virtually instantaneously, such as in the straight configuration of the second pair of audible feedback elements 4431B. In the same direction, beyond their apexes, the protrusions of the audible feedback elements 4431 may retract to the interior surfaces of chassis 4430 instantaneously.

FIG. 148A depicts shroud 4420, chassis 4430, and mandrel assembly 4480 when auto-injector 100 is in a pre-activation state, e.g., before shroud 4420 has been depressed upward and/or inward relative to chassis 4430 to activate auto-injector 100. In this example, when auto-injector 100 is in the pre-activation state, the two retention mechanisms 4482 of mandrel assembly 4480 are engaged with the first pair of audible feedback elements 4431A, which may at least partially hold mandrel assembly 4480 and/or slidable piston 4490 in its respective first position. Thus, when a pressurized medium received by release outlet 4469 exerts a downward and/or outward force on slidable piston 4490, e.g., in a post injection state of auto-injector 100, as described above, the retention mechanisms 4482 of mandrel assembly 4480 immediately begin moving against the first pair of audible feedback elements 4431A. Because of the ramped configuration of retention mechanisms 4482 and/or the ramped configuration of audible feedback elements 4431, as well as the flexibility of retention mechanisms 4482, as slidable piston 4490 and mandrel assembly 4480 move downward and/or outward, the retention mechanisms 4482 are pressed inward by the first pair of audible feedback elements 4431, until they eventually move beyond the apexes of the first pair of audible feedback elements 4431A. Because the audible feedback elements 4431 retract to the interior surfaces of chassis 4430 instantaneously, when the retention mechanisms 4482 move beyond the apexes of the first pair of audible feedback elements 4431A, the retention mechanisms 4482 immediately expand back radially outward to contact the interior surfaces of chassis 4430, as depicted in FIG. 148B, thereby producing a first audible click. As this first audible click may be produced immediately after auto-injector 100 transitions from the activated state to the post-injection state, as described in further detail above and below, the first audible click may serve as an audible indication to a user of auto-injector 100 that an injection from auto-injector 100 has finished.

FIG. 148B depicts shroud 4420, chassis 4430, and mandrel assembly 4480 when auto-injector 100 is in a post-activation, pre-lockout state, e.g., after shroud 4420 has been depressed upward and/or inward relative to chassis 4430. In the example, after the retention mechanisms 4482 move beyond the first pair of audible feedback elements 4431A, through further downward and/or outward translation of slidable piston 4490 and mandrel assembly 4480 in response to a release of a pressurized medium into release outlet 4469, mandrel assembly 4480 may be allowed to engage with shroud 4420 and urge shroud 4420 downward and/or outward. As mandrel assembly 4480 and shroud 4420 move downward and/or outward, the retention mechanisms 4482 of mandrel assembly 4480 eventually engage with the second pair of audible feedback elements 4431B, and the retention mechanisms 4482 are pressed inward by the second pair of audible feedback elements 4431B, until they eventually move beyond the apexes of the second pair of audible feedback elements 4431B. Because the audible feedback elements 4431 retract to the interior surfaces of chassis 4430 instantaneously, when the retention mechanisms 4482 move beyond the apexes of the second pair of audible feedback elements 4431B, the retention mechanisms immediately expand back radially outward to contact the interior surfaces of chassis 4430, as depicted in FIG. 148C, thereby producing a second audible click. As this second audible click may be produced at the same time that shroud 4420 returns to its first, resting position, the second audible click may serve as an audible indication that shroud 4420 has been locked out.

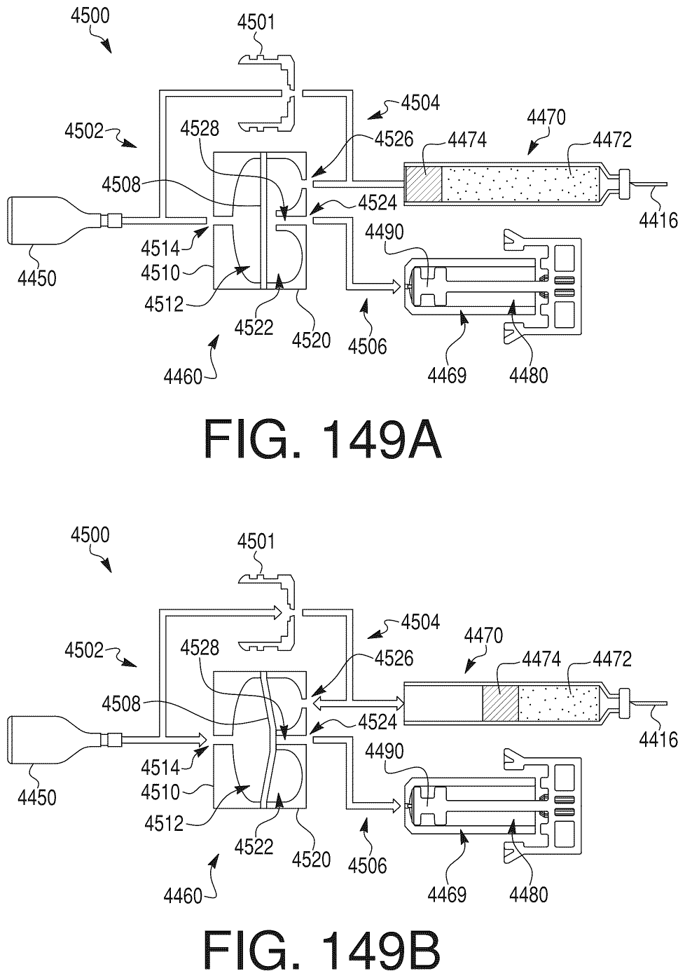

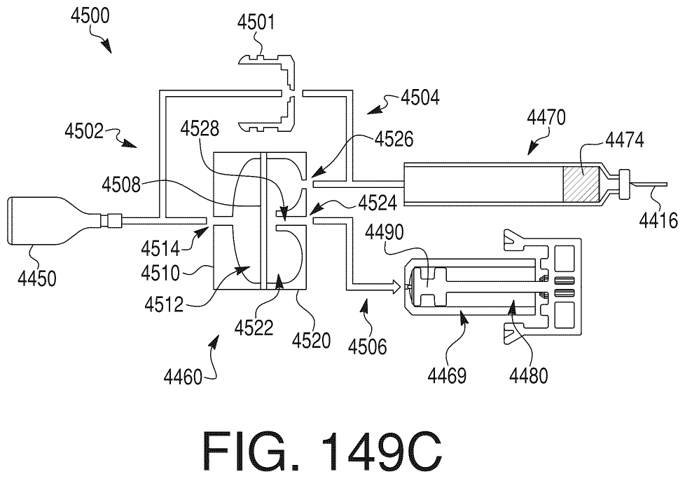

Referring now to FIGS. 149A-149C, an exemplary drive system 4500 of auto-injector 100 is schematically depicted. Drive system 4500 may be configured to provide a driving force to deliver a medicament 4472 from container 4470 to a patient. As depicted in FIGS. 149A-149C, drive system 4500 may include a canister 4450 containing a pressurized fluid and one or more components of auto-injector 100 operatively coupled to canister 4450, e.g., fluid assembly 4460, container 4470, and release outlet 4469. Drive system 4500 may further include a first flow path 4502, a second flow path 4504, and a third flow path 4506. Each of flow paths 4502, 4504, and 4506 may include one or more lines through which a pressurized fluid may be directed. Canister 4450 may be operatively coupled to, e.g., in fluid communication with, one or more other components of auto-injector 100, e.g., valve assembly 4460, container 4470, and release outlet 4469, via first flow path 4502, second flow path 4504, and third flow path 4506. For example, canister 4450 may be in fluid communication with valve assembly 4460 via the first flow path 4502; canister 4450 may be in fluid communication with container 4470 via the first flow path 4502 and the second flow path 4504; and canister 4750 may be in fluid communication with release outlet 4469 via the first flow path 4502, the second flow path 4504, and the third flow path 4506. As depicted in FIGS. 149A-149C, drive system 4500 may further include one or more flow restrictors or orifices 4501 disposed about, within, or between first flow path 4502, second flow path 4504, and third flow path 4506.

As depicted in FIGS. 149A-149C, valve assembly 4460 may include a first or high pressure body portion 4510 including a first or high pressure cavity 4512 and a first or high pressure inlet 4514. High pressure cavity 4512 may be in fluid communication with canister 4450 via the first flow path 4502 and high pressure inlet 4514. Valve assembly 4460 may further include a second or low pressure body portion 4520 including a second or low pressure cavity 4522, a second or low pressure inlet 4524, a conduit 4526, and a valve seat 4528. Low pressure cavity 4522 may be in fluid communication with canister 4450 via the first flow path 4502, the second flow path 4504, and the low pressure inlet 4524. Conduit 4526 may be formed within the valve seat 4528, which may extend into the interior of valve assembly 4460, and in particular into low pressure cavity 4522. High pressure cavity 4512 and low pressure cavity 4522 may be defined and fluidly separated by a diaphragm 4508.

FIG. 149A depicts components of drive system 4500 when auto-injector 100 is in a pre-activated state. As described above and below, in the pre-activated state, shroud 4420 has not yet been depressed to activate auto-injector 100. Thus, in the pre-activated state, pressurized fluid is not yet released from canister 4450 and the pressure within high pressure cavity 4512 is substantially equal to the pressure within low pressure cavity 4522; piston 4474 is at rest and medicament 4472 is not yet expelled from container 4470; and mandrel assembly 4480 is at rest.

FIG. 149B depicts components of drive system 4500 when auto-injector 100 is in a post-activation, pre-lockout state. As described above and below, in the post-activation, pre-lockout state, shroud 4420 has been depressed to cause a pressurized fluid to be released by canister 4450, thereby activating auto-injector 100. As depicted in FIG. 149B, when the pressurized fluid is released by canister 4450, the pressurized fluid begins flowing through the drive system 4500 via the first flow path 4502, the second flow path 4504, and the third flow path 4506. In this example, an orifice 4501 disposed between the first flow path 4502 and the second flow path 4504 restricts the flow of the pressurized fluid between the first flow path 4502 and the second flow path 4504, causing the pressure within the first flow path 4502 to increase more quickly than the pressure within the second flow path 4504, thereby generating a pressure differential between the first flow path 4502 and the second flow path 4504, and, by extension, between the high pressure cavity 4512 and the low pressure cavity 4522. The pressure differential between the high pressure cavity 4512 and the low pressure cavity 4522 in turn causes the diaphragm to move in a first direction toward conduit 4526, thereby sealing valve seat 4528. With the valve seat 4528 sealed, the pressurized fluid cannot flow into the third flow path 4504, and thus the pressure within the second flow path 4502 continues to increase until the pressurized fluid applies enough force to piston 4474 to translate piston 4474 toward needle 4416, thereby expelling medicament 4472 from container 4470. While piston 4474 is translated toward needle 4416, the pressure within the second flow path 4504 may remain substantially constant and/or less than the pressure within the first flow path 4502.

FIG. 149C depicts components of drive system 4500 when auto-injector 100 is in a post-injection, pre-lockout state. Auto-injector 100 may be in a post-injection, pre-lockout state when piston 4474 may be translated no further toward needle 4416 by the force exerted on piston 4474 by the pressurized fluid, e.g., when piston 4474 bottoms out within container 4470. As depicted in FIG. 149C, when piston 4474 may be translated no further toward needle 4416 by the force exerted on piston 4474 by the pressurized fluid, the pressure within the second flow path 4504 increases, thereby decreasing the pressure differential between first flow path 4502 and the second flow path 4504, and, by extension, the pressure differential between the high pressure cavity 4512 and the low pressure cavity 4522, until the pressure differential between the high pressure cavity 4512 and the low pressure cavity 4522 reaches a threshold pressure differential low enough to cause diaphragm 4508 to move in a second direction opposite the first direction, thereby unsealing the valve seat 4528. With the valve seat 4528 unscaled, the pressurized fluid may flow through the third flow path 4506 and into release outlet 4469. With pressurized fluid flowing through the third path 4506, the pressure within the third flow path 4506 increases until the pressurized fluid exerts enough force on slidable piston 4490 to translate slidable piston 4490 and mandrel assembly 4480 toward shroud 4420, such that mandrel assembly 4480 may engage with and urge shroud 4420 back toward a third and final lockout position (which may be similar its first, resting position, as described above). While slidable piston 4490 is translated toward shroud 4420 and/or while mandrel assembly 4480 is urging shroud 4420 toward its first, resting position, the pressure within the third flow path 4506 may remain substantially constant. When slidable piston 4490 may be translated no further, e.g., when shroud 4420 has been returned to its first, resting position by mandrel assembly 4480, pressurized fluid within the drive system 4500 may be redirected toward a venting system configured to vent drive system 4500 by releasing pressurized fluid into an interior cavity of auto-injector 100 and/or into the atmosphere outside of auto-injector 100, as described above.

As illustrated in FIG. 150, valve assembly 4460 may be configured such that canister 4450 is disposed within valve assembly 4460. Disposing canister 4450 within valve assembly 4460 may allow valve assembly 4460 to occupy a relatively larger space within housing 4402 of auto-injector 100 than when canister 4450 is disposed outside of valve assembly 4460. Occupying a relatively larger space within housing 4402 of auto-injector 100 may in turn allow valve assembly 4460 to have a relatively larger volume, which may reduce stress on the valve assembly 4460 and/or increase a duration of an injection provided by auto-injector 100. Valve assembly 4460 may include one or more retention elements 4465 configured to retain canister 4450 at a particular position within valve assembly 4460. When compare to disposing canister 4450 outside of valve assembly 4460, disposing canister 4450 within valve assembly 4460 may reduce a force (e.g., a blowback force) applied to activator 4440, actuator 4425, and/or shroud 4420 by a pressurized medium released from canister 4450. Reducing the blowback force may in turn reduce a force (e.g., a hold force) required for a user to keep shroud 4420 in its second position while medicament 4472 is released from container 4470 via needle 4416.

FIGS. 151A and 151B illustrate cross-sectional views of auto-injector 100. As mentioned above, auto-injector 100 may include a canister 4450 containing a pressurized medium and a valve assembly 4460 configured to receive the pressurized medium when the pressurized medium is released from canister 4450. As mentioned above, canister 4450 may be configured to be punctured by a piercing mechanism 4464 (e.g., a needle, an activator pin, etc.). Piercing mechanism 4464 may include or be included in an activator 4440. Activator 4440 may include a body 4442 and a head 4444. Body 4442 may include a slidable piston 4446 at least partially disposed within activator chamber 4443. Slidable piston 4446 may include one or more recesses 4407 that are sized, shaped, and/or otherwise configured to receive a seal (e.g., an O-ring) therein to inhibit a pressurized medium released by canister 4450 and received by activator chamber 4443 from passing along an exterior surface of slidable piston 4446. A biasing member 4447 (e.g., a spring) may be disposed about body 4442 and configured to engage with activator 4440, such as by exerting a downward and/or outward force on head 4444. Head 4444 may be disposed at an end of activator 4440 opposite piercing mechanism 4464 and may be configured to engage with actuator 4425 (e.g., a movable lever), such that a force exerted on activator 4440 may be translated to actuator 4425 and vice versa. Actuator 4425 may include a first end 4426 configured to engage with head 4444 of activator 4440 and a second end 4427 configured to engage with shroud 4420 (e.g., by abutting against an interior bottom surface 4421 of shroud 4420). Actuator 4425 may be configured to engage with shroud 4420 and activator 4440 simultaneously, such that actuator 4425 may be in operative contact with both shroud 4420 and activator 4440 in any state of auto-injector 100 (e.g., a pre-activation state, an activated state, a post-activation state, a lockout state, or a completion state).

When auto-injector 100 is in a pre-activation state, biasing member 4447 may be in a first compressed configuration, thereby generating and applying a downward and/or outward force on head 4444 of activator 4440. In turn, head 4444 of activator 4440 may translate the downward and/or outward force onto first end 4426 of actuator 4425, which may further translate the downward and/or outward force onto shroud 4420, thereby providing resistance to shroud 4420 being depressed upward and/or inward relative to housing 4402. Providing resistance to shroud 4420 being depressed upward and/or inward when auto-injector 100 is in the pre-activation state may help prevent premature activation of auto-injector 100 and/or may help prevent a needle 4416 of auto-injector 100 from being exposed outside of auto-injector 100 through an opening 4423 of shroud 4420 (as illustrated in FIG. 142), thereby improving operation of auto-injector 100 and/or patient safety. Providing resistance to shroud 4420 being depressed upward and/or inward when auto-injector 100 is in the pre-activation state may be referred to as providing passive needle coverage.

Thus, auto-injector 100 may include a first mechanism (e.g., biasing member 4447 and/or actuator 4425) configured to urge shroud 4420 toward its first position before auto-injector 100 is activated and a second mechanism (e.g., mandrel assembly 4480, slidable piston 4490, valve assembly 4460, canister 4450, retention mechanisms 4482, and/or lockout windows 4434) configured to urge shroud 4420 toward its first position and/or inhibit shroud 4420 from moving toward its second position after auto-injector 100 has been activated (e.g., after a pressurized medium has been released by canister 4450 and/or after medicament 4472 has been released outside of housing 4402). It will be understood that by exerting a downward and/or outward force on shroud 4420, biasing member 4447 may provide resistance to shroud 4420 being depressed upward and/or inward when auto-injector 100 is any state, not only when auto-injector 100 is in the pre-activation state. It will be further understood that although auto-injector 100 is often described herein as employing a pressurized medium to expel medicament 4472 from container 4470 and/or actuate mandrel assembly 4480, auto-injector 100 may include a first mechanism configured to urge shroud 4420 toward its first position before auto-injector 100 is activated and a second mechanism configured to urge shroud 4420 toward its first position and/or inhibit shroud 4420 from moving toward its second position after auto-injector 100 has been activated (e.g., after medicament 4472 has been released outside of housing 4402), while employing any other appropriate driving force for expelling medicament 4472 from container 4470, such as a spring or a motor.

When shroud 4420 is intentionally depressed to activate auto-injector 100, such as when shroud 4420 is pressed against the exterior surface of a subject's skin, shroud 4420 may exert an upward and/or inward force on second end 4427 of actuator 4425, which may translate the upward and/or inward force onto activator 4440 (e.g., through first end 4426), thereby translating slidable piston 4446 upward and/or inward toward canister 4450, such that piercing mechanism 4464 may puncture a seal or valve of canister 4450 and activate auto-injector 100, as described above. For example, actuator 4425 may be rotatable about a pivot joint 4468 (FIGS. 150 and 152), such that an upward and/or inward force exerted on second end 4427 causes actuator 4425 to rotate about pivot joint 4468 in a clockwise direction (relative to the perspective of FIGS. 151A and 151B). In this example, as actuator 4425 rotates in the clockwise direction, the point of first end 4426 at which actuator 4425 contacts head 4444 of activator 4440 moves upward and/or inward relative to housing 4402, thereby pushing activator 4440 upward and/or inward, until piercing mechanism 4464 punctures a seal or valve of canister 4450.

As illustrated in FIG. 151B, activator 4440 may be a two-part activator, including a first part including a slidable piston 4446 and a piercing mechanism 4464 and a second part including head 4444. The first part may be configured to receive the second part, but may not be integrally formed with the second part. In such an embodiment, biasing member 4447 may be configured to exert a downward and/or outward force on head 4444 independent of slidable piston 4446, which may aid slidable piston 4446 in preventing a pressurized medium released by canister 4450 and received by activator chamber 4443 from passing along an exterior surface of slidable piston 4446 and/or aid biasing member 4447 in providing passive needle coverage by preventing friction generated between one or more seals (e.g., O-rings) disposed within one or more recesses 4407 and an interior surface of activator chamber 4443 from against biasing member 4447.

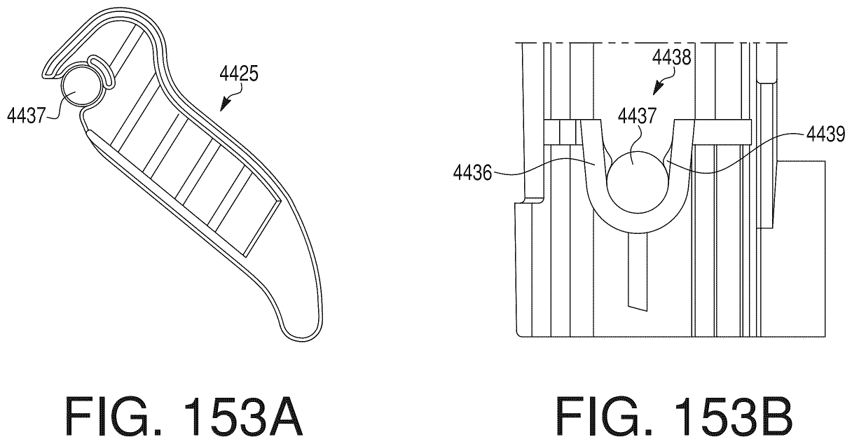

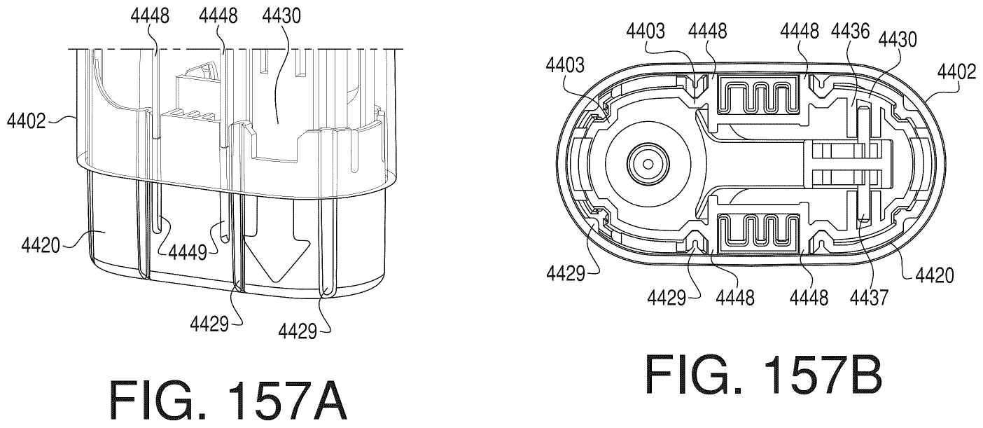

As illustrated in FIG. 152, second end 4427 of actuator 4425 may have a two-pronged form configured to simultaneously contact shroud 4420 (e.g., an interior bottom surface 4421 of shroud 4420) at two points. The two-pronged form of second end 4427 may be configured to straddle an opening 4423 of shroud 4420 as shroud 4420 is depressed toward its second position or as shroud 4420 is urged back toward its first position. Actuator 4425 may be configured to rotate within a pivot joint 4468, e.g., in response to an upward and/or inward force exerted on actuator 4425 by shroud 4420, or in response to a downward and/or outward force exerted on actuator 4425 by activator 4440, as described above. In some instances, as illustrated in FIG. 152, pivot joint 4468 may include one or more pins 4467 projecting outwardly from actuator 4425 and one or more slots or sockets 4466 configured to receive the one or more pins 4467. The one or more pins 4467 projecting outwardly from actuator 4425 may function as an axle about which actuator 4425 may rotate within the one or more slots or sockets 4466. As depicted in FIGS. 150 and 152, the one or more slots or sockets 4466 may be included in valve assembly 4460. Alternatively, some instances, as depicted in FIGS. 153A and 153B, pivot joint 4438 may include an axle 4437 that actuator 4425 can be affixed to, such as by being snapped onto, and one or more slots or sockets 4436. The axle 4437 may be affixed to pivot joint 4438 via one or more slots or sockets 4436, such as by being snapped into the one or more slots or sockets 4436. The one or more slots or sockets 4436 may include one or more stubs 4439 configured to secure axle 4437 in place after axle 4437 is affixed to the one or more slots or sockets 4436. Once actuator 4425 is affixed to axle 4437 and axle 4437 is affixed to pivot joint 4438, axle 4437 and actuator 4425 can rotate together within the one or more slots or sockets 4436, e.g., in response to an upward and/or inward force exerted on actuator 4425 by shroud 4420, or in response to a downward and/or outward force exerted on actuator 4425 by activator 4440, as described above. As depicted in FIGS. 153B and 157B, the one or more slots or sockets 4436 may be included in chassis 4430.

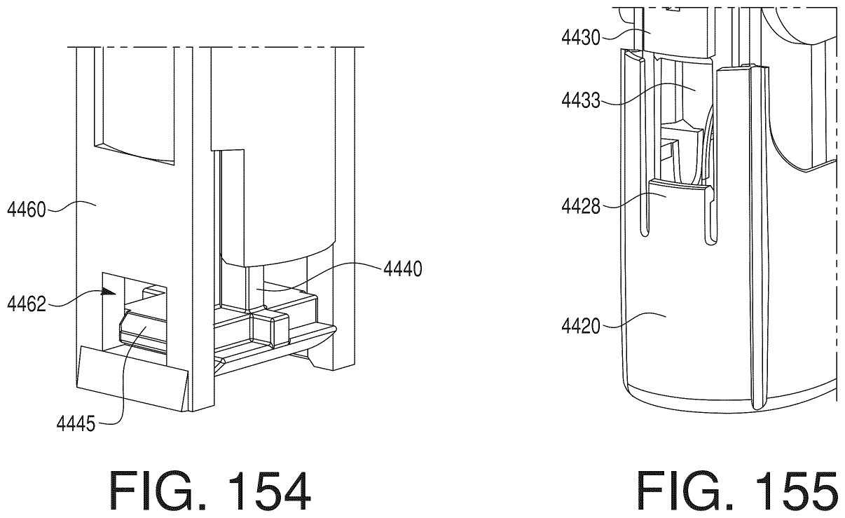

As depicted in FIG. 154, valve assembly 4460 may include one or more retention windows 4462 configured to limit the downward and/or outward translation of activator 4440, e.g., in response to a downward and/or outward force exerted on activator 4440 by biasing member 4447 (FIG. 151B), as described above. For example, activator 4440 may include one or more tabs 4445, and the one or more retention windows 4462 may be openings formed within walls of valve assembly 4460 that are configured to receive the one or more tabs 4445, such that a tab 4445 may be allowed to move up and/or down (e.g., in and/or out) within a retention window 4462, but may be prevented from moving up and/or inward beyond a top border of the retention window 4462 or from moving down and/or outward beyond a bottom border of the retention window 4462. Similarly, as depicted in FIG. 155, chassis 4430 may include one or more retention windows 4433 configured to limit the downward and/or outward translation of shroud 4420, e.g., in response to a downward and/or outward force exerted on shroud 4420 by mandrel assembly 4480 or by actuator 4425, as described above. For example, shroud 4420 may include one or more tabs 4428, and the one or more retention windows 4433 may be openings formed within walls of chassis 4430 that are configured to receive the one or more tabs 4428, such that a tab 4428 (and, by extension, shroud 4420) may be allowed to move up and/or down (e.g., in and/or out) within a retention window 4433, but may be prevented from moving up and/or inward beyond a top border of the retention window 4433 or from moving down and/or outward beyond a bottom border of the retention window 4433.

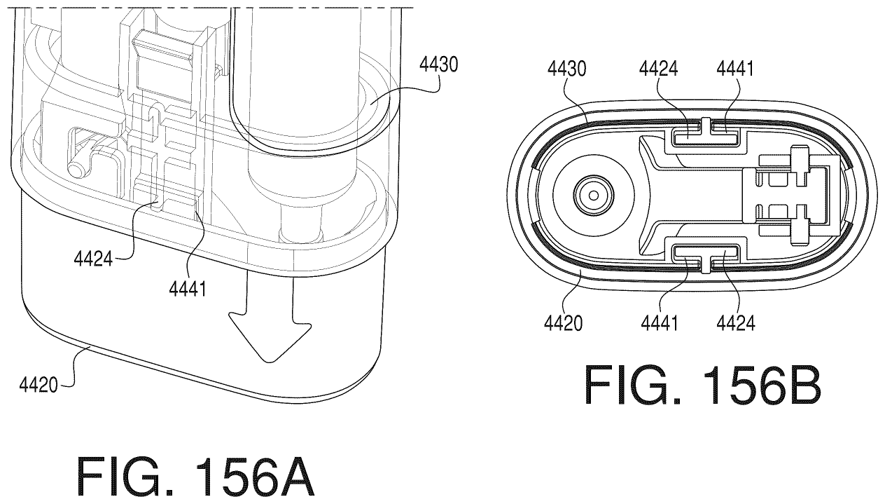

As depicted in FIGS. 156A and 156B, chassis 4430 may include one or more guide rails 4441 configured to guide the movement of shroud 4420. For example, shroud 4420 may include one or more projections 4424, and chassis 4430 may include one or more guide rails 4441 configured to receive the one or more projections 4424 and guide the one or more projections 4424 as shroud 4420 moves up or down (e.g., in or out) with respect to chassis 4430, e.g., as shroud 4420 is depressed toward its second position to activate auto-injector 100, or as shroud 4420 is urged back toward its first position in a post-activation, pre-lockout state of auto-injector 100. The one or more projections 4424 may project inward with respect to housing 4402 and extend vertically along an interior surface of shroud 4420. The one or more guide rails 4441 may be configured such that a negative interior space of the one or more guide rails 4441 is similar to the shape of the one or more projections 4424, e.g., a T-shape, such that the one or more guide rails 4441 form a slot or track configured to receive the one or more projections 4424.

Referring now to FIGS. 157A and 157B, in some instances, if housing 4402 is squeezed or otherwise depressed inward, one or more functions of auto-injector 100 may be compromised. For example, if housing 4402 is depressed inward, shroud 4420 may not be allowed to be depressed upward and/or inward to activate auto-injector 100, or shroud 4420 may not be allowed to be urged downward and/or outward by mandrel assembly 4480 to lockout shroud 4420 and prevent needle 4416 from being re-exposed, as described above. As depicted in FIGS. 157A and 157B, chassis 4430 may include one or more ribs 4448 configured to prevent housing 4402 from being squeezed or otherwise depressed inward. For example, one or more ribs 4448 may be disposed on an outer surface of chassis 4430, as depicted in FIG. 157A, and shroud 4420 may include one or more openings 4449 configured to receive the one or more ribs 4448 and to allow the one or more ribs 4448 to contact an inner surface of housing 4402, as depicted in FIG. 157B. Thus, if housing 4402 is squeezed, housing 4402 may be depressed inward no further than the one or more ribs 4448 and may be prevented from contacting shroud 4420, thereby preventing the squeezing of housing 4402 from compromising one or more functions of auto-injector 100. As depicted in FIG. 157A, ribs 4448 and openings 4449 may have a vertical shape or configuration, such that an upward or downward (e.g., inward or outward) translation of shroud 4420 may not be prevented by ribs 4448.

As depicted in FIGS. 157A and 157B, shroud 4420 may include one or more ribs 4429 configured to guide the movement of shroud 4420. For example, one or more ribs 4429 may protrude inward from shroud 4420 toward chassis 4430, and chassis 4430 may include one or more slots 4403 configured to receive the one or more ribs 4429 and guide the one or more ribs 4429 as shroud 4420 moves up or down (e.g., in or out) with respect to chassis 4430, e.g., as shroud 4420 is depressed toward its second position to activate auto-injector 100, or as shroud 4420 is urged back toward its first position in a post-activation, pre-lockout state of auto-injector 100, as described above. As depicted in FIG. 157A, ribs 4429 and corresponding slots 4403 may have a vertical shape or configuration. As depicted in FIG. 157B, ribs 4429 and corresponding slots 4403 may be disposed at or near corners of shroud 4420 and chassis 4430, respectively (e.g., corners defined by rectangles inscribed within the ovular shapes of shroud 4420 and chassis 4430), and/or alongside surfaces of shroud 4420 and chassis 4430, respectively.

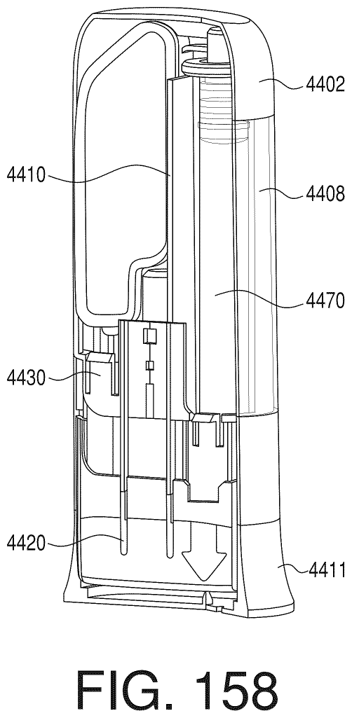

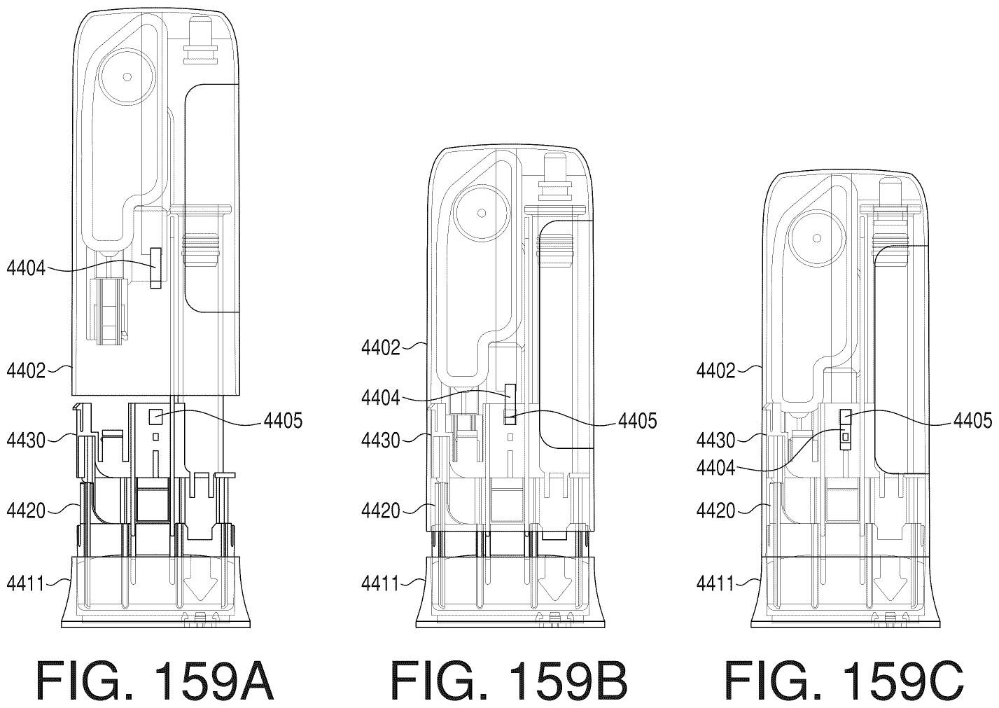

Referring now to FIG. 158, housing 4402 and chassis 4430 may include one or more features configured to allow housing 4402 and chassis 4430 to engage or interact with one another. For example, as depicted in FIG. 158, housing 4402 may include a transparent or semi-transparent window 4408 configured to allow a user of auto-injector 100 to see through and within housing 4402, such that the user may see container 4470 and an amount or level of medicament 4472 contained within container 4470, and chassis 4430 may include an opaque window shield 4410 configured to prevent a user of auto-injector 100 from seeing components of auto-injector 100 beyond container 4470, e.g., valve assembly 4460 and mandrel assembly 4480. Window shield 4410 may extend vertically from chassis 4430 toward the top of housing 4402. Or for example, as depicted in FIGS. 159A-159C, housing 4402 may include a housing slot 4404 and chassis 4430 may include a corresponding chassis slot 4405. Housing slot 4404 and chassis slot 4405 may be configured to facilitate the assembly of auto-injector 100. For example, as depicted in FIG. 159A, during the assembly of auto-injector 100, housing 4402 and chassis 4430 may be brought toward one another until housing slot 4404 at least partially overlaps with chassis slot 4405. As depicted in FIG. 159B, after housing slot 4404 at least partially overlaps with chassis slot 4405, a tool may be inserted through both housing slot 4404 and chassis slot 4405 and used to further translate chassis 4430 upward, such that housing 4402 is brought fully down over the components of auto-injector 100. In some instances, as depicted in FIG. 159C, when housing 4402 is brought fully down over the components of auto-injector 100, one or more retention elements included in housing 4402 (not shown) are engaged with one or more retention elements included in chassis 4430 (not shown), such that housing 4402 and chassis 4430 are secured to one another. In some instances, securing housing 4402 and chassis 4430 to one another completes the assembly of auto-injector 100.

Features enumerated above have been described within the context of particular embodiments. However, as one of ordinary skill in the art would understand, features and aspects of each embodiment may be combined, added to other embodiments, subtracted from an embodiment, etc. in any manner suitable to assist with controlled preparation and/or delivery of a drug. While a number of embodiments are presented herein, multiple variations on such embodiments, and combinations of elements from one or more embodiments, are possible and are contemplated to be within the scope of the present disclosure. Accordingly, it should be appreciated that any of the features of the particular auto-injectors shown and described herein may be included, combined, added, exchanged, and/or incorporated with any of the other auto-injectors in any suitable manner without departing from a scope of this disclosure. Moreover, those skilled in the art will appreciate that the conception upon which this disclosure is based may readily be used as a basis for designing other devices, methods, and systems for carrying out the several purposes of the present disclosure.

Embodiments of the present disclosure may include the following features:

Item 1. An auto-injector, comprising: a housing; a container disposed within the housing for storing a medicament; an energy source disposed within the housing for storing energy, the energy source is configured to release the energy through the housing to expel the medicament from the container; a shroud movably coupled to the housing, the shroud is configured to move between a first position and a second position relative to the housing to actuate the energy source, thereby releasing the energy towards the container; and a needle disposed within the housing and in fluid communication with the container, the needle is concealed by the shroud when the shroud is in the first position and extended out of the shroud when the shroud is in the second position; wherein the auto-injector is configured to lock the shroud towards the first position upon the energy source expelling the medicament from the container and through the needle, thereby inhibiting reactivation of the auto-injector.

Item 2. The auto-injector of item 1, wherein the energy source is a canister and the energy is a pressurized fluid.

Item 3. The auto-injector of item 1, wherein the shroud is configured to move along a vertical path relative to the housing when moving from the first position towards the second position, the shroud is extended from the housing when in the first position and at least partially retracted into the housing when in the second position.

Item 4. The auto-injector of item 1, wherein the shroud is configured to automatically return towards the first position from the second position upon the energy source expelling the medicament from the container and through the needle.

Item 5. The auto-injector of item 4, wherein the auto-injector is configured to push the housing relatively away from a user by automatically moving the shroud towards the first position, thereby generating a visual confirmation of the medicament having been expelled from the container.

Item 6. The auto-injector of item 5, wherein the shroud is configured to automatically move towards the first position to generate the visual confirmation in response to the energy source releasing the energy through the housing and pushing the shroud outwards from the housing.

Item 7. The auto-injector of item 4, further comprising a biasing mechanism that is movable from an expanded configuration to a compressed configuration upon movement of the shroud from the first position towards the second position, the biasing mechanism is configured to automatically move the shroud from the second position towards the first position upon the energy source expelling the medicament from the container and through the needle.

Item 8. The auto-injector of item 7, wherein after a threshold amount of energy is released from the energy source, the biasing mechanism is automatically moved from the compressed configuration towards the expanded configuration, thereby causing vertical translation of the shroud from the second position towards the first position.

Item 9. The auto-injector of item 1, wherein the shroud is movably coupled to the energy source, and movement of the shroud from the first position towards the second position causes the energy source to move vertically within the housing from a first location to a second location.

Item 10. The auto-injector of item 1, wherein the auto-injector is configured to lock the shroud to the first position upon the shroud initiating movement from the first position towards the second position without fully reaching the second position, thereby inhibiting reactivation of the auto-injector.

Item 11. The auto-injector of item 1, wherein the shroud is depressed at least partially into the housing or the housing is at least partially extended over the shroud when the shroud moves from the first position towards the second position.

Item 12. The auto-injector of item 1, further comprising a valve assembly fluidly coupled to the energy source and the container, the valve assembly includes a diaphragm having a central body with an outer rim extending outwardly from the central body and about a periphery of the central body, and a raised portion located at a radially center position of the central body, the raised portion having a thickness protruding outward from the central body at a distance that is greater than an extension of the outer rim relative to the central body.

Item 13. The auto-injector of item 1, further comprising an indicator visible from outside of the housing, the indicator is configured to visually indicate an operating state of the auto-injector in response to moving relative to the housing when the energy source expels the medicament from the container and through the needle.

Item 14. The auto-injector of item 13, wherein the indicator is configured to generate an audible feedback to indicate the operating state of the auto-injector in response to contacting an interior portion of the housing upon moving relative to the housing.

Item 15. The auto-injector of item 1, further comprising a slidable piston that is fluidly coupled between the energy source and the container, the slidable piston defines a dump valve that is configured to move within the housing from a first state to a second state to depressurize the auto-injector as the energy source expels the medicament from the container.

Item 16. The auto-injector of item 1, wherein the shroud is configured to lockout in response to a movement of the shroud or the housing relative to one another prior to completing delivery of the medicament from the container.

Item 17. The auto-injector of item 16, wherein the shroud is configured such that further movement of the shroud or the housing relative to one another is inhibited, thereby inhibiting the medicament from being expelled out of the container.

Item 18. The auto-injector of item 16, wherein the shroud is locked out in the first position to inhibit use of the auto-injector in delivering the medicament from the container.

Item 19. The auto-injector of item 18, wherein the needle is concealed within the shroud when the shroud is locked out in the first position.

Item 20. An auto-injector, comprising: a housing; a container configured to store a medicament; a fluid source configured to store a pressurized fluid; a needle including a first end configured to extend out of the housing and a second end configured to extend into the container, the first end is disposed inside the housing and the second end is separated from the container prior to the fluid source releasing the pressurized fluid; and an activator configured to move the fluid source into fluid communication with the container and release the pressurized fluid from the fluid source to the container, the activator includes a keyed arrangement for controlling movement of the activator relative to the fluid source; wherein the container is configured to move into fluid communication with the second end of the needle, and the first end of the needle is configured to extend out of the housing, in response to the container receiving the pressurized fluid from the fluid source, thereby delivering the medicament through the needle and out of the housing.

Item 21. The auto-injector of item 20, further comprising: a shuttle actuator movably coupled to the container, the shuttle actuator is configured to move horizontally with the container and within the housing in response to the fluid source releasing the pressurized fluid.

Item 22. The auto-injector of item 21, further comprising: a driver coupled to the needle between the first end and the second end, the driver is configured to move vertically within the housing; and a gear movably coupled to the shuttle actuator and the driver, the gear is configured to rotate within the housing in response to the shuttle actuator moving horizontally with the container, thereby moving the driver with the second end of the needle relative to the housing; wherein horizontal movement of the shuttle actuator causes the gear to rotate and the driver to move vertically relative to the housing.

Item 23. The auto-injector of item 20, wherein the activator includes a resilient member having a compressed configuration and an expanded configuration, the resilient member is maintained in the compressed configuration prior to movement of the activator relative to the housing, and transitioned towards the expanded configuration upon movement of the activator.

Item 24. The auto-injector of item 23, wherein the resilient member is configured to move the activator towards the fluid source in response to transitioning from the compressed configuration towards the expanded configuration, thereby causing the activator to move the fluid source into fluid communication with the container to release the pressurized fluid to the container.

Item 25. The auto-injector of item 20, wherein the keyed arrangement of the activator includes a plurality of splines positioned about an end of the activator.

Item 26. The auto-injector of item 25, further comprising a carrier disposed within the housing, the carrier is configured to at least partially interface with the container, the fluid source, the activator, or the needle; wherein the carrier includes a plurality of detents and a plurality of slots that are each configured to interface with the plurality of splines in response to the activator rotating relative to the carrier.

Item 27. An auto-injector, comprising: a housing; a container disposed within the housing for storing a medicament; an energy source disposed within the housing for storing energy, the energy source is configured to release the energy through the housing to expel the medicament from the container; a shroud movably coupled to the housing, the shroud is configured to move between a first position and a second position relative to the housing to actuate the energy source, thereby releasing the energy towards the container; and an actuator positioned between the energy source and the shroud, the actuator is configured to actuate the energy source to release the energy towards the container in response to the shroud pushing the actuator towards the energy source when moving from the first position towards the second position,

Item 28. The auto-injector of item 27, further comprising a needle disposed within the housing and in fluid communication with the container, the needle is concealed by the shroud when the shroud is in the first position and extended out of the shroud when the shroud is in the second position.

Item 29. The auto-injector of item 27, further comprising a mandrel assembly coupled to the shroud, the mandrel assembly including a biasing mechanism that is configured to urge the shroud outwards from the housing from the second position towards the first position.

Item 30. The auto-injector of item 27, further comprising a piston slidably disposed within the container, the piston is configured to expel the medicament from the container in response to the energy from the energy source pushing the piston through the container.

Item 31. The auto-injector of item 30, further comprising an indicator coupled to the piston, the indicator is configured to generate visual feedback of a dose state of the auto-injector.

Item 32. The auto-injector of item 27, wherein the shroud includes a bottom surface, a rounded end, and a narrowed end extending outwardly from the rounded end; wherein the rounded end includes an opening, and a needle disposed within the housing extends out of the shroud via the opening in response to the shroud moving from the first position towards the second position.

Item 33. An auto-injector, comprising: a housing; a container for storing a medicament; a valve assembly fluidly coupled to the container, the valve assembly including a diaphragm that is configured to move within the valve assembly in response to a change in pressurization within the valve assembly; a fluid source for storing a pressurized fluid, the fluid source is configured to release the pressurized fluid towards the valve assembly, thereby causing the change in pressurization, in response to the fluid source moving towards and into fluid communication with the valve assembly; and a shroud movably coupled to the housing, the shroud is configured to retract into the housing to push the fluid source towards and into fluid communication with the valve assembly, thereby causing release of the pressurized fluid into the valve assembly; wherein the valve assembly is configured to deliver the pressurized fluid towards the container to expel the medicament out of the auto-injector.

Item 34. An auto-injector, comprising: an actuator; a container configured to store a medicament; a fluid source configured to store a pressurized fluid; a valve assembly fluidly coupled to the container, the valve assembly including a diaphragm that is configured to move in a first direction in response to the valve assembly receiving a fluid for delivery towards the container; and an activator configured to urge the fluid source into fluid communication with the valve assembly, thereby causing the fluid source to release the pressurized fluid into the valve assembly, in response to the actuator engaging the activator upon actuation; wherein the container is configured to receive the pressurized fluid from the valve assembly and the pressurized fluid is operable to expel the medicament out of the container for delivery, the diaphragm is configured to move in a second direction that is opposite of the first direction upon the auto-injector completing delivery of the medicament.

Item 35. The auto-injector of item 34, wherein the activator includes a keyed arrangement defined by a plurality of splines that are misaligned with a plurality of slots of a carrier coupled to the activator prior to actuation of the actuator.

Item 36. The auto-injector of item 35, wherein the actuator is configured to move the plurality of splines into alignment with the plurality of slots upon engaging the activator, and the plurality of slots are configured to receive the plurality of splines upon alignment.

Item 37. The auto-injector of item 36, wherein the activator is configured to rotate relative to the fluid source when the actuator engages the activator, thereby causing the plurality of splines to rotate into alignment with the plurality of slots.

Item 38. The auto-injector of item 37, wherein the activator is configured to translate towards the fluid source upon rotation and when the plurality of splines are received through the plurality of slots to urge the fluid source into fluid communication with the valve assembly.

Item 39. The auto-injector of item 35, wherein the plurality of splines are aligned with a plurality of detents of the carrier prior to rotation of the activator, the plurality of detents are configured to maintain the activator in a locked state relative to the carrier by engaging the plurality of splines.

Item 40. The auto-injector of item 39, wherein the plurality of detents are configured to disengage the plurality of splines upon rotation of the activator, and the plurality of slots are configured to move the activator to an unlocked state relative to the carrier by receiving the plurality of splines.

Item 41. An auto-injector, comprising: a housing; a container disposed within the housing for storing a medicament; a canister disposed within the housing for storing a pressurized medium, the canister configured to release the pressurized medium through the housing to expel the medicament from the container; a shroud movably coupled to the housing, the shroud configured to move between a first position and a second position relative to the housing to cause the pressurized medium to be released by the canister; and a mandrel assembly disposed within the housing, the mandrel assembly configured to urge the shroud outward relative to the housing toward the first position after the pressurized medium is released by the canister.

Item 42. The auto-injector of item 41, wherein the mandrel assembly further includes a slidable piston configured to be moved by the pressurized medium.

Item 43. The auto-injector of item 42, further comprising a valve assembly fluidly coupled to the container and the canister, wherein the slidable piston is at least partially disposed within a release outlet of the valve assembly, and wherein the valve assembly is configured to direct the pressurized medium from the canister to the release outlet to cause the mandrel assembly to urge the shroud outward.

Item 44. The auto-injector of item 43, further comprising a drive system including the canister, the valve assembly, a first flow path, a second flow path, and a third flow path, wherein the drive system is configured to direct the pressurized medium from the canister to the container via the first flow path and the second flow path, and wherein the drive system is configured to direct the pressurized medium from the canister to the release outlet via the first flow path, the second flow path, and the third flow path.

Item 45. The auto-injector of item 44, further comprising an orifice disposed between the first flow path and the second flow path and configured to restrict flow of the pressurized fluid between the first flow path and the second flow path.

Item 46. The auto-injector of item 45, wherein the valve assembly further includes a high pressure cavity and a low pressure cavity fluidly separated by a diaphragm, wherein the high pressure cavity is fluidly coupled to the canister via the first flow path and the low pressure cavity is fluidly coupled to the canister via the second flow path, and wherein the restricted flow of the pressurized fluid between the first flow path and the second flow path generates a pressure differential between the high pressure cavity and the low pressure cavity that causes the diaphragm to seal a valve seat, thereby preventing flow of the pressurized fluid into the third flow path.

Item 47. The auto-injector of item 46, wherein, after the medicament has been expelled from the container, the pressure differential between the high pressure cavity and the low pressure cavity decreases, thereby causing the diaphragm to unseal the valve seat and allow flow of the pressurized fluid into the third flow path.

Item 48. The auto-injector of item 43, wherein the canister is at least partially disposed within the valve assembly.

Item 49. The auto-injector of item 48, wherein the valve assembly further includes one or more retention elements configured to retain the canister within the valve assembly.

Item 50. The auto-injector of item 41, further comprising a chassis disposed within the housing, and wherein the chassis includes one or more lockout windows configured to engage with one of more retention mechanisms of the mandrel assembly after the mandrel assembly urges the shroud toward the first position, thereby inhibiting the shroud from moving toward the second position.

Item 51. The auto-injector of item 50, wherein the chassis further includes one or more guide rails configured to receive and guide the one or more retention mechanisms of the mandrel assembly as the mandrel assembly urges the shroud toward the first position.

Item 52. The auto-injector of item 50, wherein the shroud includes one or more ribs and wherein the chassis further includes one or more slots configured to receive and guide the one or more ribs of the shroud as the shroud moves toward the second position or toward the first position.

Item 53. The auto-injector of item 50, wherein the chassis further includes one or more audible feedback elements configured to engage with the one or more retention mechanisms of the mandrel assembly to produce an audible sound.

Item 54. The auto-injector of item 53, wherein the one or more audible feedback elements are further configured to produce a first audible sound to indicate that an injection from auto-injector has finished.

Item 55. The auto-injector of item 54, wherein the one or more audible feedback elements are further configured to produce a second audible sound to indicate that shroud 4420 has been locked out.

Item 56. The auto-injector of item 50, wherein the shroud includes one or more tabs and wherein the chassis includes one or more retention windows configured to receive the one or more tabs and to prevent the one or more tabs from moving down beyond a bottom border of the one or more retention windows.

Item 57. The auto-injector of item 50, wherein the chassis further includes one or more ribs configured to prevent the housing from being depressed inward.

Item 58. The auto-injector of item 57, wherein the shroud includes one or more openings configured to receive the one or more ribs and to allow the one or more ribs to contact an inner surface of the housing.

Item 59. The auto-injector of item 50, wherein the housing includes a window configured to allow a user of the auto-injector to see container and wherein the chassis further includes a window shield configured to prevent the user of the auto-injector from seeing components of the auto-injector beyond the container.

Item 60. The auto-injector of item 50, wherein the housing includes a housing slot, wherein the chassis further includes a chassis slot, and wherein the housing slot and the chassis slot are configured to overlap and facilitate assembly of the auto-injector.

Item 61. The auto-injector of claim 41, further comprising an actuator positioned between the canister and the shroud, and wherein the shroud is configured to cause to the pressurized medium to be released by the canister by rotating the actuator when moving toward the second position.

Item 62. The auto-injector of claim 61, further comprising an activator including a piercing mechanism and configured to engage with the actuator, and wherein rotation of the actuator in response to the shroud moving toward the second position causes the activator to move toward the canister, such that the piercing mechanism may puncture the canister, thereby causing the pressurized medium to be released by the canister.

Item 63. The auto-injector of claim 62, further comprising a biasing member disposed about the activator and configured to exert a downward force on the activator, wherein the activator translates the downward force onto the actuator, and wherein the actuator translates the downward force onto the shroud.

Item 64. The auto-injector of claim 63, wherein the biasing member is configured to exert the downward force on a head of the activator, wherein the activator includes a first part including the piercing mechanism and a second part including the head, and wherein the first part is configured to receive the second part but is not integrally formed with the second part, such that the rotation of the actuator in response to the shroud moving toward the second position causes both the first part and the second part of the activator to move toward the canister and such that the biasing member may exert the downward force on the second part of the activator independent of the first part of the activator.

Item 65. The auto-injector of claim 61, wherein the actuator includes a first end configured to engage with the activator and a second end configured to engage with the shroud, and wherein the second end includes a two-pronged form configured to simultaneously contact the shroud at two points.

Item 66. The auto-injector of claim 65, wherein the two-pronged form of the second end is configured to straddle an opening of the shroud as the shroud moves toward the second position or toward the first position.

Item 67. An auto-injector, comprising: a housing; a container disposed within the housing, the container configured to store a medicament; a canister disposed within the housing, the canister configured to release a pressurized medium to expel the medicament from the container; a shroud coupled to the housing, the shroud configured to move between a first position and a second position relative to the housing to cause the pressurized medium to be released by the canister; and a mandrel assembly disposed within the housing, the mandrel assembly configured to urge the shroud outward relative to the housing toward to the first position after the pressurized medium is released by the canister.

Item 68. The auto-injector of item 67, wherein the mandrel assembly includes a slidable piston configured to be moved by the pressurized medium released by the canister.

Item 69. The auto-injector of item 67, further comprising a valve assembly fluidly coupled to the container and the canister, wherein the mandrel assembly includes a slidable piston disposed within a release outlet of the valve assembly and configured to be moved by the pressurized medium released by the canister, and wherein the valve assembly is configured to direct the pressurized medium from the canister to the release outlet to cause the mandrel assembly to urge the shroud outward.

Item 70. The auto-injector of item 67, further comprising a valve assembly fluidly coupled to the container and the canister, wherein the canister is at least partially disposed within the valve assembly, wherein the mandrel assembly includes a slidable piston disposed within a release outlet of the valve assembly and configured to be moved by the pressurized medium released by the canister, and wherein the valve assembly is configured to direct the pressurized medium from the canister to the release outlet to cause the mandrel assembly to urge the shroud outward.

Item 71. The auto-injector of item 67, further comprising a drive system including the canister, a valve assembly fluidly coupled to the container and the canister, a first flow path, a second flow path, and a third flow path, wherein the mandrel assembly includes a slidable piston disposed within a release outlet of the valve assembly and configured to be moved by the pressurized medium released by the canister, wherein the drive system is configured to direct, through the valve assembly, the pressurized medium from the canister to the container via the first flow path and the second flow path to cause the medicament to be expelled from the container, and wherein the drive system is configured to direct, through the valve assembly, the pressurized medium from the canister to the release outlet to cause the mandrel assembly to urge the shroud outward.

Item 72. The auto-injector of item 67, further comprising a drive system including the canister, a valve assembly fluidly coupled to the container and the canister, a first flow path, a second flow path, a third flow path, and a flow restrictor configured to restrict flow of the pressurized fluid between the first flow path and the second flow path, wherein the mandrel assembly includes a slidable piston disposed within a release outlet of the valve assembly and configured to be moved by the pressurized medium released by the canister, wherein the drive system is configured to direct, through the valve assembly, the pressurized medium from the canister to the container via the first flow path and the second flow path to cause the medicament to be expelled from the container, and wherein the drive system is configured to direct, through the valve assembly, the pressurized medium from the canister to the release outlet to cause the mandrel assembly to urge the shroud outward.

Item 73. The auto-injector of item 67, further comprising a chassis disposed within the housing, wherein the chassis includes a lockout window configured to engage with a retention mechanism of the mandrel assembly after the mandrel assembly urges the shroud toward the first position, inhibiting the shroud from moving toward the second position.

Item 74. The auto-injector of item 67, further comprising a chassis disposed within the housing, wherein the chassis includes a lockout window configured to engage with a retention mechanism of the mandrel assembly after the mandrel assembly urges the shroud toward the first position, inhibiting the shroud from moving toward the second position, and wherein the chassis further includes an audible feedback element configured to engage with the retention mechanism of the mandrel assembly to produce an audible sound.

Item 75. The auto-injector of item 67, further comprising a chassis disposed within the housing, wherein the chassis includes a lockout windows configured to engage with a retention mechanism of the mandrel assembly after the mandrel assembly urges the shroud toward the first position, inhibiting the shroud from moving toward the second position, and wherein the chassis further includes an audible feedback element configured to engage with the retention mechanism of the mandrel assembly to produce a first audible sound to indicate that an injection from the auto-injector has finished and a second audible sound to indicate that the shroud has been locked out.

Item 76. The auto-injector of item 67, further comprising a chassis disposed within the housing, wherein the chassis includes a plurality of ribs, and wherein the shroud includes a plurality of openings configured to receive the plurality of ribs and allow the plurality of ribs to contact an inner surface of the housing and prevent the housing from being depressed inward.

Item 77. An auto-injector, comprising: a housing; a container disposed within the housing, the container configured to store a medicament; a canister disposed within the housing, the canister configured to release a pressurized medium to expel the medicament from the container; a shroud coupled to the housing, the shroud configured to move between a first position and a second position relative to the housing to cause the pressurized medium to be released by the canister; an actuator positioned between the canister and the shroud, the actuator configured to cause the pressurized medium to be released by the canister when the shroud moves from the first position to the second position, and a mandrel assembly disposed within the housing, the mandrel assembly configured to urge the shroud outward relative to the housing toward the first position after the pressurized medium is released by the canister.

Item 78. The auto-injector of item 77, further comprising an activator including a piercing mechanism and configured to engage with the actuator, wherein the actuator is configured to rotate in response to the shroud moving toward the second position, and wherein rotation of the actuator causes the activator to move toward the canister and the piercing mechanism to puncture the canister, thereby causing the canister to release the pressurized medium.

Item 79. The auto-injector of item 77, wherein the shroud moving toward the second position causes a needle fluidly coupled to the container to be exposed outside of the housing through an opening of the shroud.

Item 80. The auto-injector of item 77, wherein the shroud moving toward the second position causes a needle fluidly coupled to the container to be exposed outside of the housing through an opening of the shroud; and further comprising a biasing member configured to exert an outward force on the actuator, wherein the actuator translates the outward force onto the shroud, providing resistance to the shroud moving toward the second position.

Item 81. The auto-injector of item 77, wherein the actuator includes a two-pronged end configured to simultaneously contact the shroud at two points.

Item 82. An auto-injector, comprising: a housing; a container disposed within the housing, the container configured to store a medicament; a canister disposed within the housing, the canister configured to release a pressurized medium to expel the medicament from the container; a shroud coupled to the housing, the shroud configured to move between a first position and a second position relative to the housing to cause the pressurized medium to be released by the canister; a mandrel assembly disposed within the housing, the mandrel assembly configured to urge the shroud outward relative to the housing toward the first position after the pressurized medium is released by the canister; and a valve assembly fluidly coupled to the canister, the container, and the mandrel assembly, the valve assembly configured to direct the pressurized medium from the canister to the container to cause the medicament to be expelled from the container and to direct the pressurized medium from the canister to the mandrel assembly to cause the mandrel assembly to urge the shroud outward.

Item 83. The auto-injector of item 82, further comprising a chassis disposed within the housing, wherein the chassis includes a lockout window configured to engage with a retention mechanism of the mandrel assembly after the mandrel assembly urges the shroud toward the first position, inhibiting the shroud from moving toward the second position.

Item 84. The auto-injector of item 82, further comprising an actuator positioned between the canister and the shroud and configured to cause the pressurized medium to be released by the canister when the shroud moves from the first position to the second position.

Item 85. The auto-injector of item 82, further comprising: an actuator positioned between the canister and the shroud and configured to cause the pressurized medium to be released by the canister when the shroud moves from the first position to the second position; and a biasing member configured to exert an outward force on the actuator, wherein the actuator translates the outward force onto the shroud, providing resistance to the shroud moving toward the second position.

Item 86. An auto-injector, comprising: a housing; a container disposed within the housing, the container configured to store a medicament; a needle coupled to the container, the needle configured to release the medicament outside of the housing when the auto-injector is activated; a shroud coupled to the housing, the shroud configured to move between a first position wherein the shroud covers the needle and a second position wherein the shroud exposes the needle outside of the housing; a first mechanism configured to urge the shroud toward the first position before the auto-injector is activated; and a second mechanism configured to inhibit the shroud from moving toward the second position after the medicament has been released outside of the housing.

Item 87. The auto-injector of item 86, wherein the first mechanism includes a biasing member configured to exert a downward pressure on the shroud.

Item 88. The auto-injector of item 87: further comprising an actuator disposed within the housing and configured to activate the auto-injector when the shroud moves from the first position to the second position, wherein the biasing member is further configured to exert a downward force on the actuator, and wherein the actuator is further configured to translate the downward force onto the shroud, providing resistance to the shroud moving toward the second position.

Item 89. The auto-injector of item 86, wherein the second mechanism includes a mandrel assembly disposed within the housing, the mandrel assembly configured to be separate from the shroud before the auto-injector is activated and to engage with the shroud after the auto-injector is activated.

Item 90. The auto-injector of item 89, wherein the second mechanism further includes a lockout window configured to engage with a retention mechanism of the mandrel assembly after the mandrel assembly engages with the shroud.

Item 91. The auto-injector of item 90, wherein the lockout window is included in a chassis disposed within the housing.

Item 92. The auto-injector of item 89: further comprising a canister disposed within the housing, the canister configured to release a pressurized medium to expel the medicament from the container via the needle when the auto-injector is activated, wherein the mandrel assembly includes a slidable piston configured to be moved by the pressurized medium.

Item 93. The auto-injector of item 92, wherein the slidable piston is configured to be moved by the pressurized medium after the medicament is expelled from the container via the needle.

Item 94. The auto-injector of item 86: further comprising a canister disposed within the housing, the canister configured to release a pressurized medium to expel the medicament from the container via the needle when the auto-injector is activated, wherein the second mechanism is configured to be actuated by the pressurized medium released from the canister.

Item 95. The auto-injector of item 86, wherein the second mechanism is configured to be actuated by the pressurized medium released from the canister after the medicament is expelled from the container via the needle.

We claim:

1. An auto-injector, comprising:
a housing;
a container disposed within the housing, the container configured to store a medicament;
a canister disposed within the housing, the canister configured to release a pressurized medium to expel the medicament from the container;
a shroud coupled to the housing, the shroud configured to move between a first position and a second position relative to the housing to cause the pressurized medium to be released by the canister; and
a mandrel assembly disposed within the housing, the mandrel assembly configured to urge the shroud outward relative to the housing toward the first position after the pressurized medium is released by the canister.

2. The auto-injector of claim 1, wherein the mandrel assembly includes a slidable piston configured to be moved by the pressurized medium released by the canister.

3. The auto-injector of claim 1:
further comprising a valve assembly fluidly coupled to the container and the canister,
wherein the mandrel assembly includes a slidable piston disposed within a release outlet of the valve assembly and configured to be moved by the pressurized medium released by the canister, and
wherein the valve assembly is configured to direct the pressurized medium from the canister to the release outlet to cause the mandrel assembly to urge the shroud outward.

4. The auto-injector of claim 1:
further comprising a valve assembly fluidly coupled to the container and the canister,
wherein the canister is at least partially disposed within the valve assembly,
wherein the mandrel assembly includes a slidable piston disposed within a release outlet of the valve assembly and configured to be moved by the pressurized medium released by the canister, and
wherein the valve assembly is configured to direct the pressurized medium from the canister to the release outlet to cause the mandrel assembly to urge the shroud outward.

5. The auto-injector of claim 1:
further comprising a drive system including the canister, a valve assembly fluidly coupled to the container and the canister, a first flow path, a second flow path, and a third flow path,
wherein the mandrel assembly includes a slidable piston disposed within a release outlet of the valve assembly and configured to be moved by the pressurized medium released by the canister,
wherein the drive system is configured to direct, through the valve assembly, the pressurized medium from the canister to the container via the first flow path and the second flow path to cause the medicament to be expelled from the container, and
wherein the drive system is configured to direct, through the valve assembly, the pressurized medium from the canister to the release outlet to cause the mandrel assembly to urge the shroud outward.

6. The auto-injector of claim 1:
further comprising a drive system including the canister, a valve assembly fluidly coupled to the container and the canister, a first flow path, a second flow path, a third flow path, and a flow restrictor configured to restrict flow of the pressurized medium between the first flow path and the second flow path,
wherein the mandrel assembly includes a slidable piston disposed within a release outlet of the valve assembly and configured to be moved by the pressurized medium released by the canister,
wherein the drive system is configured to direct, through the valve assembly, the pressurized medium from the canister to the container via the first flow path and the second flow path to cause the medicament to be expelled from the container, and
wherein the drive system is configured to direct, through the valve assembly, the pressurized medium from the canister to the release outlet to cause the mandrel assembly to urge the shroud outward.

7. The auto-injector of claim 1:
further comprising a chassis disposed within the housing,
wherein the chassis includes a lockout window configured to engage with a retention mechanism of the mandrel assembly after the mandrel assembly urges the shroud toward the first position, inhibiting the shroud from moving toward the second position.

8. The auto-injector of claim 1:
further comprising a chassis disposed within the housing,
wherein the chassis includes a lockout window configured to engage with a retention mechanism of the mandrel assembly after the mandrel assembly urges the shroud toward the first position, inhibiting the shroud from moving toward the second position, and
wherein the chassis further includes an audible feedback element configured to engage with the retention mechanism of the mandrel assembly to produce an audible sound.

9. The auto-injector of claim 1:
further comprising a chassis disposed within the housing,
wherein the chassis includes a lockout windows configured to engage with a retention mechanism of the mandrel assembly after the mandrel assembly urges the shroud toward the first position, inhibiting the shroud from moving toward the second position, and
wherein the chassis further includes an audible feedback element configured to engage with the retention mechanism of the mandrel assembly to produce a first audible sound to indicate that an injection from the auto-injector has finished and a second audible sound to indicate that the shroud has been locked out.

10. The auto-injector of claim 1:
further comprising a chassis disposed within the housing,
wherein the chassis includes a plurality of ribs, and
wherein the shroud includes a plurality of openings configured to receive the plurality of ribs and allow the plurality of ribs to contact an inner surface of the housing and prevent the housing from being depressed inward.

11. An auto-injector, comprising:
a housing;
a container disposed within the housing, the container configured to store a medicament;
a canister disposed within the housing, the canister configured to release a pressurized medium to expel the medicament from the container;
a shroud coupled to the housing, the shroud configured to move between a first position and a second position relative to the housing to cause the pressurized medium to be released by the canister;
an actuator positioned between the canister and the shroud, the actuator configured to cause the pressurized medium to be released by the canister when the shroud moves from the first position to the second position, and
a mandrel assembly disposed within the housing, the mandrel assembly configured to urge the shroud outward relative to the housing toward the first position after the pressurized medium is released by the canister.

12. The auto-injector of claim 11:
further comprising an activator including a piercing mechanism and configured to engage with the actuator,
wherein the actuator is configured to move in response to the shroud moving toward the second position, and
wherein movement of the actuator causes the activator to move toward the canister and the piercing mechanism to puncture the canister, thereby causing the canister to release the pressurized medium.

13. The auto-injector of claim 11, wherein the shroud moving toward the second position causes a needle fluidly coupled to the container to be exposed outside of the housing through an opening of the shroud.

14. The auto-injector of claim 11:
wherein the shroud moving toward the second position causes a needle fluidly coupled to the container to be exposed outside of the housing through an opening of the shroud; and
further comprising a biasing member configured to exert an outward force on the actuator,
wherein the actuator translates the outward force onto the shroud, providing resistance to the shroud moving toward the second position.

15. The auto-injector of claim 11, wherein the actuator includes a two-pronged end configured to simultaneously contact the shroud at two points.

16. An auto-injector, comprising:
a housing;
a container disposed within the housing, the container configured to store a medicament;
a canister disposed within the housing, the canister configured to release a pressurized medium to expel the medicament from the container;
a shroud coupled to the housing, the shroud configured to move between a first position and a second position relative to the housing to cause the pressurized medium to be released by the canister;
a mandrel assembly disposed within the housing, the mandrel assembly configured to urge the shroud outward relative to the housing toward the first position after the pressurized medium is released by the canister; and
a valve assembly fluidly coupled to the canister, the container, and the mandrel assembly, the valve assembly configured to direct the pressurized medium from the canister to the container to cause the medicament to be expelled from the container and to direct the pressurized medium from the canister to the mandrel assembly to cause the mandrel assembly to urge the shroud outward.

17. The auto-injector of claim 16, wherein the mandrel assembly includes a slidable piston disposed within a release outlet of the valve assembly and configured to be moved by the pressurized medium released by the canister.

18. The auto-injector of claim 16:
further comprising a chassis disposed within the housing,
wherein the chassis includes a lockout window configured to engage with a retention mechanism of the mandrel assembly after the mandrel assembly urges the shroud toward the first position, inhibiting the shroud from moving toward the second position.

19. The auto-injector of claim 16, further comprising an actuator positioned between the canister and the shroud and configured to cause the pressurized medium to be released by the canister when the shroud moves from the first position to the second position.

20. The auto-injector of claim 16, further comprising:
an actuator positioned between the canister and the shroud and configured to cause the pressurized medium to be released by the canister when the shroud moves from the first position to the second position; and
a biasing member configured to exert an outward force on the actuator,
wherein the actuator translates the outward force onto the shroud, providing resistance to the shroud moving toward the second position.
